(19) United States
(12) Patent Application Publication
(10) Pub. No.: US 2007/0196270 A1
(43) Pub. Date: Aug. 23, 2007

(54) Compounds As Ccri Antagonists

(76) Inventors: Richard Heng, Hegenheim (FR); Laszlo Revesz, Therwil (CH); Achim Schlapbach, Lorrach (DE); Rudolf Walchli, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/599,819

(22) PCT Filed: Apr. 25, 2005
(86) PCT No.: PCT/EP05/04422

§ 371 Date: Oct. 11, 2006

(30) Foreign Application Priority Data
Apr. 26, 2004 (GB) 0409236.7

(51) Int. Cl. A61K 51/00; C07D 471/02; A61K 31/495; A61K 31/4745; A61K 31/407

(52) U.S. Cl. 424/1.11; 514/255.01; 514/300; 514/412; 544/392; 546/113; 548/453

(57) ABSTRACT

A compound of formula I, or a pharmaceutically acceptable salt or ester thereof, 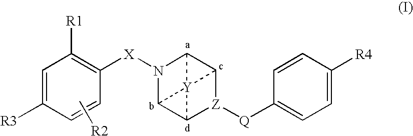 wherein the symbols have meaning as defined, which are antagonists of CCR-1 and which find use pharmaceutically for treatment of diseases and conditions in which CCR-1 is implicated, e.g. inflammatory diseases.

This application relates to bicyclic piperazines and piperidines that are antagonists of Chemokine Receptor 1 (CCR-1) and to their use in the treatment of diseases or disorders that involve migration and activation of monocytes and T-cells, including inflammatory diseases.

Accordingly the invention provides a compound of formula I, or a pharmaceutically acceptable salt or ester thereof, 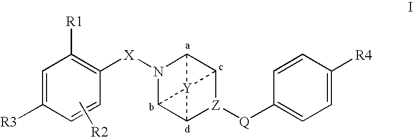
wherein
R1, R2 and R3 are independently selected from the group consisting of hydrogen, cyano, halo, nitro or optionally substituted oxy, lower alkyl, lower alkyenyl, lower alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
R4 is selected from the group consisting of hydrogen, cyano, halo, nitro or optionally substituted oxy, lower alkyl, lower alkyenyl, lower alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
X is —CH═CHCO—;
Y is —(CH₂)ₙ— where n is 1-6, —CH₂OCH₂— or —CH₂NRCH₂— and is bonded to two of the ring carbon atoms, bonding being to either the ring carbon atoms a and b or the ring carbon atoms c and d; wherein R is selected from the group consisting of H, optionally substituted: lower alkyl, carbonyl, acyl, acetyl or sulfonyl;
Z is N or —CH—;
Q is —CH₂—, —NH— or —O—;
wherein when Z is N, Q is CH, and when Z is —CH—, Q is —NH— or —O—;
The optional substitutents on R1-R4 are one or more, e.g. 1-3 substitutents, independently selected from the group consisting of hydrogen, oxo, cyano, halo, nitro or optionally substituted oxy, lower alkyl, lower alkenyl, lower alkynyl, aryl, heteroaryl, amino, sulfur, sulfinyl, sulfonyl;
wherein the optionally substituted substitutents are optionally substituted once or more by, e.g. 1-6 substitutents, a substitutent independently selected from the group consisting of hydrogen, oxo, cyano, halo, nitro, oxy, lower alkyl, lower alkyenyl, lower alkynyl, amino, sulfur, cycloalkyl, heterocyloalkyl, aryl, heteroaryl.

With respect to the compounds of the invention, preferably, R3 is halo. More preferably it is Cl. Preferably, R4 is halo. More preferably it is F. Preferably n is 2 or 3.

R1 is preferably an optionally substituted amino, amide, guanidino, sulfonyl, sulfonamide or heterocycloalkyl group, the optional substitutents being selected from the group consisting of hydrogen, oxo, cyano, halo, nitro or optionally substituted oxy, lower alkyl, lower alkenyl, lower alkynyl, heterocycloalkyl, amino, sulfur, sulfinyl, sulfonyl;

wherein the optionally substituted substitutents are optionally substituted once or more by, e.g. 1-6 substitutents, a substitutent independently selected from the group consisting of hydrogen, oxo, cyano, halo, nitro, oxy, lower alkyl, lower alkyenyl, lower alkynyl, amino, sulfur, cycloalkyl, heterocyloalkyl, aryl.

For example, R1 may be a urea group. Such urea group may optionally be substituted by any of the abovementioned optional substitutents.

Most preferably, R1 is acetamide.

R2 represents one or more groups. Preferably R2 is one group. Preferably it is located at the 4-position of the phenyl ring, relative to R1. Alternatively it is located at the 2-position. R2 may also represent two groups. In such case, the two R2 groups are preferably at the 2- and 4-positions.

Preferably, R2 is selected from the group consisting of methoxy, trifluoromethoxy, aryl, heteroaryl, lower alkyl. Preferably, R2 is methoxy. Alternatively preferably, R2 is trifluoromethoxy.

The invention further provides a compound of formula Ia, or a pharmaceutically acceptable salt or ester thereof, 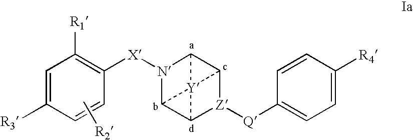
wherein

R₁′, R₂′ and R₃′ are independently selected from the group consisting of hydrogen, cyano, halo, nitro or optionally substituted oxy, lower alkyl, lower alkyenyl, lower alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;

R₄′ is selected from the group consisting of hydrogen, cyano, halo, nitro or optionally substituted oxy, lower alkyl, lower alkyenyl, lower alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;

X′ is —OCH₂CO— or —NHCH₂CO—;

Y′ is —(CH₂)ₙ— where n is 1-6, —CH₂OCH₂— or —CH₂NRCH₂— and is bonded to two of the ring carbon atoms, bonding being to either the ring carbon atoms a and b or the ring carbon atoms c and d; wherein R is selected from the group consisting of H, optionally substituted: lower alkyl, carbonyl, acyl, acetyl or sulfonyl;

Z′ is N;

Q′ is —CH₂—

The optional substitutents on R₁′-R₄′ are one or more, e.g. 1-3 substitutents, independently selected from the group consisting of hydrogen, oxo, cyano, halo, nitro or optionally substituted oxy, lower alkyl, lower alkenyl, lower alkynyl, aryl, heteroaryl, amino, sulfur, sulfinyl, sulfonyl;

wherein the optionally substituted substitutents are optionally substituted once or more by, e.g. 1-6 substitutents, a substitutent independently selected from the group consisting of hydrogen, oxo, cyano, halo, nitro, oxy, lower alkyl, lower alkyenyl, lower alkynyl, amino, sulfur, cycloalkyl, heterocyloalkyl, aryl, heteroaryl.

With respect to the compounds of the invention, preferably, R₃′ is halo. More preferably it is Cl. Preferably, R₄′ is halo. More preferably it is F. Preferably n is 2 or 3.

R₁′ is preferably an optionally substituted amino, amide, guanidino, sulfonyl, sulfonamide or heterocycloalkyl group, the optional substitutents being selected from the group consisting of hydrogen, oxo, cyano, halo, nitro or optionally substituted oxy, lower alkyl, lower alkenyl, lower alkynyl, heterocycloalkyl, amino, sulfur, sulfinyl, sulfonyl;

wherein the optionally substituted substitutents are optionally substituted once or more by, e.g. 1-6 substitutents, a substitutent independently selected from the group consisting of hydrogen, oxo, cyano, halo, nitro, oxy, lower alkyl, lower alkyenyl, lower alkynyl, amino, sulfur, cycloalkyl, heterocyloalkyl, aryl.

For example, R₁′ may be a urea group. Such urea group may optionally be substituted by any of the abovementioned optional substitutents.

Most preferably, R₁′ is acetamide.

R₂′ represents one or more groups. Preferably R₂′ is one group. Preferably it is located at the 4-position of the phenyl ring, relative to R1. Alternatively it is located at the 2-position. R₂′ may also represent two groups. In such case, the two R₂′ groups are preferably at the 2- and 4-positions.

Preferably, R₂′ is selected from the group consisting of methoxy, trifluoromethoxy, aryl, heteroaryl, lower alkyl. Preferably, R₂′ is methoxy. Alternatively preferably, R₂′ is trifluoromethoxy.

Preferably, Y′ is —CH₂OCH₂— or —CH₂NRCH₂—.

The invention further comprises a compound of formula II: 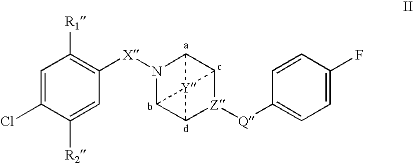
wherein R₁″, R₂″, X″, Y″, Z″ and Q″ are as defined above with respect to the corresponding groups R1, R2, X, Y, Z and Q respectively in formula I above.

Additionally the invention provides a compound of formula Ib, or a pharmaceutically acceptable salt or ester thereof, 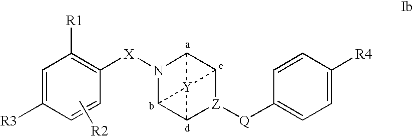
wherein
R1, R2 and R3 are independently selected from the group consisting of hydrogen, cyano, halo, nitro or optionally substituted oxy, lower alkyl, lower alkyenyl, lower alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
R4 is selected from the group consisting of hydrogen, cyano, halo, nitro or optionally substituted oxy, lower alkyl, lower alkyenyl, lower alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
X is —CH═CHCO—, —OCH₂CO— or —NHCH₂CO—;
Y is —(CH₂)ₙ— where n is 1-6, —CH₂OCH₂— or —CH₂NRCH₂— and is bonded to two of the ring carbon atoms, bonding being to either the ring carbon atoms a and b or the ring carbon atoms c and d; wherein R is selected from the group consisting of H, optionally substituted: lower alkyl, carbonyl, acyl, acetyl or sulfonyl;
Z is N or —CH—;
Q is —CH₂—, —NH— or —O—;
wherein when Z is N, Q is CH₂, and when Z is —CH—, Q is —NH— or —O—;
with the proviso that when Y is —(CH₂)ₙ— and when Z is N, X is —CH═CHCO—; and the proviso that when Q is NH or O and when X is —OCH₂CO— or —NHCH₂CO— and when Y is —(CH₂)ₙ or —CH₂OCH₂—, Y is bonded to ring carbon atoms c and d.

The optional substitutents on R1-R4 are one or more, e.g. 1-3 substitutents, independently selected from the group consisting of hydrogen, oxo, cyano, halo, nitro or optionally substituted oxy, lower alkyl, lower alkyenyl, lower alkynyl, amino, sulfur, sulfinyl, sulfonyl; wherein the optionally substituted substitutents are optionally substituted once or more by, e.g. 1-6 substitutents, a substitutent independently selected from the group consisting of hydrogen, oxo, cyano, halo, nitro, oxy, lower alkyl, lower alkyenyl, lower alkynyl, amino, sulfur, cycloalkyl, heterocyloalkyl, aryl.

The invention further comprises a compound of formula IIb: 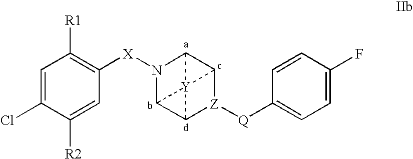
wherein R1, R2, X, Y, Z and Q are as defined above with respect to formula Ib.

With respect to the compounds Ib and IIb, preferably, R3 is halo. More preferably it is Cl. Preferably, R4 is halo. More preferably it is F. Preferably n is 2 or 3.

According to the invention in a second aspect, there is provided a compound of formula I, Ia, II, Ib or IIb wherein the compound includes a radioisotope selected from the group of ¹¹C, ¹⁸F, ⁷⁵Br, ⁷⁶Br, ⁸⁰Br, ¹²³I, ¹²⁵I, ¹²⁸I, ¹³¹I, ¹³N, ¹⁵O.

Above and elsewhere in the present description the following terms have the following meaning:

The term “lower” in connection with organic radicals or compounds means a compound or radical which may be branched or unbranched with up to and including 7 carbon atoms.

A lower alkyl group is branched or unbranched and contains from 1 to 7 carbon atoms, preferably 1 to 4 carbon atoms, and includes cycloalkyl. Lower alkyl represents for example methyl, ethyl, n-propyl, iso-propyl, n-butyl, iso-butyl, n-pentyl, t-butyl, n-heptyl, cyclopropyl. Lower alkyl is optionally substituted by hydrogen, cyano, halo, nitro, amino, oxy, alkoxy.

A lower alkenyl group is branched or unbranched, contains from 2 to 7 carbon atoms, preferably 2 to 6 carbon atoms, and contains at least one double bond. Lower alkyenyl is optionally substituted by hydrogen, cyano, halo, nitro, amino. Lower alkenyl represents for example ethenyl, prop-1-phenyl, but-1-phenyl, pent-1-phenyl, pent-1,4-dienyl.

A lower alkynyl group is branched or unbranched, contains from 2 to 7 carbon atoms, preferably 2 to 6 carbon atoms, and contains at least one triple bond. Lower alkynyl is optionally substituted by hydrogen, cyano, halo, nitro, amino. Lower alkynyl represents for example ethynyl, prop-1-ynyl, but-1-ynyl, pent-1-ynyl, pent-3-ynyl.

Amino relates to the radicals —NH₂ and ═NH and may be optionally substituted; for instance, by lower alkyl, carbonyl or sulfonyl.

Amide relates to the radical —N—CO— or —CON—.

Aryl represent carbocyclic aryl and heterocyclic aryl.

Carbocyclic aryl represents an aromatic cyclic hydrocarbon containing from 6 to 18 ring atoms. Carbocyclic aryl is mono-, bi- or tricyclic. Carbocyclic aryl represents for example phenyl, naphthyl, biphenyl. Carbocyclic aryl is optionally substituted by up to 4 substitutents.

Carbonyl refers to the radical —C(O)—

Cyano or nitrile represents the radical —CN

Cycloalkyl represents a cyclic hydrocarbon containing from 3 to 12 ring atoms preferably from 3 to 7 ring atoms and may be mono-, bi- or tricyclic and includes spiro. Cycloalkyl represents for example cyclopropyl, cyclobutyl, cyclopentyl and cyclohexyl. Cycloalkyl is optionally substituted.

Halo represents chloro, fluoro or bromo but may also be iodo.

Heterocyclic aryl represents an aromatic cyclic hydrocarbon containing from 5 to 18 ring atoms of which one or more, preferably 1 to 3, are heteroatoms selected from O, N or S. It may be mono or bicyclic. Heterocyclic aryl is optionally substituted. Heterocyclic aryl represents for example pyridyl, indoyl, quinoxalinyl, quinolinyl, isoquinolinyl, benzothienyl, benzofuranyl, benzopyranyl, benzothiopyranyl, furanyl, pyrrolyl, thiazolyl, oxazolyl, isoxazolyl, oxadiazolyl, triazolyl, thiadiazolyl, tetrazolyl, pyrazolyl, imidazolyl, thienyl.

Heterocycloalkyl represents a mono-, bi- or tricyclic hydrocarbon containing from 3 to 18 ring atoms preferably from 3 to 7 ring atoms and contains one or more, preferably 1 to 3, heteroatoms selected from O, N or S. Heterocycloalkyl is optionally substituted. Heterocycloalkyl represents for example pyrrolidinyl, piperidinyl, piperazinyl, morpholinyl, indolinylmethyl, imidazolinylmethyl and 2-Aza-bicyclo[2.2.2]octanyl

Nitro represents the radical —NO₂

Oxo represents the substitutent ═O

Oxy represents the radical —O—, and includes —OH

sulfur indicates the radicals —S—,

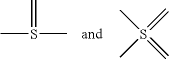

In particular the invention includes a compound selected from:
(E)-N-(5-Chloro-2-{3-[3-(4-fluorobenzyl)-3,8-diazabicyclo[3.2.1]oct-8-yl]-3-oxopropenyl}-phenyl)acetamide
(E)-N-(5-Chloro-2-{3-[3-(4-fluorobenzyl)-3,8-diazabicyclo[3.2.1]oct-8-yl]-3-oxopropenyl}-phenyl)-N′-cyanoguanidine
(E)-N-(5-Chloro-2-{3-[3-(4-fluorobenzyl)-3,8-diazabicyclo[3.2.1]oct-8-yl]-3-oxopropenyl}-phenyl)-2-dimethylaminoacetamide
(E)-(5-Chloro-2-{3-[3-(4-fluorobenzyl)-3,8-diazabicyclo[3.2.1]oct-8-yl]-3-oxopropenyl}-phenyl)-urea
N-(5-Chloro-2-{(E)-3-[3-(4-fluoro-benzyl)-3,8-diaza-bicyclo[3.2.1]oct-8-yl]-3-oxo-propenyl}-phenyl)-methanesulfonamide
N-(5-Chloro-2-{(E)-3-[3-(4-fluoro-benzyl)-3,8-diaza-bicyclo[3.2.1]oct-8-yl]-3-oxo-propenyl}-phenyl)-2-methoxy-acetamide
1-(5-Chloro-2-{(E)-3-[3-(4-fluoro-benzyl)-3,8-diaza-bicyclo[3.2.1]oct-8-yl]-3-oxo-propenyl}-phenyl)-3-methyl-urea
3-(5-Chloro-2-{(E)-3-[3-(4-fluoro-benzyl)-3,8-diaza-bicyclo[3.2.1]oct-8-yl]-3-oxo-propenyl}-phenyl)-1,1-dimethyl-urea
1-(5-Chloro-2-{(E)-3-[3-(4-fluoro-benzyl)-3,8-diaza-bicyclo[3.2.1]oct-8-yl]-3-oxo-propenyl}-phenyl)-3-ethyl-urea
1-(5-Chloro-2-{(E)-3-[3-(4-fluoro-benzyl)-3,8-diaza-bicyclo[3.2.1]oct-8-yl]-3-oxo-propenyl}-phenyl)-3-propyl-urea
1-(5-Chloro-2-{(E)-3-[3-(4-fluoro-benzyl)-3,8-diaza-bicyclo[3.2.1]oct-8-yl]-3-oxo-propenyl}-phenyl)-3-isopropyl-urea
1-(5-Chloro-2-{(E)-3-[3-(4-fluoro-benzyl)-3,8-diaza-bicyclo[3.2.1]oct-8-yl]-3-oxo-propenyl}-phenyl)-3-cyclopropyl-urea
1-(5-Chloro-2-{(E)-3-[3-(4-fluoro-benzyl)-3,8-diaza-bicyclo[3.2.1]oct-8-yl]-3-oxo-propenyl}-phenyl)-3-(tetrahydro-pyran-4-yl)-urea
3-Oxo-piperazine-1-carboxylic acid (5-chloro-2-{(E)-3-[3-(4-fluoro-benzyl)-3,8-diaza-bicyclo[3.2.1]oct-8-yl]-3-oxo-propenyl}-phenyl)-amide
2-Oxo-oxazolidine-3-sulfonic acid (5-chloro-2-{(E)-3-[3-(4-fluoro-benzyl)-3,8-diaza-bicyclo[3.2.1]oct-8-yl]-3-oxo-propenyl}-phenyl)-amide
N-(5-Chloro-2-{(E)-3-[8-(4-fluoro-benzyl)-3,8-diaza-bicyclo[3.2.1]oct-3-yl]-3-oxo-propenyl}-phenyl)-methanesulfonamide
1-(5-Chloro-2-{(E)-3-[8-(4-fluoro-benzyl)-3,8-diaza-bicyclo[3.2.1]oct-3-yl]-3-oxo-propenyl}-phenyl)-3-ethyl-urea
N-(5-Chloro-2-{(E)-3-[8-(4-fluoro-benzyl)-3,8-diaza-bicyclo[3.2.1]oct-3-yl]-3-oxo-propenyl}-phenyl)-2-methoxy-acetamide
(5-Chloro-2-{(E)-3-[8-(4-fluoro-benzyl)-3,8-diaza-bicyclo[3.2.1]oct-3-yl]-3-oxo-propenyl}-phenyl)-urea
(E)-N-(5-Chloro-2-{3-[8-(4-fluorobenzyl)-3,8-diazabicyclo[3.2.1]oct-3-yl]-3-oxopropenyl}-phenyl)acetamide
3-(5-Chloro-2-{(E)-3-[8-(4-fluoro-benzyl)-3,8-diaza-bicyclo[3.2.1]oct-3-yl]-3-oxo-propenyl}-phenyl)-1,1-dimethyl-urea
1-(5-Chloro-2-{(E)-3-[8-(4-fluoro-benzyl)-3,8-diaza-bicyclo[3.2.1]oct-3-yl]-3-oxo-propenyl}-phenyl)-3-methyl-urea
1-(5-Chloro-2-{(E)-3-[3-(4-fluorobenzyl)-3,8-diazabicyclo[3.2.1]oct-8-yl]-3-oxopropenyl}-4-methoxyphenyl)-3-methyl urea
(5-Chloro-2-{(E)-3-[3-(4-fluorobenzyl)-3,8-diazabicyclo[3.2.1]oct-8-yl]-3-oxopropenyl}-4-methoxyphenyl)-3-urea
N-(5-Chloro-2-{(E)-3-[3-(4-fluorobenzyl)-3,8-diazabicyclo[3.2.1]oct-8-yl]-3-oxopropenyl}-4-methoxyphenyl)-acetamide
1-(5-Chloro-2-{(E)-3-[3-(4-fluoro-benzyl)-3,8-diaza-bicyclo[3.2.1]oct-8-yl]-3-oxo-propenyl}-4-methoxy-phenyl)-3-cyclopropyl-urea
N-(5-Chloro-2-{(E)-3-[3-(4-fluoro-benzyl)-3,8-diaza-bicyclo[3.2.1]oct-8-yl]-3-oxo-propenyl}-4-methoxy-phenyl)-methanesulfonamide
N-(5-Chloro-2-{(E)-3-[3-(4-fluoro-benzyl)-3,8-diaza-bicyclo[3.2.1]oct-8-yl]-3-oxo-propenyl}-4-methoxy-phenyl)-2-dimethylamino-acetamide
3-(5-Chloro-2-{(E)-3-[3-(4-fluoro-benzyl)-3,8-diaza-bicyclo[3.2.1]oct-8-yl]-3-oxo-propenyl}-4-methoxy-phenyl)-1,1-dimethyl-urea
5-Chloro-2-{(E)-3-[3-(4-fluoro-benzyl)-3,8-diaza-bicyclo[3.2.1]oct-8-yl]-3-oxo-propenyl}-4-methoxy-N,N-dimethyl-benzenesulfonamide
N-[5-Chloro-2-{(E)-3-[3-(4-fluoro-benzyl)-3,8-diaza-bicyclo[3.2.1]oct-8-yl]-3-oxo-propenyl}-4-(1-hydroxy-1-methyl-ethyl)-phenyl]-acetamide
N-(5-Chloro-4-ethoxy-2-{(E)-3-[3-(4-fluoro-benzyl)-3,8-diaza-bicyclo[3.2.1]oct-8-yl]-3-oxo-propenyl}-phenyl)-acetamide
N-(5-Chloro-4-ethoxy-2-{(E)-3-[3-(4-fluoro-benzyl)-3,8-diaza-bicyclo[3.2.1]oct-8-yl]-3-oxo-propenyl}-phenyl)-methansulfonamide
N-(5-Chloro-4-ethoxy-2-{(E)-3-[3-(4-fluoro-benzyl)-3,8-diaza-bicyclo[3.2.1]oct-8-yl]-3-oxo-propenyl}-phenyl)-urea
(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-urea
1-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-3-methyl-urea
N-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-acetamide
3-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-1,1-dimethyl-urea
3-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-1,1-dimethylsulfonyl-urea
5-Chloro-2-{(E)-3-[3-(4-fluoro-benzyl)-3,8-diaza-bicyclo[3.2.1]oct-8-yl]-3-oxo-propenyl}-N,N-dimethyl-4-trifluoromethoxy-benzenesulfonamide
(5-Chloro-2-{(E)-3-[3-(4-fluorobenzyl)-3,8-diazabicyclo[3.2.1]oct-8-yl]-3-oxopropenyl}-4-methyl phenyl)-urea
N-(5-Chloro-2-{(E)-3-[3-(4-fluoro-benzyl)-3,8-diaza-bicyclo[3.2.1]oct-8-yl]-3-oxo-propenyl}-4-methyl-phenyl)-methanesulfonamide
N-(5-Chloro-2-{(E)-3-[3-(4-fluoro-benzyl)-3,8-diaza-bicyclo[3.2.1]oct-8-yl]-3-oxo-propenyl}-4-methyl-phenyl)-1,1-dimethylsulfonyl-urea
N-(5-Chloro-2-{(E)-3-[3-(4-fluoro-benzyl)-3,8-diaza-bicyclo[3.2.1]oct-8-yl]-3-oxo-propenyl}-4-methyl-phenyl)-2-methoxy-acetamide
N-(5-Chloro-2-{(E)-3-[3-(4-fluoro-benzyl)-3,8-diaza-bicyclo[3.2.1]oct-8-yl]-3-oxo-propenyl}-4-methyl-phenyl)-acetamide
1-(5-Chloro-2-{(E)-3-[3-(4-fluoro-benzyl)-3,8-diaza-bicyclo[3.2.1]oct-8-yl]-3-oxo-propenyl}-4-methyl-phenyl)-3-methyl-urea
3-(5-Chloro-2-{(E)-3-[3-(4-fluoro-benzyl)-3,8-diaza-bicyclo[3.2.1]oct-8-yl]-3-oxopropenyl}-4-methyl-phenyl)-1,1-dimethyl-urea
3-Oxo-piperazine-1-carboxylic acid (5-chloro-2-{(E)-3-[3-(4-fluoro-benzyl)-3,8-diaza-bicyclo[3.2.1]oct-8-yl]-3-oxo-propenyl}-4-methyl-phenyl)-amide
1-(5-Chloro-2-{(E)-3-[3-(4-fluoro-benzyl)-3,8-diaza-bicyclo[3.2.1]oct-8-yl]-3-oxo-propenyl}-4-methyl-phenyl)-3-cyclopropyl-urea
1-(5-Chloro-2-{(E)-3-[3-(4-fluoro-benzyl)-3,8-diaza-bicyclo[3.2.1]oct-8-yl]-3-oxo-propenyl}-4-methyl-phenyl)-tert-butylsulfonyl-urea
5-Chloro-2-{(E)-3-[3-(4-fluoro-benzyl)-3,8-diaza-bicyclo[3.2.1]oct-8-yl]-3-oxo-propenyl}-4, N,N-trimethyl-benzenesulfonamide
N-(3′-Amino-2-chloro-5-{(E)-3-[3-(4-fluoro-benzyl)-3,8-diaza-bicyclo[3.2.1]oct-8-yl]-3-oxo-propenyl}-biphenyl-4-yl)-acetamide
N-(3′-Acetylamino-2-chloro-5-{(E)-3-[3-(4-fluoro-benzyl)-3,8-diaza-bicyclo[3.2.1]oct-8-yl]-3-oxo-propenyl}-biphenyl-4-yl)-acetamide
N-(2-Chloro-5-{(E)-3-[3-(4-fluoro-benzyl)-3,8-diaza-bicyclo[3.2.1]oct-8-yl]-3-oxo-propenyl}-3′-ureido-biphenyl-4-yl)-acetamide
N-(5-Chloro-2-{(E)-3-[3-(4-fluoro-benzyl)-3,8-diaza-bicyclo[3.2.1]oct-8-yl]-3-oxo-propenyl}-4-pyrazin-2-yl-phenyl)-acetamide
N-(5-Chloro-2-{(E)-3-[3-(4-fluoro-benzyl)-3,8-diaza-bicyclo[3.2.1]oct-8-yl]-3-oxo-propenyl}-4-pyridin-3-yl-phenyl)-acetamide
(5-Chloro-2-{(E)-3-[3-(4-fluoro-benzyl)-3,8-diaza-bicyclo[3.2.1]oct-8-yl]-3-oxo-propenyl}-4-pyridin-3-yl-phenyl)-urea
N-(5-Chloro-2-{(E)-3-[3-(4-fluoro-benzyl)-3,8-diaza-bicyclo[3.2.1]oct-8-yl]-3-oxo-propenyl}-4-pyridin-2-yl-phenyl)-acetamide
N-(3-Chloro-6-{(E)-3-[3-(4-fluoro-benzyl)-3,8-diaza-bicyclo[3.2.1]oct-8-yl]-3-oxo-propenyl}-2,4-dimethoxy-phenyl)-acetamide
(E)-N-(5-Chloro-2-{3-[3-(4-fluorobenzyl)-3,9-diazabicyclo[3.3.1]non-9-yl]-3-oxopropenyl}-phenyl)-acetamide
(E)-N-(5-Chloro-2-{3-[3-(4-fluorobenzyl)-3,9-diazabicyclo[3.3.1]non-9-yl]-3-oxopropenyl}-phenyl)-urea
(E)-N-(5-Chloro-2-{3-[3-(4-fluorobenzyl)-3,9-diazabicyclo[3.3.1]non-9-yl]-3-oxopropenyl}-phenyl)-N′-cyanoguanidine
(E)-N-(5-Chloro-2-{3-[3-(4-fluorobenzyl)-3,9-diazabicyclo[3.3.1]non-9-yl]-3-oxopropenyl}-phenyl)-2-dimethylaminoacetamide
9-[2-(2-Acetylamino-4-chloro-phenoxy)-acetyl]-7-(4-fluorobenzyl)-3,7,9-triazabicyclo[3.3.1]nonane-3-carboxylic acid tert-butyl ester
N-(5-Chloro-2-{2-[3-(4-fluorobenzyl)-3,7,9-triazabicyclo[3.3.1]non-9-yl]-2-oxo-ethoxy}-phenyl)-acetamide
7-[2-(2-Acetylamino-4-chloro-phenoxy)-acetyl]-9-(4-fluorobenzyl)-3,7,9-triazabicyclo[3.3.1]nonane-3-carboxylic acid tert-butyl ester
N-(5-Chloro-2-{2-[9-(4-fluoro-benzyl)-3,7,9-triaza-bicyclo[3.3.1]non-3-yl]-2-oxo-ethoxy}-phenyl)-acetamide
(E)-N-(5-Chloro-2-{3-[3-(4-fluorobenzyl)-3,7,9-triazabicyclo[3.3.1]non-9-yl]-3-oxo-propenyl}-phenyl)-acetamide
(E)-N-(5-Chloro-2-{3-[3-(4-fluorobenzyl)-3,7,9-triazabicyclo[3.3.1]non-9-yl]-3-oxopropenyl}-phenyl)-2-dimethylaminoacetamide
(E)-N-(5-Chloro-2-{3-[3-(4-fluorobenzyl)-3,7,9-triazabicyclo[3.3.1]non-9-yl]-3-oxopropenyl}-phenyl)methanesulfonamide
(5-Chloro-2-{(E)-3-[3-(4-fluorobenzyl)-3,7,9-triazabicyclo[3.3.1]non-9-yl]-3-oxopropenyl}-phenyl)-urea hydrochloride
(E)-N-(5-Chloro-2-{3-[3-(4-fluorobenzyl)-3,7,9-triazabicyclo[3.3.1]non-9-yl]-3-oxo-propenyl}-phenyl)-acetamide
(5-Chloro-2-{(E)-3-[3-(4-fluorobenzyl)-7-methyl-3,7,9-triazabicyclo[3.3.1]non-9-yl]-3-oxopropenyl}phenyl)-urea
N-(5-Chloro-2-{(E)-3-[3-(4-fluorobenzyl)-7-methyl-3,7,9-triazabicyclo[3.3.1]non-9-yl]-3-oxopropenyl}-phenyl)-methanesulfonamide
N-(5-Chloro-2-{(E)-3-[3-(4-fluorobenzyl)-7-methyl-3,7,9-triazabicyclo[3.3.1]non-9-yl]-3-oxopropenyl}phenyl)-acetamide
N-(5-Chloro-2-{(E)-3-[3-(4-fluorobenzyl)-3,7,9-triazabicyclo[3.3.1]non-9-yl]-3-oxo-propenyl}-4-methoxyphenyl)-acetamide
N-(5-Chloro-2-{(E)-3-[3-(4-fluorobenzyl)-3,7,9-triazabicyclo[3.3.1]non-9-yl]-3-oxo-propenyl}-4-methoxyphenyl)-methanesulfonamide hydrochloride
(5-Chloro-2-{(E)-3-[3-(4-fluorobenzyl)-3,7,9-triazabicyclo[3.3.1]non-9-yl]-3-oxopropenyl}-4-methoxyphenyl)-urea hydrochloride
N-(5-Chloro-2-{(E)-3-[3-(4-fluorobenzyl)-3,7,9-triazabicyclo[3.3.1]non-9-yl]-3-oxopropenyl}-4-methoxyphenyl)-2-dimethylaminoacetamide dihydrochloride
N-(2-{(E)-3-[3-Acetyl-7-(4-fluorobenzyl)-3,7,9-triazabicyclo[3.3.1]non-9-yl]-3-oxopropenyl}-5-chloro-4-methoxyphenyl)-acetamide
9-[(E)-3-(2-Acetylamino-4-chloro-5-methoxyphenyl)-acryloyl]-7-(4-fluorobenzyl)-3,7,9-triazabicyclo[3.3.1]nonane-3-carboxylic acid methylamide
9-[(E)-3-(2-Acetylamino-4-chloro-5-methoxyphenyl)-acryloyl]-7-(4-fluorobenzyl)-3,7,9-triazabicyclo[3.3.1]nonane-3-carboxylic acid dimethylamide
9-[(E)-3-(2-Acetylamino-4-chloro-5-methoxyphenyl)-acryloyl]-7-(4-fluorobenzyl)-3,7,9-triazabicyclo[3.3.1]nonane-3-carboxylic acid methyl ester
N-(5-Chloro-2-{3-[3-(4-fluorobenzyl)-7-methanesulfonyl-3,7,9-triazabicyclo[3.3.1]non-9-yl]-3-oxopropenyl}-4-methoxyphenyl)-acetamide
5-Chloro-2-{(E)-3-[3-(4-fluorobenzyl)-7-methanesulfonyl-3,7,9-triazabicyclo[3.3.1]non-9-yl]-3-oxopropenyl}-N,N-dimethyl-4-trifluormethoxybenzenesulfonamide
N-(2-{(E)-3-[3-Acetyl-7-(4-fluorobenzyl)-3,7,9-triazabicyclo[3.3.1]non-9-yl]-3-oxopropenyl}-5-chloro-4-fluorophenyl)-acetamide
N-(5-Chloro-(2-{(E)-3-[3-(4-fluorobenzyl)-3,7,9-triazabicyclo[3.3.1]non-9-yl]-3-oxopropenyl}-4-methylphenyl)-acetamide hydrochloride
N-(5-Chloro-(2-{(E)-3-[3-(4-fluorobenzyl)-3,7,9-triazabicyclo[3.3.1]non-9-yl]-3-oxopropenyl}-4-methylphenyl)-methanesulfonamide hydrochloride
(5-Chloro-(2-{(E)-3-[3-(4-fluorobenzyl)-3,7,9-triazabicyclo[3.3.1]non-9-yl]-3-oxopropenyl}-4-methylphenyl)-urea hydrochloride
N-(5-Chloro-(2-{(E)-3-[3-(4-fluorobenzyl)-3,7,9-triazabicyclo[3.3.1]non-9-yl]-3-oxopropenyl}-4-methylphenyl)-2-dimethylaminoacetamide dihydrochloride
(5-Chloro-2-{(E)-9-[3-(4-fluorobenzyl)-7-methyl-3,7,9-triazabicyclo[3.3.1]non-3-yl]-3-oxopropenyl}-4-methylphenyl)-urea
N-(5-Chloro-2-{(E)-3-[9-(4-fluorobenzyl)-7-methyl-3,7,9-triazabicyclo[3.3.1]non-3-yl]-3-oxopropenyl}-4-methylphenyl)-methanesulfonamide
N-(5-Chloro-2-{(E)-3-[9-(4-fluorobenzyl)-7-methyl-3,7,9-triazabicyclo[3.3.1]non-3-yl]-3-oxopropenyl}-acetamide
N-(2-{(E)-3-[3-Acetyl-7-(4-fluorobenzyl)-3,7,9-triazabicyclo[3.3.1]non-9-yl]-3-oxopropenyl}-5-chloro-4-methylphenyl)-acetamide
(5-Chloro-2-{(E)-3-[3-(4-fluorobenzyl)-7-methyl-3,7,9-triazabicyclo[3.3.1]non-9-yl]-3-oxopropenyl}-4-methylphenyl)-urea hydrochloride
N-(5-Chloro-2-{(E)-3-[3-(4-fluorobenzyl)-7-methyl-3,7,9-triazabicyclo[3.3.1]non-9-yl]-3-oxopropenyl}-4-methyl phenyl)-methanesulfonamide
N-(5-Chloro-2-{(E)-3-[3-(4-fluorobenzyl)-7-methyl-3,7,9-triazabicyclo[3.3.1]non-9-yl]-3-oxopropenyl}-4-methylphenyl)-amide hydrochloride
N-(5-Chloro-2-{(E)-3-[3-(4-fluorobenzyl)-7-methyl-3,7,9-triazabicyclo[3.3.1]non-9-yl]-3-oxo-propenyl}-4-methoxyphenyl)-acetamide
N-(5-Chloro-2-{(E)-3-[3-(4-fluorobenzyl)-7-methyl-3,7,9-triazabicyclo[3.3.1]non-9-yl]-3-oxo-propenyl}-4-methoxyphenyl)-methanesulfonamide
(5-Chloro-2-{(E)-3-[3-(4-fluorobenzyl)-7-methyl-3,7,9-triazabicyclo[3.3.1]non-9-yl]-3-oxo-propenyl}-4-methoxyphenyl)-urea
N-(5-Chloro-2-{(E)-3-[3-(4-fluoro-benzyl)-7-methyl-3,7,9-triaza-bicyclo[3.3.1]non-9-yl]-3-oxo-propenyl}-4-methoxy-phenyl)-N,N-dimethylsulfonylurea
N-(5-Chloro-2-{2-[7-(4-fluorobenzyl)-3-oxa-7,9-diazabicyclo[3.3.1]non-9-yl]-2-oxoethoxy}-phenyl)acetamide
N-(5-Chloro-2-{2-[9-(4-fluorobenzyl)-3-oxa-7,9-diazabicyclo[3.3.1]non-7-yl]-2-oxoethoxy}-phenyl)acetamide
(E)-N-(5-Chloro-2-{3-[9-(4-fluorobenzyl)-3-oxa-7,9-diazabicyclo[3.3.1]non-7-yl]-3-oxopropenyl}-phenyl)-acetamide
(E)-N-(5-Chloro-2-{3-[7-(4-fluorobenzyl)-3-oxa-7,9-diazabicyclo[3.3.1]non-9-yl]-3-oxopropenyl}-phenyl)-acetamide
(E)-N-(5-Chloro-2-{3-[7-(4-fluorobenzyl)-3-oxa-7,9-diazabicyclo[3.3.1]non-9-yl]-3-oxopropenyl}-phenyl)-urea
(E)-N-(5-Chloro-2-{3-[7-(4-fluorobenzyl)-3-oxa-7,9-diazabicyclo[3.3.1]non-9-yl]-3-oxopropenyl}-phenyl)-N′cyanoguanidine
(E)-(5-Chloro-2-{3-[9-(4-fluorobenzyl)-3-oxa-7,9-diazabicyclo[3.3.1]non-7-yl]-3-oxopropenyl}-phenyl)-urea
N-(5-Chloro-2-{(E)-3-[9-(4-fluorobenzyl)-3-oxa-7,9-diazabicyclo[3.3.1]non-7-yl]-3-oxopropenyl}-4-methoxyphenyl)-acetamide
N-(5-Chloro-2-{(E)-3-[7-(4-fluorobenzyl)-3-oxa-7,9-diazabicyclo[3.3.1]non-9-yl]-3-oxopropenyl}-4-methoxyphenyl)-acetamide
N-(5-Chloro-2-{(E)-3-[7-(4-fluorobenzyl)-3-oxa-7,9-diaza-bicyclo[3.3.1]non-9-yl]-3-oxopropenyl}-4-methoxyphenyl)-methanesulfonamide
5-Chloro-2-{(E)-3-[7-(4-fluoro-benzyl)-3-oxa-7,9-diaza-bicyclo[3.3.1]non-9-yl]-3-oxo-propenyl}-4-methoxy-phenyl)-urea
1-(5-Chloro-2-{(E)-3-[7-(4-fluoro-benzyl)-3-oxa-7,9-diaza-bicyclo[3.3.1]non-9-yl]-3-oxo-propenyl}-4-methoxy-phenyl)-3-methyl-urea
1-(5-Chloro-2-{(E)-3-[7-(4-fluoro-benzyl)-3-oxa-7,9-diaza-bicyclo[3.3.1]non-9-yl]-3-oxo-propenyl}-4-methoxy-phenyl)-3-cyclopropyl-urea
5-Chloro-2-{(E)-3-[7-(4-fluoro-benzyl)-3-oxa-7,9-diaza-bicyclo[3.3.1]non-9-yl]-3-oxo-propenyl}-4-methoxy-N,N-dimethyl-benzenesulfonamide
N-(3-Chloro-6-{(E)-3-[9-(4-fluoro-benzyl)-3-oxa-7,9-diaza-bicyclo[3.3.1]non-7-yl]-3-oxo-propenyl}-2,4-dimethoxy-phenyl)-acetamide
N-(3-Chloro-6-{(E)-3-[9-(4-fluoro-benzyl)-3-oxa-7,9-diaza-bicyclo[3.3.1]non-7-yl]-3-oxo-propenyl}-2-methoxy-phenyl)-acetamide
N-(5-Chloro-2-{(E)-3-[9-(4-fluoro-benzyl)-3-oxa-7,9-diaza-bicyclo[3.3.1]non-7-yl]-3-oxo-propenyl}-4-methoxy-phenyl)-methanesulfonamide
(5-Chloro-2-{(E)-3-[9-(4-fluoro-benzyl)-3-oxa-7,9-diaza-bicyclo[3.3.1]non-7-yl]-3-oxo-propenyl}-4-methoxy-phenyl)-urea
Cyclopropanecarboxylic acid (5-chloro-2-{(E)-3-[9-(4-fluoro-benzyl)-3-oxa-7,9-diaza-bicyclo[3.3.1]non-7-yl]-3-oxo-propenyl}-4-methoxy-phenyl)-amide
N-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-acetamide
(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-urea
1-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-3-methyl-urea
N-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-isobutyramide
5-Chloro-2-{(E)-3-[7-(4-fluoro-benzyl)-3-oxa-7,9-diaza-bicyclo[3.3.1]non-9-yl]-3-oxo-propenyl}-N,N-dimethyl-4-trifluoromethoxy-benzenesulfonamide
N-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-N,N-dimethylsulfonylurea
1-(5-Chloro-4-cyclopropylmethoxy-2-{(E)-3-[7-(4-fluoro-benzyl)-3-oxa-7,9-diaza-bicyclo[3.3.1]non-9-yl]-3-oxo-propenyl}-phenyl)-3-methyl-urea
N-(5-Chloro-4-cyclopropylmethoxy-2-{(E)-3-[7-(4-fluoro-benzyl)-3-oxa-7,9-diaza-bicyclo[3.3.1]non-9-yl]-3-oxo-propenyl}-phenyl)-acetamide
N-(5-Chloro-2-{(E)-3-[7-(4-fluoro-benzyl)-3-oxa-7,9-diaza-bicyclo[3.3.1]non-9-yl]-3-oxo-propenyl}-4-methyl-phenyl)-acetamide
N-(5-Chloro-2-{(E)-3-[9-(4-fluoro-benzyl)-3-oxa-7,9-diaza-bicyclo[3.3.1]non-7-yl]-3-oxo-propenyl}-4-methyl-phenyl)-acetamide
N-(5-Chloro-2-{(E)-3-[7-(4-fluoro-benzyl)-3-oxa-7,9-diaza-bicyclo[3.3.1]non-9-yl]-3-oxo-propenyl}-4-pyrazin-2-yl-phenyl)-acetamide
N-(5-Chloro-2-{(E)-3-[9-(4-fluoro-benzyl)-3-oxa-7,9-diaza-bicyclo[3.3.1]non-7-yl]-3-oxo-propenyl}-4-pyrazin-2-yl-phenyl)-acetamide
N-(5-Chloro-2-{(E)-3-[9-(4-fluoro-benzyl)-3-oxa-7,9-diaza-bicyclo[3.3.1]non-7-yl]-3-oxo-propenyl}-4-pyridin-2-yl-phenyl)-acetamide
N-(5-Chloro-2-{(E)-3-[7-(4-fluoro-benzyl)-3-oxa-7,9-diaza-bicyclo[3.3.1]non-9-yl]-3-oxo-propenyl}-4-pyridin-2-yl-phenyl)-acetamide
N-(5-Chloro-2-{(E)-3-[(1S,3R,5R)-3-(4-fluoro-phenylamino)-8-aza-bicyclo[3.2.1]oct-8-yl]-3-oxo-propenyl}-4-pyrazin-2-yl-phenyl)-acetamide
N-(5-Chloro-2-{(E)-3-[(1S,3R,5R)-3-(4-fluoro-phenylamino)-8-aza-bicyclo[3.2.1]oct-8-yl]-3-oxo-propenyl}-4-pyridin-2-yl-phenyl)-acetamide
(5-Chloro-2-{(E)-3-[(1R,3R,5S)-3-(4-fluoro-phenylamino)-8-aza-bicyclo[3.2.1]oct-8-yl]-3-oxo-propenyl}-4-methoxy-phenyl)-urea
N-(5-Chloro-2-{(E)-3-[(1R,3R,5S)-3-(4-fluoro-phenylamino)-8-aza-bicyclo[3.2.1]oct-8-yl]-3-oxo-propenyl}-4-methoxy-phenyl)-acetamide
(5-Chloro-2-{(E)-3-[(1R,3R,5S)-3-(4-fluoro-phenylamino)-8-aza-bicyclo[3.2.1]oct-8-yl]-3-oxo-propenyl}-4-trifluoromethoxy-phenyl)-urea
N-(5-Chloro-2-{(E)-3-[(1R,3R,5S)-3-(4-fluoro-phenylamino)-8-aza-bicyclo[3.2.1]oct-8-yl]-3-oxo-propenyl}-4-trifluoromethoxy-phenyl)-acetamide
5-Chloro-2-{(E)-3-[(1R,3R,5S)-3-(4-fluoro-phenylamino)-8-aza-bicyclo[3.2.1]oct-8-yl]-3-oxo-propenyl}-4-methoxy-N,N-dimethyl-benzenesulfonamide
N-(5-Chloro-2-{(E)-3-[(1S,5R,8S)-8-(4-fluoro-phenylamino)-3-aza-bicyclo[3.2.1]oct-3-yl]-3-oxo-propenyl}-4-methoxy-phenyl)-acetamide
(5-Chloro-2-{(E)-3-[(1S,5R,9S)-9-(4-fluoro-phenylamino)-3-oxa-7-aza-bicyclo[3.3.1]non-7-yl]-3-oxo-propenyl}-4-trifluoromethoxy-phenyl)-urea
N-(5-Chloro-2-{(E)-3-[(1S,5R,9S)-9-(4-fluoro-phenylamino)-3-oxa-7-aza-bicyclo[3.3.1]non-7-yl]-3-oxo-propenyl}-4-trifluoromethoxy-phenyl)-acetamide
N-(5-Chloro-2-{(E)-3-[(1S,5R,9R)-9-(4-fluoro-phenylamino)-3-oxa-7-aza-bicyclo[3.3.1]non-7-yl]-3-oxo-propenyl}-4-trifluoromethoxy-phenyl)-acetamide
N-(5-Chloro-2-{(E)-3-[(1S,5R,9R)-9-(4-fluoro-phenylamino)-3-oxa-7-aza-bicyclo[3.3.1]non-7-yl]-3-oxo-propenyl}-4-methoxy-phenyl)-acetamide
N-(5-Chloro-2-{(E)-3-[(1S,5R,9S)-9-(4-fluoro-phenylamino)-3-oxa-7-aza-bicyclo[3.3.1]non-7-yl]-3-oxo-propenyl}-4-methoxy-phenyl)-acetamide
N-(5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-methoxy-phenyl)-acetamide
N-(5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-trifluoromethoxy-phenyl)-acetamide
3-(5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-methoxy-phenyl)-1,1-dimethyl-urea
5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-methoxy-N,N-dimethyl-benzenesulfonamide
N-(5-Chloro-4-fluoro-2-{(E)-3-[3-(4-fluoro-benzyl)-3,8-diaza-bicyclo[3.2.1]oct-8-yl]-3-oxo-propenyl}-phenyl)-acetamide
N-(5-Chloro-4-fluoro-2-{(E)-3-[3-(4-fluoro-benzyl)-7-methyl-3,7,9-triaza-bicyclo[3.3.1]non-9-yl]-3-oxo-propenyl}-phenyl)-acetamide
6-(5-Chloro-4-fluoro-2-{(E)-3-[3-(4-fluoro-benzyl)-3,8-diaza-bicyclo[3.2.1]oct-8-yl]-3-oxo-propenyl}-phenyl)-4,6-diaza-spiro[2,4]heptane-5,7-dione
6-(5-Chloro-2-{(E)-3-[3-(4-fluoro-benzyl)-3,8-diaza-bicyclo[3.2.1]oct-8-yl]-3-oxo-propenyl}-4-methoxy-phenyl)-4,6-diaza-spiro[2,4]heptane-5,7-dione
6-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-4,6-diaza-spiro[2,4]heptane-5,7-dione
3-(5-Chloro-2-{(E)-3-[3-(4-fluoro-benzyl)-3,8-diaza-bicyclo[3.2.1]oct-8-yl]-3-oxo-ropenyl}-4-trifluoromethyl-phenyl)-5-methyl-imidazolidine-2,4-dione
3-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-5-methyl-imidazolidine-2,4-dione
3-(5-Chloro-2-{(E)-3-[3-(4-fluoro-benzyl)-3,8-diaza-bicyclo[3.2.1]oct-8-yl]-3-oxo-propenyl}-4-methoxy-phenyl)-5-methyl-imidazolidine-2,4-dione
   or a pharmaceutically acceptable salt, or ester thereof.

The compounds of formula I, Ia, II, Ib and IIb and as listed above are herein after referred to as Agents of the Invention.

Pharmaceutically acceptable salts of the acidic Agents of the Invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methylammoniumn salts.

Similarly acid addition salts, such as of mineral acids, organic carboxylic and organic sulfonic acids e.g. hydrochloric acid, methanesulfonic acid, maleic acid, are also possible provided a basic group, such as pyridyl, piperazinyl, piperidinyl constitutes part of the structure.

Agents of the Invention may also exist in the form of optical isomers; for example as hereinafter described in the Examples. Thus the invention includes both individual isomeric forms as well as mixtures, e.g. racemic and diastereoisomeric mixtures thereof, unless otherwise specified. Conveniently the invention includes compounds of formula I in purified isomeric form, e.g. comprising at least 90%, or preferably at least 95%, of a single isomeric form.

Where Agents of the Invention exist in isomeric form as aforesaid, individual isomers may be obtained in conventional manner, e.g. employing optically active starting materials or by separation of initially obtained mixtures, for example using conventional chromatographic techniques.

The Agents of the Invention which comprise free hydroxyl groups may also exist in the form of pharmaceutically acceptable, physiologically cleavable esters, and as such are included within the scope of the invention. Such pharmaceutically acceptable esters are preferably prodrug ester derivatives, such being convertible by solvolysis or cleavage under physiological conditions to the corresponding Agents of the Invention which comprise free hydroxyl groups. Suitable pharmaceutically acceptable prodrug esters are those derived from a carboxylic acid, a carbonic acid monoester or a carbamic acid, advantageously esters derived from an optionally substituted lower alkanoic acid or an arylcarboxylic acid.

According to the invention in a third aspect, there is provided an Agent of the invention for use as a pharmaceutical.

According to the invention in a fourth aspect, there is provided an Agent of the invention for use in the treatment of inflammation.

According to the invention in a fifth aspect, there is provided a method of inhibiting chemokine receptors or of reducing inflammation in a mammal in need of such treatment which method comprises administering to said subject an effective amount of an Agent of the invention.

According to the invention in a sixth aspect, there is provided a pharmaceutical composition comprising an Agent of the invention in association with a pharmaceutically acceptable diluent or carrier, for use as an immunosuppressant or anti-inflammatory agent.

According to the invention in a seventh aspect there is provided the use of an Agent of the invention in the manufacture of a medicament for use as an immunosuppressant or anti-inflammatory agent or for use in the prevention, amelioration or treatment of an autoimmune of inflammatory disease or condition.

An eighth aspect of the invention provides a process for the preparation of an Agent of the invention including the step of:
(a) where the Agent is a compound of formula I or II, or of formula Ib or IIb wherein X is —CH═CHCO—, condensing a compound of formula IV with a compound of formula V in the presence of a suitable amide coupling agent, and, where Y is N, deprotection to give the desired compound of formula I (or corresponding compound of formula II, Ib or IIb): 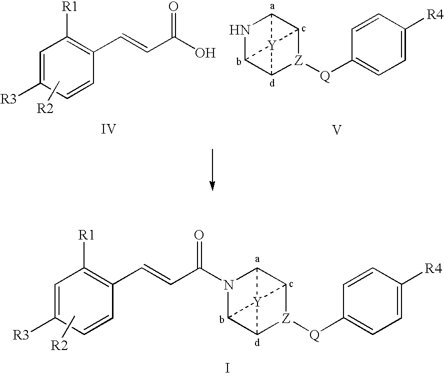
(b) where the agent is a compound of formula Ia or II, or a compound of formula Ib or IIb wherein X is —OCH₂CO—, or —NCH₂CO—, reacting a compound of formula X with a compound of formula IX in the presence of a strong base in an inert organic solvent: 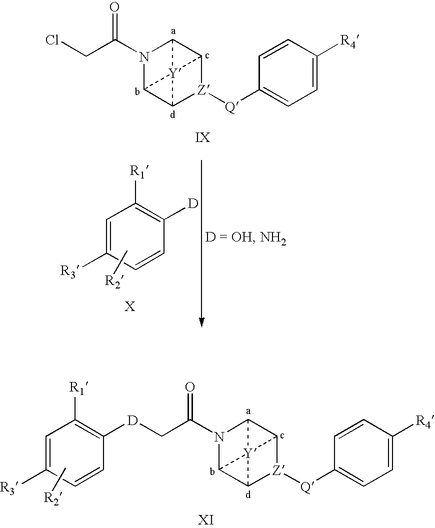
or
(c) where the agent is a compound of formula I or II, or of formula Ib or IIb wherein X is —CH═CHCO—, reacting a compound of formula X with a compound of formula XII in the presence of a suitable reagent such as a palladium catalyst and a base to produce the desired compound of formula I: 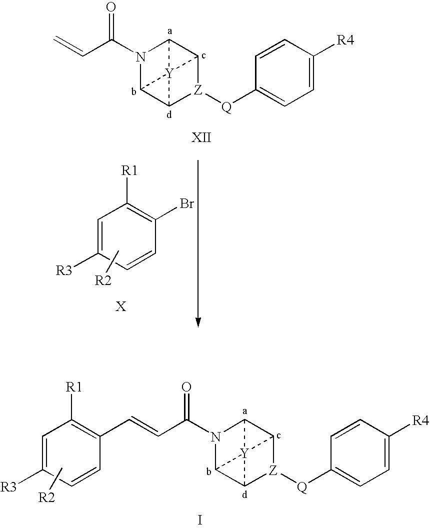
or
(d) where the agent is compound wherein R₁, R₁′ or R₁″ is denoted by a group of the following formula: 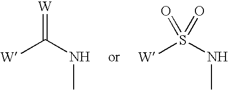
wherein W is O or a nitrogen carrying optional substitutents and W′ represents optional substitutents,
reacting a corresponding compound of formula XII or XIII: 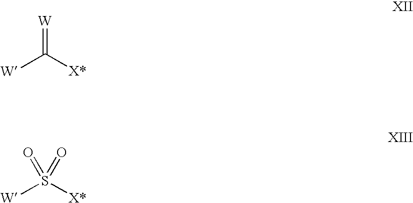
wherein X* represents a leaving group, for example chloro,
with a compound of formula XV: 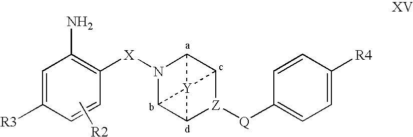
to produce the desired compound of the invention.

In step (a), a suitable amide coupling reagent is EDCl.

In each of cases (a), (b) and (c) and (d), the process may further include the step of temporarily protecting any interfering reactive groups and/or then isolating the resulting compound of the invention.

In more detail, Agents of the Invention may for example be prepared by processes as described below:
1) By condensing a compound of formula IV with a compound of formula V in the presence of a suitable agent, e.g. EDCl, followed by deprotection to give the desired compound VI: 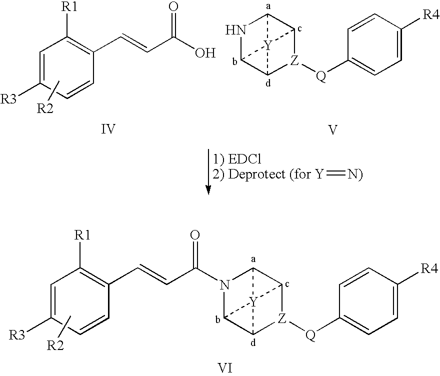
2) By reacting a compound of formula X with a compound of formula IX in the presence of a suitable reagent such as KN(TMS)₂, wherein the compound of formula IX is prepared by reacting a compound of formula VII with a compound of formula V as shown below: 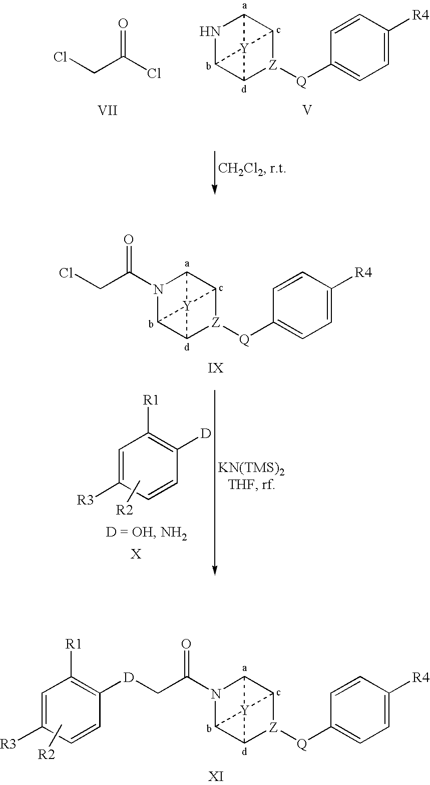
3) By reacting a compound of formula X with a compound of formula XII in the presence of a suitable reagent such as palladium acetate, triarylphosphine and a base such as triethylamine, wherein the compound of formula XII may be prepared by reaction between a compound of formula VII and a compound of formula V in the presence of a base such as triethylamine: 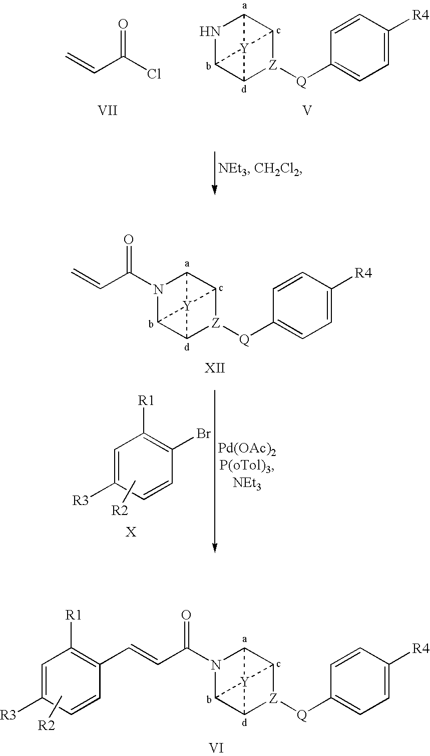
4) The compounds of formula V (Y=—CH₂OCH₂, —CH₂NRCH₂—) may themselves be prepared by the following synthesis:

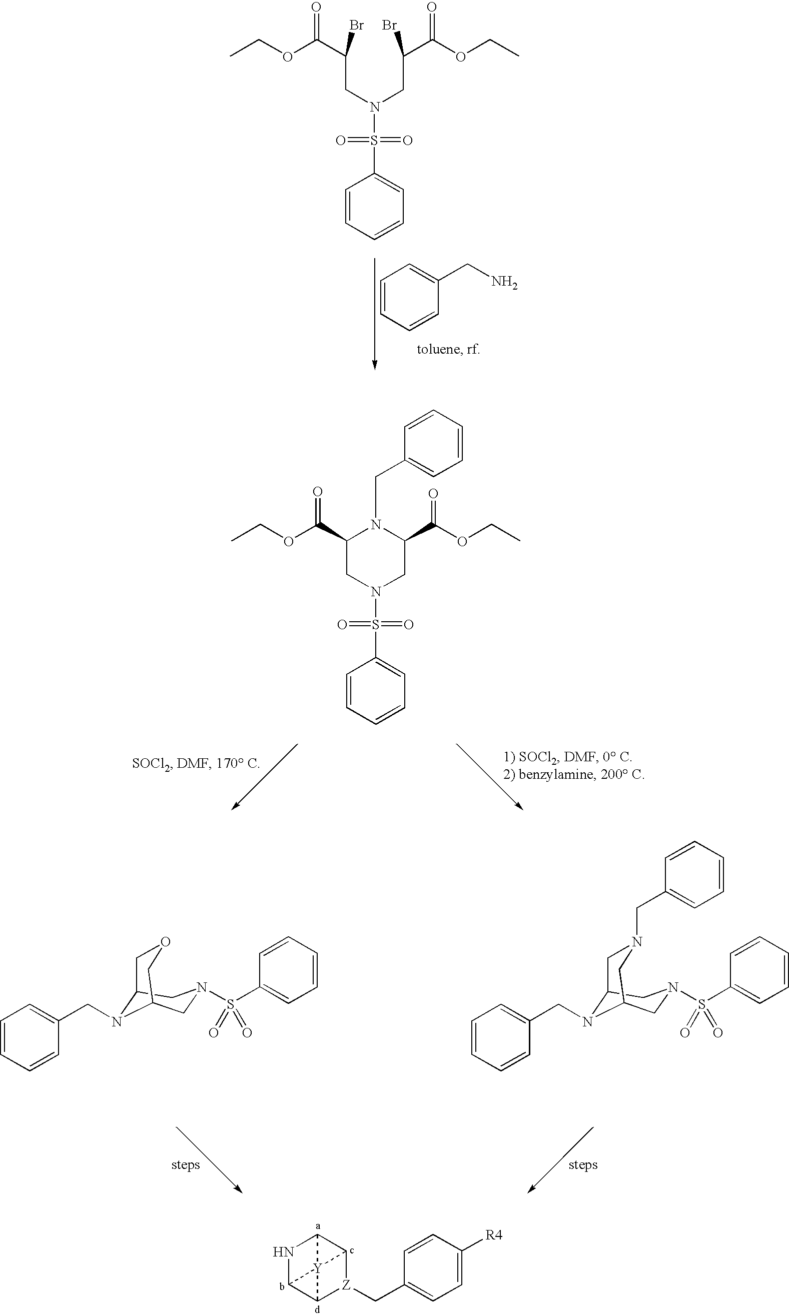

Compounds of formula V wherein Y is —(CH₂)ₙ— may be synthesized by known methods.

### EXPERIMENTAL SECTION

Abbreviations:

Ac₂O: Acetic anhydride

BOC: tert.-Butyloxycarbonyl

DCC: Dicyclohexyl-carbodiimide

DCM: Dichloromethane

DMAP: Dimethyl-pyridin-4-yl-amine

DME: 1,2-Dimethoxyethane

DMF: N,N-Dimethyl formamide

EDCl: (3-Dimethylamino-propyl)-ethyl-carbodiimide hydrochloride

HCl: Hydrochloric acid

HOBT: Benzotriazol-1-ol

NaOH: Sodium hydroxide

NEt₃: Triethylamine

TBME tert.-Butyl-methylether

TFA: Trifluoro-acetic acid

THF: Tetrahydrofuran

### EXAMPLES

The following examples are for illustrative purposes only and are not intended to limit in any way the scope of the claimed invention:

### Example 1

### (E)-N-(5-Chloro-2-{3-[3-(4-fluorobenzyl)-3,8-diazabicyclo[3.2.1]oct-8-yl]-3-oxopropenyl}-phenyl)acetamide

### a) (E)-3-(2-tert-Butoxycarbonylamino-4-chlorophenyl)-acrylic acid methyl ester

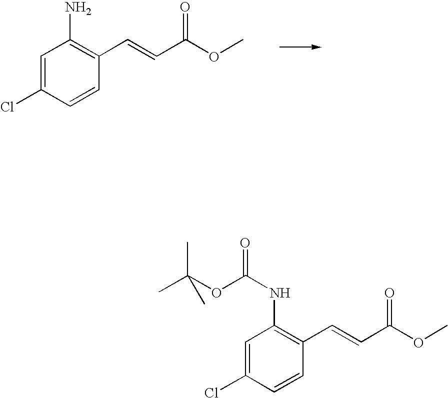

(E)-3-(2-Amino-4-chlorophenyl)-acrylic acid methyl ester (Carling, Robert W.; et al. J. Med. Chem. (1993), 36(22), 3397-408) (3.3 g, 15.6 mmol) in THF (63 ml) is combined with (BOC)2O (6.8 g, 31.2 mmol) and refluxed for 4 h. THF is evaporated and a second portion of (BOC)2O added (6.8 g, 31.2 mmol). The mixture is heated to 100° C. for 18 h. Recrystallisation from TBME/hexanes rendered the title compound as colorless crystals (4.6 g; 94%).

1H-NMR (400 MHz; DMSO-d6): 1.46 (s, 9H); 3.72 (s, 3H); 6.58 (d, 1H); 7.25 (dd, 1H); 7.47 (d, 1H); 7.72 (d, 1H); 7.82 (d, 1H); 9.33 (bs, 1H, NH).

MS (m/z) EI: 311 (M+, 20); 238 (10); 255 (20); 180 (70); 152 (65).

### b) (E)-3-(2-tert-Butoxycarbonylamino-4-chlorophenyl)-acrylic acid

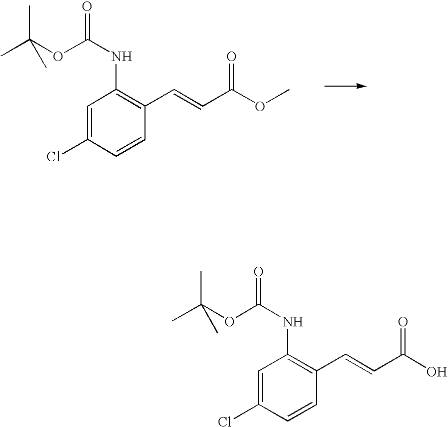

(E)-3-(2-tert-Butoxycarbonylamino-4-chlorophenyl)-acrylic acid methyl ester (4.6 g, 14.7 mmol) is dissolved in MeOH (300 ml), 2N NaOH (11 ml, 22 mmol) and water (147 ml) added and stirred at 50° C. for 1 h. The clear reaction mixture is concentrated to ˜150 ml, acidified to pH 3 and extracted twice with TBME. The combined organic phases are dried over Na2SO4 and evaporated to dryness to yield the title acid as colorless crystals (3.8 g, 87%).

### c) 3-(4-Fluorobenzyl)-3,8-diazabicyclo[3.2.1]octane and 8-(4-Fluorobenzyl)-3,8-diazabicyclo[3.2.1]

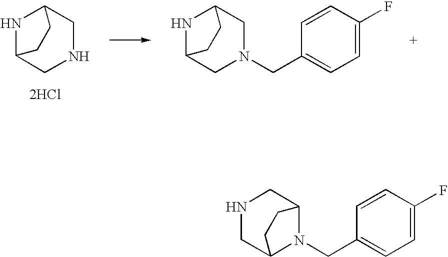

3,8-Diazabicyclo[3.2.1]octane dihydrochloride (MicroChemistry Building Blocks, Moscow) (300 mg; 1.6 mmol), 4-fluorobenzylchloride (0.18 ml; 1.6 mmol) and NaHCO3 (685 mg; 8.1 mmol) are refluxed in EtOH (6 ml) for 2.5 h. TBME (15 ml) is added, the reaction mixture is filtered, evaporated to dryness and the residue purified by chromatography (SiO2, TBME/MeOH/NH3conc 90/1512) to yield an inseparable mixture of the title compounds as light yellow oil (160 mg; 46%), which is used in the next step.

### d) (E)-(5-Chloro-2-{3-[3-(4-fluorobenzyl)-3,8-diazabicyclo[3.2.1]oct-8-yl-3-oxopropenyl}phenyl)-carbamic acid tert-butyl ester (Compound A; BL 5334-II and (E)-(5-Chloro-2-{3-[8-(4-fluorobenzyl)-3,8-diazabicyclo[3.2.1]oct-3-yl-3-oxopropenyl}phenyl)-carbamic acid tert-butyl ester (Compound B)

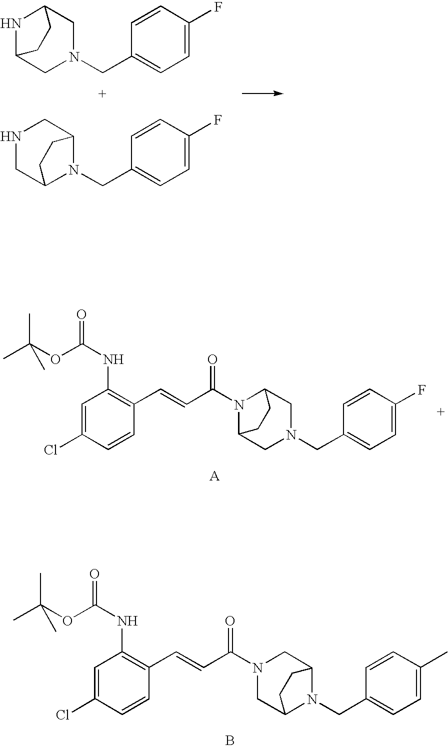

The mixture of 3-(4-fluorobenzyl)-3,8-diazabicyclo[3.2.1]octane and 8-(4-fluorobenzyl)-3,8-diazabicyclo[3.2.1] from the previous reaction (240 mg; 1.1 mmol), (E)-3-(2-tert-butoxycarbonyl amino-4-chlorophenyl)-acrylic acid (324 mg; 1.1 mmol) and EDCl.HCl (210 mg; 1.1 mmol) are dissolved in CH2Cl2 (6 ml) and stirred at room temperature for 18 h. The reaction mixture is purified via chromatography (SiO2; acetone/hexanes 15/85) to yield B (98 mg; 18%; colorless foam), which is eluted first, followed by A (371 mg; 68%) as colorless crystals.

Compound A. 1H-NMR (400 MHz; DMSO-d6), δ (ppm): 1.47 (s, 9H); 1.67-2.05 (m, 4H); 2.18 (dd, 2H); 2.68 (dd, 2H); 3.46 (s, 2H); 4.55 (d, 1H); 4.68 (bd, 1H); 7.06 (d, 1H); 7.16 (t, 2H); 7.25 (dd, 1H); 7.35 (dd, 2H); 7.46 (s, 1H); 7.66 (d, 1H); 7.89 (d, 1H); 9.23 (s, 1H).

MS (m/z) ES+: 500.2 (MH+, 100).

Compound B. 1H-NMR (400 MHz; DMSO-d6), δ (ppm): 0.81-0.91 (m, 1H); 1.48 (s, 9H); 1.53-1.62 (m, 1H); 1.95 (bs, 2H); 2.83 (d, 1H); 3.18 (bs, 2H); 3.28 (d, 1H); 3.51 (d, 2H); 3.96 (d, 1H); 4.13 (d, 1H); 7.11 (d, 1H); 7.16 (t, 2H); 7.25 (dd, 1H); 7.41-7.46 (m, 3H); 7.63 (d, 1H); 7.87 (d, 1H); 9.23 (s, 1H).

MS (m/z) ES+: 500.2 (MH+, 100).

### e) (E)-3-(2-Amino-4-chlorophenyl)-1-[3-(4-fluorobenzyl)-3,8-diazabicyclo[3.2.1]oct-8-yl]propenone

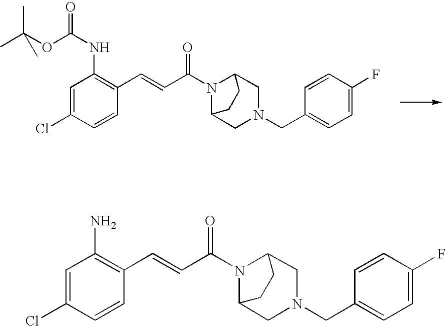

(E)-(5-Chloro-2-{3-[3-(4-fluorobenzyl)-3,8-diazabicyclo[3.2.1]oct-8-yl-3-oxopropenyl}phenyl)-carbamic acid tert-butyl ester (A from the reaction above; 365 mg; 0.7 mmol) is dissolved in EtOH/HClconc. (4 ml/4 ml) and stirred for 2 min., poured on a saturated solution of Na2CO3 and extracted with TBME three times. The combined organic phases are dried over Na2SO4 and evaporated to dryness to yield the title compound as yellow foam (292 mg; 100%).

1H-NMR (400 MHz; DMSO-d6), δ (ppm): 1.68-1.97 (m, 4H); 2.18 (dd, 2H); 2.67 (dd, 2H); 3.48 (s, 2H); 4.55 (d, 1H); 4.63 (bd, 1H); 5.75 (s, 2H, NH2); 6.54 (dd, 1H); 6.73 (d, 1H); 6.89 (d, 1H); 7.17 (t, 2H); 7.35 (dd, 2H); 7.55 (d, 1H); 7.68 (d, 1H).

MS (m/z) ES+: 400.2 (MH+, 100).

### f) (E)-N-(5-Chloro-2-{3-[3-(4-fluorobenzyl)-3,8-diazabicyclo[3.2.1]oct-8-yl]-3-oxopropenyl}-phenyl)acetamide

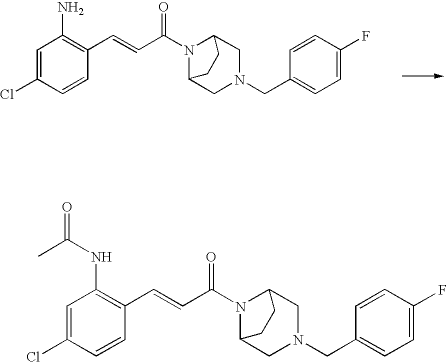

(E)-3-(2-Amino-4-chlorophenyl)-1-[3-(4-fluorobenzyl)-3,8-diazabicyclo[3.2.1]oct-8-yl]-propenone (50 mg; 0.12 mmol) and NEt3 (0.17 ml; 1.2 mmol) are dissolved in THF (4 ml) and treated with acetyl chloride (0.088 ml; 1.2 mmol). The reaction mixture is refluxed for 2 min. and kept at room temperature for 10 min., poured on a saturated solution of Na2CO3 and extracted with TBME three times. The combined organic phases are dried over Na2SO4 and evaporated to dryness. Purification via chromatography (SiO2; TBME/MeOH/NH3conc 9713/0.3) delivered the title compound as colorless crystals (31 mg; 56%).

1H-NMR (400 MHz; DMSO-d6), δ (ppm): 1.68-1.79 (m, 1H); 1.82-1.97 (m, 3H); 2.09 (s, 3H); 2.15 (dd, 2H); 2.68 (bt, 2H); 3.47 (s, 2H); 4.55 (d, 1H); 4.70 (s, 1H); 7.11 (d, 1H); 7.17 (t, 2H); 7.30 (dd, 1H); 7.36 (dd, 2H); 7.59 (d, 1H); 7.68 (d, 1H); 7.93 (d, 1H); 9.93(s, 1H).

MS (m/z) ES+: 442.2 (MH+, 50).

### Example 2

### (E)-N-(5-Chloro-2-{3-[3-(4-fluorobenzyl)-3,8-diazabicyclo[3.2.1]oct-8-yl]-3-oxopropenyl}-phenyl)-N′-cyanoguanidine

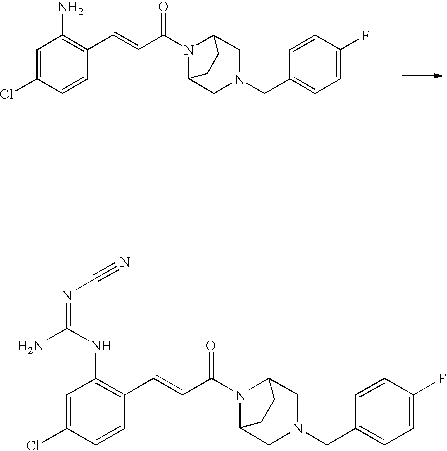

(E)-3-(2-Amino-4-chlorophenyl)-1-[3-(4-fluorobenzyl)-3,8-diazabicyclo[3.2.1]oct-8-yl]-propenone (100 mg; 0.25 mmol) is suspended in ethoxyethanol/water (4 ml/2 ml). The reaction mixture is heated to reflux and treated with NaN(CN)₂ (89 mg; 1 mmol) followed by 2N HCl (0.5 ml; 1 mmol). After 5 min. at reflux a second portion of NaN(CN)₂ (178 mg; 2 mmol) followed by 2 N HCl (1 ml; 2 mmol) is added and refluxed for 5 min. The reaction mixture is poured on a saturated solution of Na2CO3 and extracted with TBME three times. The combined organic phases are dried over Na2SO4, evaporated to dryness and purified via preparative HPLC (XTerra, RP18, 7 μm, acetonitrile/water) to deliver the title compound as colorless crystals (12 mg; 10%).

1H-NMR (400 MHz; DMSO-d6), δ (ppm): 1.71-1.98 (m, 4H); 2.18 (dd, 2H); 2.68 (dd, 2H); 3.48 (s, 2H); 4.55 (d, 1H); 4.70 (bs, 1H); 7.09-7.22 (m, 4H); 7.30-7.38 (m, 3H); 7.43 (d, 1H); 7.58 (d, 1H); 7.93 (d, 1H); 9.13 (bs, 1H).

MS (m/z) ES+: 467.1 (MH+, 100).

### Example 3

### (E)-N-(5-Chloro-2-{3-[3-(4-fluorobenzyl)-3,8-diazabicyclo[3.2.1]oct-8-yl]-3-oxopropenyl}-phenyl)-2-dimethylaminoacetamide

### a) (E)-2-Chloro-N-(5-chloro-2-{3-[3-(4-fluorobenzyl)-3,8-diazabicyclo[3.2.1]oct-8-yl]-3-oxopronenyl}-phenyl)acetamide hydrochloride

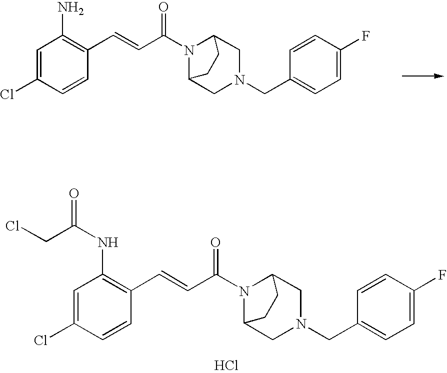

(E)-3-(2-Amino-4-chlorophenyl)-1-[3-(4-fluorobenzyl)-3,8-diazabicyclo[3.2.1]oct-8-yl]-propenone (50 mg; 0.12 mmol) is dissolved in THF (1 ml) and treated with chloroacetylchloride (0.011 ml; 0.14 mmol) and stirred at room temperature for 1 h. TBME is added to the reaction mixture, the white precipitate filtered, washed and dried to yield the desired product (55 mg; 85%).

1H-NMR (400 MHz; DMSO-d6), δ (ppm): 1.83-2.24 (m, 4H); 3.10-3.35 (m, 4H); 4.33 (bs, 2H); 4.36 (s, 2H); 4.76 (bs, 1H); 4.94 (bs, 1H); 7.18 (d, 1H); 7.30 (bt, 2H); 7.40 (bd, 1H); 7.55 (d, 1H); 7.68-7.78 (m, 3H); 7.94 (d, 1H); 10.30 (bs, 2H).

MS (m/z) ES+: 476.1 (MH+, 100).

### b) (E)-N-(5-Chloro-2-{3-[3-(4-fluorobenzyl)-3,8-diazabicyclo[3.2.1]oct-8-yl]-3-oxopropenyl}-phenyl)-2-dimethylaminoacetamide

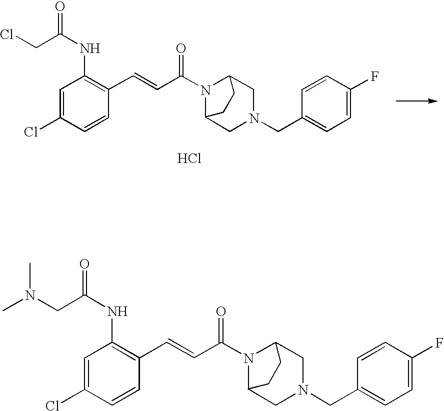

(E)-2-Chloro-N-(5-chloro-2-{3-[3-(4-fluorobenzyl)-3,8-diazabicyclo[3.2.1]oct-8-yl]-3-oxopropenyl}-phenyl)acetamide hydrochloride (50 mg; 0.1 mmol) is suspended in THF (2 ml) and treated with an excess of dimethylamine (˜0.5 ml). The reaction mixture is poured on a silica gel column and purified (TBME/MeOH/NH3conc 95/5/0.5) to give the title compound as a colorless foam (48 mg; 95%).

1H-NMR (400 MHz; DMSO-d6), δ (ppm): 1.68-1.98 (m, 4H); 2.18 (dd, 2H); 2.33 (s, 6H); 3.18 (dd, 2H); 3.12 (s, 2H); 3.48 (d, 2H); 4.55 (d, 1H); 4.70 (bs, 1H); 7.10 (d, 1H); 7.16 (t, 2H); 7.30 (dd, 1H); 7.36 (dd, 2H); 7.61 (d, 1H); 7.65 (d, 1H); 7.92 (d, 1H); 9.83 (s, 1H).

MS (m/z) ES+: 485.2 (MH+, 100).

### Example 4

### (E)-(5-Chloro-2-{3-[3-(4-fluorobenzyl)-3,8-diazabicyclo[3.2.1]oct-8-yl]-3-oxopropenyl}-phenyl)-urea

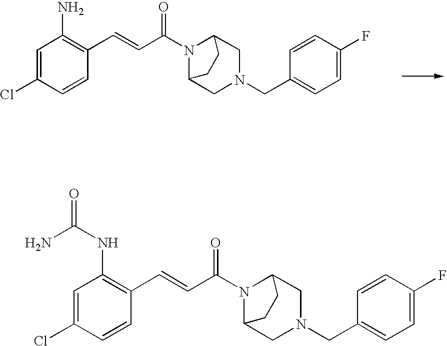

(E)-3-(2-Amino-4-chlorophenyl)-1-[3-(4-fluorobenzyl)-3,8-diazabicyclo[3.2.1]oct-8-yl]-propenone (50 mg; 0.12 mmol) is dissolved in HOAc (1 ml). Water (2 ml) is added, followed by NaOCN (100 mg; 1.5 mmol). The reaction mixture is kept at room temperature for 20 min., then poured on a saturated solution of Na2CO3. The white precipitate is filtered and purified further by chromatography (SiO2; acetone/hexanes 6/4 to 8/2) to render the target compound as colorless crystals (22 mg; 40%).

1H-NMR (400 MHz; DMSO-d6), δ (ppm): 1.58-1.98 (m, 4H); 2.17 (dd, 2H); 2.68 (dd, 2H); 3.46 (s, 2H); 4.56 (d, 1H); 4.69 (bs, 1H); 6.25 (s, 2H, NH2); 7.04 (d, 1H); 7.05 (d, 1H); 7.15(t, 2H); 7.35 (dd, 2H); 7.70 (d, 1H); 7.78 (d, 1H); 7.96 (d, 1H); 8.43 (s, 1H, NH).

MS (m/z) ES+: 443.2 (MH+, 100).

### Example 5

### N-(5-Chloro-2-{(E)-3-[3-(4-fluoro-benzyl)-3,8-diaza-bicyclo[3.2.1]oct-8-yl]-3-oxo-propenyl}-phenyl)-methanesulfonamide

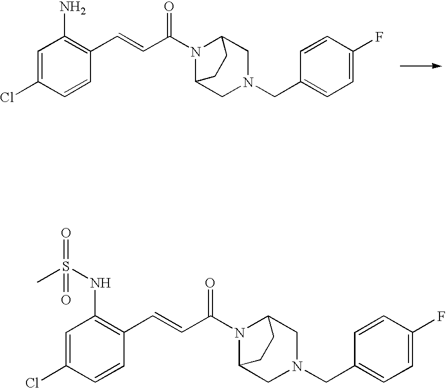

(E)-3-(2-Amino-4-chlorophenyl)-1-[3-(4-fluorobenzyl)-3,8-diazabicyclo[3.2.1]oct-8-yl]-propenone (100 mg; 0.25 mmol) in pyridine (2 ml) is treated with methanesulfonyl chloride (0.06 ml; 0.75 mmol) for 1 h at room temp. The reaction is evaporated to dryness and purified by chromatography (SiO2; TBME/MeOH/NH3conc 95/4.5/0.5) to yield the title compound as colorless crystals (20 mg; 16%).

1H-NMR (400 MHz; DMSO-d6), δ (ppm): 1.70-1.78 (m, 1H); 1.82-1.98 (m, 3H); 2.13 (d, 1H); 2.20 (d, 1H); 2.67 (dt, 2H); 3.04 (s, 3H); 3.45 (s, 2H); 4.53 (bd, 1H); 4.67 (bd, 1H); 7.10 (d, 1H); 7.12 (t, 2H); 7.30-7.40 (m, 4H); 7.81 (d, 1H); 7.96(d, 1H); 9.70 (bs, 1H).

MS (m/z) ES+: 478 (MH+).

### Example 6

### N-(5-Chloro-2-{(E)-3-[3-(4-fluoro-benzyl)-3,8-diaza-bicyclo[3.2.1]oct-8-yl]-3-oxo-propenyl}-phenyl)-2-methoxy-acetamide

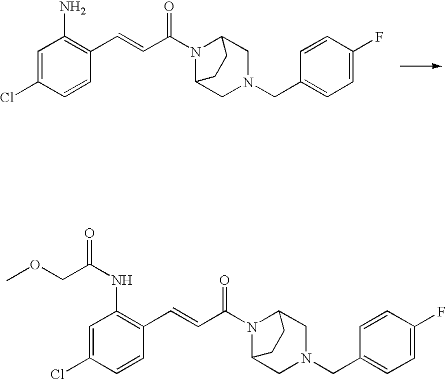

The target compound is prepared in analogy to Example 1f), replacing acetyl chloride by methoxyacetyl chloride. Purification by chromatography (SiO2; acetone/hexanes 3/7) yielded the title compound as colorless crystals (67 mg; 54%)

1H-NMR (400 MHz; DMSO-d6), δ (ppm): 1.68-1.78 (m, 1H); 1.80-1.97 (m, 3H); 2.15 (bd, 2H); 2.65 (bd, 2H); 3.40 (s, 3H); 3.45 (s, 2H); 4.03 (s, 2H); 4.52 (bd, 1H); 4.68 (bd, 1H); 7.07 (d, 1H); 7.12 (bt, 2H); 7.32 (m, 3H); 7.50 (s, 1H); 7.57 (d, 1H); 7.92 (d, 1H); 9.78 (s, 1H).

MS (m/z) ES+: 472.2 (MH+).

### Example 7

### 1-(5-Chloro-2-{(E)-3-[3-(4-fluoro-benzyl)-3,8-diaza-bicyclo[3.2.1]oct-8-yl]-3-oxo-propenyl}-phenyl)-3-methyl-urea

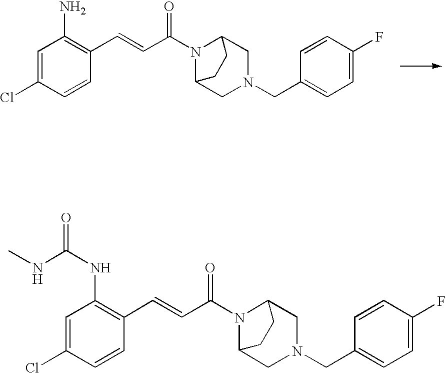

The target compound is prepared in analogy to Example 23f), purified via chromatography (SiO2; acetone/hexanes 3/7) and yielded the title compound as colorless crystals (52 mg; 57%).

1H-NMR (400 MHz; DMSO-d6), δ (ppm): 1.67-1.78 (m, 1H); 1.82-1.96 (m, 3H); 2.13 (d, 1H); 2.18 (d, 1H); 2.61-2.70 (m, 5H); 3.44 (s, 2H); 4.53 (bd, 1H); 4.65 (bd, 1H); 6.53 (m, 1H); 6.98-7.07 (m, 2H); 7.10-7.16 (m, 2H); 7.31 (m, 2H); 7.65 (d, 1H); 7.73 (d, 1H); 7.92 (d, 1H); 8.35 (s, 1H).

MS (m/z) ES+: 457 (MH+); 400 (35).

### Example 8

### 3-(5-Chloro-2-{(E)-3-[3-(4-fluoro-benzyl)-3,8-diaza-bicyclo[3.2.1]oct-8-yl]-3-oxo-propenyl}-phenyl)-1,1-dimethyl-urea

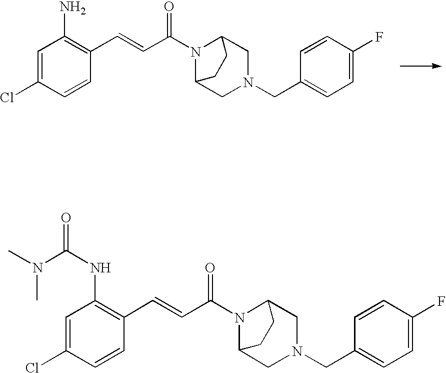

(E)-3-(2-Amino-4-chlorophenyl)-1-[3-(4-fluorobenzyl)-3,8-diazabicyclo[3.2.1]oct-8-yl]-propenone (80 mg; 0.20 mmol) is dissolved in THF (4 ml) and treated at room temp, with KN(TMS)2 (0.83 M in toluene; 0.72 ml; 0.060 mmol) for 1-2 min. Dimethylcarbamoyl chloride (0.055 ml; 0.060 mmol) is added, and 2 min. later the reaction mixture poured on a saturated solution of Na2CO3 and extracted with TBME. The combined organic phases are dried over Na2SO4, filtered, evaporated to dryness and the resulting product purified via chromatography (SiO2; acetone/hexanes 2/8 to 4/6) to yield the title compound as off-white foam (53 mg; 57%).

1H-NMR (400 MHz; DMSO-d6), δ (ppm): 1.69-1.78 (m, 1H); 1.83-1.95 (m, 3H); 2.10-2.20 (m, 2H); 2.65 (bt, 2H); 2.92 (s, 6H); 3.47 (s, 2H); 4.52 (bd, 1H); 4.66 (bd, 1H); 7.02 (d, 1H); 7.12 (t, 2H); 7.21 (dd, 1H); 7.32 (m, 3H); 7.55 (d, 1H); 7.86 (d, 1H); 8.30 (s, 1H).

MS (m/z) ES+: 471 (MH+); 426 (15); 400 (50).

### Example 9

### 1-(5-Chloro-2-{(E)-3-[3-(4-fluoro-benzyl)-3,8-diaza-bicyclo[3.2.1]oct-8-yl]-3-oxo-propenyl}-phenyl)-3-ethyl-urea

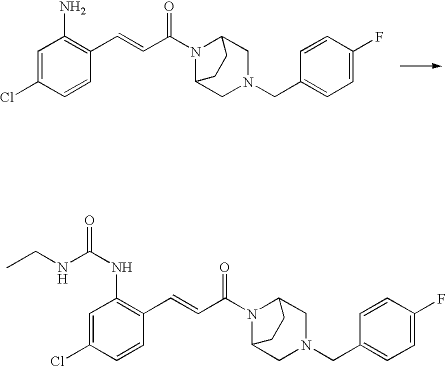

The target compound is prepared in analogy to Example 23f) substituting methylamine by ethylamine and purified via recrystallisation from TBME, yielding the title compound as colorless crystals (45 mg; 49%).

1H-NMR (400 MHz; DMSO-d6), δ (ppm): 1.08 (t, 3H); 1.68-1.77 (m, 1H); 1.81-1.95 (m, 3H); 2.13 (d, 1H); 2.20 (d, 1H); 2.67 (bt, 2H); 3.11 (m, 2H); 3.45 (s, 2H); 4.53 (bd, 1H); 4.66 (bd, 1H); 6.67 (bt, 1H); 7.00 (m, 1H); 7.05 (m, 1H); 7.13 (t, 2H); 7.32 (m, 2H); 7.66 (d, 1H); 7.73 (d, 1H); 7.97 (s, 1H); 8.27 (s, 1H).

MS (m/z) ES+: 471 (MH+); 426 (10); 400 (90).

### Example 10

### 1-(5-Chloro-2-{(E)-3-[3-(4-fluoro-benzyl)-3,8-diaza-bicyclo[3.2.1]oct-8-yl]-3-oxo-propenyl}-phenyl)-3-propyl-urea

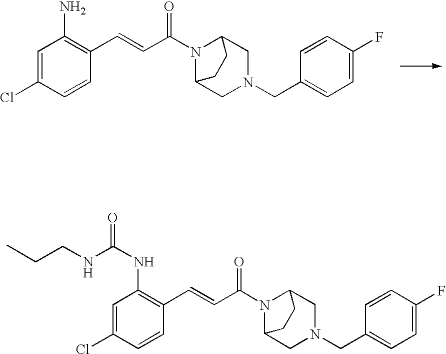

The target compound is prepared in analogy to Example 23f), substituting methylamine by 1-propylamine and purified via chromatography (SiO2; acetone/hexanes 15/85) yielding the title compound as colorless crystals (84 mg; 87%).

1H-NMR (400 MHz; DMSO-d6), δ (ppm): 0.90 (t, 3H); 1.41-1.52 (m, 2H); 1.68-1.78 (m, 1H); 1.80-1.98 (m, 3H); 2.13 (d, 1H); 2.20 (d, 1H); 2.66 (bt, 2H); 3.02-3.09 (m, 2H); 3.46 (s, 2H); 4.55 (bd, 1H); 4.66 (bd, 1H); 6.70 (t, 1H); 7.00 (m, 2H); 7.13 (t, 2H); 7.32 (dd, 2H); 7.66 (d, 1H); 7.73 (d, 1H); 7.98 (d, 1H); 8.28 (s, 1H).

MS (m/z) ES+: 485 (MH+); 426 (15); 400 (90).

### Example 11

### 1-(5-Chloro-2-{(E)-3-[3-(4-fluoro-benzyl)-3,8-diaza-bicyclo[3.2.1]oct-8-yl]-3-oxo-propenyl}-phenyl)-3-isopropyl-urea

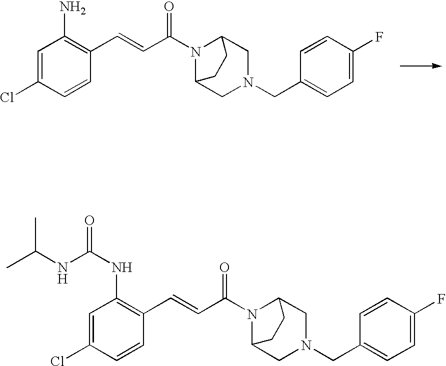

The target compound is prepared in analogy to Example 23f), substituting methylamine by isopropylamine and purified via chromatography (SiO2; acetone/hexanes 15/85) yielding the title compound as colorless crystals (45 mg; 47%).

1H-NMR (400 MHz; DMSO-d6), δ (ppm): 1.12 (d, 6H); 1.68-1.78 (m, 1H); 1.80-1.98 (m, 3H); 2.13 (d, 1H); 2.20 (d, 1H); 2.66 (bt, 2H); 3.45 (s, 2H);); 3.70-3.80 (m, 1H); 4.55 (bd, 1H); 4.66 (bd, 1H); 6.64 (d, 1H); 6.98-7.03 (m, 2H); 7.13 (t, 2H); 7.31 (dd, 2H); 7.65 (dd, 1H); 7.71 (d, 1H); 8.02 (s, 1H); 8.18 (s, 1H).

MS (m/z) ES+: 485 (MH+); 400 (60).

### Example 12

### 1-(5-Chloro-2-{(E)-3-[3-(4-fluoro-benzyl)-3,8-diaza-bicyclo[3.2.1]oct-8-yl]-3-oxo-propenyl}-phenyl)-3-cyclopropyl-urea

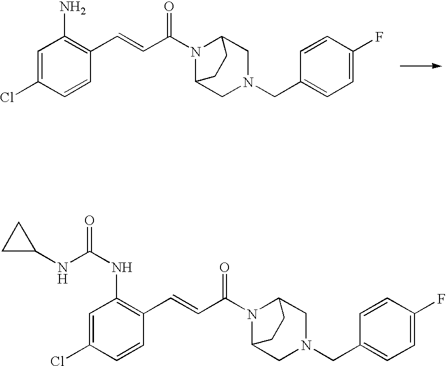

The target compound is prepared in analogy to Example 23f), substituting methylamine by cyclopropylamine and purified via recrystallisation from TBME yielding the title compound as colorless crystals (47 mg; 47%).

1H-NMR (400 MHz; DMSO-d6), δ (ppm): 0.42 (m, 2H); 0.68 (m, 2H); 1.68-1.78 (m, 1H); 1.80-1.98 (m, 3H); 2.13 (d, 1H); 2.20 (d, 1H); 2.50-2.55 (m, 1H); 2.66 (bt, 2H); 3.45 (s, 2H);); 4.55 (bd, 1H); 4.66 (bd, 1H); 6.89 (bd, 1H); 7.00-7.06 (m, 2H); 7.13 (t, 2H); 7.31 (dd, 2H); 7.63 (d, 1H); 7.75 (d, 1H); 7.98 (s, 1H); 8.18 (s, 1H).

MS (m/z) ES+: 483 (MH+); 400 (15).

### Example 13

### 1-(5-Chloro-2-{(E)-3-[3-(4-fluoro-benzyl)-3,8-diaza-bicyclo[3.2.1]oct-8-yl]-3-oxo-propenyl}-phenyl)-3-(tetrahydro-pyran-4-yl)-urea

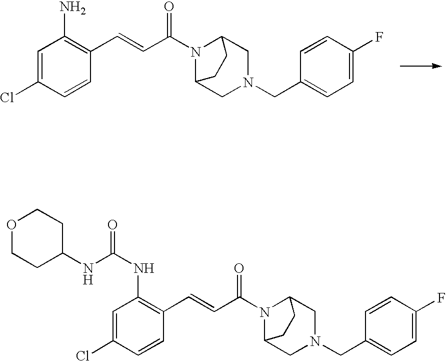

The target compound is prepared in analogy to Example 23f), substituting methylamine by tetrahydropyran-4-ylamine and purified by chromatography (SiO2; acetone/hexanes 3/7) followed by a second chromatography (XTerra, RP18, 7 μm, MeCN/water 40/60 to 100/0) to yield the title compound as a white amorphous powder (64 mg; 61%).

1H-NMR (400 MHz; DMSO-d6), δ (ppm): 1.32-1.45 (m, 2H); 1.70-1.95 (m, 6H); 2.14 (d, 1H); 2.19 (d, 1H); 2.67 (bt, 2H); 3.38 (bt, 2H); 3.45 (s, 2H); 3.60-3.70 (m, 1H); 3.78-3.85 (m, 2H); 4.55 (bd, 1H); 4.65 (bd, 1H); 6.80 (d, 1H); 7.00-7.05 (m, 2H); 7.13 (t, 2H); 7.32 (dd, 2H); 7.54 (d, 1H); 7.73 (d, 1H); 8.00 (s, 1H); 8.22 (s, 1H).

MS (m/z) ES+: 527 (MH+, 45); 400 (100).

### Example 14

### 3-Oxo-piperazine-1-carboxylic acid (5-chloro-2-{(E)-3-[3-(4-fluoro-benzyl)-3,8-diaza-bicyclo[3.2.1]oct-8-yl]-3-oxo-propenyl}-phenyl)-amide

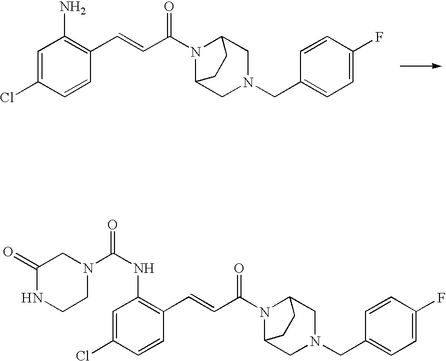

The target compound is prepared in analogy to Example 23f), substituting methylamine by piperazine-2-one and purified by chromatography (SiO2; acetone/hexanes 1/1 to 1/0) to yield the title compound as colorless crystals (50 mg; 48%).

1H-NMR (400 MHz; DMSO-d6), δ (ppm): 1.70-1.78 (m, 1H); 1.80-1.95 (m, 3H); 2.10-2.21 (m, 2H); 2.65 (bd, 1H); 2.68 (bd, 1H); 3.27 (bs, 1H); 3.45 (s, 2H); 3.62 (bt, 2H); 4.00 (s, 2H); 4.50 (bd, 1H); 4.67 (bd, 1H); 7.03 (d, 1H); 7.13 (t, 1H); 7.25 (dd, 1H); 7.30-7.50 (m, 3H); 7.56 (d, 1H); 7.88 (d, 1H); 8.08 (s, 1H).

MS (m/z) ES+: 526 (MH+).

### Example 15

### 2-Oxo-oxazolidine-3-sulfonic acid (5-chloro-2-{(E)-3-[3-(4-fluoro-benzyl)-3,8-diaza-bicyclo[3.2.1]oct-8-yl]-3-oxo-propenyl}-phenyl)-amide

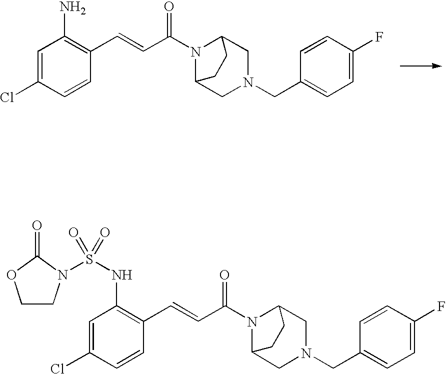

Chlorosulfonyl isocyanate (0.022 ml; 0.25 mmol) in CH2Cl2 (3 ml) is cooled to 0° C. and treated with 2-chloroethanol for 1 h at 0° C. (E)-3-(2-Amino-4-chlorophenyl)-1-[3-(4-fluorobenzyl)-3,8-diazabicyclo[3.2.1]oct-8-yl]-propenone (100 mg; 0.25 mmol) dissolved in CH2Cl2 (2 ml) and NEt3 (0.14 ml; 0.1 mmol) is added to the reaction mixture, warmed to room temp. and stirred for 12 h. The mixture is poured on brine and extracted with TBME three times. The combined organic phases are dried over Na2SO4, filtered and evaporated to dryness to yield the ring-open intermediate. The latter is dissolved in CH2Cl2 (2 ml), combined with NEt3 (0.5 ml) and left at room temp. over night, poured on brine and extracted with TBME three times. The combined organic phases are dried over Na2SO4, filtered and evaporated to dryness to deliver the desired product as yellowish crystals (86 mg; 63%).

1H-NMR (400 MHz; DMSO-d6), δ (ppm): 1.68-1.78 (m, 1H); 1.80-1.98 (m, 3H); 2.13 (d, 1H); 2.20 (d, 1H); 2.58 (bd, 1H); 2.65 (bd, 1H); 3.45 (s, 2H);); 3.70 (t, 2H); 4.12 (t, 2H); 4.50 (bd, 1H); 4.60 (bd, 1H); 6.62 (bd, 1H); 6.93 (d, 1H); 7.12 (t, 2H); 7.27 (d, 1H); 7.32 (dd, 2H); 7.52 (d, 1H); 7.97 (d, 1H).

MS (m/z) ES+: 549 (MH+, 30); 400 (75); 382 (100); 221 (30).

### Example 16

### N-(5-Chloro-2-{(E)-3-[8-(4-fluoro-benzyl)-3,8-diaza-bicyclo[3.2.1]oct-3-yl]-3-oxo-propenyl}-phenyl)-methanesulfonamide

### a) (E)-3-(2-Amino-4-chlorophenyl)-1-[8-(4-fluorobenzyl)-3,8-diazabicyclo[3.2.1]oct-3-yl]-propenone

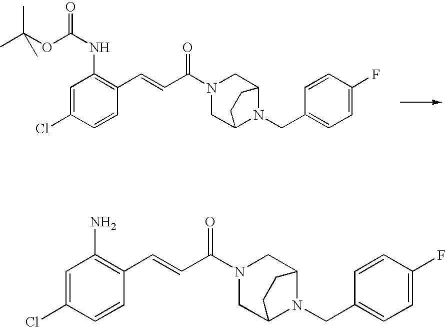

(E)-(5-Chloro-2-{3-[8-(4-fluorobenzyl)-3,8-diazabicyclo[3.2.1]oct-3-yl-3-oxopropenyl}phenyl)-carbamic acid tert-butyl ester (Compound B from Example 1d; 95 mg; 0.19 mmol) is dissolved in EtOH/HClconc (2 ml/2 ml), kept 2 min. at room temp., poured on a saturated solution of Na2CO3 and extracted with TBME three times. The combined organic phases are dried over Na2SO4, evaporated to dryness and yielded the title compound as a yellow foam (77 mg; 97%).

1H-NMR (400 MHz; DMSO-d6), δ (ppm): 1.43-1.61 (m, 2H); 1.95 (bs, 2H); 2.83 (d, 1H); 3.16 (bs, 2H); 3.26 (d, 1H); 3.50 (s, 2H); 3.92 (d, 1H); 4.15 (d, 1H); 5.74 (s, 2H, NH2); 6.54 (dd, 1H); 6.73 (d, 1H); 6.93 (d, 1H); 7.17 (t, 2H); 7.43 (dd, 2H); 7.52 (d, 1H); 7.63 (d, 1H).

MS (m/z) ES+: 400.2 (MH+, 100).

### b) N-(5-Chloro-2-{(E)-3-[8-(4-fluoro-benzyl)-3,8-diaza-bicyclo[3.2.1]oct-3-yl]-3-oxo-propenyl}-phenyl)-methanesulfonamide

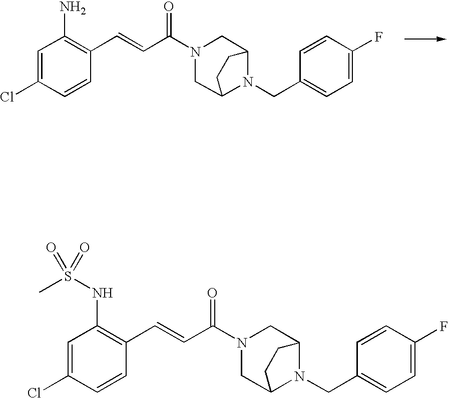

(E)-3-(2-Amino-4-chlorophenyl)-1-[8-(4-fluorobenzyl)-3,8-diazabicyclo[3.2.1]oct-3-yl]-propenone is treated with methanesulfonyl chloride as described in Example 5 and purified via chromatography (SiO2, TBME/MeOH/NH3conc 98/210.6 to 80120/0.6) to deliver the title compound as yellowish foam (40 mg; 66%).

1H-NMR (400 MHz; DMSO-d6), δ (ppm): 1.43-1.70 (m, 4H); 1.90-2.15 (m, 2H); 2.80-2.98 (bs, 1H); 3.03 (s, 3H); 3.20 (bs, 1H); 3.43-3.65 (m, 2H); 4.00 (bs, 1H); 4.18 (bs, 1H); 7.08-7.25 (m, 3H); 7.30-7.55 (m, 4H); 7.78 (d, 1H); 7.93 (d, 1H); 9.73 (bs, 1H).

MS (m/z) ES+: 478 (MH+).

### Example 17

### 1-(5-Chloro-2-{(E)-3-[8-(4-fluoro-benzyl)-3,8-diaza-bicyclo[3.2.1]oct-3-yl]-3-oxo-propenyl}-phenyl)-3-ethyl-urea

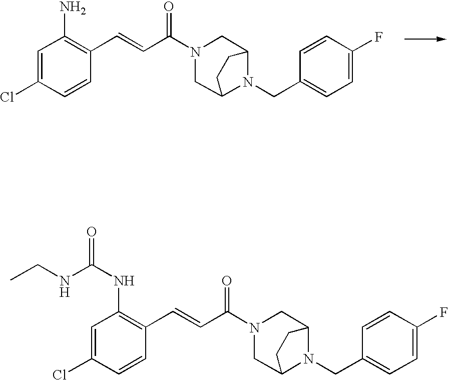

The target compound is prepared in analogy to Example 23f substituting methylamine by ethylamine and purified via chromatography (XTerra, RP18, 7 μm, MeCN/water 40/60 to 100/0) to yield the title compound as a white foam (49 mg; 60%).

1H-NMR (400 MHz; DMSO-d6), δ (ppm): 1.07 (t, 3H); 1.46 (m, 1H); 1.55 (m, 1H); 1.93 (bs, 2H); 2.83 (d, 1H); 3.11 (dd, 2H); 3.14 (m, 2H); 3.28 (d, 1H); 3.50 (bd, 2H); 3.93 (bd, 1H); 4.13 (bd, 1H); 6.66 (bt, 1H); 7.02 (dd, 1H); 7.07 (d, 1H); 7.15 (t, 2H); 7.40 (dd, 2H); 7.62 (d, 1H); 7.72 (d, 1H); 7.97 (d, 1H); 8.26 (s, 1H).

MS (m/z) ES+: 471 (MH+).

### Example 18

### N-(5-chloro-2-{(E)-3-[8-(4-fluoro-benzyl)-3,8-diaza-bicyclo[3.2.1]oct-3-yl]-3-oxo-propenyl}-phenyl)-2-methoxy-acetamide

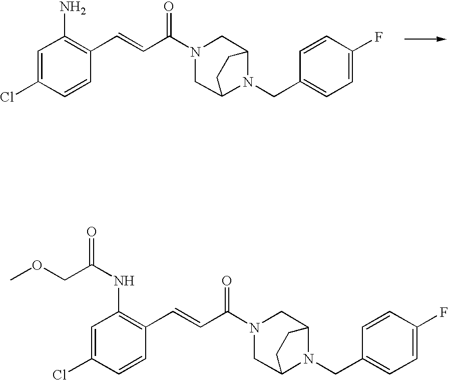

The target compound is prepared in analogy to Example 1f), replacing acetyl chloride by methoxyacetyl chloride. Purification by chromatography (SiO2; acetone/hexanes 3/7 to 8/2) yielded the title compound as colorless crystals (23 mg; 38%).

1H-NMR (400 MHz; DMSO-d6), δ (ppm): 1.46 (m, 1H); 1.55 (m, 1H); 1.93 (bs, 2H); 2.81 (d, 1H); 3.15 (bs, 2H); 3.25 (m, 1H); 3.40 (s, 3H); 3.50 (bd, 2H); 3.93 (bd, 1H); 4.03 (s, 2H); 4.13 (bd, 1H); 7.10-7.18 (m, 3H); 7.390 (dd, 1H); 7.40 (dd, 2H); 7.50 (d, 2H); 7.88 (d, 1H); 9.75 (s, 1H).

MS (m/z) ES+: 472.1 (MH+).

### Example 19

### (5-Chloro-2-{(E)-3-[8-(4-fluoro-benzyl)-3,8-diaza-bicyclo[3.2.1]oct-3-yl]-3-oxo-propenyl}-phenyl)-urea

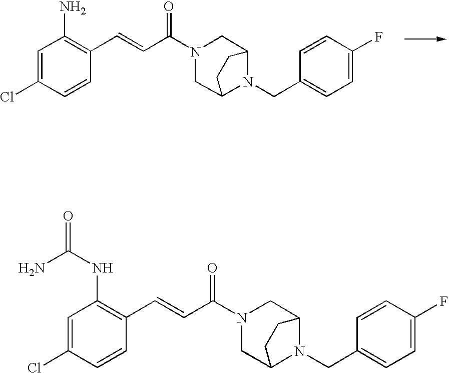

(E)-3-(2-Amino-4-chlorophenyl)-1-[8-(4-fluorobenzyl)-3,8-diazabicyclo[3.2.1]oct-3-yl]-propenone (70 mg; 0.175 mmol) in THF (3 ml) is treated with triphosgene (52 mg; 0.175 mmol). After 5 min an excess of NH3-gas is introduced, the resulting suspension poured on water and extracted with ethyl acetate three times. The combined organic phases are dried over Na2SO4, filtered and evaporated to dryness to deliver a white solid, which is washed with acetone to yield the title compound (57 mg; 74%).

1H-NMR (400 MHz; DMSO-d6), δ (ppm): 1.46 (m, 1H); 1.55 (m, 1H); 1.93 (m, 2H); 2.83 (d, 1H); 3.15 (bs, 2H); 3.25 (m, 1H); 3.50 (bd, 2H); 3.93 (d, 1H); 4.13 (d, 1H); 6.20 (bs, 2H); 7.00-7.18 (m, 4H); 7.40 (dd, 2H); 7.63 (d, 1H); 7.72 (d, 1H); 8.95 (d, 1H); 8.35 (s, 1H).

MS (m/z) ES+: 443 (MH+).

### Example 20

### (E)-N-(5-Chloro-2-{3-[8-(4-fluorobenzyl)-3,8-diazabicyclo[3.2.1]oct-3-yl]-3-oxopropenyl}-phenyl)acetamide

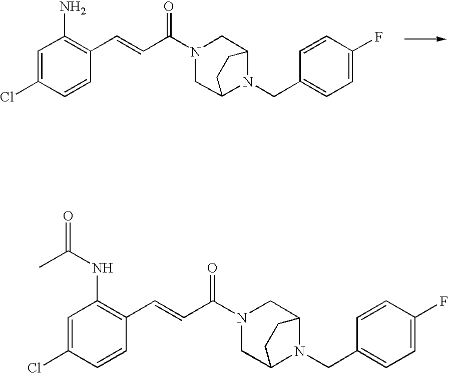

(E)-3-(2-Amino-4-chlorophenyl)-1-[8-(4-fluorobenzyl)-3,8-diazabicyclo[3.2.1]oct-3-yl]-propenone (37 mg; 0.075 mmol) is dissolved in THF (2 ml) and NEt3 (0.1 ml; 0.75 mmol). Acetylchloride (0.052 ml; 0.75 mmol) is added and the reaction mixture refluxed for 5 min., poured on a saturated solution of Na2CO3 and extracted with TBME three times. The combined organic phases are dried over Na2SO4, evaporated to dryness and purified via chromatography (SiO2, acetone/hexanes 4/6) to yield the title compound as colorless foam (23 mg; 71%).

1H-NMR (400 MHz; DMSO-d6), δ (ppm): 1.46 (bt, 1H); 1.58 (bt, 1H); 1.96 (bs, 2H); 2.10 (s, 3H); 2.85 (d, 1H); 3.18 (bs, 2H); 3.28 (d, 1H); 3.51 (d, 2H); 3.97 (d, 1H); 4.13 (d, 1H); 7.15 (d, 1H); 7.16(t, 2H); 7.28 (dd, 1H); 7.45 (dd, 2H); 7.58(d, 1H); 7.63 (d, 1H); 7.91(d, 1H); 9.91 (s, 1H).

MS (m/z) ES+: 442.2 (MH+, 100).

### Example 21

### 3-(5-Chloro-2-{(E)-3-[8-(4-fluoro-benzyl)-3,8-diaza-bicyclo[3.2.1]oct-3-yl]-3-oxo-propenyl}-phenyl)-1,1-dimethyl-urea

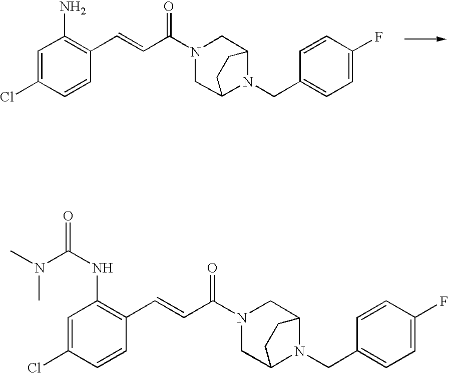

The target compound is prepared in analogy to Example 23f) substituting methylamine by dimethylamine and purified via chromatography (XTerra, RP18, 7 μm, MeCN/water 40/60 to 100/0) to yield the title compound as colorless crystals (51 mg; 62%).

1H-NMR (400 MHz; DMSO-d6), δ (ppm): 1.46 (m, 1H); 1.55 (m, 1H); 1.93 (bs, 2H); 2.81 (bdd, 1H); 2.93 (s, 6H); 3.15 (bs, 2H); 3.25 (bd, 1H); 3.50 (bd, 2H); 3.93 (bd, 1H); 4.10 (bd, 1H); 7.08 (d, 1H); 7.13 (t, 2H); 7.21 (dd, 1H); 7.31 (d, 1H); 7.40 (dd, 2H); 7.51 (d, 1H); 7.83 (d, 1H); 8.38 (s, 1H).

MS (m/z) ES+: 471 (MH+).

### Example 22

### 1-(5-Chloro-2-{(E)-3-[8-(4-fluoro-benzyl)-3,8-diaza-bicyclo[3.2.1]oct-3-yl]-3-oxo-propenyl}-phenyl)-3-methyl-urea

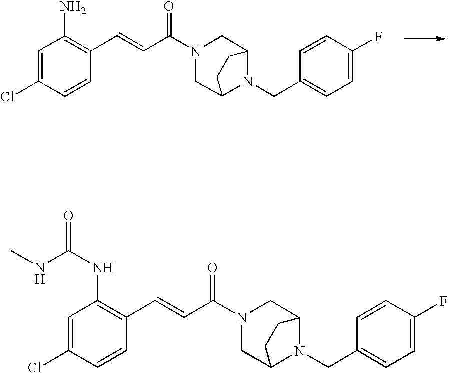

The target compound is prepared in analogy to Example 23f and purified via chromatography chromatography (XTerra, RP18, 7 μm, MeCN/water 40/60 to 100/0) to yield the title compound as colorless foam (41 mg; 51%).

1H-NMR (400 MHz; DMSO-d6), δ (ppm): 1.43 (m, 1H); 1.55 (m, 1H); 1.93 (bs, 2H); 2.63 (d, 3H); 2.80 (d, 1H); 3.15 (bs, 2H); 3.25 (bd, 1H); 3.50 (bs, 2H); 3.93 (bd, 1H); 4.13 (bd, 1H); 6.53 (m, 1H); 7.03 (dd, 1H); 7.05 (d, 1H); 7.13 (t, 2H); 7.40 (dd, 2H); 7.61 (d, 1H); 7.73 (d, 1H); 7.93 (d, 1H); 8.32 (s, 1H).

MS (m/z) ES+: 457.1 (MH+).

### Example 23

### 1-(5-Chloro-2-{(E)-3-[3-(4-fluorobenzyl)-3,8-diazabicyclo[3.2.1]oct-8-yl]-3-oxopropenyl}-4-methoxyphenyl)-3-methylurea

### a) 2-Bromo-5-chloro-4-methoxyphenylamine

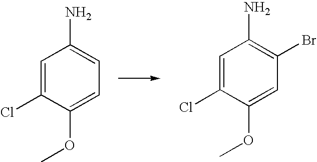

NBS (17 g; 95.5 mmol) in methylene chloride (500 ml) is slowly added to 3-chloro-p-anisidine (15 g; 95.5 mmol) dissolved in methylene chloride (30 ml). After 5 min. the reaction mixture is evaporated to half of its volume and treated with hexanes (2000 ml). The resulting precipitate is filtered off and the filtrate evaporated to dryness, taken up in TBME (30 ml) and combined with hexanes (1000 ml). After standing over night the title product crystallized and is filtered off (9.75 g; 43%). An additional amount (5.4 g; 24%) of product is obtained from the mother liquor after chromatography (SiO2; TBME/hexanes 1:9). Combined yields of title product: 15.15 g; 67%.

1H-NMR (400 MHz; DMSO-d6), δ (ppm): 3.72 (s, 3H); 5.04 (s, 2H, NH2); 6.87 (s, 1H); 7.13 (s, 1H).

MS (m/z) ES+: 237 (50; M+); 235 (45); 222 (100); 220 (80); 194 (45); 192 (40); 78 (45); 52 (50).

### b) (E)-3-(2-Amino-4-chloro-5-methoxyphenyl)-acrylic acid ethyl ester

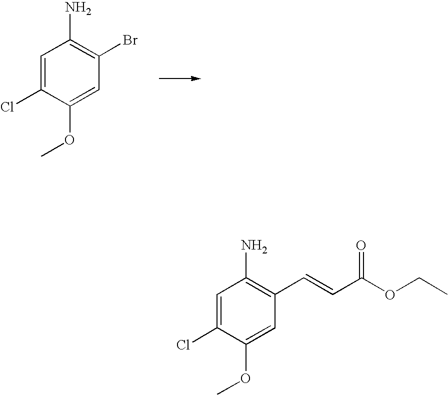

2-Bromo-5-chloro-4-methoxyphenylamine (9.25 g; 39.25 mmol) is dissolved in DMF (100 ml) and combined with ethyl-(E)-3-tributylstannyl)-propenoate (B. Fraser-Reid et al., J. Chem. Soc. Perkin Trans. I, 1994, 1689) (16.8 g; 43 mmol). PdCl2(PPh3)2 (0.55 g; 0.75 mmol) dissolved in warm DMF (50 ml) is added and the reaction mixture heated under argon at 140° C. for 20 min. TBME (50 ml) and toluene (25 ml) are added followed by hexanes (100 ml). The precipitate is filtered off and the filtrate pumped on a silica gel column and purified via chromatography (TBME/hexanes 3:7) yielding the title compound as yellow crystals (8.4 g; 80%).

1H-NMR (400 MHz; DMSO-d6), δ (ppm): 1.25 (t, 3H); 3.77 (s, 3H); 4.16 (q, 2H); 5.42 (s, 2H, NH2); 6.49 (d, 1H); 6.80 (s, 1H); 7.17 (s, 1H); 7.78 (d, 1H).

MS (m/z) ES+: 255 (M+; 55); 210 (100); 194 (45); 166 (55); 138 (40); 104 (55).

### c) (E)-3-(2-Amino-4-chloro-5-methoxyphenyl)-acrylic acid

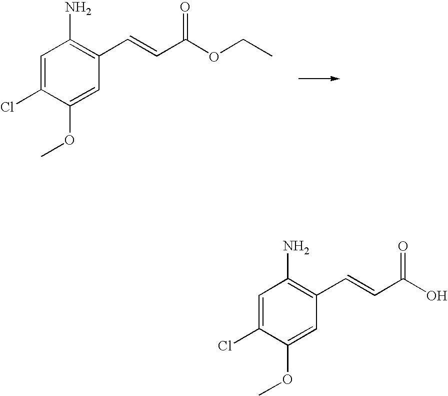

(E)-3-(2-Amino-4-chloro-5-methoxyphenyl)-acrylic acid ethyl ester (14.87 g; 58.3 mmol) is dissolved in EtOH (450 ml), 2N NaOH (58 ml) added and the reaction mixture refluxed for 10 min. 2N HCl (58 ml) is added, the mixture evaporated to a volume of ˜100 ml, poured on water and extracted with TBME. 10% aqueous acetic acid is added to the aqueous phase and extracted further with TBME. The combined organic phases are dried over Na2SO4, evaporated to dryness and purified via chromatography (SiO2; TBME/hexanes/HOAc 70:30:1) to deliver the title compound (12.3 g; 92%) as yellow crystals.

1H-NMR (400 MHz; DMSO-d6), δ (ppm): 3.78 (s, 3H); 5.36 (bs, 2H); 6.40 (d, 1H); 6.80 (s, 1H); 7.13 (s, 1H); 7.72 (d, 1H); 12.15 (bs, 1H).

MS (m/z) ES−: 226 (100; MH−).

### d) (E)-3-(2-Amino-4-chloro-5-methoxyphenyl)-1-[3-(4-fluorobenzyl)-3,8-diazabicyclo[3.2.1]oct-8-yl]-propenone

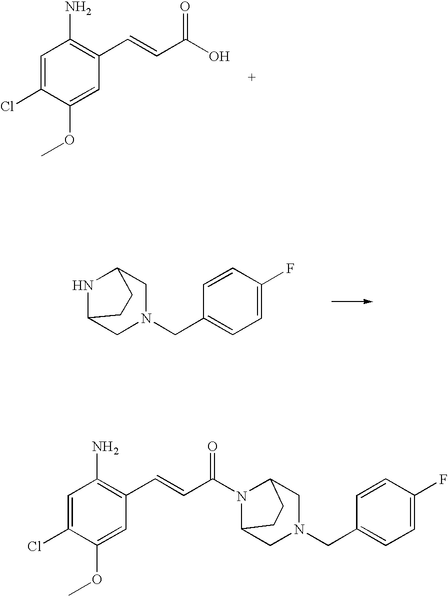

(E)-3-(2-Amino-4-chloro-5-methoxyphenyl)-acrylic acid (3 g; 13.2 mmol) and 3-(4-fluorobenzyl)-3,8-diazabicyclo[3.2.1]octane (2.9 g; 13.2 mmol) (WO 2002032901) are dissolved in DMF (40 ml), combined with HOBt (0.2 g; 1.3 mmol) and EDCl (3 g; 15.8 mmol) and left at room temp. over night. The reaction mixture is poured on water (600 ml)/10% HOAc (8 ml) and extracted with TBME three times. The combined organic phases are dried over Na2SO4, evaporated to dryness and purified via chromatography (SiO2; TBME/MeOH/NH3conc 98:2:0.3) to deliver the title compound (4.9 g; 85%) as yellow foam.

1H-NMR (400 MHz; DMSO-d6), δ (ppm): 1.65-1.75 (m, 1H); 1.83-1.95 (m, 3H); 2.15 (dd, 2H); 2.65 (dd, 2H); 3.45 (s, 2H); 3.75 (s, 3H); 4.50 (bd, 1H); 4.70 (bd; 1H); 5.25 (bs, 2H, NH2); 6.77 (s, 1H); 6.88 (d, 1H); 7.13 (t, 2H); 7.20 (s, 1H); 7.30 (dd, 2H); 7.65 (d, 1H).

MS (m/z) ES+: 430 (MH+, 100).

### e) (E)-3-(2-Acetylamino-4-chloro-5-methoxyphenyl)-acrylic acid

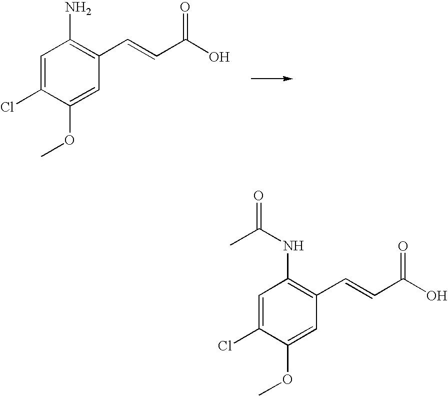

(E)-3-(2-Amino-4-chloro-5-methoxyphenyl)-acrylic acid (5 g; 22.0 mmol) is dissolved in pyridine (60 ml) and treated with acetylchloride (1.7 ml; 24.2 mmol) under vigorous stirring at room temp. After 10 min. the reaction mixture is poured on ice-water/HOAc (1000 ml/60 ml). The precipitated title product is filtered off and the filtrate extracted with EtOAC three times. The combined organic phases are dried over Na2SO4, evaporated to dryness and combined with the first batch of title product. Recrystallisation is carried out by first dissolving in acetone/EtOH (1000 ml/300 ml) followed by evaporation to a volume of ˜20 ml. The resulting crystals are washed with acetone and delivered the title acid as pale yellow crystals (4.95 g; 84%).

1H-NMR (400 MHz; DMSO-d6), δ (ppm): 2.08 (s, 3H); 3.91 (s, 3H); 6.65 (d, 1H); 7.45 (s, 2H); 7.62 (d, 1H); 9.74 (s, 1H); 12.5 (bs, 1H).

MS (m/z) ES−: 268 (100, MH−).

### f) 1-(5-Chloro-2-{(E)-3-[3-(4-fluorobenzyl)-3,8-diazabicyclo[3.2.1]oct-8-yl]-3-oxopropenyl}-4-methoxyphenyl)-3-methylurea

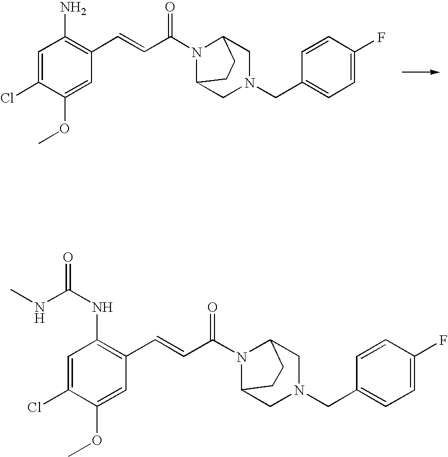

(E)-3-(2-Amino-4-chloro-5-methoxyphenyl)-1-[3-(4-fluorobenzyl)-3,8-diazabicyclo[3.2.1]oct-8-yl]-propenone (4.9 g; 11.4 mmol) is dissolved in THF (250 ml). Triphosgene (3.73 g; 12.6 mmol) is added at room temp. After 7 min. the reaction mixture is placed in a cooling water bath of ˜20° C., followed by the addition of an excess of methylamine (˜20 ml). After 5 min. the reaction mixture is poured on water (1000 ml) and filtered from the precipitated title product. An additional amount of title product is obtained by extracting the aqueous phase with EtOAc three times. The combined organic phases are evaporated. The combined batches are recrystallised from EtOAc to deliver the title compound (4.7 g; 86%) as colorless crystals.

1H-NMR (400 MHz; DMSO-d6), δ (ppm): 1.70-1.80 (m, 1H); 1.82-1.98 (m, 3H); 2.17 (dd, 2H); 2.62 (d, 3H); 2.68(dd, 2H); 3.46 (s, 2H); 3.90 (s, 3H); 4.53 (bd, 1H); 4.70 (bd, 1H); 6.27 (q, 1H, NH); 7.05 (d, 1H); 7.13 (dd, 2H); 7.32 (dd, 2H); 7.38 (s, 1H); 7.62 (d, 1H); 7.65 (s, 1H); 8.13 (s, 1H, NH).

MS (m/z) ES+: 487 (100, MH+).

### Example 24

### (5-Chloro-2-{(E)-3-[3-(4-fluorobenzyl)-3,8-diazabicyclo[3.2.1]oct-8-yl]-3-oxopropenyl}-4-methoxyphenyl)-3-urea

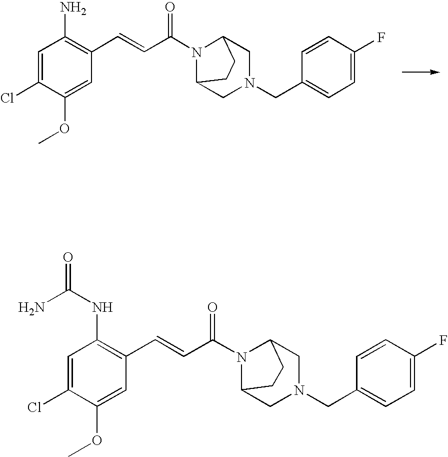

(E)-3-(2-Amino-4-chloro-5-methoxyphenyl)-1-[3-(4-fluorobenzyl)-3,8-diazabicyclo[3.2.1]oct-8-yl]-propenone (6.5 g; 15.15 mmol) dissolved in HOAc/H2O (90 ml/90 ml) is treated with NaOCN (2.95 g; 45.4 mmol) for 35 min. at room temp. The reaction mixture is poured on a saturated solution of Na2CO3 and extracted with EtOAc three times. The combined organic phases are dried over Na2SO4, evaporated to dryness and purified via chromatography (SiO2; acetone/hexanes 4:6 to 7:3) to yield a product, which is further purified by recrystallisation from acetone to yield the title compound as colorless crystals (5.0 g; 69%).

1H-NMR (400 MHz; DMSO-d6), δ (ppm): 1.75 (m, 1H); 1.84-2.03 (m, 3H); 2.18 (dd, 2H); 2.68 (dd, 2H); 3.47 (s, 2H); 3.88 (s, 3H); 4.54 (bd, 1H); 4.72 (bd, 1H); 6.03 (s, 2H, NH2); 7.08 (d, 1H); 7.13 (t, 2H); 7.32 (m, 2H); 7.40 (s, 1H); 7.65 (d, 1H); 7.70 (s, 1H); 8.19 (s, 1H, NH).

MS (m/z) ES+: 473 (20, MH+); 430 (100).

### Example 25

### N-(5-Chloro-2-{(E)-3-[3-(4-fluorobenzyl)-3,8-diazabicyclo[3.2.1]oct-8-yl]-3-oxopropenyl}-4-methoxyphenyl)-acetamide

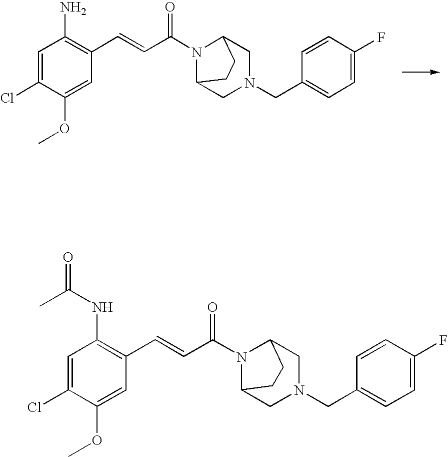

Acetylchloride (0.83 ml; 1.16 mmol) is added under vigorous stirring to a solution of (E)-3-(2-amino-4-chloro-5-methoxyphenyl)-1-[3-(4-fluorobenzyl)-3,8-diazabicyclo[3.2.1]oct-8-yl]-propenone (0.5 g; 1.16 mmol) in THF (10 ml) and NEt3 (1.62 ml; 1.16 mmol). The reaction mixture is poured after 5 min. on a saturated solution of Na2CO3 and extracted with TBME three times. The combined organic phases are dried over Na2SO4, evaporated to dryness and purified via chromatography (SiO2; acetone/hexanes 4:6 to 6:4) to yield the title compound as slightly colored foam (297 mg; 54%).

1H-NMR (400 MHz; DMSO-d6), δ (ppm): 1.75 (m, 1H); 1.83-2.00 (m, 3H); 2.05 (s, 3H); 2.17 (dd, 2H); 2.70 (dd, 2H); 3.48 (s, 2H); 3.93 (s, 3H); 4.53 (bd, 1H); 4.71 (bd, 1H); 7.08-7.17 (m, 3H); 7.32 (dd, 2H); 7.42 (s, 1H); 7.48 (s, 1H); 7.60 (d, 1H); 9.70 (s, 1H).

MS (m/z) ES+: 472 (100, MH+).

### Example 26

### 1-(5-Chloro-2-{(E)-3-[3-(4-fluoro-benzyl)-3,8-diaza-bicyclo[3.2.1]oct-8-yl]-3-oxo-propenyl}-4-methoxy-phenyl)-3-cyclopropyl-urea

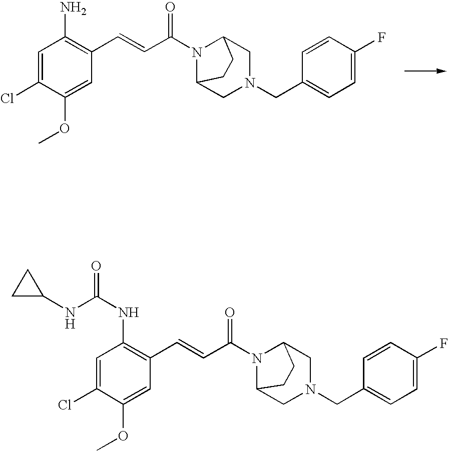

The target compound is prepared in analogy to Example 23f substituting methylamine by cyclopropylamine. Purification via chromatography (SiO2; acetone/hexanes 3/7) and recrystallisation from acetone/TBME yielded the title compound as colorless crystals (39 mg; 42).

1H-NMR (400 MHz; DMSO-d6), δ (ppm): 0.41 (m, 2H); 0.63 (m, 2H); 1.70-1.80 (m, 1H); 1.83-2.00 (m, 3H); 2.15 (dd, 2H); 2.53 (m, 1H); 2.63 (d, 1H); 2.71 (d, 1H); 3.45 (s, 2H); 3.88 (s, 3H); 4.53 (bd, 1H); 4.70 (bd, 1H); 6.64 (bs, 1H); 7.05 (d, 1H); 7.13 (t, 2H); 7.30 (dd, 2H); 7.39 (s, 1H); 7.62 (d, 1H); 7.70 (s, 1H); 8.00 (s, 1H).

MS (m/z) ES+: 513 (MH+).

### Example 27

### N-(5-Chloro-2-{(E)-3-[3-(4-fluoro-benzyl)-3,8-diaza-bicyclo[3.2.1]oct-8-yl]-3-oxo-propenyl}-4-methoxy-phenyl)-methanesulfonamide

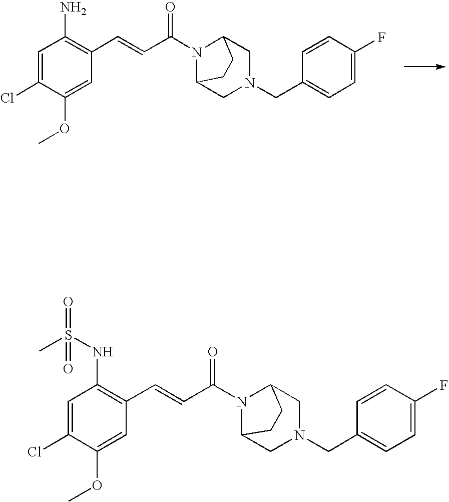

The target compound is prepared in analogy to Example 5 and purified via chromatography (SiO2, TBME/MeOH/NH3conc 95/5/1 to 90/10/1.5) to yield the title compound as colorless crystals (426 mg; 51%).

1H-NMR (400 MHz; DMSO-d6), δ (ppm): 1.75 (m, 1H); 1.83-2.00 (m, 3H); 2.17 (dd, 2H); 2.63 (d, 1H); 2.70 (d, 1H); 2.95 (s, 3H); 3.48 (s, 2H); 3.93 (s, 3H); 4.53 (bd, 1H); 4.71 (bd, 1H); 7.08-7.17 (m, 3H); 7.32 (dd, 2H); 7.38 (s, 1H); 7.51 (s, 1H); 7.81 (d, 1H); 9.42 (s, 1H).

MS (m/z) ES+: 508.2 (MH+).

### Example 28

### N-(5-Chloro-2-{(E)-3-[3-(4-fluoro-benzyl)-3,8-diaza-bicyclo[3.2.1]oct-8-yl]-3-oxo-propenyl}-4-methoxy-phenyl)-2-dimethylamino-acetamide

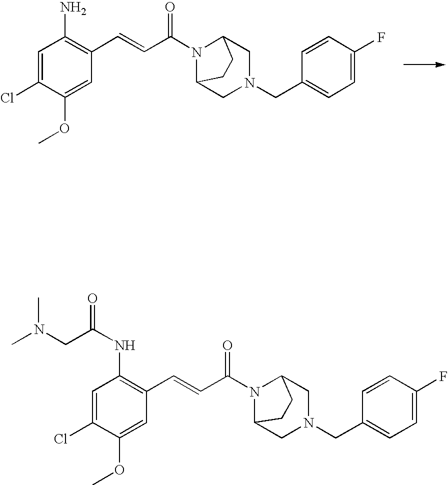

The target compound is prepared in analogy to Example 3 and purified via chromatography (SiO2, acetone/hexanes 4/6) to yield the title compound as yellowish foam (30 mg; 83%).

1H-NMR (400 MHz; DMSO-d6), δ (ppm 1.75 (m, 1H); 1.83-1.95 (m, 3H); 2.17 (bt, 2H); 2.31 (s, 6H); 2.63 (d, 1H); 2.70 (d, 1H); 3.05 (s, 2H); 3.48 (s, 2H); 3.93 (s, 3H); 4.53 (bd, 1H); 4.71 (bd, 1H); 7.08-7.17 (m, 3H); 7.32 (dd, 2H); 7.45 (s, 1H); 7.48 (s, 1H); 7.55 (s, 1H); 9.62 (s, 1H).

MS (m/z) ES+: 515.1 (MH+); 258.1 (100).

### Example 29

### 3-(5-Chloro-2-{(E)-3-[3-(4-fluoro-benzyl)-3,8-diaza-bicyclo[3.2.1]oct-8-yl]-3-oxo-propenyl}-4-methoxy-phenyl)-1,1-dimethyl-urea

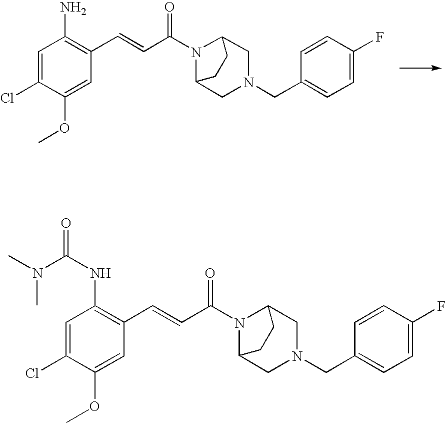

The target compound is prepared in analogy to Example 23f) substituting methylamine by dimethylamine and purified via chromatography (SiO2, acetone/hexanes 3/7 to 4/6) to yield the title compound as colorless crystals (25 mg; 27%).

1H-NMR (400 MHz; DMSO-d6), δ (ppm): 1.70-1.80 (m, 1H); 1.83-2.00 (m, 3H); 2.15 (dd, 2H); 2.63 (d, 1H); 2.71 (d, 1H); 2.90 (s, 6H); 3.30 (s, 2H);); 3.90 (s, 3H); 4.53 (bd, 1H); 4.68 (bd, 1H); 7.05 (d, 1H); 7.13 (t, 2H); 7.25 (s, 1H); 7.30 (dd, 2H); 7.45 (s, 1H); 7.58 (d, 1H); 8.13 (s, 1H).

MS (m/z) ES+: 501 (MH+); 456 (35); 430 (100).

### Example 30

### 5-Chloro-2-{(E)-3-[3-(4-fluoro-benzyl)-3,8-diaza-bicyclo[3.2.1]oct-8-yl]-3-oxo-propenyl}-4-methoxy-N,N-dimethyl-benzenesulfonamide

### a) 2-Bromo-5-chloro-4-methoxy-benzenesulfonyl chloride

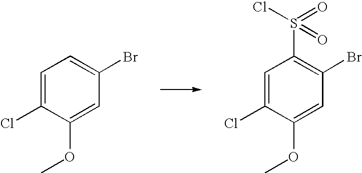

4-Bromo-1-chloro-2-methoxy-benzene (2 g; 9 mmol) is added dropwise under stirring at 0° C. to chlorosulfonic acid (4.8 ml). The mixture is warmed to room temp., poured on ice-water and extracted with TBME three times. The combined organic phases are dried over Na2SO4, evaporated to dryness and the resulting solid washed with hexanes to deliver the title compound as colorless crystals (2.08 g; 72%).

1H-NMR (400 MHz; DMSO-d6), δ (ppm): 2.78 (s, 6H); 3.97 (s, 3H); 7.60 (s, 1H); 7.90 (s, 1H).

MS (m/z) ES+: 330 (MH+, 100).

### b) 2-Bromo-5-chloro-4-methoxy-N,N-dimethyl-benzenesulfonamide

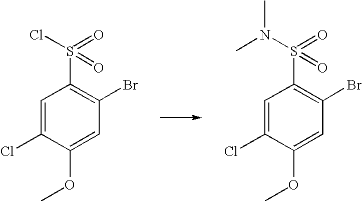

2-Bromo-5-chloro-4-methoxy-benzenesulfonyl chloride (1 g; 3.12 mmol) is dissolved in TBME (100 ml) and combined with dimethylamine (2.5 ml) at room temp. After 5 min of stirring the precipitate is filtered off and the solid purified by chromatography (SiO2, TBME/hexanes 4/6) to yield the title compound as colorless crystals (951 mg; 93%).

1H-NMR (400 MHz; DMSO-d6), δ (ppm): 2.78 (s, 6H); 3.97 (s, 3H); 7.60 (s, 1H); 7.90 (s, 1H).

MS (m/z) ES+: 330 (MH+, 100).

### c) (E)-3-(4-Chloro-2-dimethylsulfamoyl-5-methoxy-phenyl)-acrylic acid ethyl ester

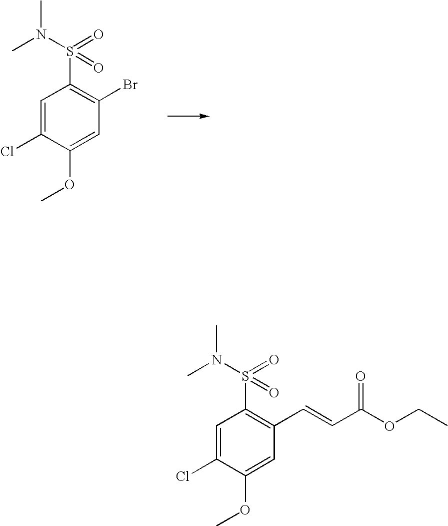

The target compound is prepared in analogy to Example 41b using ethyl-(E)-3-tributylstannyl)-propenoate and PdCl2(PPh3)2 as catalyst in the Stille coupling. Purification of the product via chromatography (SiO2, acetone/hexanes 1/9) delivered the title compound as yellowish crystals (408 mg; 77%).

1H-NMR (400 MHz; DMSO-d6), δ (ppm): 1.26 (t, 3H); 2.67 (s, 6H); 4.03 (s, 3H); 4.20 (q, 2H); 6.83 (d, 1H); 7.61 (s, 1H); 7.83 (s, 1H); 8.35 (d, 1H).

MS (m/z) ES+: 348 (MH+, 40); 302 (100).

### d) (E)-3-(4-Chloro-2-dimethylsulfamoyl-5-methoxy-phenyl)-acrylic acid

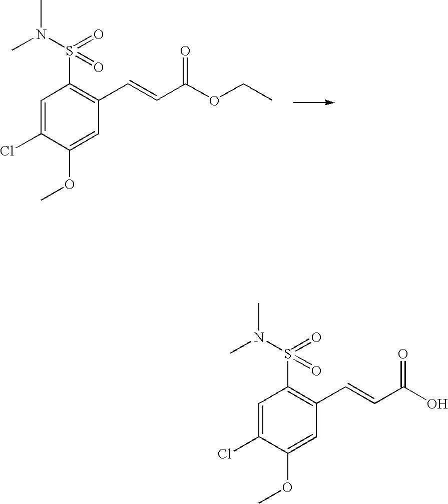

(E)-3-(4-Chloro-2-dimethylsulfamoyl-5-methoxy-phenyl)-acrylic acid ethyl ester (0.4 g; 1.14 mmol) dissolved in EtOH (15 ml) is treated with 2N NaOH under reflux for 15 min. The mixture is poured on water, acidified with 2N HCl (2 ml) and extracted with TBME three times. The combined organic phases are dried over Na2SO4, filtered and evaporated to dryness to yield the title compound as colorless crystals (372 mg; 100%).

1H-NMR (400 MHz; DMSO-d6), δ (ppm): 2.67 (s, 6H); 4.03 (s, 3H); 6.70 (d, 1H); 7.59 (s, 1H); 7.83 (s, 1H); 8.38 (d, 1H); 12.8 (s, 1H).

MS (m/z) ES+: 320 (MH+, 95); 302 (100).

### e) 5-Chloro-2-{(E)-3-[3-(4-fluoro-benzyl)-3,8-diaza-bicyclo[3.2.1]oct-8-yl]-3-oxo-propenyl}-4-methoxy-N,N-dimethyl-benzenesulfonamide

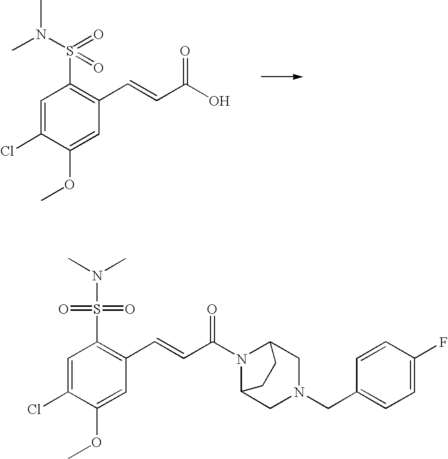

(E)-3-(4-Chloro-2-dimethylsulfamoyl-5-methoxy-phenyl)-acrylic acid (80 mg; 0.25 mmol) in xylene (4 ml) and a drop of DMF is combined with thionyl chloride (0.5 ml) and refluxed for 10 min. The reaction mixture is evaporated and delivered the off-white acid chloride as a solid, which is dissolved in CH2Cl2 (2 ml) and added under stirring to a solution of 3-(4-fluoro-benzyl)-3,8-diaza-bicyclo[3.2.1]octane (61 mg; 27.6 mmol) in CH2Cl2 (1 ml). After 10 min. of stirring, NH3conc (0.5 ml) is added, the reaction mixture diluted with acetone (2 ml) and poured on a column of SiO2 and chromatographed (acetone/hexanes 2/8) to yield the title compound as colorless crystals (105 mg; 80%).

1H-NMR (400 MHz; DMSO-d6), δ (ppm): 1.70-1.80 (m, 1H); 1.83-2.00 (m, 3H); 2.1 (d, 1H); 2.20 (d, 1H); 2.63 (d, 1H); 2.65 (s, 6H); 2.71 (d, 1H); 3.45 (s, 2H);); 4.05 (s, 3H); 4.53 (bd, 1H); 4.65 (bd, 1H); 7.13 (bt, 3H); 7.30 (dd, 2H); 7.55 (s, 1H); 7.81 (s, 1H); 8.20 (d, 1H).

MS (m/z) ES+: 522 (MH+).

### Example 31

### N-[5-Chloro-2-{(E)-3-[3-(4-fluoro-benzyl)-3,8-diaza-bicyclo[3.2.1]oct-8-yl]-3-oxo-propenyl}-4-(1-hydroxy-1-methyl-ethyl)-phenyl]-acetamide

### a) 4-Amino-5-bromo-2-chloro-benzoic acid methyl ester

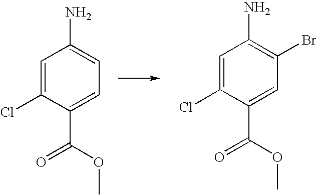

4-Amino-2-chloro-benzoic acid methyl ester (7.8 g, 42.0 mmol) is dissolved in 300 ml THF. At room temperature 8.97 g (50.4 mmol) N-bromsuccinimide are added in portions. After stirring over night at room temperature, 200 ml ethyl acetate are added and the organic layer is washed first with 10% sodium thiosulfate solution followed by 10% sodium carbonate solution and saturated sodium chloride solution. The title compound is purified by chromatography (SiO₂, ethyl acetate/c-hexane 1/9) and is isolated as a yellow solid (3.70 g, 33%)

1H-NMR (400 MHz; DMSO-d6): 3.75 (s, 3H); 3.15-3.25 (m, 1H); 6.35 (s, 1NH); 6.83 (s, 1H); 7.88 (s, 1H).

MS (m/z) ES−: 264 ([M−H]−, 100).

### b) 2-(4-Amino-5-bromo-2-chloro-phenyl)-propan-2-ol

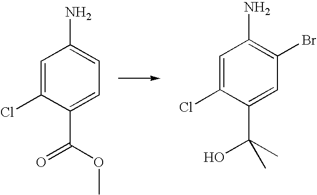

Title compound of step a) (0.79 g, 3.0 mmol) is dissolved in 30 ml THF and at 0° C. 5 ml (15.0 mmol) methylmagnesium bromide ˜3M in ethyl ether is added dropwise. Stirring is continued for 5 hours at room temperature, then 100 ml saturated ammonium chloride solution are added with caution. The organic layer is washed twice with water and once with saturated sodium chloride solution. The title compound is purified by chromatography (SiO₂, ethyl acetate/c-hexane 1/4) and is isolated as a yellow oil (0.61 g, 77%)

1H-NMR (400 MHz; DMSO-d6): 1.50 (s, 6H); 5.10 (s, 10H); 5.35 (bs, 1NH); 6.77 (s, 1H); 7.56 (s, 1H)

MS (m/z) EI: 265 (M+, 50), 250 ([M−CH3]+, 100)

### c) (E)-3-[2-Amino-4-chloro-5-(1-hydroxy-1-methyl-ethyl)-phenyl]-acrylic acid ethyl ester

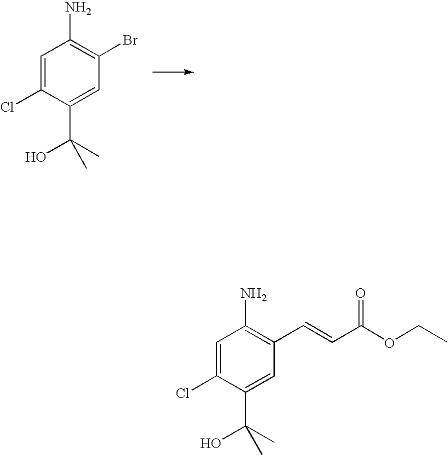

Title compound of step b) (0.58 g, 2.20 mmol) and 0.95 g (2.42 mmol) (E)-3-tributylsyannanyl-acrylic acid ethyl ester are dissolved in 12 ml DMF and 35 mg Bis (triphenylphosphine)palladium(II)dichloride are added. This mixture is stirred at 140° C. for 30 min. After evaporation the title compound is purified by chromatography (SiO₂, ethyl acetate/c-hexane 1/4) and is isolated as a pale solid (0.52 g, 83.3%).

1H-NMR (400 MHz; DMSO-d6): 1.25 (t, 3H); 1.50 (s, 6H), 4.15 (qa, 2H), 5.05 (s, 10H); 5.73 (bs, 1NH); 6.27 (d, 1H), 6.70 (s, 1H); 7.72 (s, 1H), 7.80 (d, 1H)

MS (m/z) EI: 284 (MH+, 40), 266 ([MH−H2O]+, 100)

### d) (E)-3-[2-Acetylamino-4-chloro-5-(1-hydroxy-1-methyl-ethyl)-phenyl]-acrylic acid ethyl ester

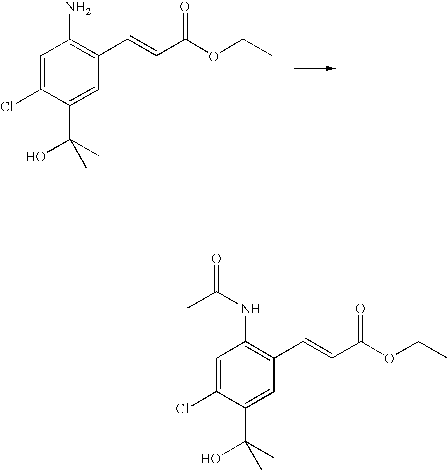

Title compound of step c) (0.51 g, 1.80 mmol) is dissolved in 30 ml THF and 1.25 ml (9.00 mmol) NEt₃ and 0.63 ml (9.00 mmol) acetyl chloride are added. This mixture is stirred for 3 hours at room temperature. Then the mixture is diluted with 100 ml 20% sodium carbonate solution and extracted with ethyl acetate. The title compound is purified by chromatography (SiO₂, ethyl acetate/c-hexane 1/2) and is isolated as a pale solid (250 mg, 42.6%)

1H-NMR (400 MHz; DMSO-d6): 1.25 (t, 3H); 1.57 (s, 6H), 2.07 (s, 3H), 4.20 (qa, 2H), 5.35 (s, 10H); 6.45 (d, 1H), 7.50 (s, 1H), 7.80 (d, 1H), 8.05 (s, 1H), 9.85 (bs, 1 NH)

MS (m/z) EI: 343 ([M+NH4]+, 100)

### e) (E)-3-[2-Acetylamino-4-chloro-5-(1-hydroxy-1-methyl-ethyl)-phenyl]-acrylic acid

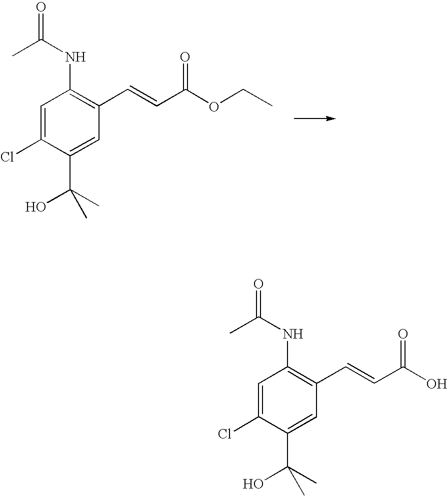

Title compound of step d) (1.0 g, 3.20 mmol) is dissolved in 25 ml MeOH and 2.4 ml 2N NaOH is added, this mixture is heated to 50° C. for 4 hours. The solution is cooled to room temperature and evaporated. The residue is dissolved in 4N HCl solution of 1,4-dioxane and evaporated. The remaining solid is used without further purification for the next step.

### f) N-[5-Chloro-2-{(E)-3-[3-(4-fluoro-benzyl)-3,8-diaza-bicyclo[3.2.1]oct-8-yl]-3-oxo-propenyl}-4-(1-hydroxy-1-methyl-ethyl)-phenyl]-acetamide

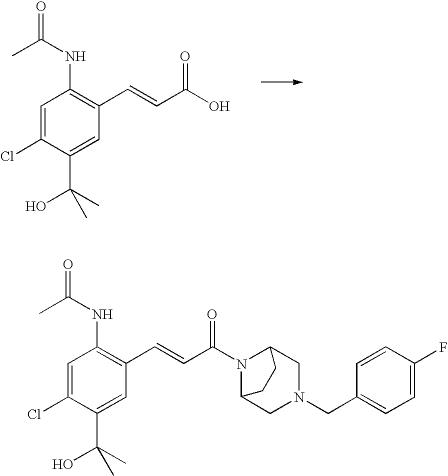

Title compound of step e) (148.9 mg, 0.50 mmol), EDCl (115 mg, 0.60 mmol); HOBT (8 mg, 0.05 mmol) and 110 mg (0.50 mmol) 3-(4-Fluoro-benzyl)-3,8-diaza-bicyclo[3.2.1]octane are dissolved in 15 ml DMF and stirred over night at room temperature. The reaction mixture is poured into 300 ml water and extracted with ethyl acetate. The title compound is purified by chromatography (SiO₂, ethyl acetate/MeOH/NH₃conc. 99/1/0.1) and is isolated as a pale solid (50 mg, 20%)

1H-NMR (400 MHz; DMSO-d6): 1.57 (s, 6H), 1.65-1.95 (m, 4H), 2.07 (s, 3H), 2.10-2.20 (m, 2H), 2.60-2.75 (m, 2H), 3.45 (s, 2H), 4.50-4.60 (m, 2H), 5.30 (s, 10H); 6.90 (d, 1H), 7.05-7.15 (m, 2H), 7.25-7.35 (m, 2H), 7.45 (s, 1H), 7.65 (d, 1H), 8.05(s, 1H), 9.80 (bs, 1 NH)

MS (m/z) EI: 500 (MH+, 100)

### Example 32

### N-(5-Chloro-4-ethoxy-2-{(E)-3-[3-(4-fluoro-benzyl)-3,8-diaza-bicyclo[3.2.1]oct-8-yl]-3-oxo-propenyl}-phenyl)-acetamide

### a) (E)-3-(4-Chloro-5-ethoxy-2-nitro-phenyl)-1-[3-(4-fluoro-benzyl)-3,8-diaza-bicyclo[3.2.1]oct-8-yl]-propene

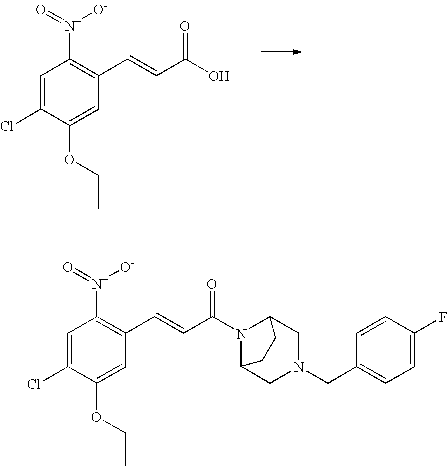

(E)-3-(4-Chloro-5-ethoxy-2-nitro-phenyl)-acrylic acid (1.10 g, 4.05 mmol) are added to 6.2 ml thionylchloride and stirred for 10 min. at 150° C. The solution is cooled to room temperature and evaporated. The remaining residue is twice dissolved in toluol and evaporated. 579 mg (2.00 mmol) of the remaining residue is dissolved in 10 ml toluol and 440 mg (2.00 mmol) 3-(4-Fluoro-benzyl)-3,8-diaza-bicyclo[3.2.1]octane dissolved in 5 ml THF are added at room temperature. After stirring for 1 hour at room temperature the mixture is diluted with 100 ml 20% sodium carbonate solution and extracted with ethyl acetate. The title compound is purified by chromatography (SiO₂, ethyl acetate/MeOH/NH₃conc. 95/510.5) and is isolated as a pale solid (540 mg, 57%)

1H-NMR (400 MHz; DMSO-d6): 1.40 (t, 3H), 1.70-1.95 (m, 4H), 2.15-2.20 (m, 2H), 2.60-2.75 (m, 2H), 3.45 (s, 2H), 4.35 (qa, 2H), 4.50-4.55 (m, 1H), 4.65-4.70 (m, 1H), 7.05-7.15 (m, 3H), 7.25-7.35 (m, 2H), 7.45 (s, 1H), 7.80 (d, 1H), 8.20 (s, 1H)

MS (m/z) EI: 474.2 (MH+, 100)

### b) (E)-3-(2-Amino-4-chloro-5-ethoxy-phenyl)-1-[3-(4-fluoro-benzyl)-3,8-diaz-bicyclo[3.2.1]oct-8-yl]-propenone

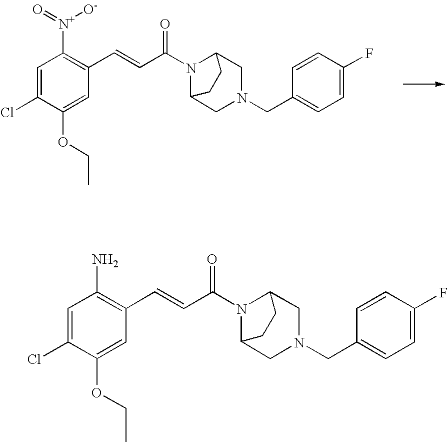

Title compound of step a) (0.52 g, 1.10 mmol) is dissolved in 15 ml THF and 3.0 ml water and 1.7 g (7.70 mmol) stannous chloride anhydrous are added. This mixture is stirred at 80 ⁰C for 15 min., then the mixture is diluted with 100 ml saturated sodium carbonate solution and extracted with ethyl acetate. The title compound is purified by chromatography (SiO₂, ethyl acetate/MeOH/NH₃conc. 95/5/0.5) and is isolated as a pale solid (0.33 g, 67%)

### c) N-(5-Chloro-4-ethoxy-2-{(E)-3-[3-(4-fluoro-benzyl)-3,8-diaza-bicyclo[3.2.1]oct-8-yl]-3-oxo-propenyl}-phenyl)-acetamide

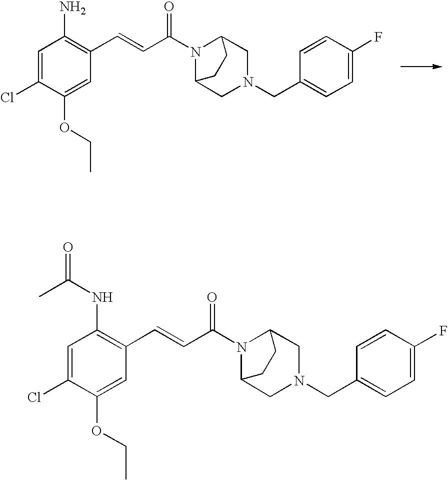

Title compound of step b) (110 mg, 0.25 mmol) is dissolved in 20 ml THF and 0.35 ml (2.50 mmol) NEt₃ and 0.18 ml (2.50 mmol) acetyl chloride are added. This mixture is stirred for 5 min. at room temperature. Then the mixture is diluted with 10 ml saturated sodium carbonate solution and extracted with ethyl acetate. The title compound is purified by chromatography (SiO₂, ethyl acetate/MeOH/NH₃conc. 98/2/0.2) and is isolated as a pale solid (60 mg, 50%)

1H-NMR (400 MHz; DMSO-d6): 1.35 (t, 3H), 1.70-1.95 (m, 4H), 2.05 (s, 3H), 2.13-2.20 (m, 2H), 2.60-2.75 (m, 2H), 3.45 (s, 2H), 4.15 (qa, 2H), 4.48-4.55 (m, 1H), 4.65-4.70 (m, 1H), 7.00-7.15 (m, 3H), 7.25-7.35 (m, 2H), 7.40 (s, 1H), 7.45 (s, 1H), 7.55 (d, 1H), 9.70 (bs, NH)

MS (m/z) EI: 486 (MH+, 100)

### Example 33

### N-(5-Chloro-4-ethoxy-2-{(E)-3-[3-(4-fluoro-benzyl)-3,8-diaza-bicyclo[3.2.1]oct-8-yl]-3-oxo-propenyl}-phenyl)-methansulfonamide

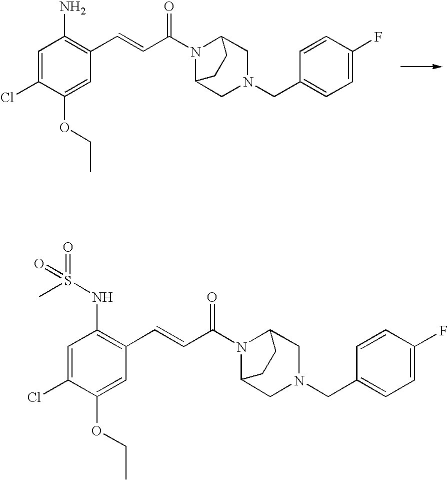

Title compound of step b) of example 2 (110 mg, 0.25 mmol) is dissolved in 20 ml THF, then 0.042 ml (0.3 mmol) NEt₃ and 0.02 ml (0.25 mmol) methanesulfonyl chloride are added at −78° C. Stirring is continued for 4 hours at −78° C., then the mixture is allowed to warm up to room temperature and is evaporated. The title compound is purified by chromatography (SiO₂, ethyl acetate/MeOH/NH₃conc. 98/2/0.2) and is isolated as a pale solid (70 mg, 54%)

1H-NMR (400 MHz; DMSO-d6): 1.35 (t, 3H), 1.70-1.95 (m, 4H), 2.13-2.20 (m, 2H), 2.60-2.75 (m, 2H), 2.95 (s, 3H), 3.45 (s, 2H), 4.20 (qa, 2H), 4.50-4.55 (m, 1H), 4.65-4.70 (m, 1H), 7.05-7.15 (m, 3H), 7.25-7.35 (m, 3H), 7.50 (s, 1H), 7.80 (d, 1H), 9.40 (bs, NH)

MS (m/z) EI: 522 (MH+, 100)

### Example 34

### N-(5-Chloro-4-ethoxy-2-{(E)-3-[3-(4-fluoro-benzyl)-3,8-diaza-bicyclo[3.2.1]oct-8-yl]-3-oxo-propenyl}-phenyl)-urea

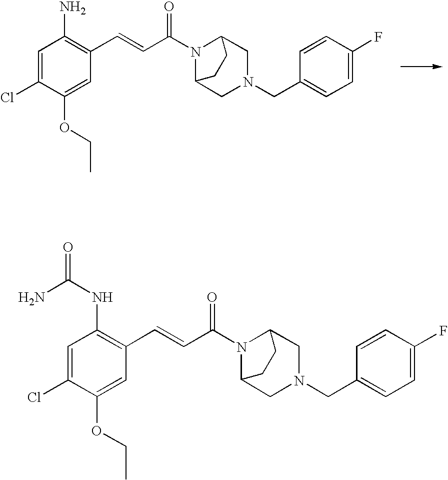

Title compound of step b) of example 2 (110 mg, 0.25 mmol) is dissolved in 0.5 ml 1N HCl and 10 ml water and 32.2 mg (0.50 mmol) sodium isocyanate are added. This mixture is stirred at 60° C. over night, then the mixture is diluted with 10 ml 2N NaOH solution and extracted with ethyl acetate. The title compound is purified by chromatography (SiO₂, ethyl acetate/MeOH/NH₃conc. 98/2/0.2) and is isolated as a pale solid (30 mg, 25%)

1H-NMR (400 MHz; DMSO-d6): 1.35 (t, 3H), 1.70-1.95 (m, 4H), 2.13-2.20 (m, 2H), 2.60-2.75 (m, 2H), 3.45 (s, 2H), 4.15 (m, 2H), 4.50-4.55 (m, 1H), 4.65-4.70 (m, 1H), 6.00 (bs, 2NH), 7.05 (d, 1H), 7.10-7.15 (m, 2H), 7.28-7.34 (m, 2H), 7.40 (s, 1H), 7.62 (d, 1H), 7.65 (s, 1H), 8.15 (bs, NH)

MS (m/z) EI: 487 (MH+, 100)

### Example 35

### (5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-urea

### a) 2-Bromo-5-chloro-4-trifluoromethoxy-phenylamine

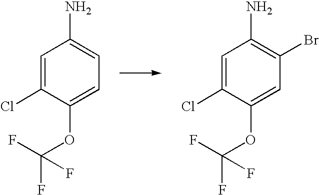

N-Bromosuccinimide (7.9 g; 44.5 mmol) in CH2Cl2 (500 ml) is added under stirring within 5 min. to a solution of 3-chloro-4-trifluoromethoxy-phenylamine (9.4 g; 44.5 mmol) in CH2Cl2 (100 ml) at room temp. After 20 min. the reaction mixture is concentrated to a volume of ˜150 ml, and hexanes (1000 ml) added to the precipitated crystals. The crystals are filtered off an purified via chromatography (SiO2; TBME/hexanes 1/9 to 2/8) to deliver the target compound as yellowish crystals (8.4 g; 42%).

1H-NMR (400 MHz; DMSO-d6), δ (ppm): 5.81 (s, 1H); 6.94 (s, 1H); 7.55 (s, 2H).

MS (m/z) ES−: 290 (MH−).

### b) (E)-3-(2-Amino-4-chloro-5-trifluoromethoxy-phenyl)-acrylic acid ethyl ester

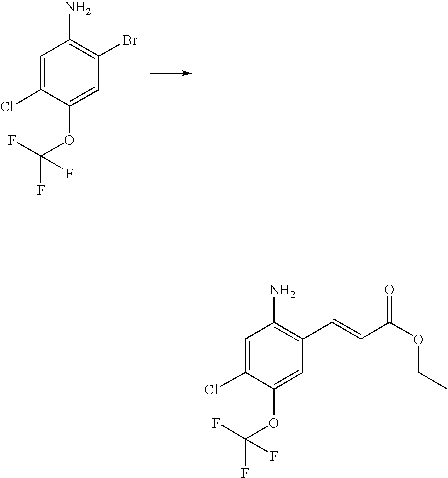

The target compound is prepared in analogy to Example 23b and purified via chromatography (SiO2; TBME/hexanes 317 to 4/6) to deliver the target compound as yellow crystals (1.65 g; 77%).

MS (m/z) ES+: 310 (MH+).

### c) (E)-3-(2-Amino-4-chloro-5-trifluoromethoxy-phenyl)-acrylic acid

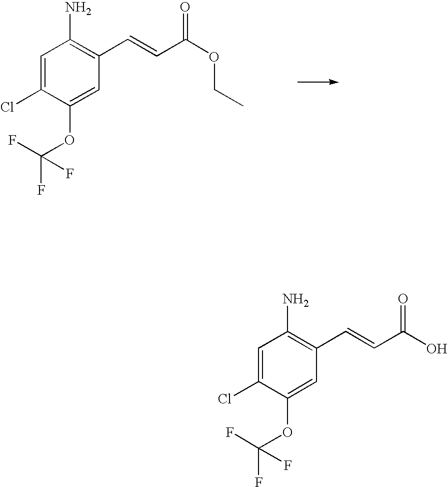

(E)-3-(2-Amino-4-chloro-5-trifluoromethoxy-phenyl)-acrylic acid ethyl ester (1.65 g; 5.33 mol) is dissolved in EtOH (40 ml), 2N NaOH (5.3 ml) added and the mixture refluxed for 10 min. The reaction mixture is diluted with water, washed with TBME, the aqueous phase acidified with 2N HCl and extracted with TBME three times. The combined organic phases are dried over Na2SO4, filtered and evaporated to dryness yielding the title compound as yellow crystals (1.49 g; 99%).

1H-NMR (400 MHz; DMSO-d6), δ (ppm): 6.08 (bs, 2H); 6.39 (d, 1H); 6.86 (s, 1H); 7.58 (s, 1H); 7.69 (d, 1H); 12.3 (s, 1H).

MS (m/z) ES+: 280 (MH−).

### d) E)-3-(2-Amino-4-chloro-5-trifluoromethoxy-phenyl)-acryloyl chloride

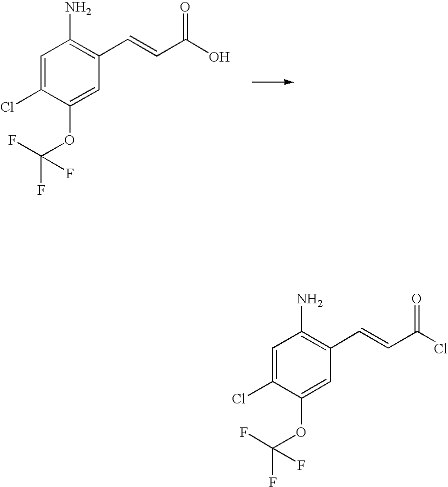

(E)-3-(2-Amino-4-chloro-5-trifluoromethoxy-phenyl)-acrylic acid 0.4 g; 1.4 mmol) is dissolved in toluene (30 ml), combined under cooling with 1N HCl in ether (2.8 ml) resulting in a fine precipitate. Ether is evaporated, thionyl chloride (6 ml) added to the HCl-salt and the mixture refluxed for 10 min. Toluene is evaporated yielding crystalline title product.

### e) (E)-3-(2-Amino-4-chloro-5-trifluoromethoxy-phenyl)-1-[3-(4-fluoro-benzyl)-3,8-diaza-bicyclo[3.2.1]oct-8-yl]-propenone

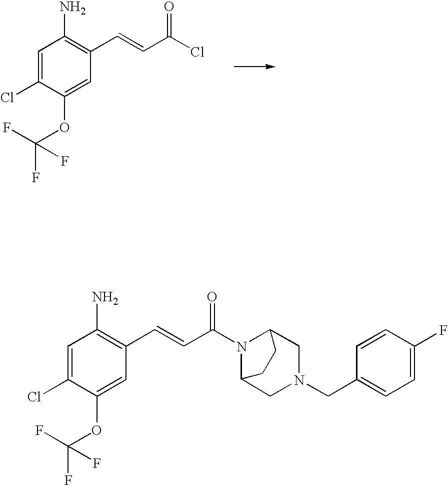

3-(4-Fluoro-benzyl)-3,8-diaza-bicyclo[3.2.1]octane (0.313 g; 1.42 mmol) in THF (4 ml) is combined at room temp. with (E)-3-(2-amino-4-chloro-5-trifluoromethoxy-phenyl)-acryloyl chloride hydrochloride (0.427 g; 1.42 c mmol) dissolved in toluene (4 ml). After 10 min. the fine precipitate is filtered off, washed with toluene, taken up in TBME and washed with a saturated solution of Na2CO3. The TBME phase is partially evaporated and poured on a column of SiO2 (TBME/MeOH/NH3conc 98/2/0.2) to yield the title compound as yellow foam (268 mg; 39%).

1H-NMR (400 MHz; DMSO-d6), δ (ppm): 1.70-1.80 (m, 1H); 1.83-1.95 (m, 3H); 2.10 (d, 1H); 2.18 (d, 1H); 2.60 (d, 1H); 2.65 (d, 1H); 3.45 (s, 2H);); 4.50 (bd, 1H); 4.65 (bd, 1H); 5.95 (s, 2H); 6.85 (s, 1H); 6.95 (s, 1H); 7.12 (t, 2H); 7.32 (m, 2H); 7.60 (d, 1H); 7.70 (s, 1H).

MS (m/z) ES+: 484 (MH+).

### f) (5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-urea

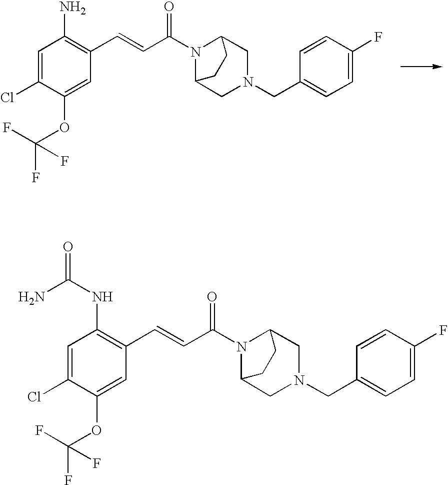

The title compound is prepared in analogy to Example 4 and purified via chromatography (SiO2; acetone/hexanes 4/6 to 1/1) followed by recrystallisation from TBME/hexanes delivering the target compound as colorless crystals (37 mg; 43%).

1H-NMR (400 MHz; DMSO-d6), δ (ppm): 1.70-1.80 (m, 1H); 1.83-1.95 (m, 3H); 2.16 (dd, 2H); 2.65 (d, 1H); 2.70 (d, 1H); 3.47 (s, 2H);); 4.53 (bd, 1H); 4.70 (bd, 1H); 6.31 (s, 2H); 7.10-7.18 (m, 3H); 7.29-7.35 (m, 2H); 7.63 (d, 1H); 7.95 (s, 1H); 8.13 (s, 1H); 8.55 (s, 1H).

MS (m/z) ES+: 527 (MH+, 70); 484 (100).

### Example 36

### 1-(5-chloro-2-{(E)-3-[3-(4-fluoro-benzyl)-3,8-diaza-bicyclo[3.2.1]oct-8-yl]-3-oxo-propenyl}-4-trifluoromethoxy-phenyl)-3-methyl-urea

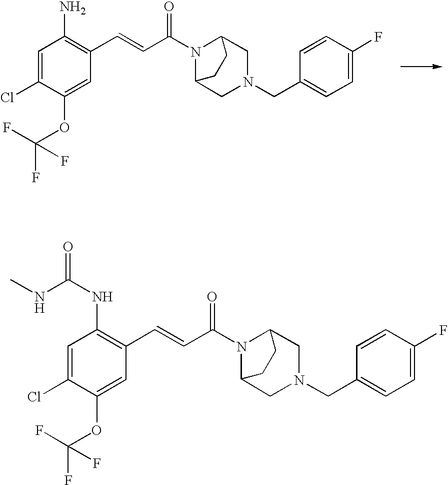

The target compound is prepared in analogy to Example 23f), purified via chromatography (XTerra, RP18, 7 μm, MeCN/water 40/60 to 100/0) and yielded the title compound as colorless crystals (40 mg; 40%).

1H-NMR (400 MHz; DMSO-d6), δ(ppm): 1.70-1.80 (m, 1H); 1.83-1.95 (m, 3H); 2.16 (dd, 2H); 2.61-2.71 (m, 5H); 3.47 (s, 2H);); 4.53 (bd, 1H); 4.70 (bd, 1H); 6.60 (s, 1H); 7.10-7.17 (m, 3H); 7.29-7.35 (m, 2H); 7.62 (d, 1H); 7.95 (s, 1H); 8.13 (s, 1H); 8.52 (s, 1H).

MS (m/z) ES+: 541 (MH+, 100); 484 (20).

### Example 37

### N-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-acetamide

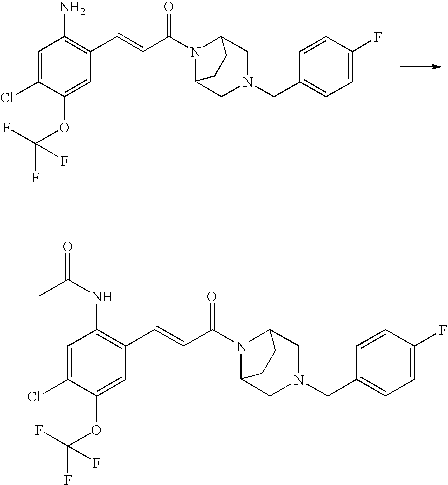

The target compound is prepared in analogy to Example 25, purified via chromatography (XTerra, RP18, 7 μm, MeCN/water 40/60 to 100/0) and yielded the title compound as colorless crystals (36 mg; 36%).

1H-NMR (400 MHz; DMSO-d6), δ (ppm): 1.65-1.80 (m, 1H); 1.83-1.95 (m, 3H); 2.10 (s, 3H); 2.16 (bt, 2H); 2.65 (d, 1H); 2.71 (d, 1H); 3.47 (s, 2H);); 4.53 (bd, 1H); 4.70 (bd, 1H); 7.11 (t, 2H); 7.20 (d, 1H); 7.32 (dd, 2H); 7.60 (d, 1H); 7.80 (s, 1H); 8.10 (s, 1H); 10.03 (s, 1H).

MS (m/z) ES+: 526 (MH+).

### Example 38

### 3-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-1,1-dimethyl-urea

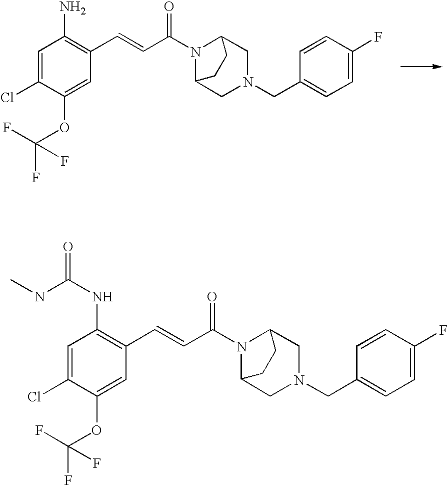

The target compound is prepared in analogy to Example 29, purified via chromatography (XTerra, RP18, 7 μm, MeCN/water 40/60 to 100/0) and yielded the title compound as colorless crystals (30 mg; 29%).

1H-NMR (400 MHz; DMSO-d6), δ (ppm): 1.70-1.80 (m, 1H); 1.83-1.95 (m, 3H); 2.15 (bt, 2H); 2.61-2.71 (m, 2H); 2.95 (s, 6H); 3.47 (s, 2H);); 4.51 (bd, 1H); 4.70 (bd, 1H); 7.10-7.17 (m, 3H); 7.29-7.35 (m, 2H); 7.52 (d, 1H); 7.58 (s, 1H); 8.08 (s, 1H); 8.41 (s, 1H).

MS (m/z) ES+: 555 (MH+, 100); 484 (45).

### Example 39

### 3-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-1,1-dimethylsulfonyl-urea

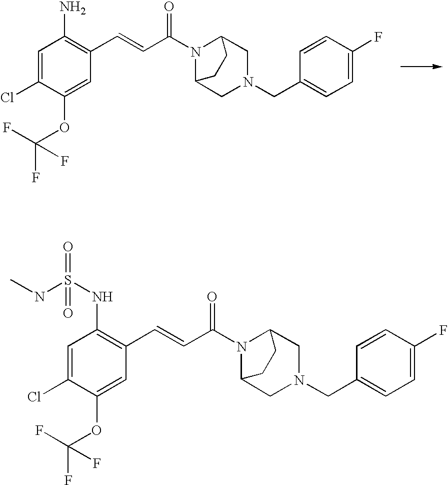

(E)-3-(2-Amino-4-chloro-5-trifluoromethoxy-phenyl)-1-[3-(4-fluoro-benzyl)-3,8-diaza-bicyclo[3.2.1]oct-8-yl]-propenone (100 mg; 0.20 mmol) are dissolved in pyridine (4 ml), N,N-dimethylsulfamoyl chloride (177 mg; 1.2 mmol) added and the mixture heated to 60° C. for 24 h. The reaction mixture is poured on water and extracted with TBME three times. The combined organic phases are dried over Na2SO4, filtered and evaporated to dryness delivering a product which is purified via chromatography (XTerra, RP18, 7 μm, MeCN/water 40/60 to 100/0) to yield the title compound as colorless foam (20 mg; 15%).

1H-NMR (400 MHz; DMSO-d6), δ (ppm): 1.70-1.80 (m, 1H); 1.83-1.95 (m, 3H); 2.15 (bt, 2H); 2.61-2.69 (m, 2H); 2.71 (bs, 6H); 3.47 (s, 2H);); 4.51 (bd, 1H); 4.70 (bd, 1H); 7.10-7.17 (m, 3H); 7.29-7.35 (m, 2H); 7.55 (s, 1H); 7.83 (d, 1H); 8.12 (bs, 1H); 10.00 (s, 1H).

MS (m/z) ES+: 591 (MH+, 100).

### Example 40

### 5-Chloro-2-{(E)-3-[3-(4-fluoro-benzyl)-3,8-diaza-bicyclo[3.2.1]oct-8-yl]-3-oxo-propenyl}-N,N-dimethyl-4-trifluoromethoxy-benzenesulfonamide

### a) 2-Bromo-5-chloro-N,N-dimethyl-4-trifluoromethoxy-benzenesulfonamide

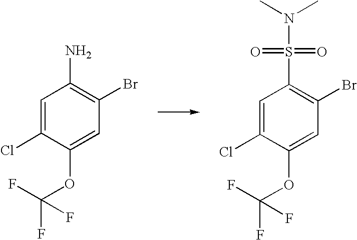

2-Bromo-5-chloro-4-trifluoromethoxy-phenylamine (Example 35a) (100 mg; 0.344 mmol) is dissolved in HOAc (0.5 ml) and added to HCl conc (1 ml). The resulting suspension is cooled to 0-5° C., NaNO2 (24 mg; 0.38 mmol) in water (0.2 ml) is added to generate a yellow solution of the diazonium salt. In a separate round bottom, SO2-gas is introduced into HOAc (4 ml) and cooled to 0° C.-20° C. CuCl (10 mg) is added at 0° C., followed by the diazonium salt solution prepared above. After the gas evolution had ceased, the reaction mixture is poured on water and extracted with TBME three times. The combined organic phases are dried over Na2SO4 and evaporated to dryness to deliver the intermediate sulfonyl chloride, which is dissolved in THF (4 ml) and treated with an excess of gaseous dimethylamine. The reaction mixture is concentrated and poured on a column of SiO2 (TBME/hexanes 3/7) to yield the title compound as colorless crystals (40 mg; 33%).

1H-NMR (400 MHz; DMSO-d6), δ (ppm): 2.87 (s, 6H); 8.14 (s, 1H); 8.15 (s, 1H).

MS (m/z) ES+: 384 (MH+).

### b) (E)-3-(4-Chloro-2-dimethylsulfamoyl-5-trifluoromethoxy-phenyl)-acrylic acid ethyl ester

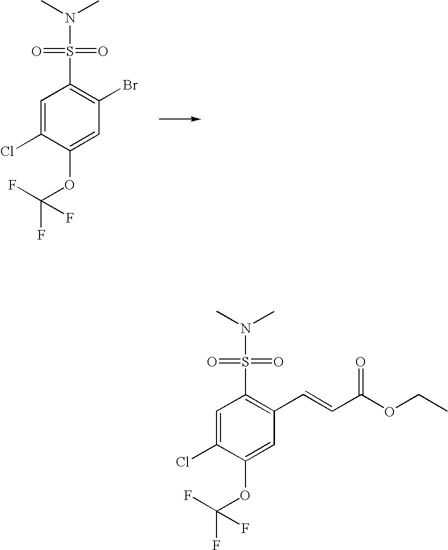

The title compound is obtained according to the method described in Example 30c and purified via chromatography (SiO2, TBME/hexanes 2/8) to deliver the desired product (40 mg; 96%) as colorless crystals.

1H-NMR (400 MHz; DMSO-d6), δ (ppm): 1.25 (t, 3H); 2.76 (s, 6H); 4.21 (q, 1H); 6.80 (d, 1H); 8.08 (s, 1H); 8.20 (s, 1H); 8.37 (d, 1H).

MS (m/z) ES+: 402 (MH+, 40); 356 (100).

### c) (E)-3-(4-Chloro-2-dimethylsulfamoyl-5-trifluoromethoxy-phenyl)-acrylic acid

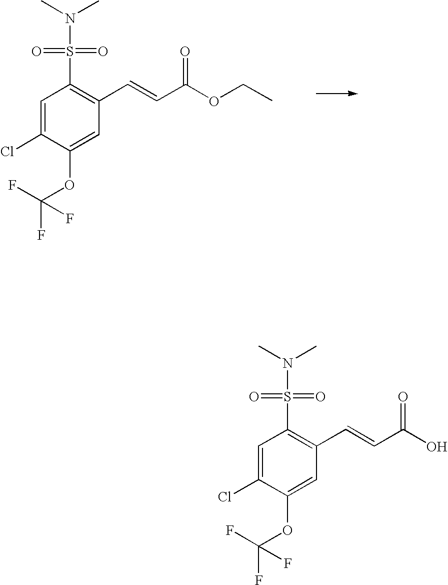

The title compound is obtained according to the method described in Example 30d and is obtained as colorless crystals (32 mg; 88%).

1H-NMR (400 MHz; DMSO-d6), δ (ppm): 2.76 (s, 6H); 6.66 (d, 1H); 8.06 (s, 1H); 8.15 (s, 1H); 8.21 (d, 1H); 12.8 (s, 1H).

MS (m/z) ES−: 372 (MH−).

### d) 5-Chloro-2-{(E)-3-[3-(4-fluoro-benzyl)-3,8-diaza-bicyclo[3.2.1]oct-8-yl]-3-oxo-propenyl}-N,N-dimethyl-4-trifluoromethoxy-benzenesulfonamide

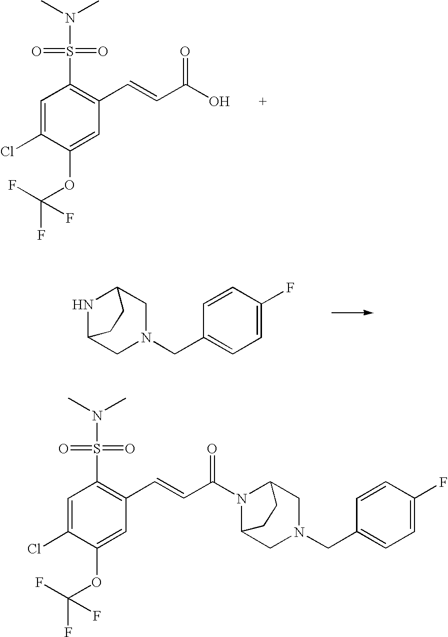

The title compound is obtained according to the method described in Example 30e and is purified via chromatography (SiO2, TBME/hexanes 2/8) to deliver the desired product as colorless crystals (31 mg; 67%).

1H-NMR (400 MHz; DMSO-d6), δ (ppm): 1.70-1.80 (m, 1H); 1.83-1.95 (m, 3H); 2.12 (d, 1H); 2.19 (d, 1H); 2.63 (d, 1H); 2.70 (d, 1H); 2.76 (s, 6H); 3.47 (s, 2H);); 4.51 (bd, 1H); 4.68 (bd, 1H); 7.13 (t, 2H); 7.21 (d, 1H); 7.29-7.35 (m, 2H); 8.05 (s, 1H); 8.15 (d, 1H); 8.28 (s, 1H).

MS (m/z) ES+: 576 (MH+, 100).

### Example 41

### (5-Chloro-2-{(E)-3-[3-(4-fluorobenzyl)-3,8-diazabicyclo[3.2.1]oct-8-yl]-3-oxopropenyl}-4-methylphenyl)-urea

### a) 2-Bromo-5-chloro-4-methylphenylamine

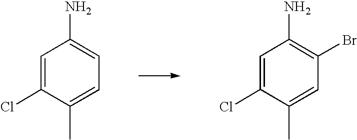

3-Chloro-4-methylphenylamine (20.0 g; 0.141 mmol) is dissolved in CH2Cl2 (200 ml) and combined within 5 min. with a solution of NBS (25.1 g; 0.141 mmol) in CH2Cl2 (800 ml). The reaction mixture is stirred for 5 min at room temp., evaporated to a volume of ˜200 ml and diluted with hexanes (1000 ml). The resulting precipitate is filtered off, the filtrate evaporated to dryness and purified via chromatography (SiO2, hexanes/TBME 10:1) to render the title compound as yellowish crystals (12.3 g; 40%). A second batch of title compound is obtained by re-chromatography of mixed fractions (7.5 g; 24%).

1H-NMR (400 MHz; DMSO-d6), δ (ppm): 2.13 (s, 3H); 5.32 (s, 2H, NH2); 6.81 (s, 1H); 7.31 (s, 1H).

MS (m/z) ES+: 221 (100, M+); 219 (80); 184 (35); 140 (100); 104 (50); 77 (65); 52 (58); 51 (60).

### b) (2-Amino-4-chloro-5-methylphenyl)-acrylic acid ethyl ester

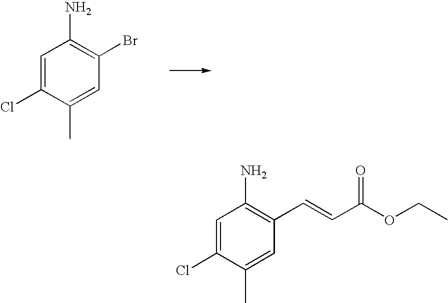

2-Bromo-5-chloro-4-methylphenylamine (3.0 g; 13.6 mmol) and ethyl-(E)-3-tributylstannyl)-propenoate (6.35 g; 16.3 mmol) are dissolved in DMF (60 ml). PdCl2(PPh3)2 (0.19 g; 0.27 mmol) in DMF (15 ml) is added and the reaction mixture heated to 140° C. for 60 min. under argon. The reaction mixture is evaporated and purified via chromatography (SiO2; hexanes/TBME 2:1) to yield the desired compound which is recrystallised from hexanes to yield the title compound as yellow crystals (2.21 g; 68%).

1H-NMR (400 MHz; DMSO-d6), δ (ppm): 1.25 (t, 3H); 2.15 (s, 3H); 4.15 (q, 2H); 5.65 (s, 2H, NH2); 6.38 (d, 1H); 6.75 (s, 1H); 7.43 (s, 1H); 7.77 (d, 1H).

MS (m/z) ES+: 239 (40, M+); 194 (100); 166 (60); 130 (70); 103 (20); 77 (30).

### c) (2-Amino-4-chloro-5-methylphenyl)-acrylic acid

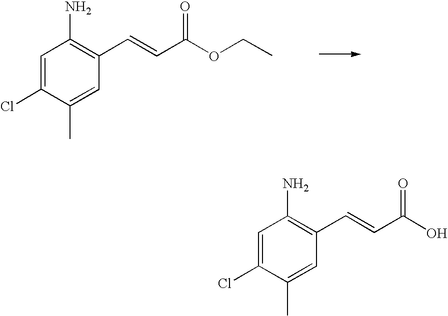

(2-Amino-4-chloro-5-methylphenyl)-acrylic acid ethyl ester (2.21 g; 9.22 mmol) is suspended in EtOH (100 ml) and 2N NaOH (6.9 ml; 13.83 mmol) and kept at 50° C. for 45 min. The reaction mixture is diluted with water and extracted with TBME twice. The aqueous phase is acidified with 2N HCl (20 ml) and extracted with TBME three times. The combined organic phases are dried over Na2SO4, filtered and evaporated to dryness to yield the title compound as yellow crystals (1.90 g; 97%).

1H-NMR (400 MHz; DMSO-d6), δ (ppm): 2.17 (s, 3H); 5.60 (s, 2H, NH2); 6.28 (d, 1H); 6.75 (s, 1H); 7.40 (s, 1H); 7.71 (d, 1H); 12.17 (s, 1H).

MS (m/z) ES+: 210 (100, MH−).

### d) (E)-3-(2-Amino-4-chloro-5-methylphenyl)-1-[3-(4-fluorobenzyl)-3,8-diazabicyclo[3.2.1]oct-8-yl]-propenone

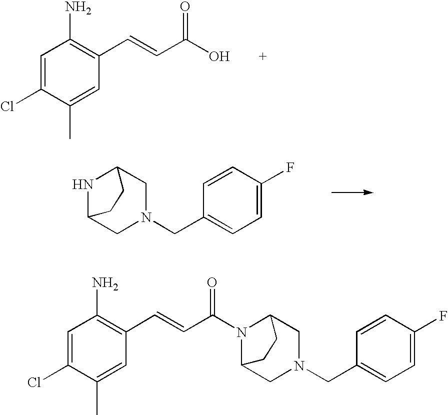

(2-Amino-4-chloro-5-methylphenyl)-acrylic acid (0.98 g; 4.63 mmol), 3-(4-fluorobenzyl)-3,8-diazabicyclo[3.2.1]octane (1.02 g; 4.63 mmol), EDCl (1.06 g; 5.56 mmol) and HOBt (0.07 g; 0.46 mmol) in DMF (20 ml) are kept at room temp. over night. The reaction mixture is poured on 20% aqueous HOAc and extracted with TBME three times. The combined organic phases are dried over Na2SO4, evaporated to dryness and purified via chromatography (hexanes/acetone 4:1) to yield the title compound as yellow crystals (1.7 g; 90%).

1H-NMR (400 MHz; DMSO-d6), δ (ppm): 1.65-1.75 (m, 1H); 1.80-1.95 (m, 3H); 2.10-2.20 (m, 2H); 2.15 (s, 3H); 2.60-2.70 (m, 2H); 3.45 (s, 2H); 4.50 (bd, 1H); 4.65 (bd, 1H); 5.46 (s, 2H, NH2); 6.72 (s, 1H); 6.86 (d, 1H); 7.12 (t, 2H); 7.31 (m, 2H); 7.47 (s, 1H); 7.58 (d, 1H).

MS (m/z) ES+: 414 (100, M+).

### e) (5-Chloro-2-{(E)-3-[3-(4-fluorobenzyl)-3,8-diazabicyclo[3.2.1]oct-8-yl]-3-oxopropenyl}-4-methylphenyl)-urea

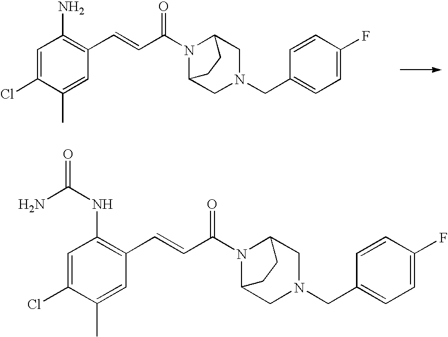

(E)-3-(2-Amino-4-chloro-5-methylphenyl)-1-[3-(4-fluorobenzyl)-3,8-diazabicyclo[3.2.1]oct-8-yl]-propenone (1.73 g; 4.18 mmol) in THF (65 ml) is treated with triphosgene (1.24 g; 4.18 mmol) at room temp. under stirring for 10 min. An excess of NH3-gas is introduced, stirring continued for 20 min., the reaction mixture poured on water and extracted with TBME twice. The combined organic phases are dried over Na2SO4, evaporated to dryness and purified via chromatography (SiO2; hexanes/acetone 7:3 to 1:1) to yield the title compound, which contained a considerable amount of undesired bis-urea derivative. Pure title compound (436 mg; 22%) is obtained after HPLC-chromatography (Gilson; XTerra; MeCN/water 40/60 to 100/0).

1H-NMR (400 MHz; DMSO-d6), δ (ppm): 1.68-1.78 (m, 1H); 1.82-1.97 (m, 3H); 2.15 (dd, 2H); 2.38 (s, 3H); 2.62-2.72 (m, 2H); 3.46 (s, 2H); 4.55 (bd, 1H); 4.67 (bd, 1H); 6.13 (s, 2H, NH2); 7.03 (d, 1H); 7.12 (t, 2H); 7.32 (d, 2H); 7.64 (d, 1H); 7.73 (s, 1H); 7.85 (s, 1H); 8.29 (s, 1H).

MS (m/z) ES+: 457 (100, MH+).

### Example 42

### N-(5-Chloro-2-{(E)-3-[3-(4-fluoro-benzyl)-3,8-diaza-bicyclo[3.2.1]oct-8-yl]-3-oxo-propenyl}-4-methyl-phenyl)-methanesulfonamide

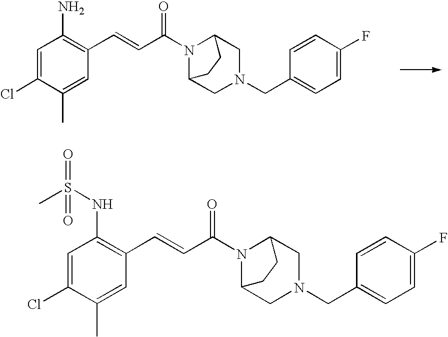

The title compound is prepared in analogy to Example 5, purified via chromatography (SiO2, TBME/MeOH/NH3conc 98/2/0.1) to yield the desired product as colorless crystals after recrystallisation from TBME (54 mg; 57%).

1H-NMR (400 MHz; DMSO-d6), δ (ppm): 1.68-1.78 (m, 1H); 1.82-1.97 (m, 3H); 2.15 (dd, 2H); 2.35 (s, 3H); 2.62-2.72 (m, 2H); 2.97 (s, 3H); 3.46 (s, 2H); 4.52 (bd, 1H); 4.67 (bd, 1H); 7.07 (d, 1H); 7.12 (t, 2H); 7.30-7.40 (m, 3H); 7.80 (d, 1H); 8.04 (s, 1H); 9.58 (bs, 1H).

MS (m/z) ES+: 492 (MH+, 100).

### Example 43

### N-(5-Chloro-2-{(E)-3-[3-(4-fluoro-benzyl)-3,8-diaza-bicyclo[3.2.1]oct-8-yl]-3-oxo-propenyl}-4-methyl-phenyl)-1,1-dimethylsulfonyl-urea

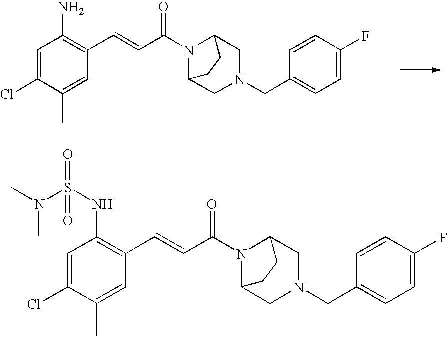

The title compound is prepared in analogy to Example 39, purified via chromatography (SiO2, TBME/acetone 20/1) to yield the desired product as yellowish foam (94 mg; 48%).

1H-NMR (400 MHz; DMSO-d6), δ (ppm): 1.68-1.78 (m, 1H); 1.82-1.97 (m, 3H); 2.15 (dd, 2H); 2.30 (s, 3H); 2.62-2.70 (m, 8H); 3.46 (s, 2H); 4.52 (bd, 1H); 4.67 (bd, 1H); 7.07 (d, 1H); 7.12 (t, 2H); 7.30-7.40 (m, 4H); 7.85 (d, 1H); 9.58 (bs, 1H).

MS (m/z) ES+: 521 (MH+, 100).

### Example 44

### N-(5-Chloro-2-{(E)-3-[3-(4-fluoro-benzyl)-3,8-diaza-bicyclo[3.2.1]oct-8-yl]-3-oxo-propenyl}-4-methyl-phenyl)-2-methoxy-acetamide

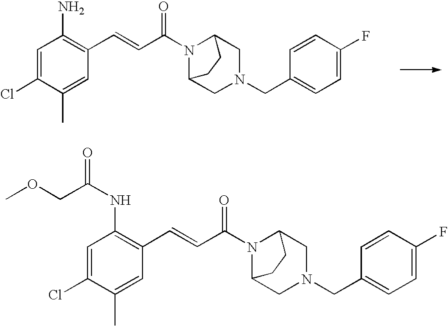

The title compound is prepared in analogy to Example 6 and purified via recrystallisation from TBME to yield the desired product as colorless crystals (29 mg; 31%).

1H-NMR (400 MHz; DMSO-d6), δ (ppm): 1.68-1.78 (m, 1H); 1.82-1.97 (m, 3H); 2.15 (dd, 2H); 2.33 (s, 3H); 2.62 (d, 1H); 2.68 (d, 1H); 3.40 (s, 3H); 3.46 (s, 2H); 4.03 (s, 2H); 4.52 (bd, 1H); 4.67 (bd, 1H); 7.07 (d, 1H); 7.12 (t, 2H); 7.30 (m, 2H); 7.45 (s, 1H); 7.53 (d, 1H); 7.89 (s, 1H); 9.70 (bs, 1H).

MS (m/z) ES+: 486 (MH+, 100).

### Example 45

### N-(5-Chloro-2-{(E)-3-[3-(4-fluoro-benzyl)-3,8-diaza-bicyclo[3.2.1]oct-8-yl]-3-oxo-propenyl}-4-methyl-phenyl)-acetamide

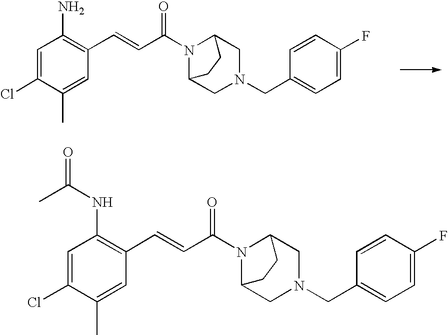

The title compound is prepared in analogy to Example if and is obtained colorless crystals (70 mg; 80%).

1H-NMR (400 MHz; DMSO-d6), δ (ppm): 1.68-1.78 (m, 1H); 1.82-1.97 (m, 3H); 2.06 (s, 3H); 2.15 (dd, 2H); 2.32 (s, 3H); 2.62 (d, 1H); 2.70 (d, 1H); 3.46 (s, 2H); 4.52 (bd, 1H); 4.67 (bd, 1H); 7.06 (d, 1H); 7.13 (t, 2H); 7.30 (dd, 2H); 7.40 (s, 1H); 7.60 (d, 1H); 7.86 (s, 1H); 9.80 (s, 1H).

MS (m/z) ES+: 456 (MH+).

### Example 46

### 1-(5-Chloro-2-{(E)-3-[3-(4-fluoro-benzyl)-3,8-diaza-bicyclo[3.2.1]oct-8-yl]-3-oxo-propenyl}-4-methyl-phenyl)-3-methyl-urea

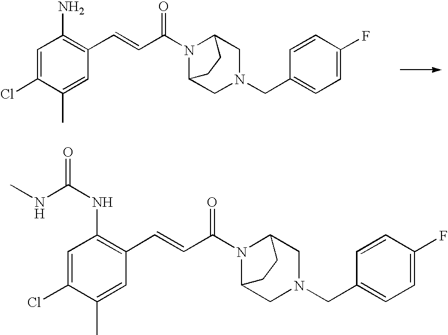

The title compound is prepared in analogy to Example 23f, purified via chromatography (SiO2, acetone/hexanes 2/8 to 25/75) and crystallized from TBME to yield the desired product as colorless crystals (47 mg; 50%).

1H-NMR (400 MHz; DMSO-d6), δ (ppm): 1.68-1.78 (m, 1H); 1.82-1.97 (m, 3H); 2.15 (dd, 2H); 2.29 (s, 3H); 2.60-2.72 (m, 5H); 3.46 (s, 2H); 4.52 (bd, 1H); 4.67 (bd, 1H); 6.42 (bq, 1H); 7.00 (d, 1H); 7.13 (t, 2H); 7.30 (dd, 2H); 7.60 (d, 1H); 7.73 (s, 1H); 7.83 (s, 1H); 8.35 (s, 1H).

MS (m/z) ES+: 471 (MH+, 45); 440 (15); 414 (100).

### Example 47

### 3-(5-Chloro-2-{(E)-3-[3-(4-fluoro-benzyl)-3,8-diaza-bicyclo[3.2.1]oct-8-yl-3-oxopropenyl}-4-methyl-phenyl)-1,1-dimethyl-urea

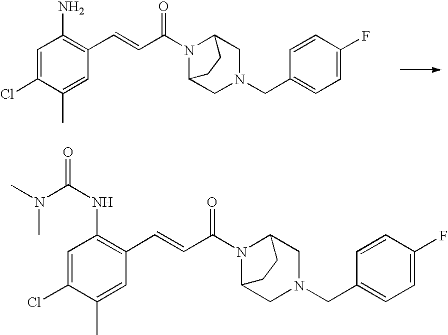

The title compound is prepared in analogy to Example 29, purified via chromatography (SiO2, acetone/hexanes 2/8 to 25/75) and crystallized from TBME to yield the desired product as colorless crystals (48 mg; 51%).

1H-NMR (400 MHz; DMSO-d6), δ (ppm): 1.68-1.78 (m, 1H); 1.82-1.97 (m, 3H); 2.15 (dd, 2H); 2.32 (s, 3H); 2.60-2.72 (m, 2H); 2.92 (s, 6H); 3.46 (s, 2H); 4.52 (bd, 1H); 4.67 (bd, 1H); 7.00 (d, 1H); 7.13 (t, 2H); 7.28 (s, 1H); 7.32 (dd, 2H); 7.53 (d, 1H); 7.83 (s, 1H); 8.20 (s, 1H).

MS (m/z) ES+: 485 MH+, 30); 440 (30); 414 (100).

### Example 48

### 3-Oxo-piperazine-1-carboxylic acid (5-chloro-2-{(E)-3-[3-(4-fluoro-benzyl)-3,8-diaza-bicyclo[3.2.1]oct-8-yl]-3-oxo-propenyl}-4-methyl-phenyl)-amide

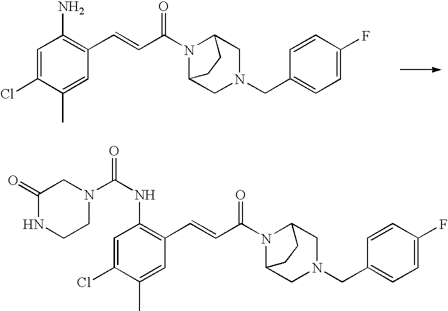

The title compound is prepared in analogy to Example 14, purified via chromatography (SiO2, acetone/hexanes 1/1 to 1/0) and crystallized from EtOAc to yield the desired product as colorless crystals (52 mg; 50%).

1H-NMR (400 MHz; DMSO-d6), δ (ppm): 1.68-1.78 (m, 1H); 1.82-1.97 (m, 3H); 2.15 (dd, 2H); 2.32 (s, 3H); 2.60-2.72 (m, 2H); 3.25 (bs, 2H); 3.42 (bs, 2H); 3.60 (s, 2H); 4.00 (s, 2H); 4.52 (bd, 1H); 4.67 (bd, 1H); 7.03 (d, 1H); 7.13 (t, 2H); 7.30 (m, 3H); 7.53 (d, 1H); 7.85 (s, 1H); 8.08 (s, 1H); 8.50 (s, 1H).

MS (m/z) ES+: 540 (MH+, 15); 414 (100).

### Example 49

### 1-(5-Chloro-2-{(E)-3-[3-(4-fluoro-benzyl)-3,8-diaza-bicyclo[3.2.1]oct-8-yl]-3-oxo-propenyl}-4-methyl-phenyl)-3-cyclopropyl-urea

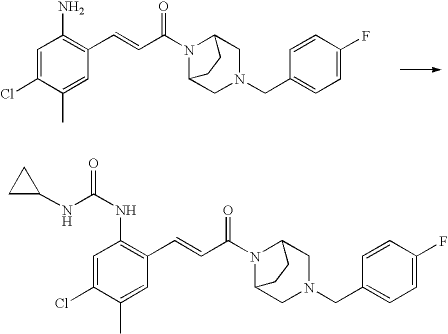

The title compound is prepared in analogy to Example 12, purified via chromatography (XTerra, RP18, 7 μm, MeCN/water 40160 to 100/0) to yield the title compound as yellowish foam (32 mg; 34%).

1H-NMR (400 MHz; DMSO-d6), δ (ppm): 0.42 (bs, 2H); 0.65 (bd, 2H); 1.68-1.78 (m, 1H); 1.82-1.97 (m, 3H); 2.15 (dd, 2H); 2.28 (s, 3H); 2.60-2.72 (m, 2H); 3.43 (s, 2H); 4.52 (bd, 1H); 4.67 (bd, 1H); 6.80 (bs, 1H); 7.03 (d, 1H); 7.13 (t, 2H); 7.30 (m, 3H); 7.60 (d, 1H); 7.73 (s, 1H); 7.88 (s, 1H); 8.13 (s, 1H).

MS (m/z) ES+: 497 (MH+, 100); 440 (20); 414 (75); 396 (15).

### Example 50

### 1-(5-Chloro-2-{(E)-3-[3-(4-fluoro-benzyl)-3,8-diaza-bicyclo[3.2.1]oct-8-yl]-3-oxo-propenyl}-4-methyl-phenyl)-tert-butylsulfonyl-urea

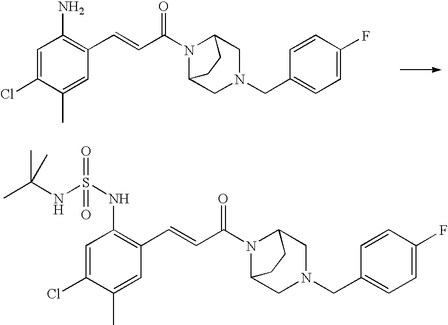

tert-Butylsulfamoyl chloride (41 mg; 0.24 mmol) (J. Org. Chem. (1976), 41, 4028-9) in THF (0.2 ml) is added to a solution of (E)-3-(2-amino-4-chloro-5-methylphenyl)-1-[3-(4-fluorobenzyl)-3,8-diazabicyclo[3.2.1]oct-8-yl]-propenone (100 mg; 0.24 mmol) and NEt3 (0.034 ml; 0.24 mmol) in THF (0.3 ml). The reaction mixture is stirred for 1 h at room temp., poured on a saturated solution of Na2CO3 and extracted with TBME three times. The organic phases are diered over Na2SO4, filtered and evaporated to dryness to deliver a product which is purified via chromatography (SiO2, acetone/hexanes 2/8) to yield the desired product as colorless foam (49 mg; 37%).

1H-NMR (400 MHz; DMSO-d6), δ (ppm): 1.20 (s, 9H); 1.68-1.78 (m, 1H); 1.82-1.97 (m, 3H); 2.15 (dd, 2H); 2.30 (s, 3H); 2.60-2.72 (m, 2H); 3.45 (s, 2H); 4.52 (bd, 1H); 4.67 (bd, 1H); 7.00 (d, 1H); 7.13 (bt, 2H); 7.23 (s, 1H); 7.32 (dd, 2H); 7.52 (s, 1H); 7.77 (s, 1H); 7.80 (s, 1H); 9.35 (s, 1H).

MS (m/z) ES+: 549 (MH+, 70); 493 (10); 414 (100); 396 (40).

### Example 51

### 5-Chloro-2-{(E)-3-[3-(4-fluoro-benzyl)-3,8-diaza-bicyclo[3.2.1]oct-8-yl]-3-oxo-propenyl}-4,N,N-trimethyl-benzenesulfonamide

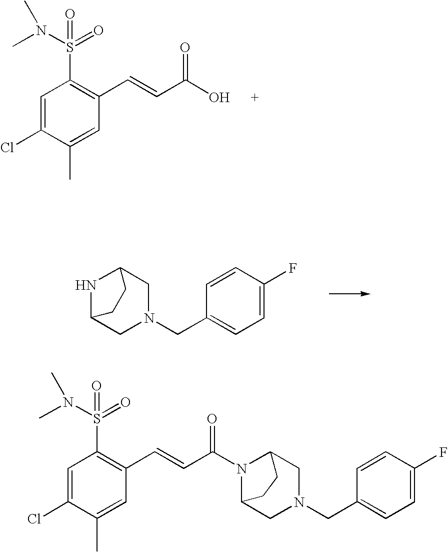

(E)-3-(4-Chloro-2-dimethylsulfamoyl-5-methyl-phenyl)-acrylic acid (prepared in analogy to Example 40c from 2-bromo-5-chloro-4-methylphenylamine) is converted to the title compound using the methodology described in Example 23d. Purification via chromatography (SiO2, acetone/hexanes 1/3) yielded the desired product as colorless crystals (144 mg; 86%).

1H-NMR (400 MHz; DMSO-d6), δ (ppm): 1.68-1.78 (m, 1H); 1.82-1.97 (m, 3H); 2.15 (dd, 2H); 2.45 (s, 3H); 2.62-2.70 (m, 8H); 3.48 (s, 2H); 4.52 (bd, 1H); 4.67 (bd, 1H); 7.07-7.15 (m, 3H); 7.30 (dd, 2H); 7.78 (s, 1H); 8.08 (s, 1H); 8.20 (d, 1H).

MS (m/z) ES+: 506 (MH+, 100).

### Example 52

### N-(3′-Amino-2-chloro-5-{(E)-3-[3-(4-fluoro-benzyl)-3,8-diaza-bicyclo[3.2.1]oct-8-yl]-3-oxo-propenyl}-biphenyl-4-yl)-acetamide

### a) (E)-3-(5-Bromo-4-chloro-2-nitro-phenyl)-acrylic acid methyl ester

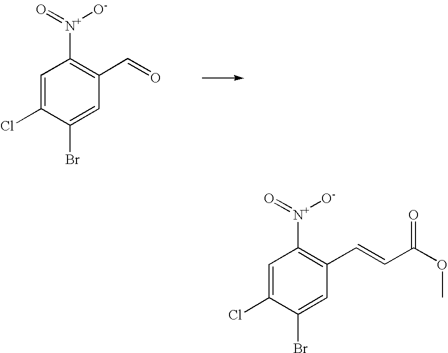

7.40 g (27.98 mmol) 5-Bromo-4-chloro-2-nitrobenzaldehyde (WO 9804556A1) and 10.30 g (30.77 mmol) (Methoxycarbonylmethylene)triphenylphosphorane are mixed in 150 ml toluol and stirred at 120° C. for 30 min. Then the mixture is diluted with 200 ml water and extracted with ethyl acetate. The title compound is purified by chromatography (SiO₂, ethyl acetate/c-hexane 1/19 to 1/9) and is isolated as a pale solid (5.0 g, 55%)

1H-NMR (400 MHz; DMSO-d6): 3.75 (s, 3H), 6.75 (d, 1H), 7.80 (d, 1H), 8.35 (s, 1H), 8.37 (s, 1H)

MS (m/z) EI: 321 (M+, 10), 275 ([M−NO2]+, 100)

### b) (E)-3-(5-Bromo-4-chloro-2-nitro-phenyl)-acrylic acid

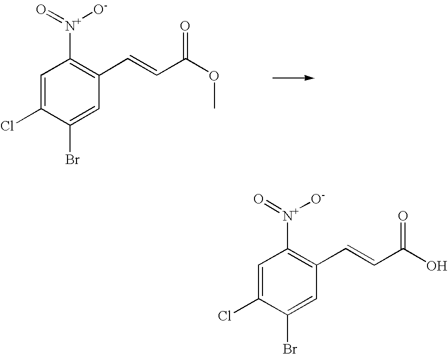

Title compound of step a) (8.20 g, 25.58 mmol) is dissolved in 220 ml MeOH and 20.0 ml 2N NaOH is added, this mixture is heated to 50° C. for 3 hours. The solution is cooled to room temperature and 2N HCl is added to reach pH˜1. The title compounds is isolated as a pale solid (4.0 g, 51%).

1H-NMR (400 MHz; DMSO-d6): 6.65 (d, 1H), 7.70 (d, 1H), 8.35 (s, 1H), 8.37 (s, 1H), 12.80 (s, 10H)

MS (m/z) EI: 306 ([M−H]−, 100)

### c) (E)-3-(5-Bromo-4-chloro-2-nitro-phenyl)1-1[3-(4fluoro-benzyl)-3,8-diaza-bicyclo[3.2.1]oct-8-yl]-propenone

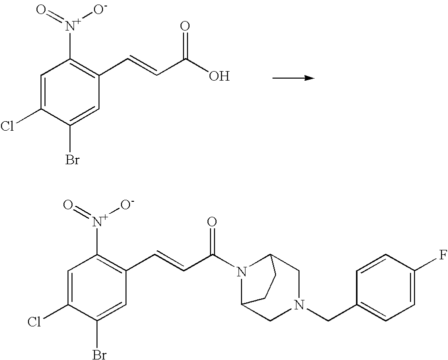

Title compound of step b) (1.00 g, 3.26 mmol), EDCl (688 mg, 3.58 mmol); HOBT (484 mg, 3.58 mmol) and 790 mg (3.58 mmol) 3-(4-Fluoro-benzyl)-3,8-diaza-bicyclo[3.2.1]octane are dissolved in 150 ml THF and stirred for 5 hours at room temperature. The reaction mixture is diluted with 400 ml water and extracted with ethyl acetate. The title compound is purified by chromatography (SiO₂, ethyl acetate/MeOH/NH₃conc. 98/2/0.2) and is isolated as a pale solid (670 mg, 40%) 1H-NMR (400 MHz; DMSO-d6): 1.70-1.78 (m, 1H), 1.80-1.95 (m, 3H), 2.15-2.25 (m, 2H), 2.65-2.75 (m, 2H), 3.45 (s, 2H), 4.45-4.55 (m, 1H), 4.70-4.75 (m, 1H), 7.05-7.15 (m, 2H), 7.25-7.35 (m, 3H), 8.30 (s, 1H), 8.50 (s, 1H)

MS (m/z) EI: 510 ([MH]+, 100)

### d) (E)-3-(2-Amino-5-bromo-4-chloro-phenyl)-1-[3-(4-fluoro-benzyl)-3,8-diaza-bicyclo[3.2.1]oct-8-yl]-propenone

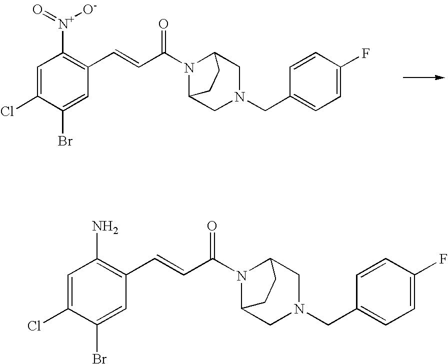

Title compound of step c) (0.67 g, 1.31 mmol) is dissolved in 25 ml THF and 5.0 ml water and 1.25 g (6.58 mmol) stannous chloride anhydrous are added. This mixture is stirred at 80 ⁰C for 30 min., then the mixture is diluted with 100 ml saturated sodium carbonate solution and extracted with ethyl acetate. The title compound is purified by chromatography (SiO₂, ethyl acetate/MeOH/NH₃conc. 98/2/0.2) and is isolated as a pale solid (0.41 g, 67%)

1H-NMR (400 MHz; DMSO-d6): 1.65-1.75 (m, 1H), 1.80-1.95 (m, 3H), 2.10-2.20 (m, 2H), 2.60-2.70 (m, 2H), 3.45 (s, 2H), 4.45-4.55 (m, 1H), 4.65-4.70 (m, 1H), 5.85 (bs, 2NH), 6.85 (s, 1H), 6.95 (d, 1H), 7.05-7.15 (m, 2H), 7.25-7.35 (m, 2H), 7.55 (d, 1H), 7.85 (s, 1H)

MS (m/z) EI: 480 ([MH]+, 100)

### e) N-(4-Bromo-5-chloro-2-{(E)-3-[3-(4-fluoro-benzyl)-3,8-diaz-bicyclo[3.2.1]oct-8-yl]-3-oxo-propenyl}-phenyl)-acetamide

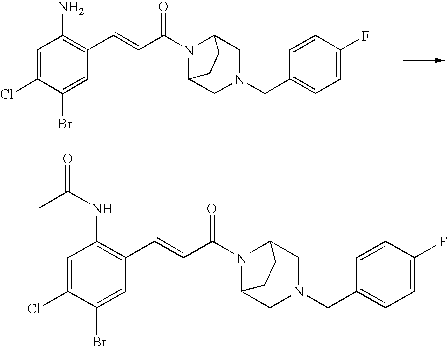

Title compound of step d) (410 mg, 0.86 mmol) is dissolved in 20 ml THF and 0.60 ml (4.28 mmol) NEt₃ and 0.30 ml (4.28 mmol) acetyl chloride are added. This mixture is stirred for 2 hours at room temperature. Then the mixture is diluted with 100 ml saturated sodium carbonate solution and extracted with ethyl acetate. The title compound is purified by chromatography (SiO₂, ethyl acetate/MeOH/NH₃conc. 98/2/0.2) and is isolated as a pale solid (300 mg, 67%).

1H-NMR (400 MHz; DMSO-d6): 1.65-1.75 (m, 1H), 1.80-1.95 (m, 3H), 2.07 (s, 3H), 2.10-2.20 (m, 2H), 2.60-2.70 (m, 2H), 3.45 (s, 2H), 4.45-4.55 (m, 1H), 4.70-4.75 (m, 1H), 7.05-7.15 (m, 2H), 7.20 (d, 1H), 7.25-7.35 (m, 2H), 7.55 (d, 1H), 7.75 (s, 1H), 8.30 (s, 1H), 9.95 (bs, 1 NH)

MS (m/z) EI: 522 ([MH]+, 100)

### f) N-(3′-Amino-2-chloro-5-{(E)-3-[3-(4-fluoro-benzyl)-3,8-diaza-bicyclo[3.2.1]oct-8-yl]-3-oxo-propenyl}-biphenyl-4-yl)-acetamide

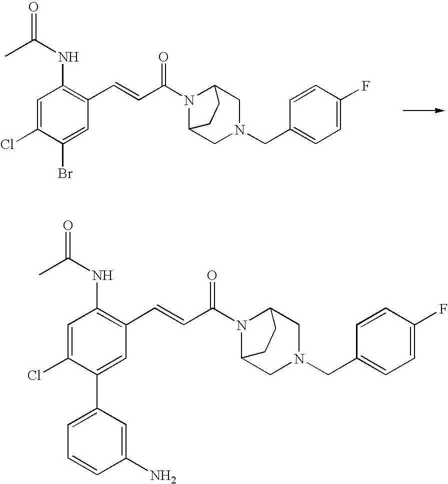

Title compound of step e) 300.0 mg (0.58 mmol) is dissolved in 15 ml DMF. After 5 min. stirring under argon at room temperature 30.0 mg (0.026 mmol) tetrakis(triphenylphosphie)palladium(0) are added. Stirring is continued at room temperature for 5 min. under argon then 250.0 mg (1.44 mmol) 3-Aminophenylboronic acid hydrochloride are added followed by the addition of 7.5 ml 1N sodiumbicarbonate solution. This mixture is stirred at 90° C. for 2 hours and then poured into 300 ml water and extracted with ethyl acetate. The title compound is purified by chromatography (SiO₂, ethyl acetate/MeOH/NH₃conc. 98/2/0.2) and is isolated as a pale solid (205 mg, 66%).

1H-NMR (400 MHz; DMSO-d6): 1.65-1.75 (m, 1H), 1.80-1.95 (m, 3H), 2.08 (s, 3H), 2.10-2.20 (m, 2H), 2.60-2.70 (m, 2H), 3.45 (s, 2H), 4.45-4.55 (m, 1H), 4.70-4.75 (m, 1H), 5.15 (bs, 2NH), 6.45-6.55 (m, 2H), 7.05-7.15 (m, 3H), 7.25-7.35 (m, 2H), 7.55-7.65 (m, 4H), 7.85 (s, 1H), 9.90 (bs, 1NH)

MS (m/z) EI: 533 ([MH]+, 100)

### Example 53

### N-(3′-Acetylamino-2-chloro-5-{(E)-3-[3-(4-fluoro-benzyl)-3,8-diaza-bicyclo[3.2.1]oct-8-yl]-3-oxo-propenyl}-biphenyl-4-yl)-acetamide

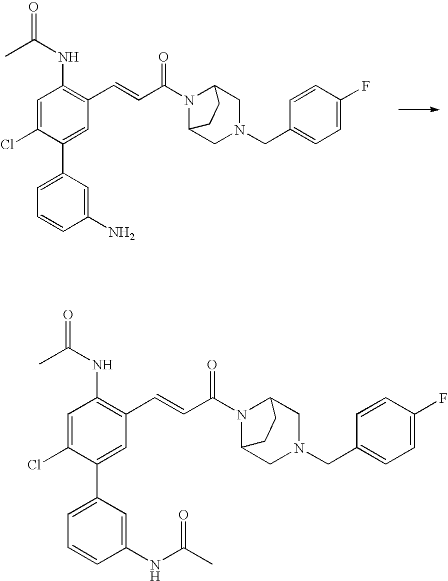

Title compound of example 5 (80 mg, 0.15 mmol) is dissolved in 3 ml THF and 0.10 ml (0.75 mmol) NEt₃ and 0.05 ml (0.75 mmol) acetyl chloride are added. This mixture is stirred for 1 hour at room temperature. Then the mixture is diluted with 10 ml saturated sodium carbonate solution and extracted with ethyl acetate. The title compound is purified by chromatography (SiO₂, ethyl acetate/MeOH/NH₃conc. 95/5/0.5) and is isolated as a pale solid (64 mg, 74%).

1H-NMR (400 MHz; DMSO-d6): 1.65-1.75 (m, 1H), 1.80-1.95 (m, 3H), 2.03 (s, 3H), 2.05-2.20 (m, 5H), 2.60-2.65 (m, 2H), 3.45 (s, 2H), 4.45-4.55 (m, 1H), 4.65-4.70 (m, 1H), 7.05-7.15 (m, 4H), 7.25-7.35 (m, 2H), 7.36-7.40 (t, 1H), 7.55-7.65 (m, 4H), 7.90 (s, 1H), 9.90 (bs, 1NH), 10.00 (bs, 1NH)

MS (m/z) EI: 575 ([MH]+, 100)

### Example 54

### N-(2-Chloro-5-{(E)-3-[3-(4-fluoro-benzyl)-3,8-diaza-bicyclo[3.2.1]oct-8-yl]-3-oxo-propenyl}-3′-ureido-biphenyl-4-yl)-acetamide

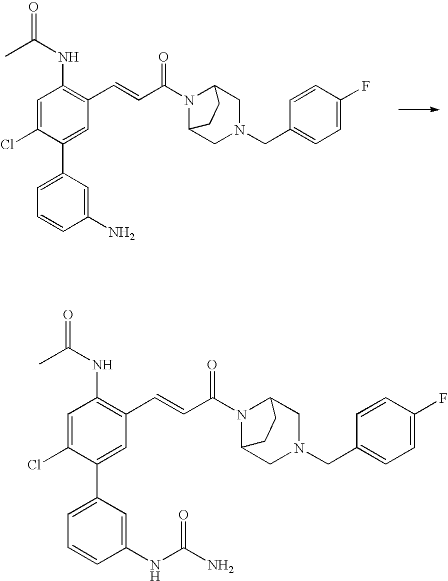

Title compound of example 5 (80 mg, 0.15 mmol) is dissolved in 0.5 ml acetic acid and 0.5 ml water and 19.5 mg (0.30 mmol) sodium isocyanate are added. This mixture is stirred at room temperature for 1 hour then the mixture is diluted with 10 ml saturated sodium carbonate solution and extracted with ethyl acetate. The title compound is purified by chromatography (SiO₂, ethyl acetate/MeOH/NH₃conc. 95/510.5) and is isolated as a pale solid (36 mg, 41%)

1H-NMR (400 MHz; DMSO-d6): 1.65-1.75 (m, 1H), 1.80-1.95 (m, 3H), 2.05-2.20 (m, 5H), 2.60-2.65 (m, 2H), 3.45 (s, 2H), 4.45-4.55 (m, 1H), 4.65-4.70 (m, 1H), 5.85 (bs, 2NH), 6.95 (d, 1H), 7.05-7.15 (m, 3H), 7.25-7.35 (m, 3H), 7.40-7.45 (m, 2H), 7.55-7.60 (m, 2H), 7.85 (s, 1H), 8.10 (bs, 1NH), 9.95 (bs, 1NH)

MS (m/z) EI: 576 ([MH]+, 100)

### Example 55

### N-(5-Chloro-2-{(E)-3-[3-(4-fluoro-benzyl)-3,8-diaza-bicyclo[3.2.1]oct-8-yl]-3-oxo-propenyl}-4-pyrazin-2-yl-phenyl)-acetamide

### a) 3-Chloro-4-pyrazin-2-yl-phenylamine

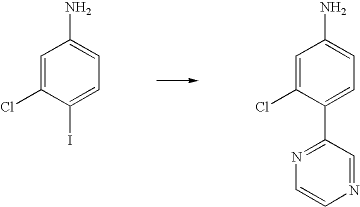

3-Chloro-4-iodo aniline (0.349 g; 1.375 mmol), 2-(tri-n-butylstannyl)pyrazine and (1.015 g; 2.75 mmol) PdCl2(PPh3)2 (0.193 g; 0.16 mmol) are dissolved in xylene (5 ml) and refluxed for 2.5 h. The reaction mixture is taken up in TBME and extracted with 2N HCl three times. The combined HC-phases are poured on a saturated solution of saturated Na2CO3 and extracted with TBME three times. The combined organic phases are dried over Na2SO4, filtered and evaporated to dryness and purified via chromatography (SiO2, acetone/hexanes 1/3) to yield the desired product as yellow crystals (162 mg; 57%).

1H-NMR (400 MHz; DMSO-d6), δ (ppm): 5.78 (s, 2H); 6.62 (dd, 1H); 6.70 (d, 1H); 7.34 (d, 1H); 8.50 (d, 1H); 8.63 (m, 1H); 9.48 (s, 1H).

MS (m/z) ES+: 206 (MH+).

### b) 2-Bromo-5-chloro-4-pyrazin-2-yl-phenylamine

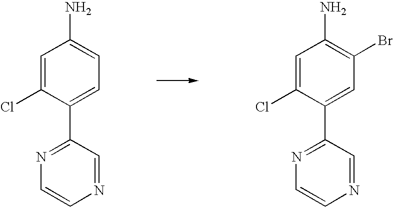

3-Chloro-4-pyrazin-2-yl-phenylamine (160 mg; 0.778 mmol) and N-bromosuccinimid (139 mg; 0.778 mmol) in CH2Cl2 (6 ml) are stirred for 10 min. at room temp. The reaction mixture is poured on a column of SiO2 (hexanes/TBME 3/1) to yield the title product as off-white crystals (161 mg; 73%).

1H-NMR (400 MHz; DMSO-d6), δ (ppm): 5.96 (bs, 2H); 6.94 (s, 1H); 7.64 (s, 1H); 8.54 (d, 1H); 8.66 (m, 1H); 8.86 (m, 1H).

MS (m/z) ES+: 286 (MH+).

### c) (E)-3-(2-Amino-4-chloro-5-pyrazin-2-yl-phenyl)-acrylic acid ethyl ester

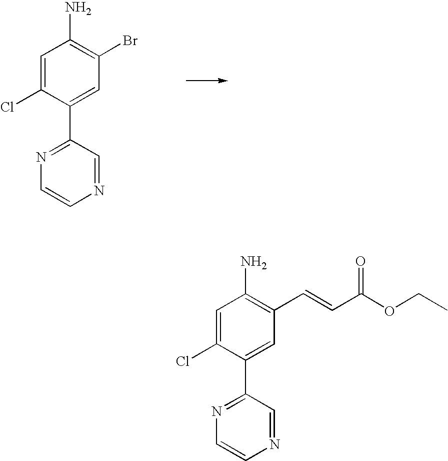

The reaction is performed in analogy to Example 23b and the title product purified via chromatography (SiO2, hexanes/TBME 1/1) to yield the desired product as yellow crystals (171 mg; 56%).

1H-NMR (400 MHz; DMSO-d6), δ (ppm): 1.24 (t, 3H); 4.17 (q, 2H); 6.23 (bs, 2H); 6.45 (d, 1H); 6.88 (s, 1H); 7.74 (s, 1H); 7.82 (d, 1H); 8.54 (d, 1H); 8.67 (m, 1H); 8.85 (s, 1H).

MS (m/z) ES+: 304 (MH+, 100); 258 (55).

### d) (E)-3-(2-Acetylamino-4-chloro-5-pyrazin-2-yl-phenyl)-acrylic acid ethyl ester

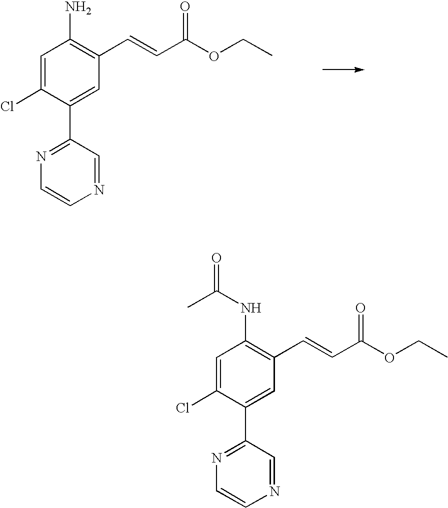

The reaction is performed in analogy to Example if and the title product purified via chromatography (SiO2, hexanes/TBME 1/1) to yield the desired product as colorless crystals (145 mg; 77%).

1H-NMR (400 MHz; DMSO-d6), δ (ppm): 1.25 (t, 3H); 2.13 (s, 3H); 4.17 (q, 2H); 6.66 (d, 1H); 7.78 (d, 1H); 7.82 (s, 1H); 8.06 (s, 1H); 8.68 (d, 1H); 8.75 (m, 1H); 8.94 (s, 1H); 10.07 (bs, 1H).

MS (m/z) ES+: 346 (MH+).

### e) (E)-3-(2-Acetylamino-4-chloro-5-pyrazin-2-yl-phenyl)-acrylic acid

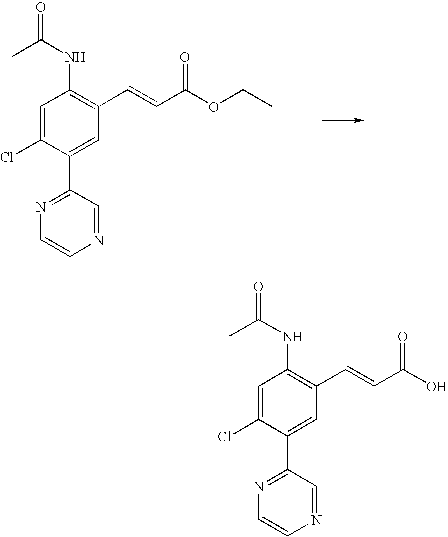

The reaction is performed in analogy to Example 23c and the title product obtained after acidification as yellowish crystals (151 mg; 100%).

1H-NMR (400 MHz; DMSO-d6), δ (ppm): 2.13 (s, 3H); 6.55 (d, 1H); 7.63 (d, 1H); 7.80 (s, 1H); 7.97 (s, 1H); 8.66 (s, 1H); 8.76 (s, 1H); 8.94 (s, 1H); 10.03 (bs, 1H).

MS (m/z) ES−: 316 (MH−, 100); 272 (100); 230 (40).

### f) N-(5-Chloro-2-{(E)-3-[3-(4-fluoro-benzyl)-3,8-diaza-bicyclo[3.2.1]oct-8-yl]-3-oxo-propenyl}-4-pyrazin-2-yl-phenyl)-acetamide

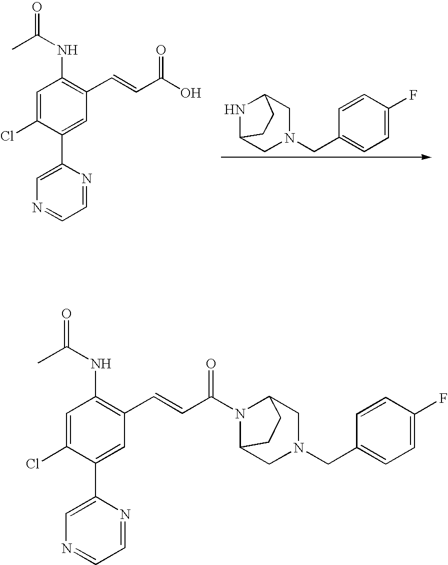

The reaction is performed in analogy to Example 23d and the title product purified via chromatography (SiO2, ethyl acetate/acetone 10/1) to yield the desired product as yellow crystals (70 mg; 65%).

1H-NMR (400 MHz; DMSO-d6), δ (ppm): 1.68-1.78 (m, 1H); 1.82-1.97 (m, 3H); 2.15-2.20 (m, 5H); 2.65 (bd, 2H); 3.45 (s, 2H); 4.52 (bd, 1H); 4.67 (bd, 1H); 7.10-7.17 (m, 3H); 7.30 (dd, 2H); 7.70 (d, 1H); 7.80 (s, 1H); 8.10 (s, 1H); 8.68 (d, 1H); 8.78 (d, 1H); 8.93 (d, 1H); 10.03 (bs, 1H).

MS (m/z) ES+: 520 (MH+).

### Example 56

### N-(5-Chloro-2-{(E)-3-[3-(4-fluoro-benzyl)-3,8-diaza-bicyclo[3.2.1]oct-8-yl]-3-oxo-propenyl}-4-pyridin-3-yl-phenyl)-acetamide

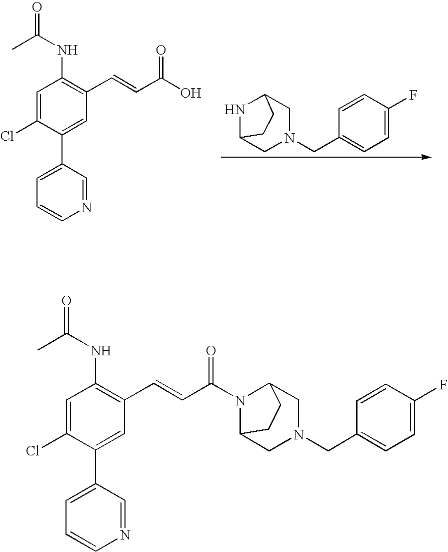

(E)-3-(2-Acetylamino-4-chloro-5-pyridin-3-yl-phenyl)-acrylic acid (obtained in analogy to Example 55e from 3-chloro-4-iodoaniline and 3-(tri-n-butylstannyl)pyridine) is treated in analogy to Example 23d to yield the title product purified via chromatography (SiO2, acetone/TBME 1/2) as colorless foam (77 mg; 70%).

1H-NMR (400 MHz; DMSO-d6), δ (ppm): 1.68-1.78 (m, 1H); 1.82-1.97 (m, 3H); 2.05-2.20 (m, 5H); 2.65 (bt, 2H); 3.45 (s, 2H); 4.52 (bd, 1H); 4.67 (bd, 1H); 7.10-7.17 (m, 3H); 7.30 (dd, 2H); 7.50 (dd, 1H); 7.67 (d, 1H); 7.72 (s, 1H); 8.92 (ss, 1H); 8.00 (s, 1H); 8.60 (d, 1H); 8.67 (d, 1H); 10.00 (s, 1H).

MS (m/z) ES+: 519 (MH+).

### Example 57

### (5-Chloro-2-{(E)-3-[3-(4-fluoro-benzyl)-3,8-diaza-bicyclo[3.2.1]oct-8-yl]-3-oxo-propenyl}-4-pyridin-3-yl-phenyl)-urea

### a) (E)-3-(4-Chloro-2-nitro-5-pyridin-3-yl-phenyl)-1-[3-(4-fluoro-benzyl)-3,8-diaza-bicyclo[3.2.1]oct-8-yl]-propenone

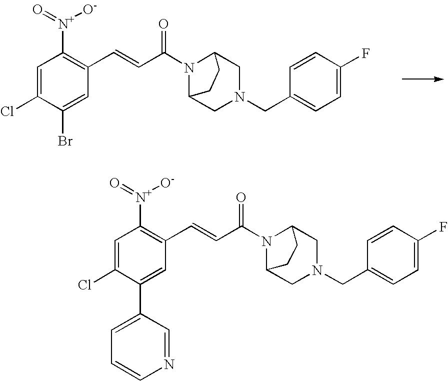

The reaction is performed in analogy to Example 55a employing 3-(tri-n-butylstannyl)pyridine) and the title product purified via chromatography (SiO2, hexanes/acetone 3/1) to yield the desired product as yellow foam (794 mg; 40%).

1H-NMR (400 MHz; DMSO-d6), δ (ppm): 1.70-1.94 (m, 4H); 2.12 (d, 1H); 2.18 (d, 1H); 2.65 (bt, 2H); 3.45 (s, 2H); 4.52 (bd, 1H); 4.68 (bd, 1H); 7.12 (t, 2H); 7.28-7.33 (m, 3H); 7.56 (m, 1H); 7.70 (d, 1H); 7.98 (td, 1H); 8.17 (s, 1H); 8.31 (s, 1H); 8.66 (bd, 1H); 8.71 (bs, 1H).

MS (m/z) ES+: 539 (MH+).

### b) (E)-3-(2-Amino-4-chloro-5-pyridin-3-yl-phenyl)-1-[3-(4-fluoro-benzyl)-3,8-diaza-bicyclo[3.2.1]oct-8-yl]-propenone

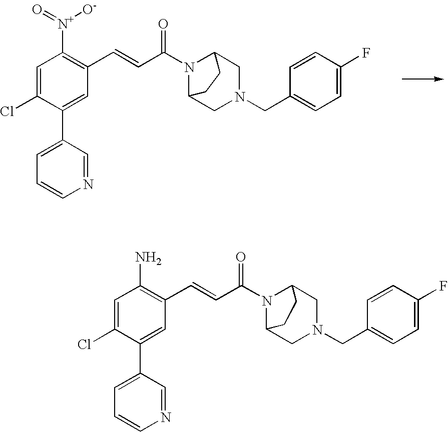

The reaction is performed in analogy to Example 32b and the title product purified via chromatography (SiO2, hexanes/acetone 1/1) to yield the desired product as yellow foam (346 mg; 47%).

1H-NMR (400 MHz; DMSO-d6), δ (ppm): 1.67-1.77 (m, 1H); 1.80-1.92 (m, 3H); 2.10-2.20 (m, 2H); 2.59-2.67 (bt, 2H); 3.45 (s, 2H); 4.52 (bd, 1H); 4.64 (bd, 1H); 5.91 (bs, 2H); 6.88 (s, 1H); 6.97 (d, 1H); 7.12 (t, 2H); 7.30 (dd, 2H); 7.41 (dd, 1H); 7.60 (s, 1H); 7.67 (d, 1H); 7.80 (td, 1H); 8.50 (dd, 1H); 8.58 (d, 1H).

MS (m/z) ES+: 477 (MH+).

### c) (5-Chloro-2-{(E)-3-[3-(4-fluoro-benzyl)-3,8-diaza-bicyclo[3.2.1]oct-8-yl]-3-oxo-propenyl}-4-pyridin-3-yl-phenyl)-urea

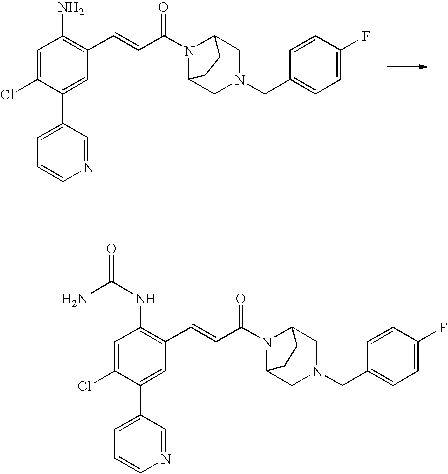

The reaction is performed in analogy to Example 4 and the title product purified via chromatography (SiO2, hexanes/acetone 1/2) to yield the desired product as colorless foam (48 mg; 44%).

1H-NMR (400 MHz; DMSO-d6), δ (ppm): 1.68-1.78 (m, 1H); 1.82-1.97 (m, 3H); 2.10 (d, 1H); 2.18 (d, 1H); 2.63 (bd, 2H); 3.45 (s, 2H); 4.52 (bd, 1H); 4.67 (bd, 1H); 6.30 (s, 2H); 7.09-7.15 (m, 3H); 7.30 (dd, 2H); 7.48 (dd, 1H); 7.70 (d, 1H); 7.83 (s, 1H); 7.88 (ss, 1H); 8.13 (s, 1H); 8.52 (s, 1H); 8.58 (dd, 1H); 8.65 (d, 1H).

MS (m/z) ES+: 518 (MH−, 50); 503 (100).

### Example 58

### N-(5-Chloro-2-{(E)-3-[3-(4-fluoro-benzyl)-3,8-diaza-bicyclo[3.2.1]oct-8-yl]-3-oxo-propenyl}-4-pyridin-2-yl-phenyl)-acetamide

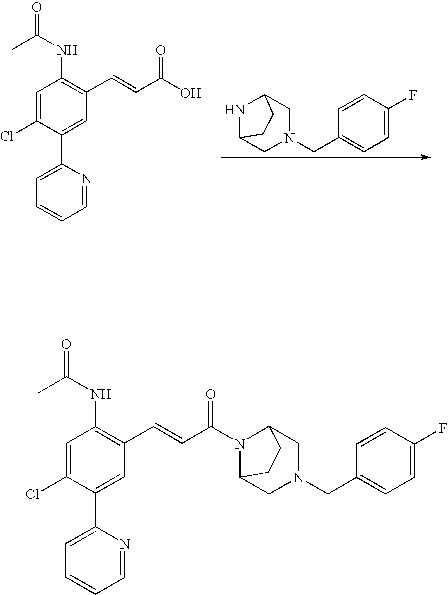

(E)-3-(2-Acetylamino-4-chloro-5-pyridin-2-yl-phenyl)-acrylic acid (obtained in analogy to Example 55e from 3-chloro-4-iodoaniline and 2-(tri-n-butylstannyl)pyridine) is treated in analogy to Example 23d and the title product purified via chromatography (SiO2, TBME/acetone 2/1) to yield the desired product as colorless foam (16 mg; 42%).

1H-NMR (400 MHz; DMSO-d6), δ (ppm): 1.68-1.78 (m, 1H); 1.82-1.97 (m, 3H); 2.11 (s, 3H); 2.15 (d, 2H); 2.63 (bd, 2H); 3.45 (s, 2H); 4.52 (bd, 1H); 4.67 (bd, 1H); 7.10-7.15 (m, 3H); 7.30 (dd, 2H); 7.42 (dd, 1H); 7.62 (t, 2H); 7.70 (s, 1H); 7.90 (dt, 1H); 8.03 (s, 1H); 8.68 (d, 1H); 9.97 (s, 1H).

MS (m/z) ES+: 517 (MH+).

### Example 59

### N-(3-Chloro-6-{(E)-3-[3-(4-fluoro-benzyl)-3,8-diaza-bicyclo[3.2.1]oct-8-yl]-3-oxo-propenyl}-2,4-dimethoxy-phenyl)-acetamide

### a) 6-Bromo-3-chloro-2,4-dimethoxy-phenylamine

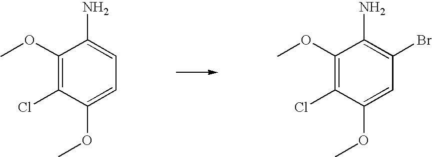

3-Chloro-2,4-dimethoxy-phenylamine (Synthesis 1984, 7, 581) (1.49 g; 7.9 mmol) and N-bromosuccinimid (1.41 g; 7.9 mmol) are stirred in CH2Cl2 for 30 min., evaporated and purified via chromatography (SiO2, hexanes/EtOAc 100/0 to 80/20) to yield the desired product as brownish crystals (657 mg; 31%).

1H-NMR (400 MHz; DMSO-d6), δ (ppm): 3.72 (s, 3H); 3.73 (s, 3H); 4.79 (bs, 2H); 7.00 (s, 1H).

MS (m/z) ES+: 268 (MH+).

### b) (E)-3-(2-Amino-4-chloro-3,5-dimethoxy-phenyl)-acrylic acid ethyl ester

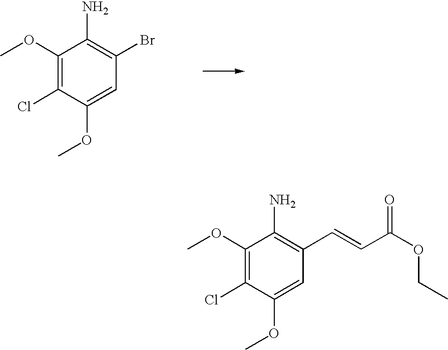

The reaction is performed in analogy to Example 23b and the title product purified via chromatography (SiO2, hexanes/EtOAc 100/0 to 80/20) to yield the desired product as brownish crystals (433 mg; 76%).

1H-NMR (400 MHz; DMSO-d6), δ (ppm): 1.26 (t, 3H); 3.68 (s, 3H); 3.77 (s, 3H); 4.17 (q, 2H); 5.24 (s, 2H); 6.51 (d, 1H); 7.01 (s, 1H); 7.84 (d, 1H).

MS (m/z) ES+: 286 (MH+, 80); 240 (100); 225 (50).

### c) (E)-3-(2-Acetylamino-4-chloro-3,5-dimethoxy-phenyl)-acrylic acid ethyl ester

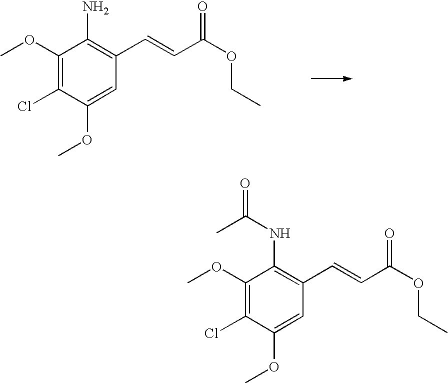

The reaction is performed in analogy to Example if to yield the desired product as yellow crystals recrystallised from TBME (382 mg; 67%).

1H-NMR (400 MHz; DMSO-d6), δ (ppm): 1.26 (t, 3H); 2.08 (s, 3H); 3.70 (s, 3H); 3.92 (s, 3H); 4.18 (q, 2H); 6.79 (d, 1H); 7.35 (s, 1H); 7.53 (d, 1H); 9.52 (s, 1H).

MS (m/z) ES+: 328.1 (MH+, 80); 240.2 (100).

### d) (E)-3-(2-Acetylamino-4-chloro-3,5-dimethoxy-phenyl)-acrylic acid

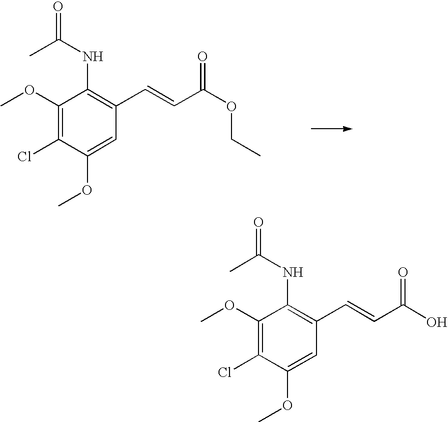

The reaction is performed in analogy to Example 23c and yielded the title product after acidification of the reaction mixture as yellowish crystals (297 mg; 86%).

1H-NMR (400 MHz; DMSO-d6), δ (ppm): 2.08 (s, 3H); 3.69 (s, 3H); 3.93 (s, 3H); 6.66 (d, 1H); 7.30 (s, 1H); 7.48 (d, 1H); 9.48 (s, 1H); 12.5 (bs, 1H).

MS (m/z) ES+: 300.1 (MH+, 100); 240.2 (70).

### e) N-(3-Chloro-6-{(E)-3-[3-(4-fluoro-benzyl)-3,8-diaza-bicyclo[3.2.1]oct-8-yl]-3-oxo-propenyl}-2,4-dimethoxy-Phenyl)-acetamide

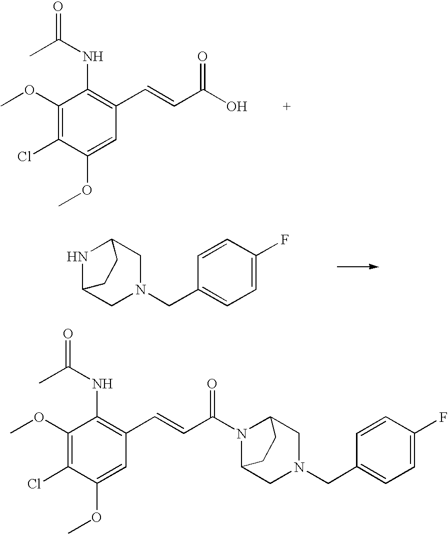

The reaction is performed in analogy to Example 23d and the title product purified via chromatography (SiO2, CH2Cl2/MeOH 100/0 to 96/4) to yield the desired product as colorless foam (83 mg; 83%).

1H-NMR (400 MHz; DMSO-d6), δ (ppm): 1.68-1.78 (m, 1H); 1.82-1.97 (m, 3H); 2.08 (s, 3H); 2.15 (dd, 2H); 2.63 (bd, 1H); 2.71 (bd, 1H); 3.45 (s, 2H); 3.70 (s, 3H); 4.04 (s, 3H); 4.52 (bd, 1H); 4.67 (bd, 1H); 7.08-7.16 (m, 3H); 7.30-7.35 (m, 3H); 7.45 (d, 1H); 9.43 (s, 1H).

MS (m/z) ES+: 502.4 (MH+).

### Example 60

### (E)-N-(5-Chloro-2-{3-[3-(4-fluorobenzyl)-3,9-diazabicyclo[3.3.1]non-9-yl]-3-oxopropenyl}-phenyl)-acetamide

### a) (E)-(5-Chloro-2-{3-[3-(4-fluorobenzyl)-3,9-diazabicyclo[3.3.1]non-9-yl]-3-oxopropenyl}-phenyl)-carbamic acid tert-butyl ester

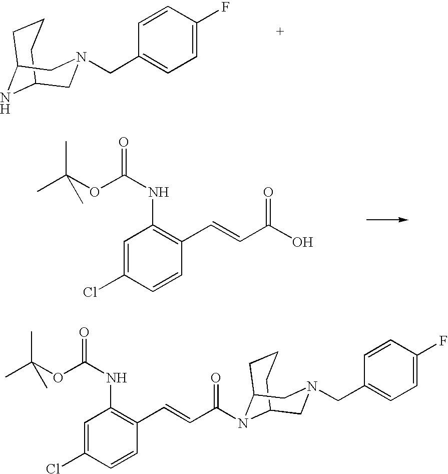

3-(4-Fluorobenzyl)-3,9-diazabicyclo[3.3.1]nonane (Blumberg, L. C. et al., WO 0232901) (394 mg; 1.5 mmol) and (E)-3-(2-tert-butoxycarbonylamino-4-chlorophenyl)-acrylic acid (450 mg; 1.5 mmol) are dissolved in CH2Cl2 (15 ml) and treated with EDCl.HCl (288 mg; 1.5 mmol) for 12 h. The reaction mixture is poured on a column of SiO2 and chromatographed (TBME/MeOH/NH3conc 95/4.5/0.5 to 90/9/1) to give the desired product as a colorless foam (450 mg; 58%).

1H-NMR (400 MHz; DMSO-d6), δ (ppm): 1.48 (s, 9H); 1.50-1.60 (m, 2H); 1.67-1.80 (m, 4H); 2.19 (d, 1H); 2.29 (d, 1H); 2.82-2.95 (m, 2H); 3.40 (d, 2H); 4.47 (bs, 1H); 4.60 (bs, 1H); 7.11-7.27(m, 4H); 7.33-7.39(m, 2H); 7.47 (s, 1H); 7.67 (d, 1H); 7.89(d, 1H); 9.22 (s, 1H).

MS (m/z) ES+: 514.2 (MH+, 100).

### b) (E)-3-(2-Amino-4-chlorophenyl)-1-[3-(4-fluorobenzyl)-3,9-diaza-bicyclo[3.3.1]non-9-yl]-propenone

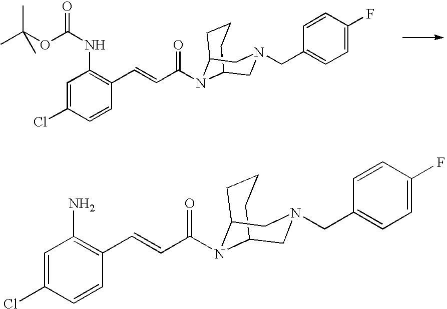

(E)-(5-Chloro-2-{3-[3-(4-fluorobenzyl)-3,9-diazabicyclo[3.3.1]non-9-yl]-3-oxopropenyl}-phenyl)-carbamic acid tert-butyl ester (450 mg; 0.875 mmol) is dissolved in EtOH/HClconc (3.5 ml/3.5 ml), kept at room temp. for 1 h, poured on a column of SiO2 and chromatographed (TBME/MeOH/NH3conc 95/4.5/0.1) to give the desired product as a yellow foam (350 mg; 95%).

1H-NMR (400 MHz; DMSO-d6), δ (ppm): 1.50-1.58 (m, 1H); 1.66-1.82 (m, 4H); 2.18(dd, 1H); 2.28(dd, 1H); 2.76-2.93(m, 3H); 3.40 (d, 2H); 4.42 (bs, 1H); 4.60(bs, 1H); 5.73 (s, 2H); 6.53(d, 1H); 6.73 (d, 1H); 6.96(d, 1H); 7.19(t, 2H); 7.36(dd, 2H); 7.53(d, 1H); 7.68(d, 1H).

MS (m/z) ES+: 414.2 (MH+, 100).

### c) (E)-N-(5-Chloro-2-{3-[3-(4-fluorobenzyl)-3,9-diazabicyclo[3.3.1]non-9-yl]-3-oxopropenyl}-phenyl)-acetamide

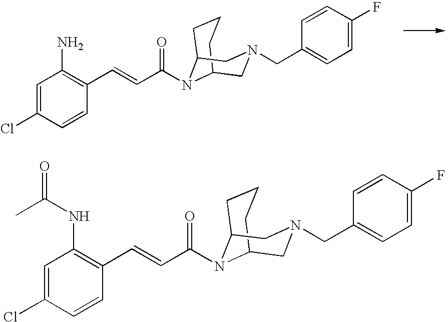

(E)-3-(2-Amino-4-chlorophenyl)-1-[3-(4-fluorobenzyl)-3,9-diaza-bicyclo[3.3.1]non-9-yl]-propenone (62 mg; 0.15 mmol) (62 mg; 0.15 mmol) is treated as in Example if and purified via chromatography (SiO2; TBME/MeOH/NH3conc 98/1.8/0.2) to yield the title compound as a colorless foam (50 mg; 73%).

1H-NMR (500 MHz; DMSO-d6), δ (ppm): 1.53 (m, 1H); 1.60-1.80(m, 4H); 2.08 (s, 3H); 2.15(d, 1H); 2.25(d, 1H); 2.80-2.93(m, 3H); 3.42(d, 2H); 4.48(s, 1-H); 4.58(s, 1H); 7.17(m, 3H); 7.25(d, 1H); 7.32(d, 2H); 7.55(s, 1H); 7.65(d, 1H); 7.92(d, 1H); 9.93(s, 1H).

MS (m/z) ES+: 478.1 (MH+, 100).

### Example 61

### (E)-N-(5-Chloro-2-{3-[3-(4-fluorobenzyl)-3,9-diazabicyclo[3.3.1]non-9-yl]-3-oxopropenyl}-phenyl)-urea

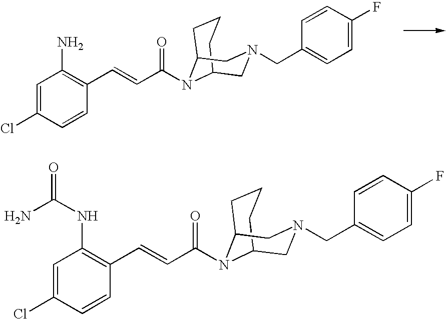

3-(2-Amino-4-chlorophenyl)-1-[3-(4-fluorobenzyl)-3,9-diazabicyclo[3.3.1]non-9-yl]-propenone (62 mg; 0.15 mmol) is treated as in Example 4 and purified by chromatography (SiO2; TBME/MeOH/NH3conc 98/1.8/0.2) to render the target compound as colorless foam (50 mg; 72%).

1H-NMR (500 MHz; DMSO-d6), δ (ppm): 1.51 (m, 1H); 1.60-1.81(m, 4H); 2.15(dd, 1H); 2.25(d, 1H); 2.75-2.92(m, 3H); 3.40(d, 2H); 4.47(s, 1H); 4.58(s, 1H); 6.30(s, 2H, NH2); 7.05 (d, 1H); 7.18(m, 3H); 7.32(m, 2H); 7.69(d, 1H); 7.78(d, 1H); 7.98(s, 1H); 8.41(s, 1H, NH).

MS (m/z) ES+: 457.1 (MH+, 100).

### Example 62

### (E)-N-(5-Chloro-2-{3-[3-(4-fluorobenzyl)-3,9-diazabicyclo[3.3.1]non-9-yl]-3-oxopropenyl}-phenyl)-N′-cyanoguanidine

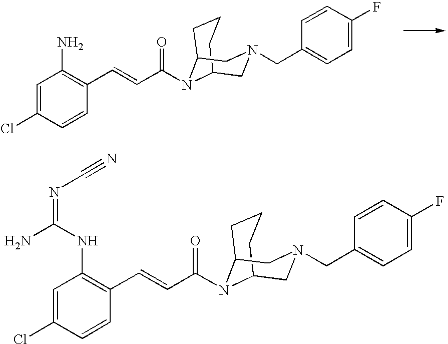

(E)-3-(2-Amino-4-chlorophenyl)-1-[3-(4-fluorobenzyl)-3,9-diazabicyclo[3.3.1]non-9-yl]-propenone (62 mg; 0.15 mmol) is treated as in Example 2 and purified by chromatography (SiO2; TBME/MeOH/NH3conc 98/1.8/0.2) to yield the target compound as colorless crystals (35 mg; 36%).

1H-NMR (500 MHz; DMSO-d6), δ (ppm): 1.51(m, 1H); 1.61-1.81(m, 4H); 2.15(d, 1H); 2.25(d, 1H); 2.75-2.94(m, 3H); 3.40(d, 2H); 4.48(s, 1H); 4.59(s, 1H); 7.20(t, 2H); 7.23(d, 1H); 7.30(d, 3H); 7.40(s, 1H); 7.50(d, 1H); 7.92(d, 1H); 9.02 (s, 1H).

MS (m/z) ES+: 481.2 (MH+, 100).

### Example 63

### (E)-N-(5-Chloro-2-{3-[3-(4-fluorobenzyl)-3,9-diazabicyclo[3.3.1]non-9-yl]-3-oxopropenyl}-phenyl)-2-dimethylaminoacetamide

### a) (E)-2-Chloro-N-(5-chloro-2-{3-[3-(4-fluorobenzyl)-3,9-diazabicyclo[3.3.1]non-9-yl]-3-oxopropenyl}-phenyl)-acetamide

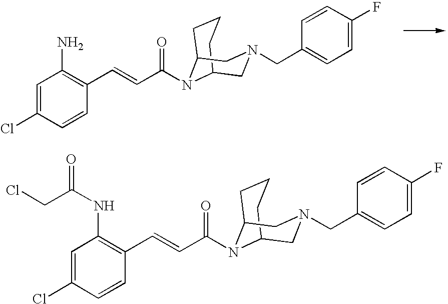

(E)-3-(2-Amino-4-chlorophenyl)-1-[3-(4-fluorobenzyl)-3,9-diaza-bicyclo[3.3.1]non-9-yl]-propenone (83 mg; 0.2 mmol) is dissolved in THF (4 ml) and NEt3 (0.034 ml; 0.48 mmol) and treated with chloroacetylchloride (0.019 ml; 0.24 mmol) at room temp. for 1 h. The reaction mixture is poured on a saturated solution of Na2CO3 and extracted with EtOAc three times. The combined organic phases are dried over Na2SO4, evaporated to dryness and purified via chromatography (SiO2, TBME/hexanes 6/4) to yield the tile compound as colorless foam (80 mg; 81%).

1H-NMR (400 MHz; DMSO-d6), δ(ppm): 1.51-1.59(m, 1H); 1.67-1.85(m, 4H); 2.18(bd, 1H); 2.28(bd, 1H); 2.78-2.95(m, 3H); 3.40(dd, 2H); 4.35(s, 2H); 4.46(bs, 1H); 4.58(bs, 1H); 7.18(t, 2H); 7.24(d, 1H); 7.42-7.49(m, 3H); 7.60(d, 1H); 7.65(d, 1H); 7.96(d, 1H); 10.23(s, 1H).

MS (m/z) ES+: 490.1 (MH+, 100).

### b) (E)-N-(5-Chloro-2-{3-[3-(4-fluorobenzyl)-3,9-diazabicyclo[3.3.1]non-9-yl]-3-oxopropenyl}-phenyl)-2-dimethylaminoacetamide

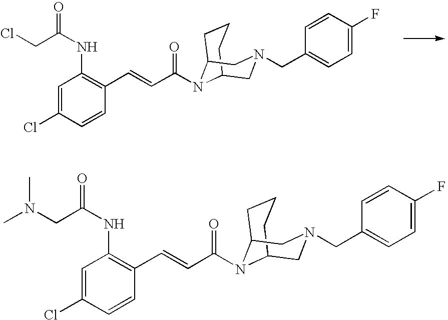

(E)-2-Chloro-N-(5-chloro-2-{3-[3-(4-fluorobenzyl)-3,9-diazabicyclo[3.3.1]non-9-yl]-3-oxopropenyl}-phenyl)-acetamide (80 mg; 0.16 mmol) is treated with dimethylamine in THF as in Example 3. The product is purified by via chromatography (SiO2, TBME/MeOH/NH3conc, 97/2.7/0.3) to yield the title compound as colorless foam (60 mg; 75%).

1H-NMP (400 MHz; DMSO-d6), δ (ppm): 1.51-1.61(m, 1H); 1.67-1.86(m, 4H); 2.19(bd, 1H); 2.28(bd, 1H); 2.35(s, 6H); 2.80-2.96(m, 3H); 3.33(s, 2H); 3.41(dd, 2H); 4.48(bs, 1H); 4.61(bs, 1H); 7.15-7.23(m, 3H); 7.30(d, 1H); 7.37(dd, 2H); 7.60(d, 1H); 7.65(s, 1H); 7.93(d, 1H); 9.82 (s, 1H).

MS (m/z) ES+: 499.1 (MH+, 100).

### Example 64

### 9-[2-(2-Acetylamino-4-chloro-phenoxy)-acetyl]-7-(4-fluorobenzyl)-3,7,9-triazabicyclo[3.3.1]nonane-3-carboxylic acid tert-butyl ester

### a) (meso)-4-Benzenesulfonyl-1-benzylpiperazine-2,6-dicarboxylic acid diethyl ester

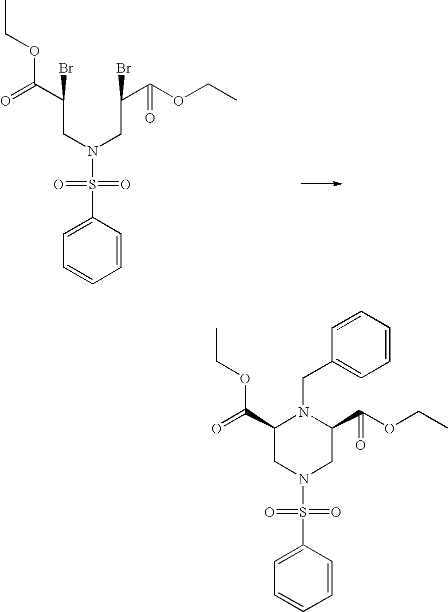

(meso)-3-[Benzenesulfonyl-(2-bromo-2-ethoxycarbonylethyl)-amino]-2-bromopropionic acid ethyl ester (Terauchi Hiromi et al., Chem. Pharm. Bull. (1975), 23(12), 3162-9) (8 g; 15.5 mmol) and benzylamine (5.1 ml; 46.6 mmol) are heated in toluene (30 ml) at 90° C. for 1.5 h. The precipitated benzylamine.HBr is filtered, the filtrate evaporated to dryness and purified via chromatography (TBME/hexanes 3/7) to yield the title compound as colorless crystals (4.35 g; 61%).

1H-NMR (400 MHz; DMSO-d6), δ (ppm): 1.17(t, 6H); 2.86(dd, 2H); 3.35(dd, 2H); 3.48(dd, 2H); 3.97(s, 2H); 4.00(q, 4H); 7.20-7.30(m, 5H); 7.63-7.80(m, 5H).

MS (m/z) ES+: 461.2 (MH+, 30).

### b) (meso)-(4-Benzenesulfonyl-1-benzyl-6-hydroxymethyl-piperazin-2-yl)-methanol

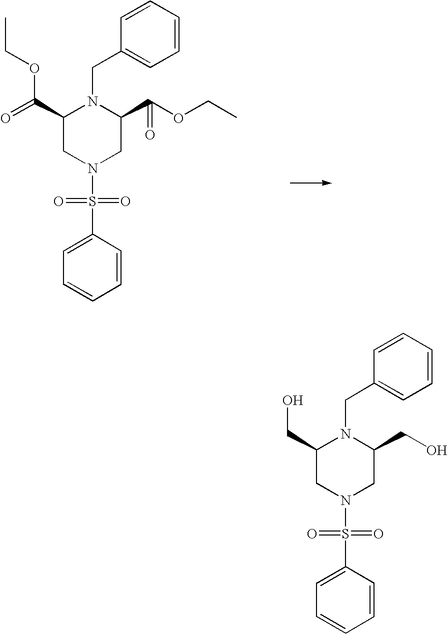

A 1M solution of LiAlH4 in THF (28 ml; 28 mmol) is added dropwise under cooling and stirring to (meso)-4-benzenesulfonyl-1-benzylpiperazine-2,6-dicarboxylic acid diethyl ester (4.34 g; 9.4 mmol) in THF (110 ml). The reaction mixture is refluxed for 20 min., poured on a saturated solution of Na2CO3 and extracted with TBME three times. The combined organic phases are dried over Na2SO4 and evaporated to dryness to yield a colorless solid, which is washed with TBME to yield the title compound as white crystals (2.88 g; 81%).

1H-NMR (400 MHz; DMSO-d6), δ (ppm): 2.48-2.58(m, 2H); 2.62-2.69(m, 2H); 3.05-3.2(m, 2H); 3.24-3.30(dd, 2H); 3.43-3.50(m, 2H); 3.80(s, 2H); 4.65(t, 2H); 7.20-7.35(m, 5H); 7.65-7.80(m, 5H).

MS (m/z) ES−: 375.3 (M−H, 100).

### c) (meso)-4-Benzenesulfonyl-1-benzyl-2,6-bis-chloromethylpiperazine

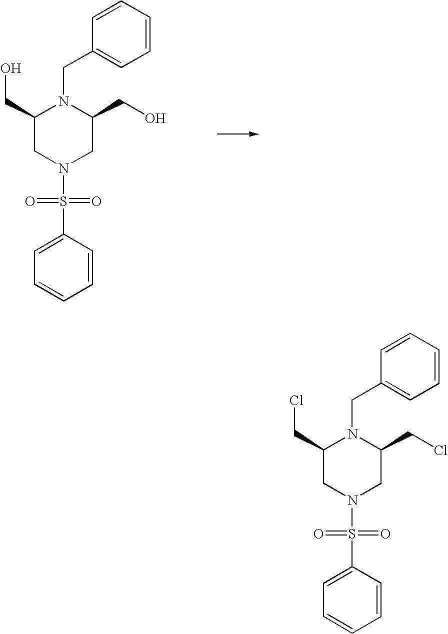

Thionylchloride (10 ml; 137 mmol) is rapidly added under stirring to an ice-cooled solution of (meso)-(4-benzenesulfonyl-1-benzyl-6-hydroxymethyl-piperazin-2-yl)-methanol (10 g; 26 mmol) in DMF (200 ml). The reaction mixture is warmed to room temp., stirred for 1 h and poured on a saturated solution of Na2CO3 (1000 ml). The precipitated solid is filtered off, washed with water and recrystallised from TBME to yield the title compound as colorless crystals (8.5 g; 77%).

1H-NMR (400 MHz; DMSO-d6), δ (ppm): 2.50-2.60(m, 2H); 2.93-3.00(m, 2H); 3.51-3.58(m, 4H); 3.77(d, 2H); 3.93(s, 2H); 7.22-7.33(m, 5H); 7.68(t, 2H); 7.74-7.83(m, 3H).

MS (m/z) EI-MS: 412(M+, 50); 377(20); 271(55); 235(30); 91(100); 77(20).

### d) 3-Benzenesulfonyl-7,9-dibenzyl-3,7,9-triazabicyclo[3.3.1]nonane and 3-benzenesulfonyl-6,8-dibenzyl-3,6,8-triazabicyclo[3.2.2]nonane

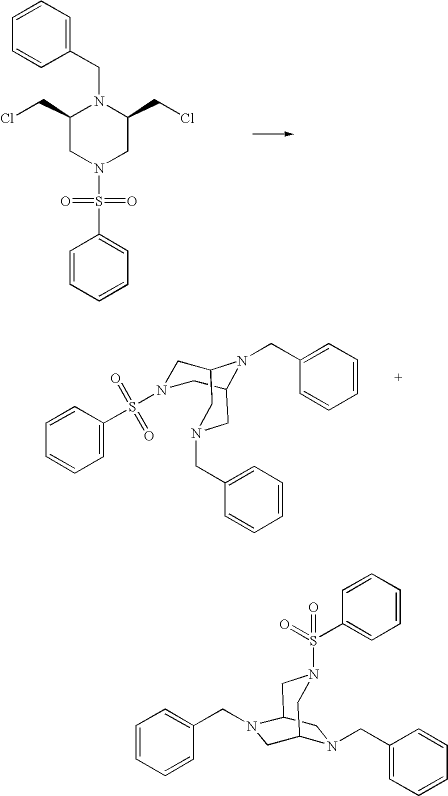

(meso)-4-Benzenesulfonyl-1-benzyl-2,6-bis-chloromethylpiperazine (610 mg; 1.5 mmol) and benzylamine (12 ml) are refluxed in an oil bath (200° C.) for 15 min. The reaction mixture is poured on water and extracted with EtOAc three times. The combined organic phases are dried over Na2SO4, filtered and evaporated to dryness and purified via chromatography (SiO2; TBME/hexanes 2/8) to yield 3-benzenesulfonyl-7,9-dibenzyl-3,7,9-triazabicyclo[3.3.1]nonane, which is eluted first as colorless crystals (488 mg; 74%) followed by 3-benzenesulfonyl-6,8-dibenzyl-3,6,8-triazabicyclo[3.2.2]nonane as colorless crystals (48 mg; 7.4%)

1H-NMR (400 MHz; DMSO-d6), δ (ppm): 2.53(d, 2H); 2.69(d, 2H); 2.81 (d, 2H); 2.83(s, 2H); 3.41(d, 2H); 3.44(s, 2H); 3.67(s, 2H); 7.16-7.32(m, 8H); 7.38(m, 2H); 7.65-7.78(m, 5H).

MS (m/z) ES+: 448.2 (MH+, 100).

### 3-benzenesulfonyl-6,8-dibenzyl-3,6,8-triazabicyclo[3.2.2]nonane

1H-NMR (400 MHz; DMSO-d6), δ (ppm): 2.71 (d, 2H); 2.98 (dd, 2H); 3.08 (m, 2H); 3.21 (dd, 2H); 3.43 (dd, 2H); 3.72 (dd, 4H); 7.17-7.30 (m, 10H); 7.53 (t, 2H); 7.71 (m, 1H); 7.28 (d, 2H).

MS (m/z) ES+: 448.2 (MH+).

### e) 3,9-Dibenzyl-3,7,9-triazabicyclo[3.3.1]nonane

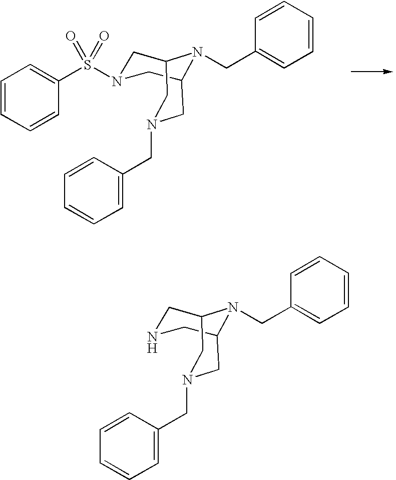

3-Benzenesulfonyl-7,9-dibenzyl-3,7,9-triazabicyclo[3.3.1]nonane (488 mg; 1.1 mmol) is dissolved in xylene (10 ml), Red-Al (˜3.5 M in toluene; 1.25 ml; 4.4 mmol) added and refluxed for 1 h. The reaction mixture is poured on NaOH conc. and extracted with THF three times. The combined organic phases are dried with K2CO3, filtered, evaporated to dryness and purified via chromatography (SiO2, EtOAc/MeOH/NH3conc 80/20/4) to yield the title compound as a yellowish oil, which slowly crystallised on standing (276 mg; 82%).

MS (m/z) ES+: 308.2(MH+, 100).

### f) 7,9-Dibenzyl-3,7,9-triazabicyclo[3.3.1]nonane-3-carboxylic acid tert-butyl ester

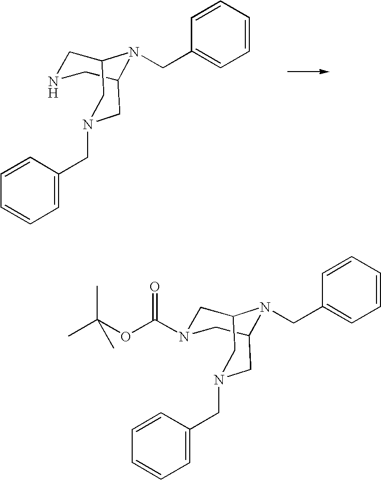

3,9-Dibenzyl-3,7,9-triazabicyclo[3.3.1]nonane (276 mg; 0.9 mmol) is dissolved in TBME (4 ml) and treated with (BOC)2O (216 mg; 1 mmol) for 10 min at room temp. The reaction mixture is diluted with hexanes and purified via chromatography (SiO2, TBME/hexanes 2/8) to yield the title compound as colorless crystals (285 mg; 78%).

1H-NMR (400 MHz; DMSO-d6), δ (ppm): 1.45(s, 9H); 2.43(bt, 2H); 2.65-2.76(m, 3H); 3.23(bd, 1H); 3.35(s, 2H); 3.35-3.43(m, 2H); 3.70(d, 1H); 3.78(d, 1H); 3.88(s, 2H); 7.20-7.40(m, 10H).

MS (m/z) ES+: 408.3(MH+, 100).

### g) 3,7,9-Triazabicyclo[3.3.1]nonane-3-carboxylic acid tert-butyl ester

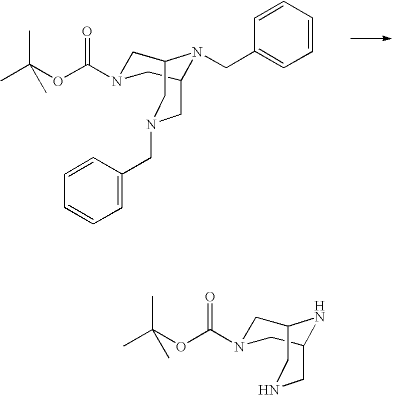

7,9-Dibenzyl-3,7,9-triazabicyclo[3.3.1]nonane-3-carboxylic acid tert-butyl ester (285 mg; 0.7 mmol) in EtOH (150 ml) is hydrogenated over Pd/C (10%; 1 g) at 1 atm and room temp. for 4 h. Filtration, evaporation and chromatography (SiO2; TBME/MeOH/NH3conc 80/20/4 to 60/40/10) yielded the title compound (109 mg; 69%) as a colorless resin.

MS (m/z) ES+: 228(MH+, 100).

### h) 7-(4-Fluorobenzyl)-3,7,9-triazabicyclo[3.3.1]nonane-3-carboxylic acid tert-butyl ester

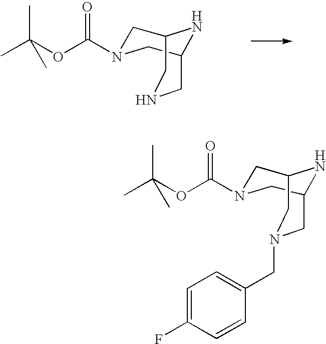

3,7,9-Triazabicyclo[3.3.1]nonane-3-carboxylic acid tert-butyl ester (39 mg; 0.17 mmol), 4-fluorobenzylchloride (0.02 ml; 0.17 mmol) and NaHCO3 (72 mg; 0.85 mmol) are combined and refluxed in EtOH for 2 h. Evaporation and chromatography (SiO2; TBME/MeOH/NH3conc 90/10/2) yielded the title compound as yellow crystals (32 mg; 55%).

1H-NMR (400 MHz; DMSO-d6), δ (ppm): 1.43(s, 9H); 2.18(d, 1H); 2.23(d, 1H); 2.78(d, 1H); 2.87(d, 1H); 2.98(d, 1H); 3.11(d, 1H); 3.25(d, 1H); 3.30(s, 2H); 3.22(d, 1H); 3.83(d, 1H); 3.90(d, 1H); 7.06(t, 2H); 7.33(dd, 2H).

MS (m/z) ES+: 336.3 (MH+, 100).

### i) 9-(2-Chloroacetyl)-7-(4-fluorobenzyl)-3,7,9-triazabicyclo[3.3.1]nonane-3-carboxylic acid tert-butyl ester

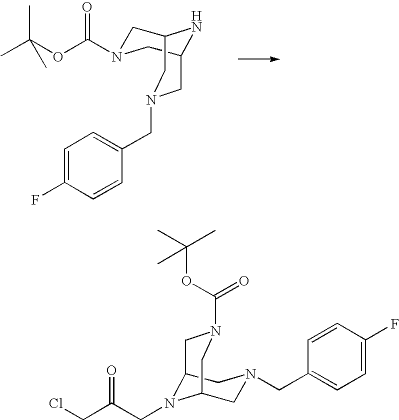

Chloroacetylchloride (0.008 ml; 0.1 mmol) is added to 7-(4-fluorobenzyl)-3,7,9-triazabicyclo[3.3.1]nonane-3-carboxylic acid tert-butyl ester (30 mg; 0.09 mmol) dissolved in THF (1 ml). After 5 min. at room temp. the reaction mixture is poured on 2N Na2CO3 and extracted with TBME three times. The combined organic phases are dried over Na2SO4, filtered, evaporated to dryness and yielded the title compound as a resin (38 mg; 100%) used in the next step without further purification.

MS (m/z) ES+: 412.2(MH+, 100).

### j) 9-[2-(2-Acetylamino-4-chloro-phenoxy)-acetyl]-7-(4-fluorobenzyl)-3,7,9-triazabicyclo[3.3.1]nonane-3-carboxylic acid tert-butyl ester

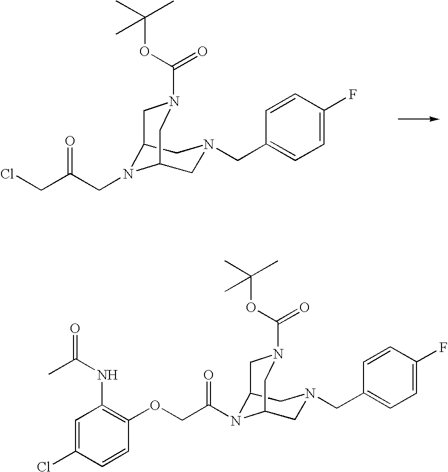

9-(2-Chloroacetyl)-7-(4-fluorobenzyl)-3,7,9-triazabicyclo[3.3.1]nonane-3-carboxylic acid tert-butyl ester (35 mg; 0.08 mmol) is reacted with N-(5-chloro-2-hydroxyphenyl)-acetamide as described in Example 102f to yield the title compound as colorless foam (32 mg; 65%).

1H-NMR (400 MHz; DMSO-d6), δ (ppm): 1.46(s, 9H); 2.10(s, 3H); 2.20(m, 1H); 2.33(bd, 1H); 2.90(bt, 2H); 3.03-3.17(m, 2H); 3.28(bd, 2H); 3.43(bd, 1H); 3.93-4.10(m, 3H); 4.95(s, 2H); 6.98(d, 1H); 7.05-7.15(m, 3H); 7.33(m, 2H); 8.10(s, 1H); 9.52(bd, 1H).

MS (m/z) ES+: 561.2(MH+, 30).

### Example 65

### N-(5-Chloro-2-{2-[3-(4-fluorobenzyl)-3,7,9-triazabicyclo[3.3.1]non-9-yl]-2-oxo-ethoxy}-phenyl)-acetamide

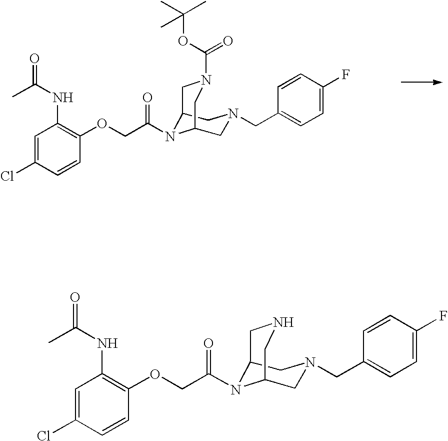

9-[2-(2-Acetylamino-4-chloro-phenoxy)-acetyl]-7-(4-fluorobenzyl)-3,7,9-triazabicyclo[3.3.1]nonane-3-carboxylic acid tert-butyl ester (23 mg; 0.04 mmol) in EtOH (1 ml) is treated with HClconc (1 ml) for 5 min. at room temp., poured on Na2CO3 conc. and extracted with TBME three times. The combined organic phases are dried over Na2SO4, filtered, evaporated to dryness and yielded the title compound as yellowish crystals (13 mg; 73%).

1H-NMR (400 MHz; DMSO-d6), δ (ppm): 2.10 (s, 3H); 2.25(bd, 1H); 2.30(bd, 1H); 2.67-3.00(m, 6H); 3.35(d, 2H); 3.78(s, 1H); 4.20(s, 1H); 4.91(s, 2H); 7.00(d, 1H); 7.08(dd, 1H); 7.17(t, 2H); 7.35(dd, 2H); 8.12(bs, 1H); 9.59(s, 1H).

MS (m/z) ES+: 461.2(MH+, 100).

### Example 66

### 7-[2-(2-Acetylamino-4-chloro-phenoxy)-acetyl]-9-(4-fluorobenzyl)-3,7,9-triazabicyclo[3.3.1]nonane-3-carboxylic acid tert-butyl ester

### a) 7-(2-Chloroacetyl)-3,7,9-triazabicyclo[3.3.1]nonane-3-carboxylic acid tert-butyl ester

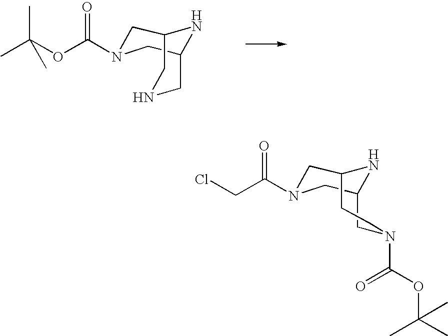

3,7,9-Triazabicyclo[3.3.1]nonane-3-carboxylic acid tert-butyl ester (20 mg; 0.09 mmol) in CH2Cl2 (2 ml) is treated with chloroacetylchloride (0.007 ml; 0.09 mmol) for 5 min. at room temp., evaporated and used in the next step without further purification.

### b) 7-[2-(2-Acetylamino-4-chloro-phenoxy)-acetyl]-3,7,9-triazabicyclo[3.3.1]nonane-3-carboxylic acid tert-butyl ester

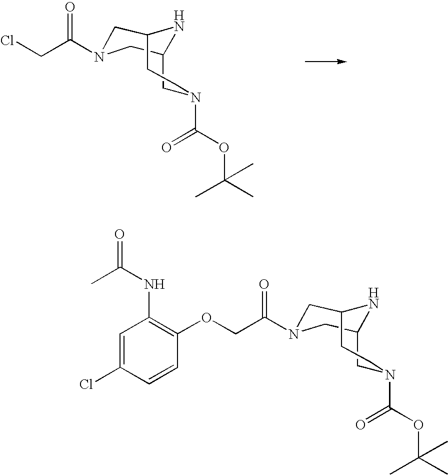

7-(2-Chloroacetyl)-3,7,9-triazabicyclo[3.3.1]nonane-3-carboxylic acid tert-butyl ester (27 mg; 0.09 mmol) is reacted with N-(5-chloro-2-hydroxyphenyl)-acetamide as described in Example 102f and purified via chromatography (SiO2; TBME/MeOH 9/1 then TBME/MeOH/NH3conc 80/20/4) to yield the title compound as almost colorless foam (23 mg; 58%).

1H-NMR (400 MHz; DMSO-d6), δ (ppm): 1.30(s, 9H); 2.10(s, 3H); 2.87-3.13(m, 5H); 3.33(d, 1H); 3.71(d, 1H); 3.88(d, 1H); 4.10(d, 1H); 4.28(d, 1H); 4.76(d, 1H); 4.84(d, 1H); 7.09(s, 2H); 8.13(bs, 1H); 9.90(bs, 1H).

MS (m/z) ES+: 453.2(MH+, 100).

### c) 7-[2-(2-Acetylamino-4-chloro-phenoxy)-acetyl]-9-(4-fluorobenzyl)-3,7,9-triazabicyclo[3.3.1]nonane-3-carboxylic acid tert-butyl ester

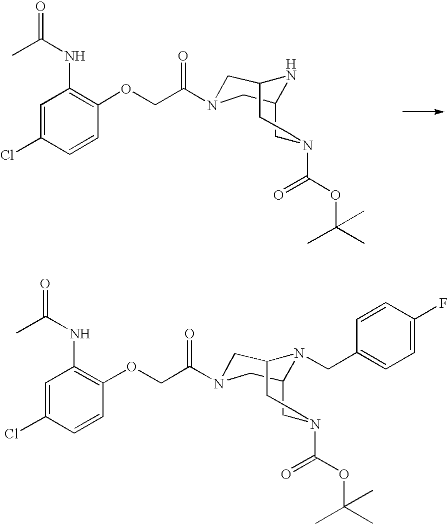

7-[2-(2-Acetylamino-4-chloro-phenoxy)-acetyl]-3,7,9-triazabicyclo[3.3.1]nonane-3-carboxylic acid tert-butyl ester (20 mg; 0.04 mmol), 4-fluorobenzylchloride (0.022 ml; 0.16 mmol) and K2CO3 (200 mg; 1.44 mmol) are combined and refluxed in EtOH (2 ml) for 14 h. The reaction mixture is poured on water and extracted with TBME three times. The combined organic phases are dried over Na2SO4, filtered, evaporated to dryness and purified via chromatography (SiO2; acetone/hexanes 3/7) to yield the title compound as colorless solid (14 mg; 56%).

1H-NMR (400 MHz; DMSO-d6), δ (ppm): 1.30(s, 9H); 2.12(s, 3H); 2.73(bs, 2H); 3.08-3.22(m, 3H); 3.50-3.65(m, 2H); 3.78(d, 1H); 3.92(s, 2H); 4.03(d, 1H); 4.17(d, 1H); 4.76(d, 1H); 4.86(d, 1H); 7.06(s, 2H); 7.15(t, 2H); 7.40(dd, 2H); 8.12(bd, 1H); 9.88(bd, 1H).

MS (m/z) ES+: 561.1(MH+, 30).

### Example 67

### N-(5-Chloro-2-{2-[9-(4-fluoro-benzyl)-3,7,9-triaza-bicyclo[3.3.1]non-3-yl]-2-oxo-ethoxy}-phenyl)-acetamide

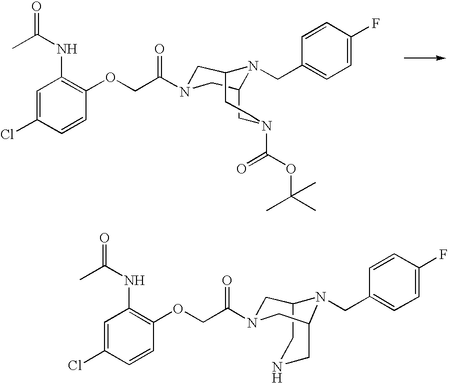

7-[2-(2-Acetylamino-4-chloro-phenoxy)-acetyl]-9-(4-fluorobenzyl)-3,7,9-triazabicyclo[3.3.1]nonane-3-carboxylic acid tert-butyl ester (10 mg; 0.001 mmol) is dissolved in EtOH (0.5 ml) and treated with HCl conc. (1 ml) for 2 min at room temp. The reaction mixture is poured on Na2CO3 conc. and extracted with TBME three times. The combined organic phases are dried over Na2SO4, filtered and evaporated to dryness to yield the title compound as yellow resin (5 mg; 65%).

1H-NMR (400 MHz; DMSO-d6), δ (ppm): 2.13(s, 3H); 2.58(bd, 2H); 2.76(bd, 1H); 2.88-3.00(m, 2H); 3.08(m, 1H); 3.5(d, 2H); 3.65(bd, 1H); 3.90(s, 2H); 4.03(s, 1H); 4.82(d, 1H); 5.03(d, 1H); 7.08(s, 2H); 7.15(t, 2H); 7.41(dd, 2H); 8.22(s, 1H); 9.88(s, 1H).

MS (m/z) ES+: 461.2(MH+, 100).

### Example 68

### (E)-N-(5-Chloro-2-{3-[3-(4-fluorobenzyl)-3,7,9-triazabicyclo[3.3.1]non-9-yl]-3-oxo-propenyl}-phenyl)-acetamide

### a) (E)-3-[4-Chloro-2-(2,2,2-trifluoroacetylamino)-phenyl]-acrylic acid

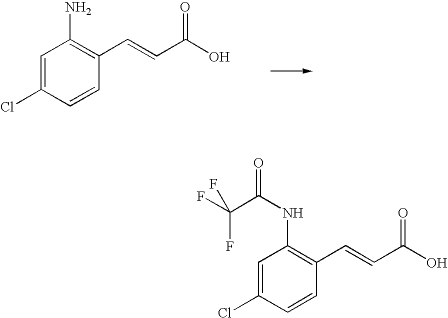

(E)-3-(2-amino-4-chlorophenyl)-acrylic acid (200 mg; 0.67 mmol) (R. W. Carling et al., J. Med. Chem. (1997), 40(5), 754-765) in CH2Cl2 (6 ml) and NEt3 (0.19 ml; 1.3 mmol) is stirred, cooled to 0° C. and combined with TFAA (0.096 ml; 0.67 mmol). The reaction mixture is warmed to room temp., stirred for 10 min. poured on 2N HCl and extracted with TBME three times. The combined organic phases are dried over Na2SO4 and evaporated to dryness to yield the title compound as slightly yellow crystals (205 mg; 100%).

1H-NMR (400 MHz; DMSO-d6), δ (ppm): 6.58(d, 1H); 7.46-7.50(m, 2H); 7.54(55, 1H); 7.93(d, 1H); 11.40(s, 1H); 12.5(s, 1H).

MS (m/z) ES−: 292.0 (M−H−; 100).

### b) (E)-9-{3-[4-Chloro-2-(2,2,2-trifluoroacetylamino)-phenyl]-acryloyl}-7-(4-fluorobenzyl)-3,7,9-triazabicyclo[3.3.1]nonane-3-carboxylic acid tert-butyl ester

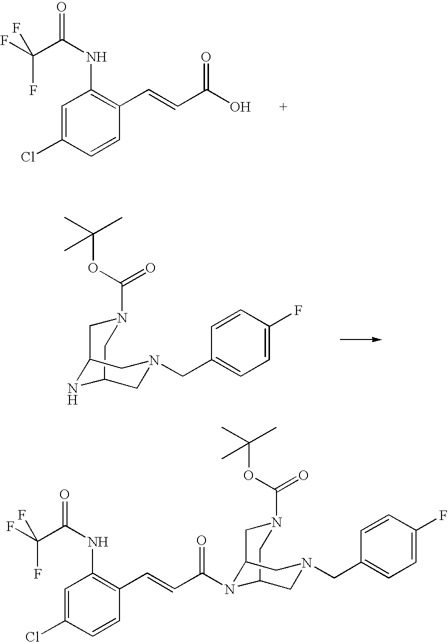

7-(4-Fluorobenzyl)-3,7,9-triazabicyclo[3.3.1]nonane-3-carboxylic acid tert-butyl ester (Example 64h, 230 mg; 0.7 mmol), (E)-3-[4-chloro-2-(2,2,2-trifluoroacetylamino)-phenyl]-acrylic acid (205 mg; 0.7 mmol) and EDCl.HCl (134 mg; 0.7 mmol) in CH2Cl2 (4 ml) are stirred for 2 h at room temp., poured on a silica gel column and chromatographed (acetone/hexanes 15/85) to yield the title compound as colorless crystals (294 mg; 69%).

1H-NMR (400 MHz; DMSO-d6), δ (ppm): 1.47(s, 9H); 2.08(bd, 0.5H); 2.18(bt, 1H); 2.29(bd, 0.5H); 2.88-3.22(m, 4H); 3.27(d, 1H); 3.45(dd, 1H); 3.98(m, 2H); 4.48(m, 2H); 7.08(t, 2H); 7.23-7.37(m, 3H); 7.45-7.53(m, 3H); 8.03(dd, 1H); 11.4(s, 1H).

MS (m/z) ES+: 611.0(MH+, 100).

### c) (E)-9-[3-(2-Amino-4-chlorophenyl)-acryloyl]-7-(4-fluorobenzyl-3,7,9-triazabicyclo[3.3.1]nonane-3-carboxylic acid tert-butyl ester

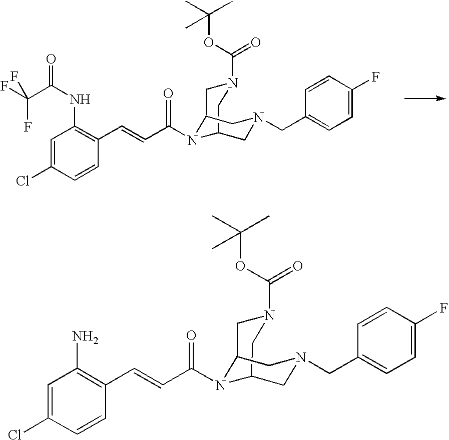

(E)-9-{3-[4-Chloro-2-(2,2,2-trifluoroacetylamino)-phenyl]-acryloyl}-7-(4-fluorobenzyl)-3,7,9-triazabicyclo[3.3.1]nonane-3-carboxylic acid tert-butyl ester (240 mg; 0.39 mmol) in EtOH (14 ml) and 2N NaOH (5 ml) is refluxed for 1.5 h, poured on brine and extracted with TBME three times. The combined organic phases are dried over Na2SO4 and evaporated to dryness to yield the title compound as slightly yellow crystals (199 mg; 99%).

1H-NMR (400 MHz; DMSO-d6), δ (ppm): 1.45(s, 9H); 2.08(bd, 0.5H); 2.17(bt, 1H); 2.28(bd, 0.5H); 2.92(d, 1H); 2.96(d, 1H); 3.05(bt, 1H); 3.17(bd, 1H); 3.26(d, 1H); 3.42(d, 1H); 4.04(dd, 2H); 4.46(bs, 1H); 4.58(bs, 1H); 5.76(s, 2H, NH2). 6.52(bd, 1H); 6.71 (d, 1H); 6.96(dd, 1H); 7.08(t, 2H); 7.33(dd, 2H); 7.51(dd, 1H); 7.68(1H).

MS (m/z) ES+: 515.1(MH+, 100).

### d) (E)-9-[3-(2-Acetylamino-4-chlorophenyl)-acryloyl]-7-(4-fluorobenzyl)-3,7,9-triazabicyclo[3.3.1]nonane-3-carboxylic acid tert-butyl ester

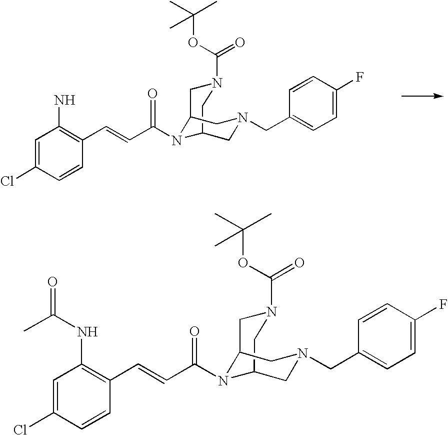

(E)-9-[3-(2-Amino-4-chlorophenyl)-acryloyl]-7-(4-fluorobenzyl)-3,7,9-triazabicyclo[3.3.1]nonane-3-carboxylic acid tert-butyl ester (43 mg; 0.08 mmol) in THF (4 ml) and NEt3 (0.12 ml; 0.83 mmol) is treated with acetylchloride (0.059 ml; 0.83 mmol) at reflux for 30 min., poured on 2N Na2CO3 and extracted with TBME three times. The combined organic phases are dried over Na2SO4, evaporated and purified by chromatography (SiO2, acetone/hexanes 2/8 to 3/7) to yield the title compound as colorless crystals (29 mg; 62%).

1H-NMR (400 MHz; DMSO-d6), δ (ppm): 1.45(s, 9H); 2.09(bs, 3.5H); 2.18(bt, 1H); 2.28(bd, 0.5H); 2.90-3.21(m, 4H); 3.38(d, 1H); 3.43(d, 1H); 4.06(dd, 2H); 4.50(bd, 1H); 4.58(bs, 1H); 7.08(t, 2H); 7.17(dd, 1H); 7.28(m, 1H); 7.33(dd, 2H); 7.56(bs, 1H); 7.68(bd, 1H); 7.90(m, 1H); 9.90(s, 1H).

MS (m/z) ES+: 557.1(MH+, 100).

### e) (E)-N-(5-Chloro-2-{3-[3-(4-fluorobenzyl)-3,7,9-triazabicyclo[3.3.1]non-9-yl]-3-oxo-propenyl}-phenyl)-acetamide

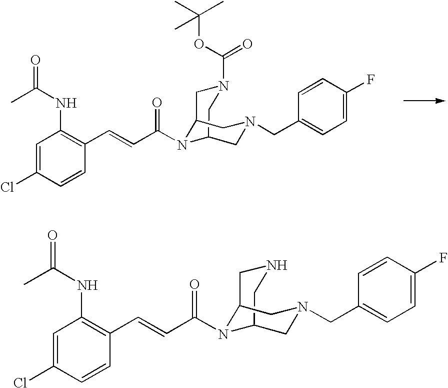

(E)-N-(5-Chloro-2-{3-[3-(4-fluorobenzyl)-3,7,9-triazabicyclo[3.3.1]non-9-yl]-3-oxopropenyl}-phenyl)-acetamide (26 mg; 0.046 mmol) in CH2Cl2/TFA (1 ml/1 ml) is kept at room temp for 5 min. and then evaporated to dryness, taken up in EtOH, 2N Na2CO3/2N NaOH and extracted with TBME three times. The combined organic phases are dried over Na2SO4, evaporated and the remaining resin dissolved in a few drops of EtOH, diluted with TBME and filtered from some precipitated impurity. Evaporation delivered the title compound as a colorless foam (20 mg; 90%).

1H-NMR (400 MHz; DMSO-d6), δ (ppm): 2.10(s, 3H); 2.28(m, 2H); 2.37(m, 2H); 2.75-3.06(m, 4H); 3.37(d, 2H); 4.28(s, 1H); 4.48(s, 1H); 7.08-7.18(m, 4H); 7.33-7.40(m, 2H); 7.55(s, 1H); 7.65(d, 1H); 7.90(m, 1H); 9.90(s, 1H).

MS (m/z) ES+: 457.1(MH+, 100).

### Example 69

### (E)-N-(5-Chloro-2-{3-[3-(4-fluorobenzyl)-3,7,9-triazabicyclo[3.3.1]non-9-yl]-3-oxopropenyl}-phenyl)-2-dimethylaminoacetamide

### a) (E)-9-{3-[4-Chloro-2-(2-dimethylaminoacetylamino)-phenyl]-acryloyl}-7-(4-fluorobenzyl)-3,7,9-triazabicyclo[3.3.1]nonane-3-carboxylic acid tert-butyl ester

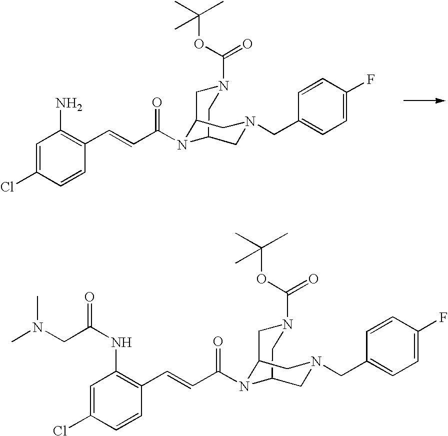

(E)-N-(5-Chloro-2-{3-[3-(4-fluorobenzyl)-3,7,9-triazabicyclo[3.3.1]non-9-yl]-3-oxopropenyl}-phenyl)-acetamide (80 mg; 0.16 mmol) dissolved in THF (1 ml) is treated with chloroacetylchloride (0.015 ml; 0.19 mmol) for 15 min at room temp. Dimethylamine (˜0.2 ml) is introduced, and the reaction mixture kept at room temp for 20 min., evaporated to dryness and purified via chromatography (SiO2; acetone/hexanes 4/6 to 8/2) to yield the title compound as colorless foam (59 mg; 64%).

1H-NMR (400 MHz; DMSO-d6), δ (ppm): 1.45(s, 9H); 2.08(bd, 0.5H); 2.18(bt, 1H); 2.25(bd, 0.5H); 2.32(s, 6H); 2.88-3.00(m, 2H); 3.00-3.22(m, 2H); 3.12(s, 2H); 3.28(d, 1H); 3.43(d, 1H); 3.98-4.13(m, 2H); 4.52(bd, 1H); 4.57(bs, 1H); 7.09(t, 2H); 7.20(dd, 1H); 7.30(m, 1H); 7.35(dd, 2H); 7.60(dd, 2H); 7.98(m, 1H); 9.82(s, 1H).

MS (m/z) ES+: 600.1(MH+, 100).

### b) (E)-N-(5-Chloro-2-{3-[3-(4-fluorobenzyl)-3,7,9-triazabicyclo[3.3.1]non-9-yl]-3-oxopropenyl}-phenyl)-2-dimethylaminoacetamide

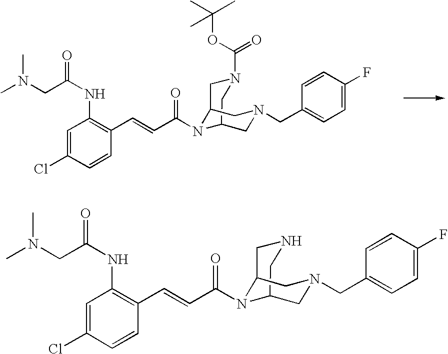

(E)-9-{3-[4-Chloro-2-(2-dimethylaminoacetylamino)-phenyl]-acryloyl}-7-(4-fluorobenzyl)-3,7,9-triazabicyclo[3.3.1]nonane-3-carboxylic acid tert-butyl ester (55 mg; 0.09 mmol) in CH2Cl2/TFA (1 ml/1 ml) is kept at room temp. for 5 min., poured on 2N Na2CO3/2N NaOH and extracted with TBME/EtOH (˜10:1) three times. The combined organic phases are dried over Na2SO4, filtered and evaporated to dryness. The resulting solid is washed with TBME/hexanes to deliver the target compound as slightly yellow solid (30 mg; 57%).

1H-NMR (400 MHz; DMSO-d6), δ (ppm): 2.28(d, 2H); 2.33(s, 6H); 2.36(d, 2H); 2.73-2.90(m, 2H); 2.97-3.05(m, 2H); 3.11(s, 2H); 3.35(dd, 2H); 4.28(s, 1H); 4.37(s, 1H); 7.12-7.20(m, 3H); 7.28(dd, 1H); 7.36(dd, 2H); 7.60(m, 2H); 7.89(d, 1H); 9.81(s, 1H).

MS (m/z) ES+: 500.2(MH+, 100).

### Example 70

### (E)-N-(5-Chloro-2-{3-[3-(4-fluorobenzyl)-3,7,9-triazabicyclo[3.3.1]non-9-yl]-3-oxopropenyl}-Phenyl)methanesulfonamide

### a) (E)-9-[3-(4-Chloro-2-methanesulfonylamino-Phenyl)-acryloyl]-7-(4-fluoro-benzyl)-3,7,9-triazabicyclo[3.3.1]nonane-3-carboxylic acid tert-butyl ester

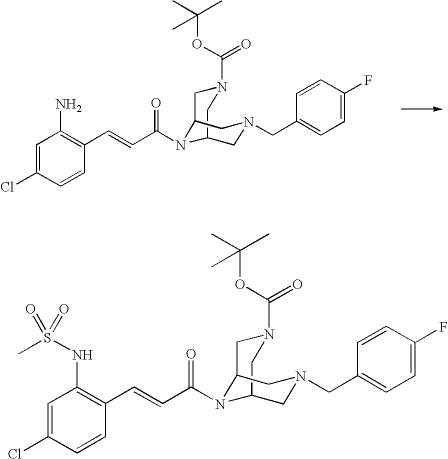

(E)-N-(5-Chloro-2-{3-[3-(4-fluorobenzyl)-3,7,9-triazabicyclo[3.3.1]non-9-yl]-3-oxopropenyl}-phenyl)-acetamide (37 mg; 0.07 mmol) in THF (2 ml) and NEt3 (0.06 ml; 0.43 mmol) is treated with CH3SO2Cl (0.017 ml; 0.21 mmol). After 10 min. at room temp. a second portion of NEt3 (0.06 ml; 0.43 mmol) and CH3SO2Cl (0.017 ml; 0.21 mmol) is added. After 10 min. the reaction is poured on EtOH/2N NaOH, kept for 5 min. and extracted with TBME three times. The combined organic phases are dried over Na2SO4, evaporated and purified by chromatography (SiO2, TBME/MeOH/NH3conc 90/10/1 then EtOAc/MeOH 8/2) to yield the title compound as yellow foam (15 mg; 35%).

1H-NMR (400 MHz; DMSO-d6), δ (ppm): 1.48(s, 9H); 2.05-2.33(m, 2H); 2.90-3.20(m, 4H); 3.02(s, 3H); 3.30(d, 1H); 3.42(d, 1H); 4.05(dd, 2H); 4.50(bd, 1H); 4.58(bs, 1H); 7.08(t, 2H); 7.18(dd, 1H); 7.32-7.40(m, 4H); 7.83(d, 1H); 7.93(dd, 1H)

MS (m/z) ES+: 593.1(MH+, 100).

### b) (E)-N-(5-Chloro-2-{3-[3-(4-fluorobenzyl)-3,7,9-triazabicyclo[3.3.1]non-9-yl]-3-oxopropenyl}-phenyl)methanesulfonamide

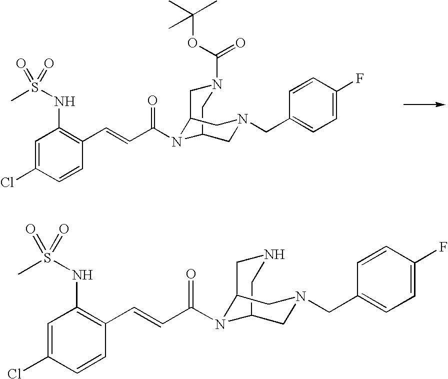

(E)-9-[3-(4-Chloro-2-methanesulfonylamino-phenyl)-acryloyl]-7-(4-fluoro-benzyl)-3,7,9-triazabicyclo[3.3.1]nonane-3-carboxylic acid tert-butyl ester (15 mg; 0.02 mmol) is dissolved in CH2Cl2/TFA (1 ml/1 ml) and kept at room temp. for 10 min. 2N Na2CO3/2N NaOH is added and the reaction mixture extracted with TBME/EtOH (˜10/1) three times. The combined organic phases are dried over Na2SO4, evaporated and the resulting solid triturated with TBME to yield the title compound as yellowish crystals (7 mg; 58%).

1H-NMR (400 MHz; DMSO-d6), δ (ppm): 2.24-2.42(m, 4H); 2.71 (s, 3H); 2.82-3.20(m, 4H); 3.40(dd, 2H); 4.37(s, 1H); 4.51(s, 1H); 7.08(d, 1H); 7.12-7.20(m, 2H); 7.23-7.32(m, 2H); 7.40(dd, 2H); 7.55(d, 1H); 7.93(d, 1H).

MS (m/z) ES+: 494.2(MH+, 100).

### Example 71

### (5-Chloro-2-{(E)-3-[3-(4-fluorobenzyl)-3,7,9-triazabicyclo[3.3.1]non-9-yl]-3-oxopropenyl}-phenyl)-urea hydrochloride

### a) 9-[(E)-3-(4-Chloro-2-ureidophenyl]-acryloyl}-7-(4-fluorobenzyl)-3,7,9-triazabicyclo[3.3.1]nonane-3-carboxylic acid tert-butyl ester

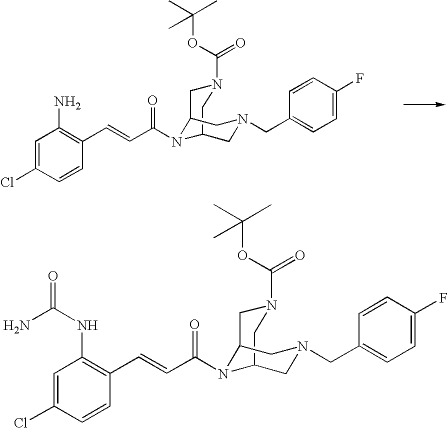

The reaction is performed in analogy to Example 4 and the title product purified via chromatography (SiO2, acetone/hexanes 4/6 to 7/3) to yield the desired product as colorless foam (88 mg; 82%).

1H-NMR (400 MHz; DMSO-d6), δ (ppm): 1.46 (s, 9H); 2.08 (bd, 0.5H); 2.18 (bt, 1H); 2.28 (bd, 0.5H); 2.90-3.11 (m, 3H); 3.30 (dd, 2H); 3.45 (t, 1H); 3.97-4.14 (m, 2H); 4.48 (bd, 1H); 4.59 (bs, 1H); 6.23 (s, 2H); 7.02-7.15 (m, 4H); 7.33 (dd, 2H); 7.68 (s, 1H); 7.73 (dd, 1H); 7.93 (d, 1H); 8.38 (s, 1H).

MS (m/z) ES+: 558.1 (MH+).

### b) (5-Chloro-2-{(E)-3-[3-(4-fluorobenzyl)-3,7,9-triazabicyclo[3.3.1]non-9-yl]-3-oxopropenyl}-phenyl)-urea hydrochloride (AST391)

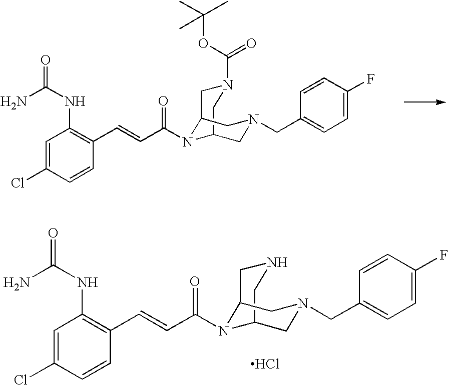

9-[(E)-3-(4-Chloro-2-ureidophenyl]-acryloyl}-7-(4-fluorobenzyl)-3,7,9-triazabicyclo[3.3.1]nonane-3-carboxylic acid tert-butyl ester (80 mg; 0.143 mmol) is dissolved in EtOH/HClconc (1 ml/1 ml) and is carefully evaporated after 2 min., and recrystallised from hot EtOH to yield the title compound as colorless crystals (50 mg; 71%).

1H-NMR (400 MHz; DMSO-d6), δ (ppm): 2.25 (d, 1H); 2.32 (d, 1H); 3.00-3.15 (m, 2H); 3.24 (bs, 1H); 3.41-3.54 (m, 5H); 4.75 (bd, 2H); 6.26 (bs, 2H); 7.05-7.21 (m, 4H); 7.46 (m, 2H); 7.70-7.78 (m, 2H); 7.93 (s, 1H); 8.30 (bs, 1H); 8.48 (s, 1H); 9.48 (bs, 1H).

MS (m/z) ES+: 458.0 (MH+).

### Example 72

### (E)-N-(5-Chloro-2-{3-[3-(4-fluorobenzyl)-3,7,9-triazabicyclo[3.3.1]non-9-yl]-3-oxo-propenyl}-phenyl)-acetamide

### a) (E)-9-[3-(2-Acetylamino-4-chloro-5-fluorophenyl)-acryloyl]-7-(4-fluorobenzyl)-3,7,9-triazabicyclo[3.3.1]nonane-3-carboxylic acid tert-butyl ester

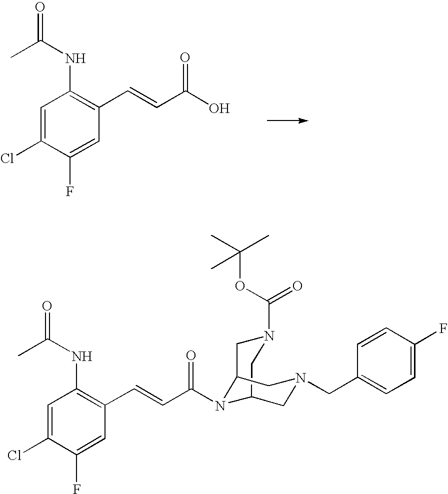

(E)-3-(2-Acetylamino-4-chloro-5-fluoro-phenyl)-acrylic acid (prepared as described in Example 153) is treated in analogy to Example 68b and purified via chromatography (SiO2, acetone/hexanes 317) to yield the desired product after recrystallisation from TBME (305 mg; 89%).

1H-NMR (400 MHz; DMSO-d6), δ (ppm): 1.46 (s, 9H); 2.03 (s, 3H); 2.18 (bt, 2H); 2.30 (bd, 1H); 2.90-3.00 (m, 2H); 3.30 (s, 2H); 3.40-3.50 (dd, 1H); 4.00-4.12 (m, 2H); 4.48-4.58 (m, 2H); 7.08 (t, 2H); 7.25 (dd, 1H); 7.33 (dd, 2H); 7.58 (m, 2H); 8.00 (m, 1H); 9.88 (s, 1H).

MS (m/z) ES−: 573.2 (MH−).

### b) (E)-N-(5-Chloro-2-{3-[3-(4-fluorobenzyl)-3,7,9-triazabicyclo[3.3.1]non-9-yl]-3-oxo-propenyl}-phenyl)-acetamide hydrochloride

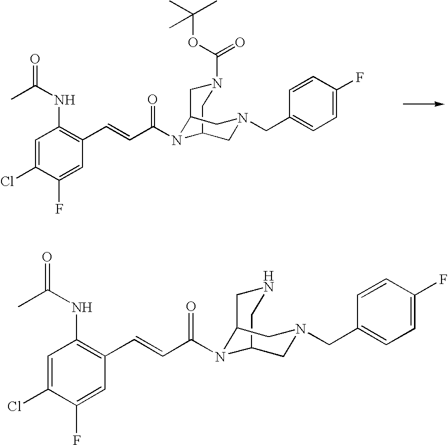

The title compound is obtained following the procedure described in Example 71b, rendering the desired compound as colorless crystals (162 mg; 61%).

1H-NMR (400 MHz; DMSO-d6), δ (ppm): 2.08 (s, 3H); 2.25 (d, 1H); 2.32 (d, 1H); 3.00-3.28 (m, 2H); 3.51 (bd, 2H); 4.62 (bs, 4H); 4.75 (s, 2H); 7.16 (t, 2H); 7.25 (d, 1H); 7.45 (bt, 2H); 7.58-7.65 (m, 2H); 7.97 (d, 1H); 8.32 (bs, 1H); 9.53 (bs, 1H); 9.93 (s, 1H).

MS (m/z) ES+: 473.2 (MH+).

### Example 73

### (5-Chloro-2-{(E)-3-[3-(4-fluorobenzyl)-7-methyl-3,7,9-triazabicyclo[3.3.1]non-9-yl]-3-oxopropenyl}phenyl)-urea

### a) 3,9-Dibenzyl-7-methyl-3,7,9-triazabicyclo[3.3.1]nonane

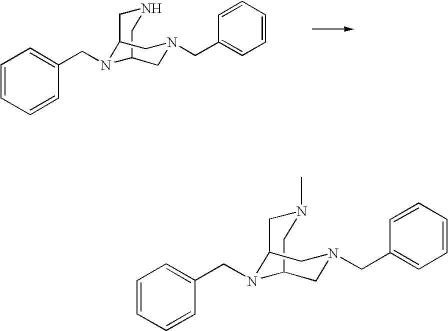

3,9-Dibenzyl-3,7,9-triaza-bicyclo[3.3.1]nonane (0.5 g; 1.63 mmol) is dissolved in MeOH (20 ml), formaldehyde (aq. 35%; 0.56 ml; 6.5 mmol) added and the mixture kept at 50° C. for 15 min. NaBH4 (186 mg; 4.8 mmol) is added and stirring continued for 15 min. The reaction is quenched with 2N HCl, saturated solution of K2CO3 is added and the mixture extracted with TBME three times. The combined organic phases are dried over Na2SO4, filtered and evaporated to dryness to deliver the title product as yellowish oil (550 mg; 100%).

1H-NMR (400 MHz; DMSO-d6), δ (ppm): 2.18 (s, 3H); 2.42-2.65 (m, 8H); 2.72 (bs, 2H); 3.43 (s, 2H); 3.88 (s, 2H); 7.20 (bt, 2H); 7.26-7.36 (m, 8H).

MS (m/z) ES+: 322 (MH+).

### b) 3-Methyl-3,7,9-triazabicyclo[3.3.1]nonane dihydrochloride

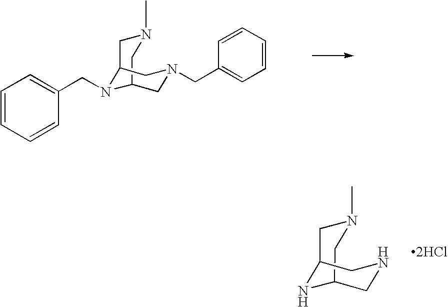

3,9-Dibenzyl-7-methyl-3,7,9-triazabicyclo[3.3.1]nonane (520 mg; 1.62 mmol) is dissolved in HOAc (150 ml) and hydrogenated over Pd/C (1 g) for 1 h. HCl (3.3 mmol) in ether (20 ml) is added and the mixture evaporated to dryness delivering the title product as a colorless solid (345 mg; 98%).

1H-NMR (400 MHz; DMSO-d6), δ (ppm): 2.20 (s, 3H); 2.70 (bd, 2H); 3.08 (bd, 2H); 3.52 (bd, 2H); 3.58 (bd, 2H); 3.80 (bs, 2H); 8.45 (bs, 1H); 9.90 (bs, 1H); 10.11 (bs, 1H); 10.48 (bs, 1H).

MS (m/z) ES+: 142 (MH+).

### c) 3-(4-Fluorobenzyl)-7-methyl-3,7,9-triazabicyclo[3.3.1]nonane

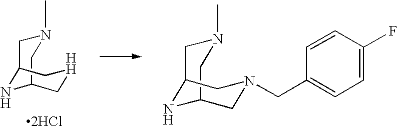

3-Methyl-3,7,9-triazabicyclo[3.3.1]nonane dihydrochloride (340 mg; 1.6 mmol) is dissolved in EtOH (18 ml) 4-fluorobenzyl chloride (0.19 ml; 1.6 mmol) and NaHCO3 (670 mg; 7.9 mmol) added and the mixture refluxed for 2.5 h. The solvent is evaporated, the residue taken up in TBME, filtered and purified via chromatography (SiO2, TBME/MeOH/NH3conc 90/10/1 to 80/20/2) to yield the desired product as a yellow oil (240 mg; 61%).

1H-NMR (400 MHz; DMSO-d6), δ (ppm): 2.17 (s, 3H); 2.25-2.35 (m, 4H); 2.58-2.65 (bd, 4H); 2.91 (bs, 2H); 3.39 (s, 2H); 4.35 (bs, 1H); 7.10 (t, 2H); 7.35 (dd, 2H).

MS (m/z) ES+: 250.1 (MH+).

### d) (5-Chloro-2-{(E)-3-[3-(4-fluorobenzyl)-7-methyl-3,7,9-triazabicyclo[3.3.1]non-9-yl]-3-oxopropenyl}phenyl)-carbamic acid tert-butyl ester

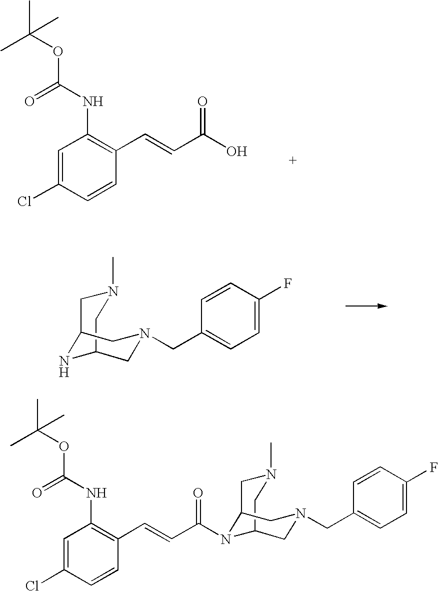

The reaction is performed in analogy to Example 68b and the title product purified via chromatography (SiO2, TBME/MeOH/NH3conc 90/10/1) to yield the desired product as yellow foam (480 mg; 90%).

MS (m/z) ES+: 530 (MH+).

### e) (E)-3-(2-Amino-4-chlorophenyl)-1-[3-(4-fluorobenzyl)-7-methyl-3,7,9-triazabicyclo[3.3.1]non-9-yl]-propenone

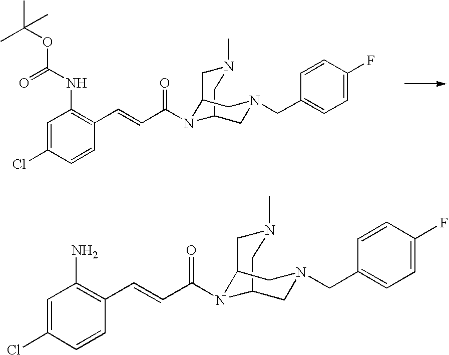

(5-Chloro-2-{(E)-3-[3-(4-fluorobenzyl)-7-methyl-3,7,9-triazabicyclo[3.3.1]non-9-yl]-3-oxopropenyl}phenyl)-carbamic acid tert-butyl ester (480 mg; 0.76 mmol) is dissolved in EtOH (4 ml) and HClconc (6 ml) and kept at room temp. for 1-2 min. Water is added and the mixture washed with TBME. The aq. phase is adjusted to pH ˜10 by adding a saturated solution of Na2CO3 and then extracted with EtOAc three times. The combined organic phases are dried over Na2SO4, filtered and evaporated to dryness to deliver a the title product as a yellow foam (330 mg; 100%).

1H-NMR (400 MHz; DMSO-d6), δ (ppm): 2.15 (s, 3H); 2.20 (dd, 1H); 2.25 (dd, 2H); 2.35 (dd, 1H); 2.75-2.85 (m, 4H); 3.44 (q, 2H); 4.47 (bs, 1H); 4.60 (bs, 1H); 5.73 (bs, 2H); 6.50 (dd, 1H); 6.70 (d, 1H); 6.95 (d, 1H); 7.11 (t, 2H); 7.36 (dd, 2H); 7.52 (d, 1H); 7.65 (d, 1H).

MS (m/z) ES+: 429 (MH+).

### f) (5-Chloro-2-{(E)-3-[3-(4-fluorobenzyl)-7-methyl-3,7,9-triazabicyclo[3.3.1]non-9-yl]-3-oxopropenyl}phenyl)-urea

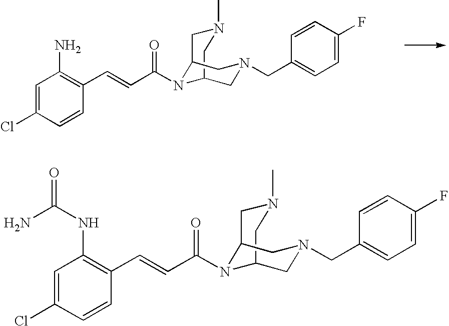

The reaction is performed in analogy to Example 4 and the title product purified via chromatography (XTerra, RP18, 7 μm, MeCN/water 40/60 to 100/0) to yield the title compound as yellowish foam (42 mg; 55%).

1H-NMR (400 MHz; DMSO-d6), δ (ppm): 2.18 (s, 3H); 2.24 (dd, 1H); 2.30 (dd, 2H); 2.48 (dd, 1H); 2.75-2.85 (m, 4H); 3.44 (q, 2H); 4.50 (bs, 1H); 4.63 (bs, 1H); 6.25 (bs, 2H); 7.05 (dd, 1H); 7.10-7.17 (m, 3H); 7.36 (dd, 2H); 7.69 (d, 1H); 7.36 (d, 1H); 7.95 (d, 1H); 8.40 (s, 1H).

MS (m/z) ES+: 472 (MH+).

### Example 74

### N-(5-Chloro-2-{(E)-3-[3-(4-fluorobenzyl)-7-methyl-3,7,9-triazabicyclo[3.3.1]non-9-yl]-3-oxopropenyl}-phenyl)-methanesulfonamide

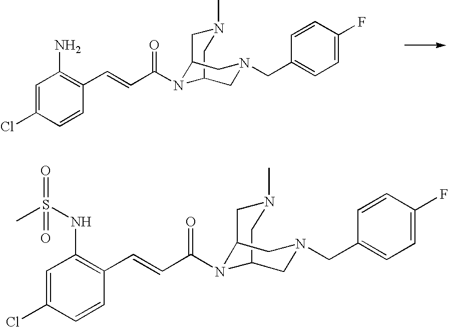

The reaction is performed in analogy to Example 70a and the title product purified via chromatography (XTerra, RP18, 7 μm, MeCN/water 40/60 to 100/0) to yield the title compound as yellowish foam, which is further purified via chromatography (SiO2, EtOAc/MeOH/NH3conc 90/10/0.5 to 80/20/1) to yield the desired product as a yellow foam (25 mg; 35%).

1H-NMR (400 MHz; CDCl₃), δ (ppm): 2.45-2.60 (m, 4H); 2.70-2.90 (m, 3H); 3.00-3.400 (m, 4H); 3.08 (s, 3H); 3.65 (bd, 2H); 4.48 (bs, 1H); 4.83 (bs, 1H); 6.78 (d, 1H); 7.00 (t, 2H); 7.16 (d, 1H); 7.16 (d, 1H); 7.37 (bt, 2H); 7.47 (d, 1H); 7.55 (s, 1H); 7.83 (d, 1H).

MS (m/z) ES+: 507.1 (MH+).

### Example 75

### N-(5-Chloro-2-{(E)-3-[3-(4-fluorobenzyl)-7-methyl-3,7,9-triazabicyclo[3.3.1]non-9-yl]-3-oxopropenyl}phenyl)-acetamide

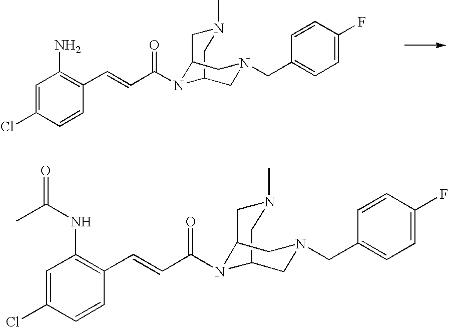

The reaction is performed in analogy to Example 1f and the title product purified via chromatography (XTerra, RP18, 7 μm, MeCN/water 40/60 to 100/0) to yield the title compound as yellow foam (21 mg; 28%).

1H-NMR (400 MHz; DMSO-d6), δ (ppm): 2.100 (s, 3H); 2.16 (s, 3H); 2.22 (d, 1H); 2.28 (m, 2H); 2.37 (dd, 1H); 2.76-2.85 (m, 4H); 3.45 (q, 2H); 4.52 (bs, 1H); 4.60 (bs, 1H); 7.12 (t, 2H); 7.17 (d, 1H); 7.25 (dd, 1H); 7.37 (dd, 2H); 7.54 (bs, 1H); 7.65 (d, 1H); 7.90 (d, 1H); 9.90 (s, 1H).

MS (m/z) ES+: 471.1 (MH+).

### Example 76

### N-(5-Chloro-2-{(E)-3-[3-(4-fluorobenzyl)-3,7,9-triazabicyclo[3.3.1]non-9-yl]-3-oxo-propenyl}-4-methoxyphenyl)-acetamide

### a) (E)-9-[3-(2-Amino-4-chloro-5-methoxyphenyl)-acryloyl]-7-(4-fluorobenzyl)-3,7,9-triazabicyclo[3.3.1]nonane-3-carboxylic acid tert-butyl ester

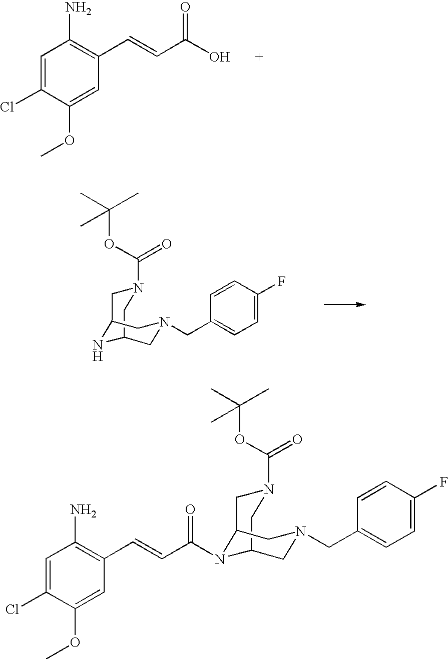

Acid (Example 23c) and amine (Example 64h) are coupled in analogy to Example 23d and the title product purified via chromatography (SiO2, acetone/hexanes 5/6) to yield the desired product as yellow foam (40 mg; 52%).

MS (m/z) ES+: 545.1 (MH+).

### b) (E)-9-[3-(2-Acetylamino-4-chloro-5-methoxyphenyl)-acryloyl]-7-(4-fluorobenzyl)-3,7,9-triazabicyclo[3.3.1]nonane-3-carboxylic acid tert-butyl ester

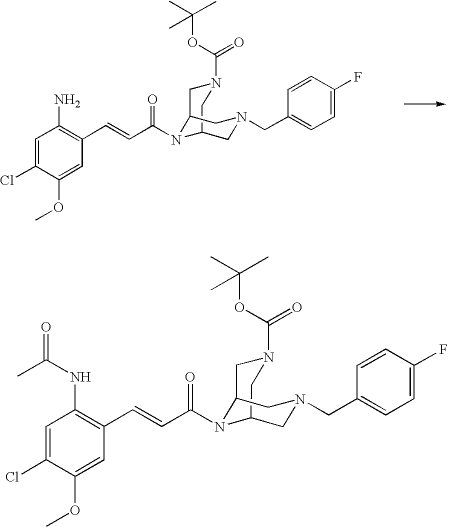

The reaction is performed in analogy to Example 1f and the title product purified via chromatography (SiO2, acetone/hexanes 4/6 to 1/1) to yield the desired product as yellow crystals (54 mg; 50%).

1H-NMR (400 MHz; DMSO-d6), δ (ppm): 1.46 (s, 9H); 2.05 (s, 3H); 2.10 (bd, 0.5H); 2.20 (bd, 1H); 2.32 (bd, 0.5H); 2.90-3.13 (m, 4H); 3.42 (d, 1H); 3.47 (d, 1H); 3.91 (s, 3H); 4.08 (d, 1H); 4.13 (d, 1H); 4.52 (bd, 1H); 4.59 (bs, 1H); 7.10 (t, 2H); 7.21 (dd, 1H); 7.36 (bt, 2H); 7.46 (m, 2H); 7.62 (d, 1H); 9.75 (d, 1H).

MS (m/z) ES+: 587.1 (MH+).

### c) N-(5-Chloro-2-{(E)-3-[3-(4-fluorobenzyl)-3,7,9-triazabicyclo[3.3.1]non-9-yl]-3-oxo-propenyl}-4-methoxyphenyl)-acetamide hydrochloride

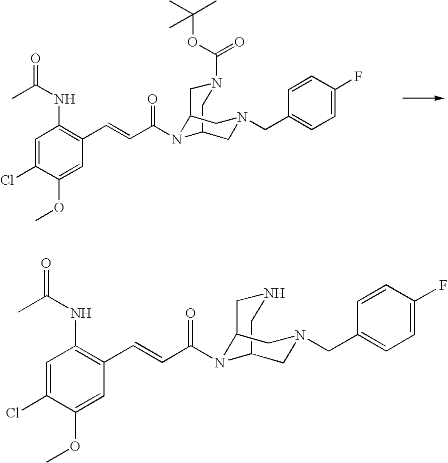

The title compound is obtained following the procedure described in Example 71b, rendering the desired compound as colorless crystals (162 mg; 61%).

1H-NMR (400 MHz; DMSO-d6), δ (ppm): 2.03 (s, 3H); 2.26 (d, 1H); 2.35 (d, 1H); 3.00-3.12 (m, 3H); 3.24 (bs, 1H); 3.50 (bs, 4H), 3.93 (s, 3H); 4.78 (s, 2H); 7.17-7.25 (m, 3H); 7.45-7.50 (m, 4H); 7.65 (d, 1H); 8.32 (bs, 1H); 9.46 (bs, 1H); 9.80 (s, 1H).

MS (m/z) ES+: 487 (MH+).

### Example 77

### N-(5-Chloro-2-{(E)-3-[3-(4-fluorobenzyl)-3,7,9-triazabicyclo[3.3.1]non-9-yl]-3-oxo-propenyl}-4-methoxyphenyl)-methanesulfonamide hydrochloride

### a) 9-[(E)-3-(4-Chloro-2-methanesulfonylamino-5-methoxyphenyl)-acryloyl]-7-(4-fluorobenzyl)-3,7,9-triazabicyclo[3.3.1]nonane-3-carboxylic acid tert-butyl ester

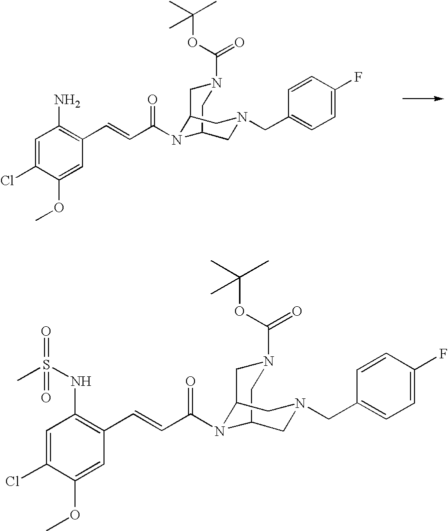

The reaction is performed in analogy to Example 70a and the title product purified via chromatography (SiO2, TBME/MeOH/NH3conc 95/5/2 to 95/5/0) to yield the desired product as yellow foam (84 mg; 62%).

1H-NMR (400 MHz; DMSO-d6), δ (ppm): 1.48 (s, 9H); 2.10 (bd, 0.5H); 2.20 (bt, 1H); 2.32 (bd, 0.5H); 2.90-3.00 (m, 3H); 3.07 (s, 3H); 3.20 (bd, 1H); 3.52 (bd, 1H); 3.49 (bd, 1H); 3.95 (s, 3H); 4.05 (d, 1H); 4.12 (d, 1H); 4.50 (bs, 1H); 4.57 (bs, 1H); 7.08 (t, 2H); 7.20 (dd, 1H); 7.30-7.38 (m, 3H); 7.50 (bs, 1H); 7.82 (d, 1H); 9.42 (s, 1H).

MS (m/z) ES+: 623.1 (MH+).

### b) N-(5-Chloro-2-{(E)-3-[3-(4-fluorobenzyl)-3,7,9-triazabicyclo[3.3.1]non-9-yl]-3-oxo-propenyl}-4-methoxyphenyl)-methanesulfonamide hydrochloride

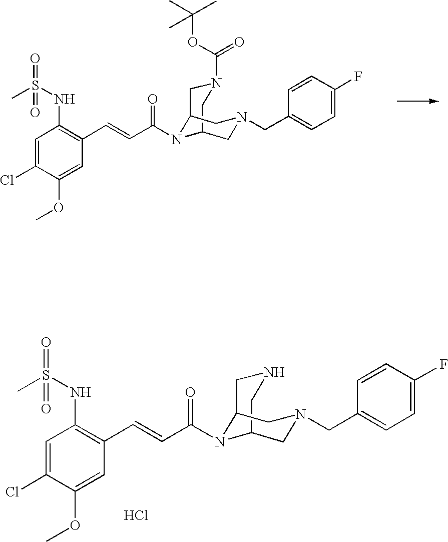

The title compound is obtained following the procedure described in Example 71b, rendering the desired compound as colorless crystals (51 mg; 81%).

1H-NMR (400 MHz; DMSO-d6), δ (ppm): 2.28 (bd, 1H); 2.46 (bd, 1H); 2.96 (s, 3H); 3.03-3.30 (m, 4H); 3.48 (bs, 5H); 3.97 (s, 3H); 4.75 (s, 2H); 7.13-7.23 (m, 2H); 7.39 (s, 1H); 7.46 (m, 2H); 7.53 (s, 1H); 7.86 (d, 1H); 8.35 (bd, 1H); 9.43 (bs, 1H).

MS (m/z) ES+: 523 (MH+).

### Example 78

### (5-Chloro-2-{(E)-3-[3-(4-fluorobenzyl)-3,7,9-triazabicyclo[3.3.1]non-9-yl]-3-oxopropenyl}-4-methoxyphenyl)-urea hydrochloride

### a) 9-[(E)-3-(4-Chloro-5-methoxy-2-ureidophenyl)-acryloyl]-7-(4-fluorobenzyl)-3,7,9-triazabicyclo[3.3.1]nonane-3-carboxylic acid tert-butyl ester

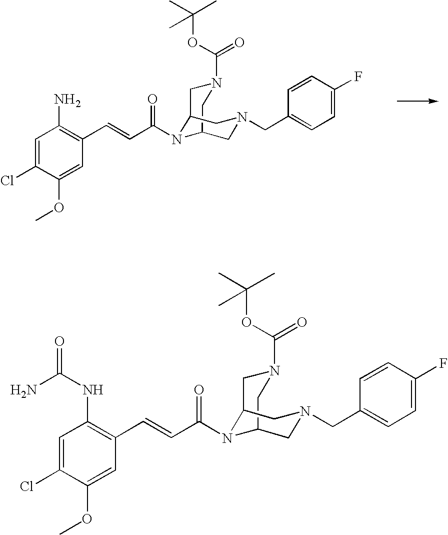

The reaction is performed in analogy to Example 4 and the title product purified via chromatography (SiO2, acetone/hexanes 1/1 to 7/3) to yield the desired product as yellow foam (98 mg; 66%).

1H-NMR (400 MHz; DMSO-d6), δ (ppm): 1.46 (s, 9H); 2.09 (bd, 0.5H); 2.20 (bs, 1H); 2.30 (bd, 0.5H); 2.90-3.11 (m, 3H); 3.18-3.35 (m, 2H); 3.47 (dd, 1H); 3.90 (s, 3H); 4.05 (d, 1H); 4.13 (d, 1H); 4.50 (bs, 1H); 4.60 (bs, 1H); 6.03 (s, 2H); 7.08 (t, 2H); 7.15 (dd, 1H); 7.31-7.39 (m, 3H); 7.67-7.72 (m, 2H); 8.20 (s, 1H).

MS (m/z) ES+: 588.2 (MH+, 70); 488.2 (100).

### b) (5-Chloro-2-{(E)-3-[3-(4-fluorobenzyl)-3,7,9-triazabicyclo[3.3.1]non-9-yl]-3-oxopropenyl}-4-methoxyphenyl)-urea hydrochloride

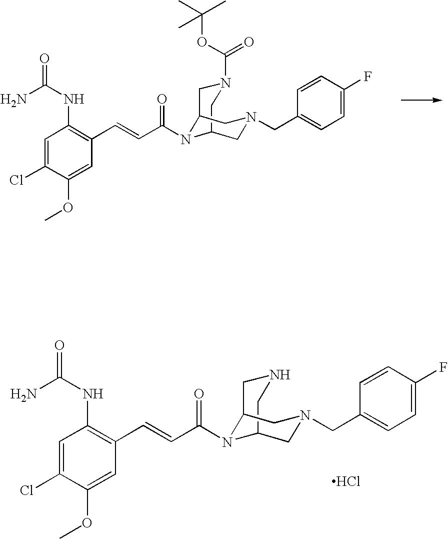

The title compound is obtained following the procedure described in Example 71b, rendering the desired compound as yellowish crystals (62 mg; 76%).

1H-NMR (400 MHz; DMSO-d6), δ (ppm): 2.28 (d, 1H); 2.35 (d, 1H); 3.03-3.12 (m, 3H); 3.23 (m, 1H); 3.45-3.53 (m, 4H); 3.89 (s, 3H); 4.75 (bd, 2H); 6.02 (bs, 2H); 7.18 (bt, 3H); 7.39 (s, 1H); 7.46 (bt, 2H); 7.69 (s, 1H); 7.73 (s, 1H); 8.26 (s, 1H); 8.35 (bs, 1H); 9.45 (bs, 1H).

MS (m/z) ES+: 488.0 (MH+).

### Example 79

### N-(5-Chloro-2-{(E)-3-[3-(4-fluorobenzyl)-3,7,9-triazabicyclo[3.3.1]non-9-yl]-3-oxopropenyl}-4-methoxyphenyl)-2-dimethylaminoacetamide dihydrochloride

### a) 9-{(E)-3-[4-Chloro-2-(2-dimethylamino-acetylamino)-5-methoxy-Phenyl]-acryloyl}-7-(4-fluoro-benzyl)-3,7,9-triaza-bicyclo[3.3.1]nonane-3-carboxylic acid tert-butyl ester

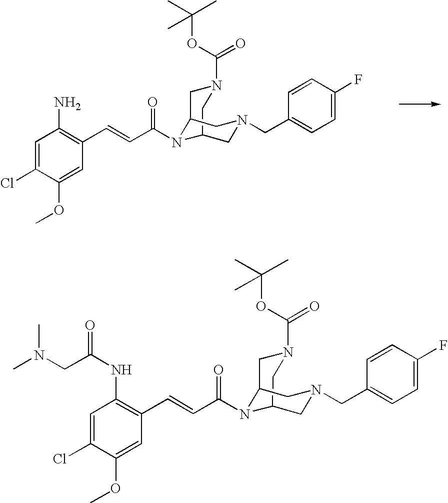

The reaction is performed in analogy to Example 69a and the title product purified via chromatography (SiO2, acetone/hexanes 1/1) to yield the desired product as colorless foam (67 mg; 42%).

MS (m/z) ES+: 631.0 (MH+).

### b) N-(5-Chloro-2-{(E)-3-[3-(4-fluorobenzyl)-3,7,9-triazabicyclo[3.3.1]non-9-yl]-3-oxopropenyl}-4-methoxyphenyl)-2-dimethylaminoacetamide dihydrochloride

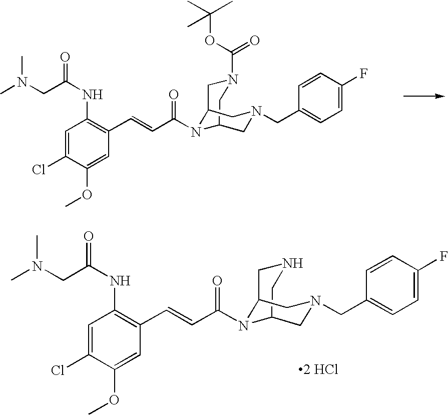

The title compound is obtained following the procedure described in Example 69b, rendering the desired compound as almost colorless crystals (51 mg; 86%).

1H-NMR (400 MHz; DMSO-d6), δ (ppm): 2.22 (d, 1H); 2.33 (d, 1H); 2.88 (s, 6H); 3.00-3.11 (m, 3H); 3.22 (bs, 1H); 3.42-3.54 (m, 4H); 3.95 (s, 3H); 4.18 (d, 2H); 4.73 (bs, 1H); 4.80 (bs, 1H); 7.18 (t, 2H); 7.28 (d, 1H); 7.47 (bt, 2H); 7.51 (s, 1H); 7.53 (s, 1H); 7.61 (d, 1H); 8.30 (bs, 1H); 9.80 (bs, 1H); 9.93 (bs, 1H); 10.57 (s, 1H).

MS (m/z) ES+: 530.1 (MH+).

### Example 80

### N-(2-{(E)-3-[3-Acetyl-7-(4-fluorobenzyl)-3,7,9-triazabicyclo[3.3.1]non-9-yl]-3-oxopropenyl}-5-chloro-4-methoxyphenyl)-acetamide

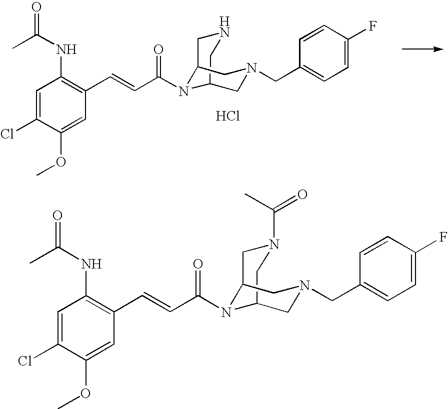

N-(5-Chloro-2-{(E)-3-[3-(4-fluorobenzyl)-3,7,9-triazabicyclo[3.3.1]non-9-yl]-3-oxo-propenyl}-4-methoxyphenyl)-acetamide hydrochloride (100 mg; 1.92 mmol) dissolved in (TBME/THF/2N NaOH 2 ml/4 ml/2 ml) is treated under stirring with acetyl chloride (0.020 ml; 0.28 mmol) for 5 min. at room temp. The reaction mixture is poured on a saturated solution of Na2CO3 and extracted with TBME three times. The combined organic phases are dried over Na2SO4, filtered and evaporated to dryness and purified via chromatography (SiO2, acetone/hexanes 1/1 to 8/2) to yield the desired product as colorless foam, which is crystallised from acetone (37 mg; 37%)

1H-NMR (400 MHz; DMSO-d6), δ (ppm): 1.96 (d, 3H); 2.05 (s, 3H); 2.72-3.05 (m, 3H); 3.15-3.40 (m, 3H); 3.90 (m, 4H); 4.48-4.60 (m, 3H); 7.10 (t, 2H); 7.18-7.27 (m, 3H); 7.43 (d, 1H); 7.48 (s, 1H); 7.62 (d, 1H); 9.70 (s, 1H).

MS (m/z) ES+: 529.03 (MH+).

### Example 81

### 9-[(E)-3-(2-Acetylamino-4-chloro-5-methoxyphenyl)-acryloyl]-7-(4-fluorobenzyl)-3,7,9-triazabicyclo[3.3.1]nonane-3-carboxylic acid methylamide

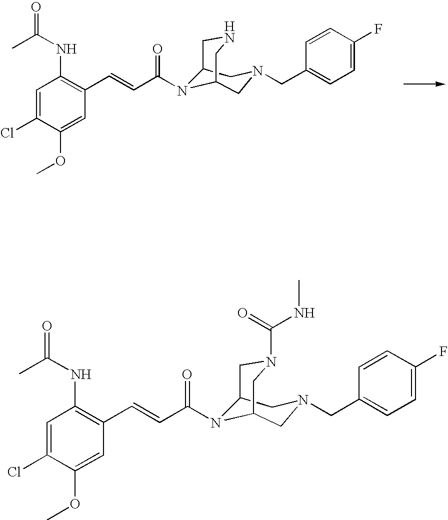

N-(5-Chloro-2-{(E)-3-[3-(4-fluorobenzyl)-3,7,9-triazabicyclo[3.3.1]non-9-yl]-3-oxo-propenyl}-4-methoxyphenyl)-acetamide (free base of Example 76) is treated in analogy to Example 23f and the product purified via chromatography (SiO2, TBME/MeOH/NH3conc 95/5/0.5 to 90/10/1) to yield the desired product crystallized from acetone/TBME (23 mg; 68%).

1H-NMR (400 MHz; DMSO-d6), δ (ppm): 2.03 (s, 3H); 2.16 (bd, 1H); 2.27 (bd, 1H); 2.67 (d, 3H); 2.88-3.00 (m, 3H); 3.06 (bd, 1H); 3.30 (q, 2H); 3.92 (s, 3H); 4.10 (bt, 2H); 4.47 (bs, 1H); 4.53 (bs, 1H); 6.25 (q, 1H); 7.07 (t, 2H); 7.21 (d, 1); 7.27 (dd, 2H); 7.42 (s, 1H); 7.47 (s, 1H); 7.62 (d, 1H); 9.68 (s, 1H).

MS (m/z) ES+: 544 (MH+).

### Example 82

### 9-[(E)-3-(2-Acetylamino-4-chloro-5-methoxyphenyl)-acryloyl]-7-(4-fluorobenzyl)-3,7,9-triazabicyclo[3.3.1]nonane-3-carboxylic acid dimethylamide

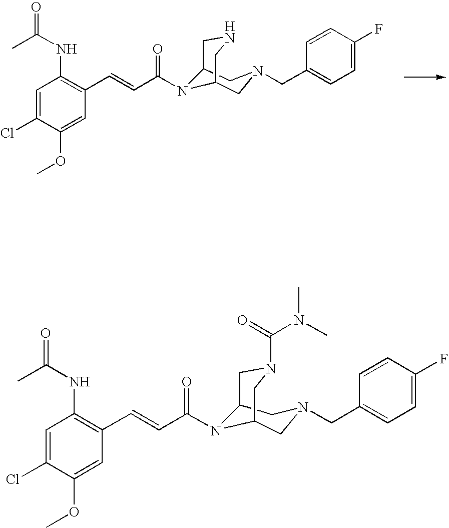

N-(5-Chloro-2-{(E)-3-[3-(4-fluorobenzyl)-3,7,9-triazabicyclo[3.3.1]non-9-yl]-3-oxo-propenyl}-4-methoxyphenyl)-acetamide (free base of Example 76c) is treated in analogy to Example 8 and the product purified via chromatography (SiO2, TBME/MeOH/NH3conc 95/5/0.5) to yield the desired product crystallized from TBME (17 mg; 49%).

1H-NMR (400 MHz; DMSO-d6), δ (ppm): 2.03 (s, 3H); 2.14 (bd, 1H); 2.27 (bd, 1H); 2.70 (s, 6H); 2.91 (d, 1H); 2.97 (bd, 1H); 3.07 (s, 2H); 3.10-3.32 (m, 3H); 3.85 (t, 1H); 3.93 (s, 3H); 4.50 (bs, 1H); 4.55 (bs, 1H); 7.08 (t, 2H); 7.20 (d, 1H); 7.28 (dd, 2H); 7.43 (s, 1H); 7.47 (s, 1H); 7.62 (d, 1H); 9.71 (s, 1H).

MS (m/z) ES+: 558 (MH+).

### Example 83

### 9-[(E)-3-(2-Acetylamino-4-chloro-5-methoxyphenyl)-acryloyl]-7-(4-fluorobenzyl)-3,7,9-triazabicyclo[3.3.1]nonane-3-carboxylic acid methyl ester

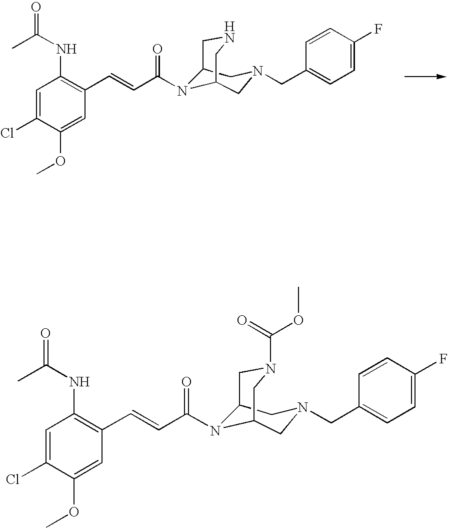

The reaction is performed in analogy to Example 80 using methyl chloroformate and the title product purified via chromatography (SiO2, TBME/MeOH/NH3conc 95/5/2 to 95/5/0) to yield the desired product as colorless crystals (24 mg; 72%).

1H-NMR (400 MHz; DMSO-d6), δ (ppm): 2.03 (s, 3H); 2.11 (bd, 0.5H); 2.22 (bt, 1H); 2.32 (bd, 0.5H); 2.90 (bt, 1H); 2.95-3.45 (m, 5H); 3.69 (s, 3H); 3.92 (d, 3H); 4.03 (d, 1H); 4.20 (d, 1H); 4.45-4.61 (m, 2H); 7.09-7.27 (m, 5H); 7.42-7.48 (m, 2H); 7.63 (d, 1H); 9.70 (s, 1H).

MS (m/z) ES+: 545 (MH+).

### Example 84

### N-(5-Chloro-2-{3-[3-(4-fluorobenzyl)-7-methanesulfonyl-3,7,9-triazabicyclo[3.3.1]non-9-yl]-3-oxopropenyl}-4-methoxyphenyl)-acetamide

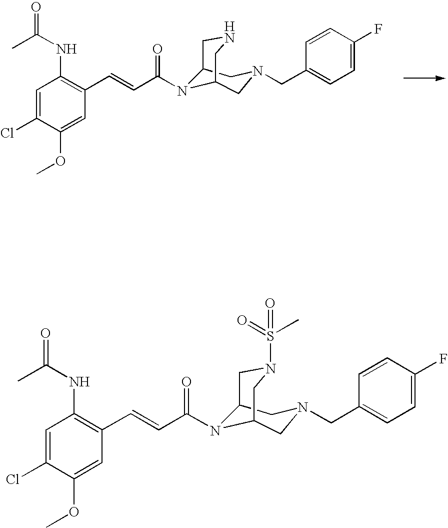

The reaction is performed in analogy to Example 80 employing methanesulfonyl chloride and the title product purified via chromatography (SiO2, TBME/MeOH/NH3conc 95/5/0.5) to yield the desired product as colorless glass (22 mg; 67%).

1H-NMR (400 MHz; DMSO-d6), δ (ppm): 2.04 (s, 3H); 2.25 (d, 1H); 2.38 (d, 1H); 2.83 (s, 3H); 2.92-3.08 (m, 3H); 3.17 (d, 1H); 3.42 (q, 2H); 3.59-3.66 (m, 2H); 3.93 (s, 3H); 4.70 (d, 2H); 7.07 (t, 2H); 7.21 (d, 1H); 7.35-7.48 (m, 4H); 7.65 (d, 1H); 9.72 (s, 1H).

MS (m/z) ES+: 565 (MH+).

### Example 85

### 5-Chloro-2-{(E)-3-[3-(4-fluorobenzyl)-7-methanesulfonyl-3,7,9-triazabicyclo[3.3.1]non-9-yl]-3-oxopropenyl}-N,N-dimethyl-4-trifluormethoxybenzenesulfonamide

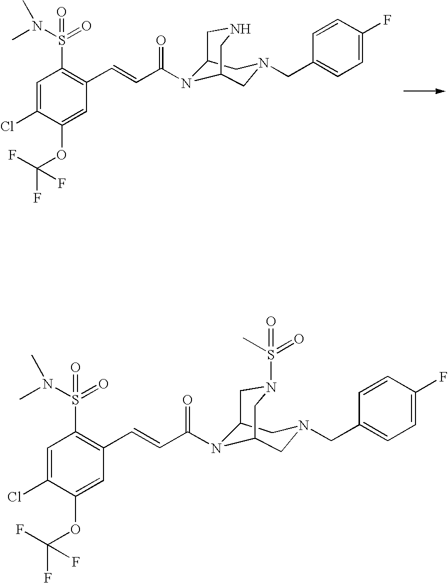

5-Chloro-2-{(E)-3-[3-(4-fluoro-benzyl)-3,7,9-triaza-bicyclo[3.3.1]non-9-yl]-3-oxo-propenyl}-N,N-dimethyl-4-trifluoromethoxy-benzenesulfonamide (obtained from (E)-3-(4-chloro-2-dimethylsulfamoyl-5-trifluoromethoxy-phenyl)-acrylic acid following the procedures described in Examples 40, 70b) is treated with methanesulfonyl chloride according to the conditions described in Example 80 to yield the title compound purified via chromatography (SiO2, acetone/hexanes 3/7) as colorless foam crystallized from TBME/hexanes (33 mg; 69%).

1H-NMR (400 MHz; DMSO-d6), δ (ppm): 2.25 (bd, 1H); 2.36 (bd, 1H); 2.76 (s, 6H); 2.83 (s, 3H); 2.93 (d, 1H); 2.98 (d, 1H); 3.08 (d, 1H); 3.16 (d, 1H); 3.42 (q, 2H); 3.60 (d, 2H); 4.68 (d, 2H); 7.07 (t, 2H); 7.33 (d, 1H); 7.38 (dt, 2H); 8.05 (s, 1H); 8.21 (d, 1H); 8.26 (s, 1H).

MS (m/z) ES+: 669 (MH+).

### Example 86

### N-(2-{(E)-3-[3-Acetyl-7-(4-fluorobenzyl)-3,7,9-triazabicyclo[3.3.1]non-9-yl]-3-oxopropenyl}-5-chloro-4-fluorophenyl)-acetamide

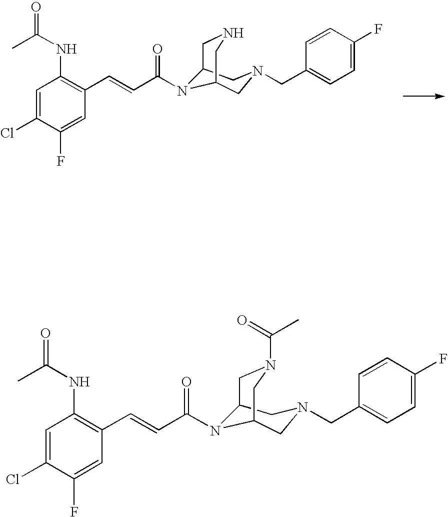

Example 72b is treated according to the conditions described in Example 80 to yield the title compound, which after purification via chromatography (SiO2, acetone/hexanes 1/1 to 1/0) rendered the desired product as yellow foam crystallized from acetone (31 mg; 78%).

1H-NMR (400 MHz; DMSO-d6), δ (ppm): 1.95 (d, 3H); 2.08 (s, 3H); 2.15 (d, 1H); 2.25 (d, 1H); 2.72 (bd, 1H); 2.82 (bd, 1H); 2.93 (bq, 4H); 3.43 (bt, 1H); 3.90 (bt, 1H); 4.55 (bd, 2H); 7.11 (bt, 2H); 7.21-7.32 (m, 3H); 7.60-7.68 (bd, 2H); 8.02 (bd, 1H); 9.90 (bs, 1H).

MS (m/z) ES+: 517 (MH+).

### Example 87

### N-(5-Chloro-(2-{(E)-3-[3-(4-fluorobenzyl)-3,7,9-triazabicyclo[3.3.1]non-9-yl]-3-oxopropenyl}-4-methylphenyl)-acetamide hydrochloride

### a) 9-[(E)-3-(2-Amino-4-chloro-5-methylphenyl)acryloyl]-7-(4-fluorobenzyl)-3,7,9-triazabicyclo[3.3.1]nonane-3-carboxylic acid tert-butyl ester

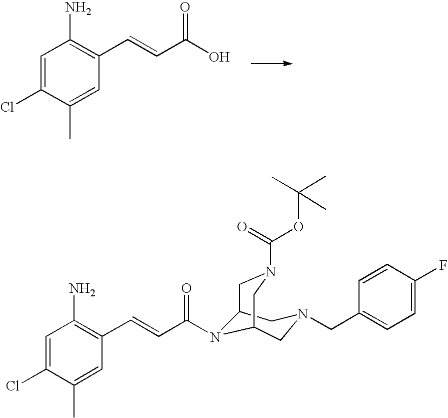

(E)-3-(2-Amino-4-chloro-5-methylphenyl)acrylic acid (Example 41c) is treated according to the conditions described in Example 76a to yield the target compound as yellow foam (342 mg; 75%).

1H-NMR (400 MHz; DMSO-d6), δ (ppm): 1.46 (s, 9H); 2.21 (s, 2H); 2.93 (bt, 4H); 3.31 (s, 2H); 3.43 (m, 2H); 3.70 (m, 2H); 4.00-4.12 (m, 2H); 4.47 (m, 1H); 4.58 (bs, 1H); 6.38 (bd, 2H); 6.90 (m, 1H); 7.08 (t, 2H); 7.33 (bt, 2H); 7.55 bd, 1H); 7.75 (d, 1H).

LC-MS (m/z) ES+: 528 (M+).

### b) 9-[(E)-3-(2-Amino-4-chloro-5-methylphenyl)acryloyl]-7-(4-fluorobenzyl)-3,7,9-triazabicyclo[3.3.1]nonane-3-carboxylic acid tert-butyl ester

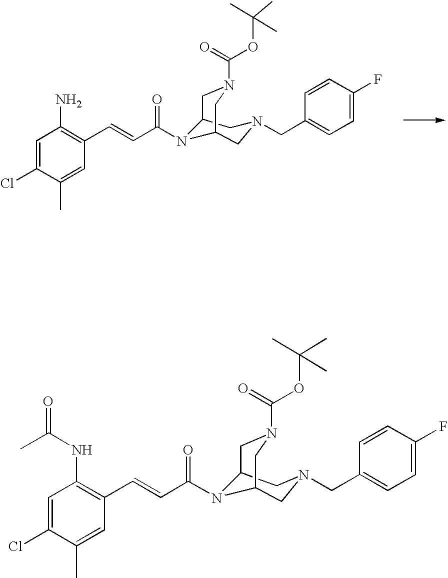

The reaction is performed in analogy to Example 1f and the title product purified via chromatography (SiO2, acetone/hexanes 2/8 to 4/6) to yield the desired product as colorless solid (40 mg; 37%).

1H-NMR (400 MHz; DMSO-d6), δ (ppm): 1.47 (s, 9H); 2.07 (s, 3H); 2.17 (bt, 1H); 2.33 (s, 3H); 2.90-3.00 (m, 3H); 3.01-3.11 (m, 2H); 3.30 (bs, 2H); 4.02-4.13 (m, 2H); 4.50 (bs, 1H); 4.57 (bs, 1H); 7.08 (t, 2H); 7.17 (dd, 1H); 7.34 (dd, 2H); 7.50 (d, 1H); 7.63 (d, 1H); 7.87 (d, 1H); 9.80 (s, 1H).

MS (m/z) ES+: 570 (M+).

### c) N-(5-Chloro-(2-{(E)-3-[3-(4-fluorobenzyl)-3,7,9-triazabicyclo[3.3.1]non-9-yl]-3-oxopropenyl}-4-methylphenyl)-acetamide hydrochloride

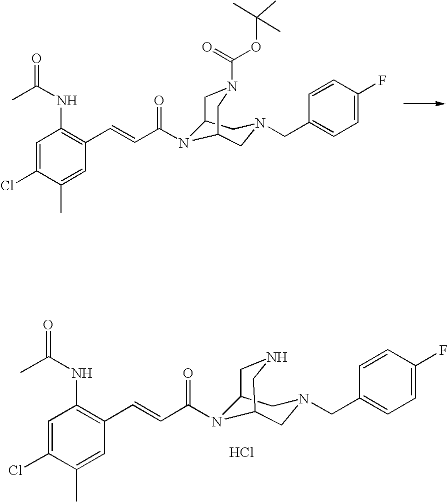

The title compound is obtained following the procedure described in Example 71b, rendering the desired compound as colorless crystals (33 mg; 98%).

1H-NMR (400 MHz; DMSO-d6), δ (ppm): 2.08 (s, 3H); 2.21-2.30 (m, 2H); 2.31 (s, 3H); 3.02-3.10 (m, 4H); 3.20 (bs, 1H); 3.41-3.52 (m, 4H); 4.73 (bs, 2H); 7.12-7.22 (m, 2H); 7.41-7.50 (m, 2H); 7.51 (s, 1H); 7.65 (d, 1H); 7.85 (s, 1H); 8.32 (bs, 1H); 9.56 (bs, 1H); 9.85 (s, 1H).

MS (m/z) ES+: 471.3 (MH+).

### Example 88

### N-(5-Chloro-(2-{(E)-3-[3-(4-fluorobenzyl)-3,7,9-triazabicyclo[3.3.1]non-9-yl]-3-oxopropenyl}-4-methylphenyl)-methanesulfonamide hydrochloride

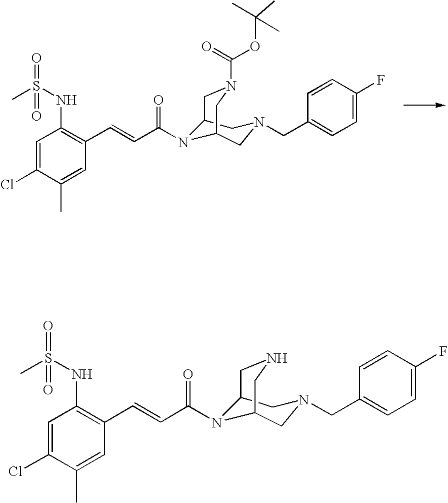

9-[(E)-3-(4-Chloro-2-methanesulfonylamino-5-methyl-phenyl)-acryloyl]-7-(4-fluoro-benzyl)-3,7,9-triaza-bicyclo[3.3.1]nonane-3-carboxylic acid tert-butyl ester (obtained from 87a treated with methanesulfonyl chloride according to the conditions described in Example 70a) is deprotected in analogy to Example 71b to yield the title compound as colorless crystals (30 mg; 57%).

1H-NMR (400 MHz; DMSO-d6), δ (ppm): 2.25-2.35 (m, 2H); 2.37 (s, 3H); 2.97 (s, 3H); 3.03-3.10 (m, 4H); 3.43-3.53 (m, 4H); 4.73 (bs, 2H); 7.18 (dd, 2H); 7.38 (s, 1H); 7.46 (m, 2H); 7.85 (d, 1H); 7.91 (s, 1H); 8.32 (bs, 1H); 9.36 (bs, 1H); 9.60 (s, 1H).

MS (m/z) ES+: 507.2 (MH+).

### Example 89

### (5-Chloro-(2-{(E)-3-[3-(4-fluorobenzyl)-3,7,9-triazabicyclo[3.3.1]non-9-yl]-3-oxopropenyl}-4-methylphenyl)-urea hydrochloride

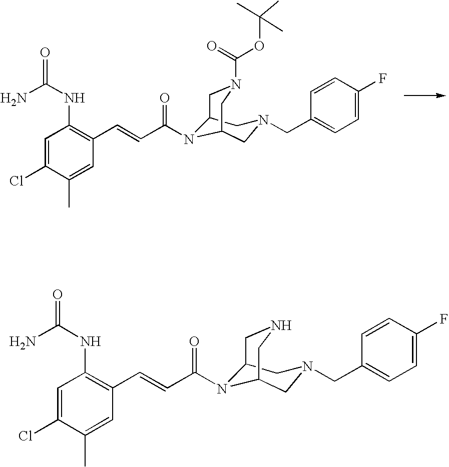

9-[(E)-3-(4-Chloro-5-methyl-2-ureido-phenyl)-acryloyl]-7-(4-fluoro-benzyl)-3,7,9-triaza-bicyclo[3.3.1]nonane-3-carboxylic acid tert-butyl ester (obtained from Example 87a treated with NaOCN according to the conditions described in Example 4) is deprotected in analogy to Example 71b to yield the title compound as colorless crystals recrystallised from hot EtOH (68 mg; 69%).

1H-NMR (400 MHz; DMSO-d6), δ (ppm): 2.13-2.38 (m, 5H); 3.02-3.11 (m, 4H); 3.45-3.52 (m, 4H); 4.74 (bd, 2H); 6.15 (bs, 2H); 7.12 (d, 1H); 7.17 (t, 2H); 7.45 (t, 2H); 7.70 (m, 2H); 7.83 (s, 1H); 8.30 (bs, 1H); 8.37 (s, 1H); 9.40 (bs, 1H).

MS (m/z) ES+: 472.3 (MH+).

### Example 90

### N-(5-Chloro-(2-{(E)-3-[3-(4-fluorobenzyl)-3,7,9-triazabicyclo[3.3.1]non-9-yl]-3-oxopropenyl}-4-methyl phenyl)-2-dimethylaminoacetamide dihydrochloride

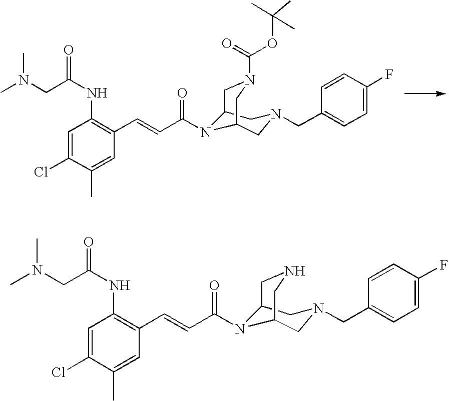

9-{(E)-3-[4-Chloro-2-(2-dimethylamino-acetylamino)-5-methyl-phenyl]-acryloyl}-7-(4-fluoro-benzyl)-3,7,9-triaza-bicyclo[3.3.1]nonane-3-carboxylic acid tert-butyl ester (obtained from Example 87a treated according to conditions described in Example 69a) is deprotected in analogy to Example 71b to yield the title compound as colorless crystals recrystallised from hot EtOH (46 mg; 72%).

1H-NMR (400 MHz; DMSO-d6), δ (ppm): 2.23 (d, 1H); 2.32 (d, 1H); 2.37 (s, 3H); 2.90 (s, 6H); 3.06 (m, 4H); 3.49 (m, 4H); 4.21 (s, 2H); 4.75 (bs, 2H); 7.19 (t, 2H); 7.25 (d, 1H); 7.49 (t, 2H); 7.58 (s, 1H); 7.63 (d, 1H); 7.96 (s, 1H); 8.32 (bs, 1H); 9.70 (bs, 1H); 9.93 (bs, 1H); 10.12 (s, 1H).

MS (m/z) ES+: 514.3 (MH+).

### Example 91

### (5-Chloro-2-{(E)-9-[3-(4-fluorobenzyl)-7-methyl-3,7,9-triazabicyclo[3.3.1]non-3-yl]-3-oxopropenyl}-4-methylphenyl)-urea

### a) 7-Methyl-3,7,9-triazabicyclo[3.3.1]nonane-3-carboxylic acid tert-butyl ester

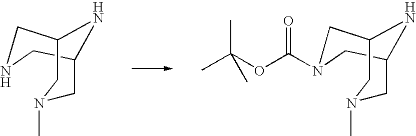

3-Methyl-3,7,9-triaza-bicyclo[3.3.1]nonane dihydrochloride (Example 73b) (800 mg; 3.7 mmol) in 2N NaOH (12 ml) and THF (20 ml) is treated with (BOC)2O (830 mg; 3.7 mmol) for 30 min at room temp. Solid K2CO3 is added, filtered and the residue purified via chromatography (SiO2, TBME/MeOH/NH3conc 95/5/0.5 to 90/10/1) to yield the desired product as colorless crystals (285 mg; 20%).

MS (m/z) ES+: 242 (MH+).

### b) 9-(4-Fluorobenzyl)-7-methyl-3,7,9-triazabicyclo[3.3.1]nonane-3-carboxylic acid tert-butyl ester

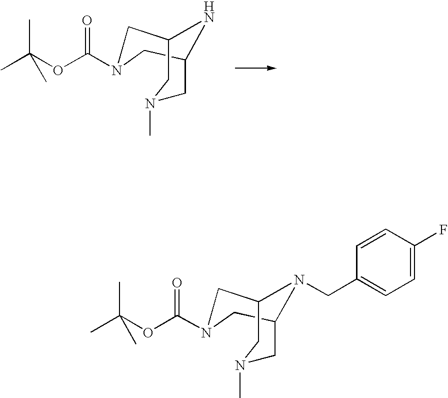

7-Methyl-3,7,9-triazabicyclo[3.3.1]nonane-3-carboxylic acid tert-butyl ester (1.5 g; 6.22 mmol), 4-fluorobenzyl chloride (0.821 ml; 6.8 mmol) and NaHCO3 (2.6 g; 31.1 mmol) in EtOH (20 ml) are refluxed for 4 h. EtOH is evaporated, the residue taken up in TBME, filtered and purified via chromatography (SiO2, TBME/hexanes 1/1) to yield the desired product as colorless crystals (1.44 g; 66%).

1H-NMR (400 MHz; DMSO-d6), δ (ppm): 1.39 (s, 9H); 2.03 (s, 3H); 2.36 (bt, 2H); 2.60 (m, 4H); 3.12 (bd, 1H); 3.29 (bd, 1H); 3.75 (d, 1H); 3.82 (d, 1H); 3.83 (s, 2H); 7.10 (t, 2H); 7.38 (dd, 2H).

MS (m/z) ES+: 350 (MH+, 100); 250 (55).

### c) 9-(4-Fluorobenzyl)-3-methyl-3,7,9-triazabicyclo[3.3.1]nonane

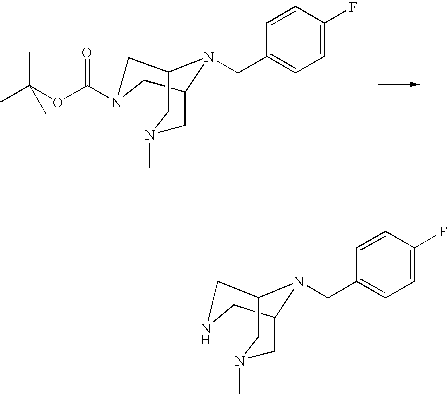

9-(4-Fluorobenzyl)-7-methyl-3,7,9-triazabicyclo[3.3.1]nonane-3-carboxylic acid tert-butyl ester (750 mg; 2.1 mmol) is dissolved in HClconc (4 ml) and after 5 min. the precipitate formed filtered off. The base is set free by washing the TBME suspension with 2N NaOH. The organic phase is dried over Na2SO4, filtered and evaporated to dryness to yield the desired product as a yellow oil (532 mg; 99%).

1H-NMR (400 MHz; DMSO-d6), δ (ppm): 2.10 (s, 3H); 2.40 (bs, 2H); 2.53 (bd, 3H); 2.69 (bd, 2H); 2.75 (bd, 2H); 3.03 (bd, 2H); 3.83 (s, 2H); 7.10 (t, 2H); 7.36 (dd, 2H).

MS (m/z) ES+: 250 (MH+).

### d) (E)-3-(2-Amino-4-chloro-5-methylphenyl)-1-[9-(4-fluorobenzyl)-7-methyl-3,7,9-triazabicyclo[3.3.1]non-3-yl]-Propenone

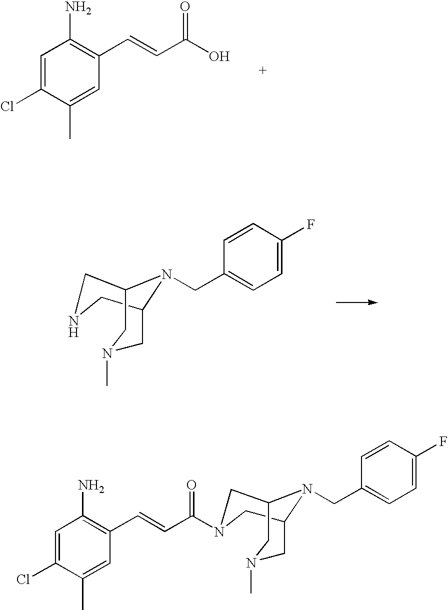

The condensation reaction is performed according to Example 23d yielding the desired product as a yellow foam (634 mg; 76%).

1H-NMR (400 MHz; DMSO-d6), δ (ppm): 2.02 (s, 3H); 2.18 (s, 3H); 2.32-2.39 (m, 2H); 2.63 (d, 1H); 2.71-2.82 (m, 3H); 3.18 (dd, 1H); 3.63 (dd, 1H); 3.87 (s, 2H); 4.00 (d, 1H); 4.14 (d, 1H); 5.32 (bs, 2H); 6.73 (s, 1H); 6.92 (d, 1H); 7.13 (t, 2H); 7.40 (dd, 2H); 7.45 (s, 1H); 7.54 (d, 1H).

MS (m/z) ES+: 443 (MH+).

### e) (5-Chloro-2-{(E)-9-[3-(4-fluorobenzyl)-7-methyl-3,7,9-triazabicyclo[3.3.1]non-3-yl]-3-oxopropenyl}-4-methylphenyl)-urea

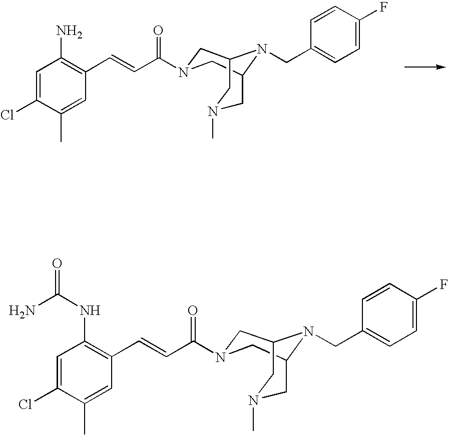

The reaction is performed in analogy to Example 4 and the title product purified via chromatography (SiO2, TBME/MeOH/NH3conc 981210.2 to 95/5/0.5) to yield the desired product as colorless crystals (74 mg; 56%).

1H-NMR (400 MHz; DMSO-d6), δ (ppm): 2.03 (s, 3H); 2.29 (s, 3H); 2.32-2.40 (m, 2H); 2.63 (d, 1H); 2.72-2.83 (m, 3H); 3.20 (bd, 1H); 3.52 (bd, 1H); 3.88 (s, 2H); 4.03 (d, 1H); 4.18 (d, 1H); 6.13 (bs, 2H); 7.08 (d, 1H); 7.15 (d, 2H); 7.40 (dd, 2H); 7.58 (d, 1H); 7.70 (s, 1H); 7.86 (s, 1H); 8.25 (s, 1H).

MS (m/z) ES+: 486 (MH+).

### Example 92

### N-(5-Chloro-2-{(E)-3-[9-(4-fluorobenzyl)-7-methyl-3,7,9-triazabicyclo[3.3.1]non-3-yl]-3-oxopropenyl}-4-methylphenyl)-methanesulfonamide

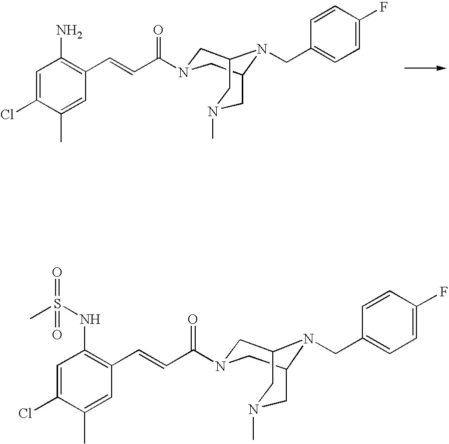

The reaction is performed in analogy to Example 70a and the title product purified via chromatography (SiO2, TBME/MeOH/NH3conc 100/0/0 to 85/15/1.5) to yield the desired product as yellow foam, recrystallised from TBME/acetone (38 mg; 27%).

1H-NMR (400 MHz; DMSO-d6), δ (ppm): 2.05 (bs, 3H); 2.35 (s, 3H); 2.32-2.42 (m, 2H); 2.63 (bd, 1H); 2.73-2.83 (m, 3H); 2.95 (s, 3H); 3.20 (dd, 1H); 3.65 (bd, 1H); 3.88 (s, 2H); 4.05 (d, 1H); 4.15 (d, 1H); 7.12 (t, 3H); 7.33 (s, 1H); 7.40 (dd, 2H); 7.71 (d, 1H); 7.90 (s, 1H); 9.55 (bs, 1H).

MS (m/z) ES+: 521 (MH+).

### Example 93

### N-(5-Chloro-2-{(E)-3-[9-(4-fluorobenzyl)-7-methyl-3,7,9-triazabicyclo[3.3.1]non-3-yl]-3-oxopropenyl}-acetamide

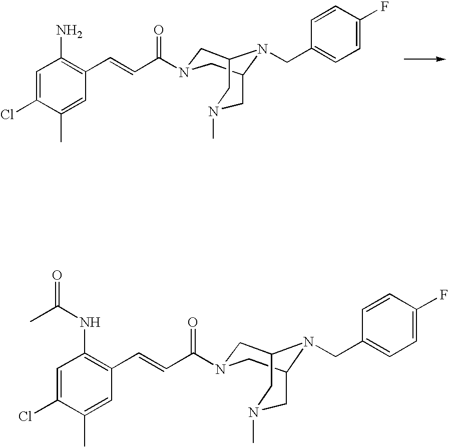

The reaction is performed in analogy to Example 1f and the title product purified via chromatography (SiO2, TBME/MeOH/NH3conc 97/3/0.3) to yield the desired product as yellow foam, which is recrystallised from acetone (67 mg; 51%).

1H-NMR (400 MHz; DMSO-d6), δ (ppm): 2.02 (s, 3H); 2.07 (s, 3H); 2.34 (s, 3H); 2.33-2.39 (m, 2H); 2.52 (d, 1H); 2.73 (d, 1H); 2.80 (bs, 2H); 3.20 (dd, 1H); 3.65 (dd, 1H); 3.88 (s, 2H); 4.03 (d, 1H); 4.15 (d, 1H); 7.09-7.17 (m, 3H); 7.40 (dd, 2H); 7.47 (s, 1H); 7.52 (d, 1H); 7.84 (s, 1H); 9.87 (s, 1H).

MS (m/z) ES+: 485 (MH+).

### Example 94

### N-(2-{(E)-3-[3-Acetyl-7-(4-fluorobenzyl)-3,7,9-triazabicyclo[3.3.1]non-9-yl]-3-oxopropenyl}-5-chloro-4-methylphenyl)-acetamide

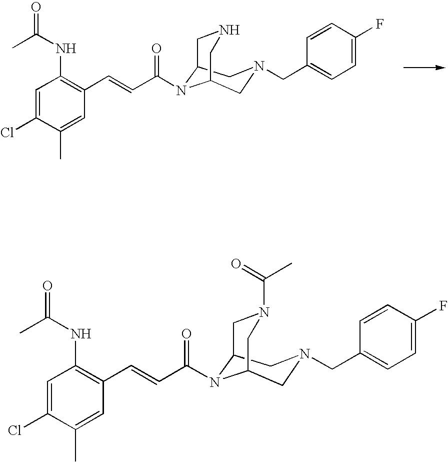

The title compound is obtained following the procedure described in Example 80, rendering the desired compound as colorless crystals crystallized from acetone/TBME (11 mg; 58%).

1H-NMR (400 MHz; DMSO-d6), δ (ppm): 1.96 (d, 3H); 2.07 (s, 3H); 2.17 (bd, 1H); 2.25 (bd, 1H); 2.32 (s, 3H); 2.73 (bd, 1H); 2.83 (bd, 1H); 2.96 (dd, 2H); 3.32 (bd, 1H); 3.30 (dd, 2H); 3.90 (t, 1H); 4.47-4.62 (m, 2H); 7.11 (t, 2H); 7.18 (d, 1H); 7.25 (dd, 2H); 7.50 (d, 1H); 7.65 (d, 1H); 7.89 (d, 1H); 9.80 (s, 1H).

MS (m/z) ES+: 513 (MH+).

### Example 95

### (5-Chloro-2-{(E)-3-[3-(4-fluorobenzyl)-7-methyl-3,7,9-triazabicyclo[3.3.1]non-9-yl]-3-oxopropenyl}-4-methylphenyl)-urea hydrochloride

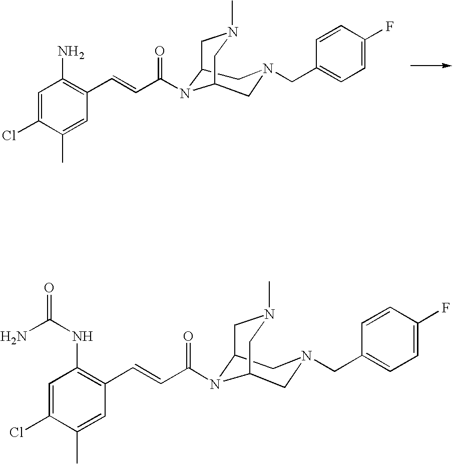

(E)-3-(2-Amino-4-chloro-5-methyl-phenyl)-1-[3-(4-fluoro-benzyl)-7-methyl-3,7,9-triaza-bicyclo[3.3.1]non-9-yl]-propenone (obtained from Example 41c and Example 73c which are coupled according to the conditions described for Example 23d) is treated according to Example 4 and purified via chromatography (XTerra, RP18, 7 μm, MeCN/water 40/60 to 100/0) to yield the title compound as colorless foam, which is crystallized from ether/HCl (48 mg; 22%).

1H-NMR (400 MHz; DMSO-d6), δ (ppm): 2.28 (s, 3H); 2.33 (bd, 1H); 2.40 (bd, 1H); 2.80 (s, 3H); 3.03-3.18 (m, 4H); 3.28 (bd, 2H); 3.59-3.57 (m, 3H); 4.81 (bs, 1H); 6.20 (bs, 2H); 7.10 (d, 1H); 7.19 (t, 2H); 7.41 (dd, 2H); 7.71 (s, 1H); 7.12 (d, 1H); 7.83 (s, 1H); 8.36 (bs, 1H); 9.40 (bs, 1H).

MS (m/z) ES+: 486.2 (MH+).

### Example 96

### N-(5-Chloro-2-{(E)-3-[3-(4-fluorobenzyl)-7-methyl-3,7,9-triazabicyclo[3.3.1]non-9-yl]-3-oxopropenyl}-4-methylphenyl)-methanesulfonamide hydrochloride

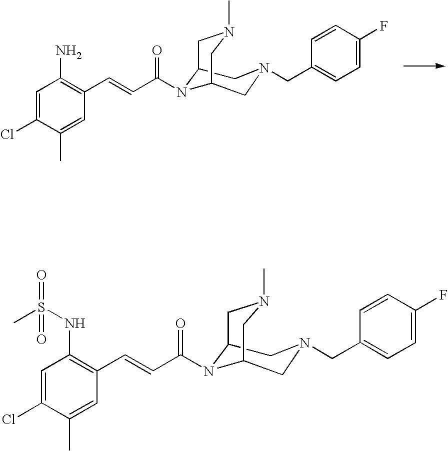

The reaction is performed in analogy to Example 70a and the title product purified via chromatography (SiO2, TBME/MeOH/NH3conc 90/10/1 to 80/20/1.5) to yield the desired product which is crystallized from ether/HCl (123 mg; 49%).

1H-NMR (400 MHz; DMSO-d6), δ (ppm): 2.34 (d, 1H); 2.35 (s, 3H); 2.40 (d, 1H); 2.80 (s, 3H); 3.00 (s, 3H); 3.08 (bt, 2H); 3.16 (d, 1H); 3.23 (d, 1H); 3.58-3.70 (m, 4H); 4.82 (bd, 2H); 7.13-7.22 (m, 3H); 7.38 (s, 1H); 7.43 (dd, 2H); 7.87 (d, 1H); 7.95 (s, 1H); 9.48 (bs, 1H); 9.61 (s, 1H).

MS (m/z) ES+: 521.2 (MH+).

### Example 97

### N-(5-Chloro-2-{(E)-3-[3-(4-fluorobenzyl)-7-methyl-3,7,9-triazabicyclo[3.3.1]non-9-yl]-3-oxopropenyl}-4-methylphenyl)-amide hydrochloride

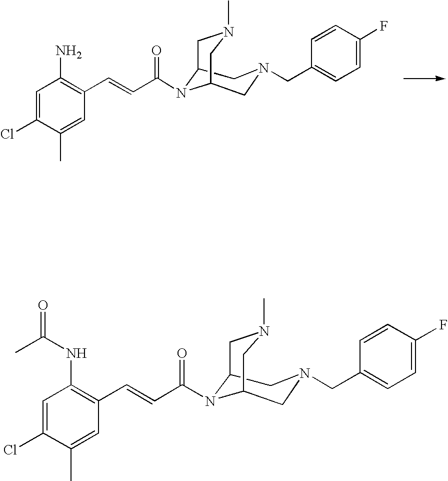

The reaction is performed in analogy to Example 1f to yield the title compound after chromatography (SiO2, TBME/MeOH/NH3conc 97/3/0.3) as yellow foam (45 mg; 67%).

1H-NMR (400 MHz; DMSO-d6), δ (ppm): 2.04 (s, 3H); 2.31 (bd, 1H); 2.32 (s, 3H); 2.40 (bd, 1H); 2.70 (s, 3H); 3.03-3.18 (m, 3H); 3.25 (bd, 1H); 3.58-3.70 (m, 4H); 4.70 (bs, 2H); 7.11-7.22 (m, 3H); 7.40-7.47(m, 3H); 7.65 (d, 1H); 7.85 (s, 1H); 9.45 (bs, 1H); 9.83 (s, 1H).

MS (m/z) ES+: 485.2 (MH+).

### Example 98

### N-(5-Chloro-2-{(E)-3-[3-(4-fluorobenzyl)-7-methyl-3,7,9-triazabicyclo[3.3.1]non-9-yl]-3-oxo-propenyl}-4-methoxyphenyl)-acetamide

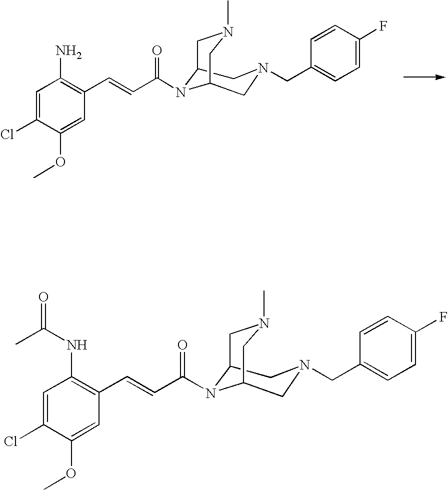

(E)-3-(2-Amino-4-chloro-5-methoxy-phenyl)-1-[3-(4-fluoro-benzyl)-7-methyl-3,7,9-triaza-bicyclo[3.3.1]non-9-yl]-propenone (obtained by coupling Example 23c and Example 73c employing the conditions described in Example 23d) is treated as described in Example 1f to yield the title product. Purification via chromatography (SiO2, TBME/MeOH/NH3conc 90/10/1) yielded the desired product, which is crystallized from ether/HCl (106 mg; 45%).

1H-NMR (400 MHz; DMSO-d6), δ (ppm): 2.05 (s, 3H); 2.16 (s, 3H); 2.20-2.33 (m, 3H); 2.40 (bd, 1H); 2.75-2.88 (m, 4H); 3.46 (dd, 2H); 3.92 (s, 3H); 4.53 (bs, 1H); 4.60 (bs, 1H); 7.12 (t, 2H); 7.20 (d, 1H); 7.35 (dd, 2H); 7.42 (s, 1H); 7.48 (s, 1H); 7.60 (d, 1H); 9.72 (s, 1H).

MS (m/z) ES+: 501.1 (MH+).

### Example 99

### N-(5-Chloro-2-{(E)-3-[3-(4-fluorobenzyl)-7-methyl-3,7,9-triazabicyclo[3.3.1]non-9-yl]-3-oxo-propenyl}-4-methoxyphenyl)-methanesulfonamide

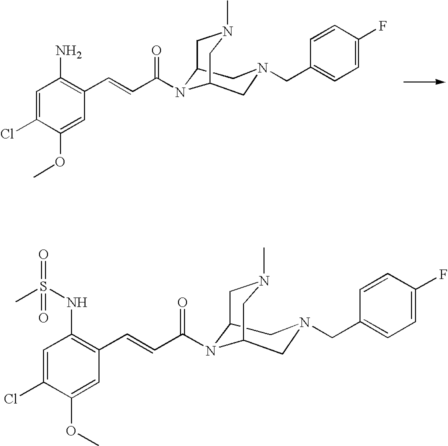

(E)-3-(2-Amino-4-chloro-5-methoxy-phenyl)-1-[3-(4-fluoro-benzyl)-7-methyl-3,7,9-triaza-bicyclo[3.3.1]non-9-yl]-propenone (obtained by coupling Example 23c and Example 73c employing the conditions described in Example 23d) is treated as described in Example 70a to yield the title product. Purification via chromatography (SiO2, TBME/MeOH/NH3conc 90/10/1 to 80/20/1.5) yielded the desired product, as a yellow foam (153 mg; 65%).

1H-NMR (400 MHz; CDCl₃), δ (ppm): 2.30 (s, 3H); 2.47-2.58 (m, 4H); 2.83-2.98 (m, 4H); 3.03 (s, 3H); 3.52 (s, 2H); 4.03 (s, 3H); 4.21 (bs, 1H); 4.80 (bs, 1H); 6.75 (d, 1H); 6.97-7.02 (m, 3H); 7.31 (dd, 2H); 7.53 (s, 1H); 7.83 (d, 1H).

MS (m/z) ES+: 537.1 (MH+).

### Example 100

### (5-Chloro-2-{(E)-3-[3-(4-fluorobenzyl)-7-methyl-3,7,9-triazabicyclo[3.3.1]non-9-yl]-3-oxo-propenyl}-4-methoxyphenyl)-urea

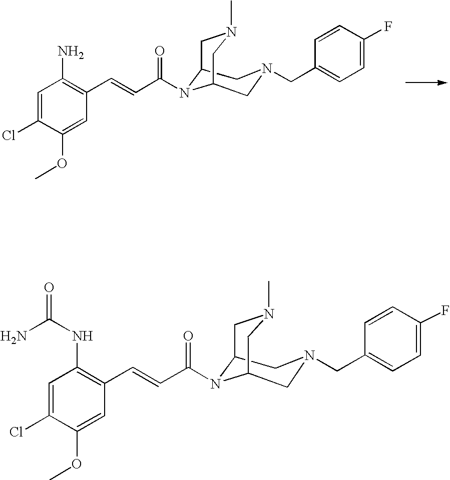

The reaction is performed in analogy to Example 4 and the title product purified via chromatography (SiO2, TBME/MeOH/NH3conc 90/10/1) to yield the desired product as yellow foam, which is recrystallised from hot TBME (70 mg; 43%).

1H-NMR (400 MHz; DMSO-d6), δ (ppm): 2.15 (bs, 3H); 2.17-2.42 (m, 4H); 2.75-2.90 (m, 4H); 3.45 (bq, 2H); 3.90 (s, 3H); 4.52 (bs, 1H); 4.63 (bs, 1H); 6.03 (bs, 2H); 7.09-7.20 (m, 3H); 7.32-7.40 (m, 3H); 7.65-7.70 (d, 2H); 8.20 (s, 1H).

MS (m/z) ES+: 502.1 (MH+, 90); 459.1 (40); 441.1 (60); 292 (100); 250 (60).

### Example 101

### N-(5-Chloro-2-{(E)-3-[3-(4-fluoro-benzyl)-7-methyl-3,7,9-triaza-bicyclo[3.3.1]non-9-yl]-3-oxo-propenyl}-4-methoxy-phenyl)-N,N-dimethylsulfonylurea

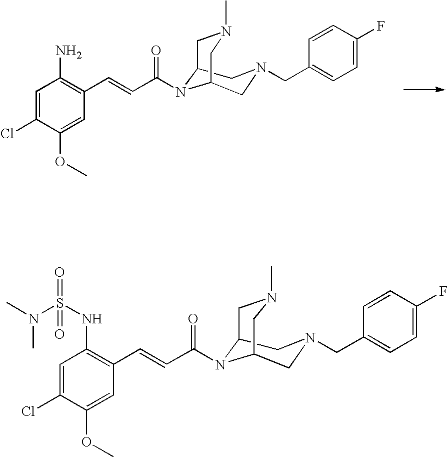

The reaction is performed in analogy to Example 39 and the title product purified via chromatography (XTerra, RP18, 7 μm, MeCN/water 40/60 to 100/0) to yield the title compound as yellow foam (50 mg; 42%).

1H-NMR (400 MHz; DMSO-d6), δ (ppm): 2.30 (dd, 2H); 2.40 (dd, 2H); 2.67 (bs, 9H); 2.80-2.95 (m, 4H); 3.50 (bs, 2H); 3.91 (s, 3H); 4.57 (bs, 1H); 4.65 (bs, 1H); 7.13 (t, 2H); 7.18 (d, 1H); 7.35 (s, 1H); 7.38 (dd, 2H); 7.43 (bs, 1H); 7.95 (d, 1H); 9.40 (bs, 1H) 566.2 (MH+).

MS (m/z) ES+:

### Example 102

### N-(5-Chloro-2-{2-[7-(4-fluorobenzyl)-3-oxa-7,9-diazabicyclo[3.3.1]non-9-yl]-2-oxoethoxy}-phenyl)acetamide

### a) 7-Benzenesulfonyl-9-benzyl-3-oxa-7,9-diazabicyclo[3.3.1]nonane

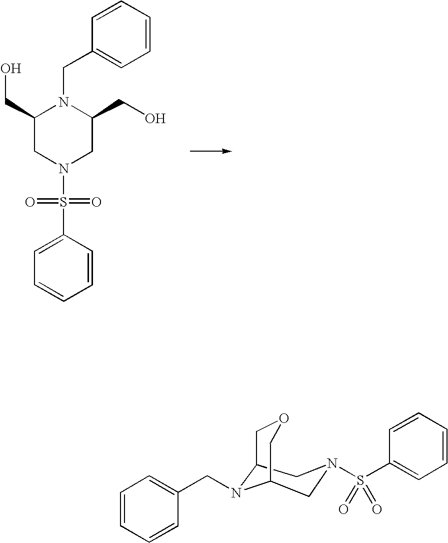

SOCl2 (0.97 ml; 13.4 mmol) in toluene (10 ml) is added under stirring at room temp. rapidly but dropwise to a solution of (meso)-(4-benzenesulfonyl-1-benzyl-6-hydroxymethylpiperazin-2-yl)-methanol (Example 64b; 5.05 g; 13.4 mmol) in DMF (200 ml). The reaction mixture is heated in an oil bath (170° C.) under reflux for 75 min. The reaction mixture is evaporated, taken up in a saturated solution of Na2CO3 and extracted with TBME three times. The combined organic phases are dried over Na2SO4, filtered, evaporated to dryness and purified via chromatography (SiO2, acetone/hexanes 1/9 to 3/7) to yield the title product as colorless crystals (2.1 g; 43%).

1H-NMR (400 MHz; DMSO-d6), δ (ppm): 2.65(bs, 2H); 2.83(bd, 2H); 3.49(d, 2H); 3.68(s, 2H); 3.70(d, 2H); 3.88(d, 2H); 7.20(m, 2H); 7.28(m, 3H); 7.68(m, 2H); 7.75(m, 3H).

COSY and HSQC spectra are in agreement with the structure.

MS (m/z) ES+: 359.2(MH+, 100).

### b) 9-Benzyl-3-oxa-7,9-diazabicyclo[3.3.1]nonane

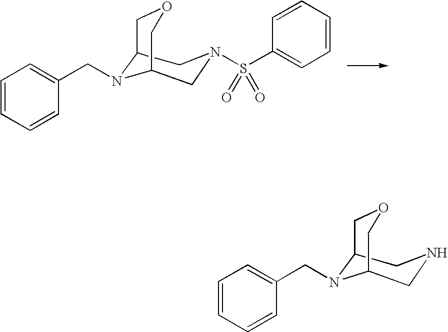

7-Benzenesulfonyl-9-benzyl-3-oxa-7,9-diazabicyclo[3.3.1]nonane (400 mg; 1.1 mmol) is dissolved in xylene (8 ml), Red-Al (˜3.5 M in toluene; 0.8 ml; 2.8 mmol) added and refluxed for 1.5 h. A second portion of Red-Al (0.4 ml; 1.4 mmol) is added and refluxed for another 30 min. The reaction mixture is poured on 2N HCl (100 ml) and washed twice with TBME. NaOH conc is added to the aqueous phase and extracted with TBME/EtOH (50:1) three times. The combined organic phases are dried with K2CO3, filtered, evaporated to dryness and purified via chromatography (SiO2, EtOAc/MeOH/NH3conc 80/20/4) to yield the title compound as yellowish oil, which crystallised in needles (206 mg; 84%).

1H-NMR (400 MHz; DMSO-d6), δ (ppm): 2.27(s, 2H); 2.84(d, 2H); 3.16(bd, 2H); 3.79(d, 2H); 3.98(s, 2H); 4.03(d, 2H); 7.13-7.40(m, 5H).

MS (m/z) ES+: 219.1(MH+, 100).

### c) 3-Oxa-7,9-diazabicyclo[3.3.1]nonane-7,9-dicarboxylic acid di-tert-butyl ester

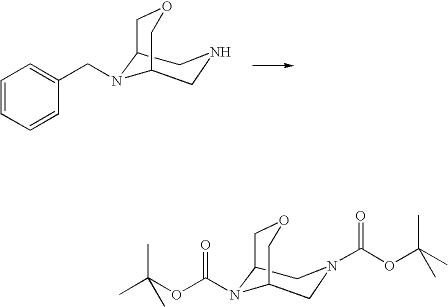

9-Benzyl-3-oxa-7,9-diazabicyclo[3.3.1]nonane (205 mg; 0.94 mmol) is dissolved in TBME (4 ml) and treated with (BOC)2O (500 mg; 2.2 mmol) in TBME (2 ml) at room temp. for 10 min. The reaction mixture is evaporated, taken up in EtOH (150 ml), Pd/C (10%; 350 mg) is added and hydrogenated for 2 h at 1 atm of H2. After filtration, evaporation to dryness and chromatography (SiO2, TBME/MeOH/NH3conc 90/10/2), the title compound is obtained as colorless crystals (186 mg; 60%).

1H-NMR (400 MHz; DMSO-d6), δ (ppm), mixture of rotamers: 1.40(s, 9H); 1.52(s, 9H); 2.95(bt, 1H); 3.10(bd, 1H); 3.58(bt, 2H); 3.82(bt, 4H); 4.05(bd, 1H); 4.15(bd, 1H).

The HSQC spectrum is in agreement with the structure.

MS (m/z) ES+: 351.2 (M+Na, 100).

### d) 7-(4-Fluorobenzyl)-3-oxa-7,9-diazabicyclo[3.3.1]nonane

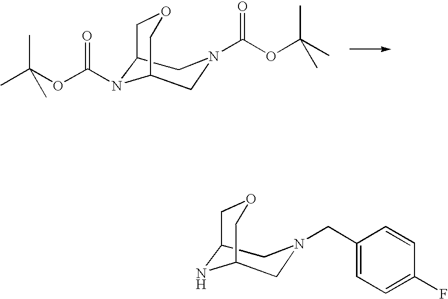

3-Oxa-7,9-diazabicyclo[3.3.1]nonane-7,9-dicarboxylic acid di-tert-butyl ester (80 mg; 0.24 mmol) is dissolved in EtOH (0.5 ml) and treated with HClconc (0.5 ml) for 30 min. The reaction mixture is evaporated, taken up in EtOH (4 ml), NaHCO3 (102 mg; 1.2 mmol) added, followed by 4-fluorobenzylchloride (0.029 ml; 0.24 mml) and refluxed for 1.5 h. The reaction mixture is evaporated and purified by chromatography (SiO2; TBME>TBME/MeOH/NH3conc 90/10/2) to yield the title compound as a yellowish foam (42 mg; 72%).

1H-NMR (400 MHz; DMSO-d6), δ (ppm): 2.33(bd, 2H); 2.72(bs, 2H); 2.80(d, 2H); 3.47(s, 2H); 3.63-3.74(m, 4H); 7.13(t, 2H); 7.38(dd, 2H).

The ROESY spectrum is in agreement with the structure.

MS (m/z) ES+: 237.2(MH+, 100).

### e) 2-Chloro-1-[7-(4-fluorobenzyl)-3-oxa-7,9-diazabicyclo[3.3.1]non-9-yl]-ethanone

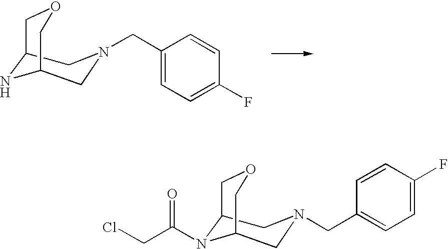

7-(4-Fluorobenzyl)-3-oxa-7,9-diazabicyclo[3.3.1]nonane (40 mg; 0.16 mmol) is dissolved in CH2Cl2 and treated with chloroacetylchloride (0.014 ml; 0.16 mmol). After 5 min. at room temp. the reaction mixture is poured on 2N Na2CO3 and extracted with TBME three times. The combined organic phases are dried over Na2SO4, filtered and evaporated to yield the title compound (53 mg; 98%).

1H-NMR (400 MHz; DMSO-d6), δ (ppm): 2.22(bd, 1H); 2.42(bd, 1H); 2.92(dd, 2H); 3.42(dd, 2H); 3.58(bd, 1H); 3.73(bd, 1H); 3.82(d, 2H); 3.96(bs, 1H); 4.28(s, 1H); 4.38(s, 2H); 7.15(t, 2H); 7.37(dd, 2H).

MS (m/z) ES+: 313.1(MH+, 100).

### f) N-(5-Chloro-2-{2-[7-(4-fluorobenzyl)-3-oxa-7,9-diazabicyclo[3.3.1]non-9-yl]-2-oxoethoxy}-phenyl)acetamide

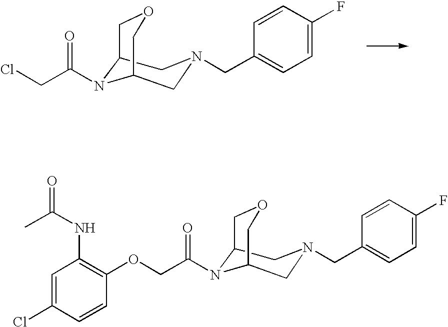

N-(5-Chloro-2-hydroxyphenyl)-acetamide (59 mg; 0.32 mmol) in THF (4 ml) is deprotonated with KN(TMS)2 (˜0.8 M in toluene; 0.38 ml; 0.32 mmol) at room temp. for 10 min. 2-Chloro-1-[7-(4-fluorobenzyl)-3-oxa-7,9-diazabicyclo[3.3.1]non-9-yl]-ethanone (50 mg; 0.16 mmol) in THF (1 ml) is added to the resulting suspension and the mixture refluxed for 1 h, poured on 2N NaOH and extracted with TBME three times. The combined organic phases are dried over Na2SO4, filtered, evaporated and purified via chromatography (acetone/hexanes (3/7 to 4/6) to yield the title compound as colorless crystals (52 mg; 70%).

1H-NMR (400 MHz; DMSO-d6), δ (ppm): 2.11(s, 3H); 2.26(d, 1H); 2.40(d, 1H); 2.89(d, 2H); 3.41(s, 2H); 3.60(d, 1H); 3.75(d, 1H); 3.80(d, 2H); 3.94(s, 1H); 4.29(s, 1H); 4.95(s, 2H); 7.00(d, 1H); 7.06(dd, 1H); 7.15(t, 2H); 7.36(dd, 2H); 8.12(bs, 1H); 9.54(s, 1H).

MS (m/z) ES+: 462.2(MH+, 100).

### Example 103

### N-(5-Chloro-2-{2-[9-(4-fluorobenzyl)-3-oxa-7,9-diazabicyclo[3.3.1]non-7-yl]-2-oxoethoxy}-phenyl)acetamide

### a) 1-(tert-Butyldimethylsilanyloxy)-3-[3-(tert-butyldimethylsilanyloxy)-2-hydroxypropoxy]propan-2-ol

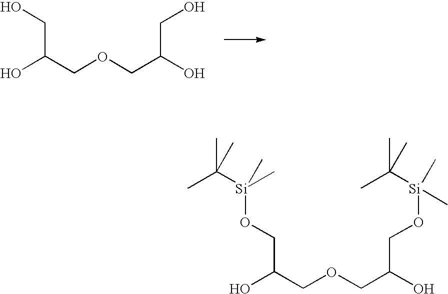

Imidazol (591.4 g; 8.696 mol), followed by tert.-butyldimethylchlorsilane (1000 g; 6.667 mol) are added under cooling and stirring to a solution of a,a′-diglycerol (481.2 g; 2.899 mol) in DMF (2.8 l), whereby the temperature is not allowed to exceed 29° C. After 10 min. a precipitate is formed and the reaction left over night at room temp. Water/HOAc (3000 ml/250 ml) are added and the product extracted 4 times with hexanes, the combined organic phases washed with water (600 ml) followed by a saturated solution of NaHCO3 (400 ml), dried over Na2SO4 and evaporated to dryness. Purification via chromatography (SiO2, hexanes/TBME 1/0 to 0/1) yielded the desired product as yellowish oil (777 g; 68%).

1H-NMR (400 MHz; DMSO-d6), δ (ppm): 0.04 (s, 12H); 0.87 (s, 18H); 3.28-3.33 (m, 2H); 3.38-3.43 (m, 2H); 3.48-3.52 (m, 4H); 3.54-3.60 (m, 2H); 4.65 (d, 2H).

MS (m/z) ES−: 393 (MH−).

### b) 1-(tert-Butyldimethylsilanyloxy)-3-[3-(tert-butyldimethylsilanyloxy)-2-tolylsulfonyloxypropoxy]prop-2-yl-toluenesulfonate

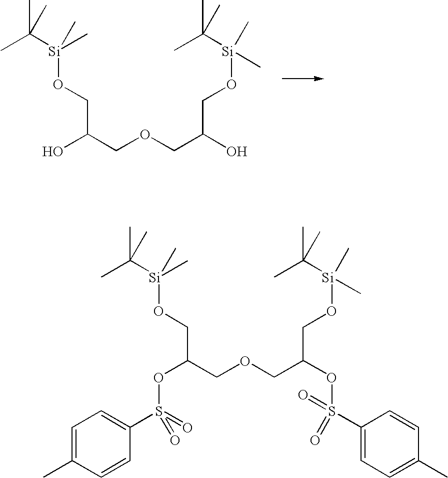

p-Toluenesulfonyl chloride (302 g; 1.58 mol) is added to a solution of 1-(tert-butyldimethylsilanyloxy)-3-[3-(tert-butyldimethylsilanyloxy)-2-hydroxypropoxy]propan-2-ol (260 g; 0.66 mot) in CH2Cl2 (390 ml). NEt3 (220 ml; 1.58 mol) and DMAP (8.1 g; 66 mmol) are added under stirring and cooling, keeping the temperature below 33° C. The reaction mixture is kept at room temp. over night, NEt3 (160 ml) added and the mixture heated on a rotary evaporator, evaporating CH2Cl2 slowly over 1 h. TBME (1000 ml) is added and the organic phase washed with water, dried over Na2SO4, filtered and evaporated to dryness to render the title compound as brownish oil (480.0 g; 100%).

### c) Cis- and trans-3,5-Bis-(tert-butyldimethylsilanyloxymethyl)-4-(4-fluorobenzyl)-morpholine (1:1).

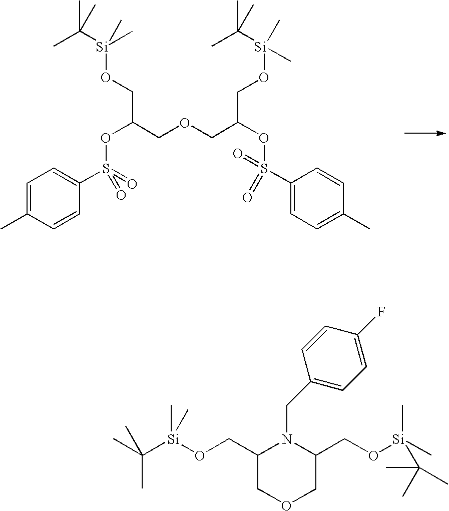

1-(tert-Butyldimethylsilanyloxy)-3-[3-(tert-butyldimethylsilanyloxy)-2-tolylsulfonyloxypropoxy]prop-2-yl-toluenesulfonate (405 g; 0.576 mol) and 4-fluorobenzylamine (262 ml; 2.3 mol) in diglyme (600 ml) are heated to 170° C. for 2.5 h. TBME (1000 ml) is added and filtered from precipitated sulfonate salt. The mother liquor is evaporated to dryness, taken up in hexanes (2000 ml) and washed with water. The organic phase is dried over Na2SO4 and evaporated to dryness and filtered from eventually precipitating additional sulfonate salt during the evaporation. Purification via chromatography (SiO2, hexanes) delivered the title compound as yellow viscous oil (167.2 g; 60%).

1H-NMR (400 MHz; DMSO-d6), δ (ppm): −0.05 (s, 3H); −0.045 (s, 3H); −0.03 (s, 3H); 0.00 (s, 3H); 0.81 (s, 9H); 0.83 (s, 9H); 2.55 (m, 1H); 2.69 (m, 1H); 3.40-3.75 (m, 10H); 7.06-7.13 (m, 2H); 7.33-7.39 (m, 2H).

MS (m/z) ES+: 484 (MH+).

d) Cis- and trans-3,5-Bis-(hydroxymethyl)-4-(4-fluorobenzyl)-morpholine (1:1)

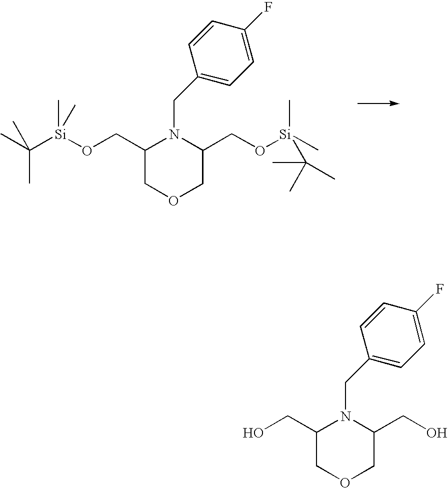

Cis- and trans-3,5-Bis-(tert-butyldimethylsilanyloxymethyl)-4-(4-fluorobenzyl)-morpholine (1:1) (8.9 g; 18.4 mmol) is dissolved in 2N HCl (10 ml) and EtOH (40 ml) and heated to 70° C. for 30 min. EtOH is evaporated, water (50 ml) and 2N HCl (10 ml) added and the aq. Phase washed with TBME twice. Na2CO3 is added to the aq. phase until pH ˜10, which is then extracted three times with TBME. The combined organic phases are dried over Na2SO4, filtered and evaporated to dryness to deliver the title product as slightly yellow oil (4.5 g; 96%).

1H-NMR (400 MHz; DMSO-d6), δ (ppm): 2.62 (m, 1H); 3.12 (m, 1H); 3.30-3.65 (m, 8H); 3.76 (d, 1H); 3.82 (s, 1H); 4.45 (t, 1H); 4.50 (t, 1H); 7.10 (dt, 2H); 7.35 (dq, 2H).

MS (m/z) ES+: 256 (MH+).

### e) cis-3,5-Bis-(chloromethyl)-4-(4-fluorobenzyl)-morpholine

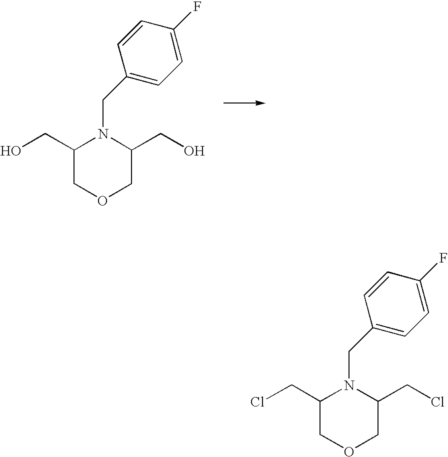

Thionyl chloride (301 ml; 4.15 mol) is added under cooling to 20° C. to DMF (1200 ml) Cis- and trans-3,5-Bis-(hydroxymethyl)-4-(4-fluorobenzyl)-morpholine (1:1) (176.5 g; 0.692 mol) in DMF (200 ml) is added under stirring and cooling to 5-12° C. within 5 min. The reaction is warmed to 42° C. for 1 h, poured on water (3000 ml) containing Na2CO3 (1100 g) and extracted with TBME three times. The combined organic phases are dried over Na2SO4, filtered and evaporated to dryness to deliver the desired products (cis/trans ˜1:1) as brownish oil (195 g; 97%). This mixture is used in the next step. Only the cis-analogue is able to cyclise with benzylamine, whereas the trans-analogue decomposed at the elevated temperature. A sample of the above brownish oil is chromatographed (SiO2, TBME/hexanes 5/95 to 20/80) to yield the pure cis-analogue (680 mg) as yellow viscous oil.

1H-NMR (400 MHz; DMSO-d6), δ (ppm): 2.71-2.78 (m, 2H); 3.55-3.62 (m, 4H); 3.70-3.75 (m, 4H); 3.95 (s, 2H); 7.13 (t, 2H); 7.39 (dd, 2H).

MS (m/z) ES+: 291 (M+, 40); 242 (60); 109 (100).

### f) 7-Benzyl-9-(4-fluorobenzyl)-3-oxa-7,9-diazabicyclo[3.3.1]nonane and 6-Benzyl-8-(4-fluoro-benzyl)-3-oxa-6,8-diaza-bicyclo[3.2.2]nonane

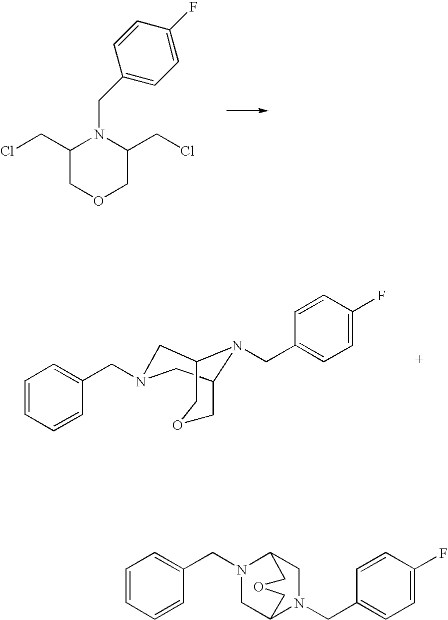

Cis- and trans-3,5-Bis-(hydroxymethyl)-4-(4-fluorobenzyl)-morpholine (1:1) (86 g; 0.29 mol) and benzylamine (322 ml; 2.9 mol) are heated to 180° C. for 30 min. Excess benzylamine is distilled off at the rotary evaporator, TBME (2000 ml) is added and solid CO2 introduced into the reaction mixture until pH 8-9. The precipitate is filtered off and the mother liquor evaporated and purified via chromatography (SiO2, acetone/hexanes 2-98 to 4/96). 6-Benzyl-8-(4-fluoro-benzyl)-3-oxa-6,8-diaza-bicyclo[3.2.2]nonane and the desired 7-benzyl-9-(4-fluorobenzyl)-3-oxa-7,9-diazabicyclo[3.3.1]nonane are eluted together (45 g; 47%) as yellow oil. The mixture is combined with hexanes (100 ml) and left over night to deliver pure crystals of the desired 7-benzyl-9-(4-fluorobenzyl)-3-oxa-7,9-diazabicyclo[3.3.1]nonane (28 g; 29%).

1H-NMR (400 MHz; DMSO-d6), δ (ppm): 2.52-2.68 (m, 6H); 3.43 (s, 2H); 3.65 (t, 2H); 3.87 (bd, 2H); 3.91 (s, 2H); 7.11 (t, 2H); 7.20 (bt, 1H); 7.28-7.40 (m, 6H).

MS (m/z) ES+: 327 (MH+).

Purification of the above mother liquor via chromatography (SiO2, TBME/MeOH/NH3 conc 1/0/0 to 98/2/0.6) delivered the isomeric 6-benzyl-8-(4-fluoro-benzyl)-3-oxa-6,8-diaza-bicyclo[3.2.2]nonane as a yellow oil (2.8 g; 2.9%).

1H-NMR (400 MHz; DMSO-d6), δ (ppm): 2.80-2.90 (m, 4H); 3.00-3.08 (m, 2H); 3.63(dd, 2H); 3.73-3.82 (m, 4H); 3.90 (bd, 2H); 7.11 (t, 2H); 7.21 (t, 1H); 7.27-7.39 (m, 6H).

MS (m/z) ES+: 327 (MH+).

### p) 9-(4-Fluorobenzyl)-3-oxa-7,9-diazabicyclo[3.3.1]nonane BL6010

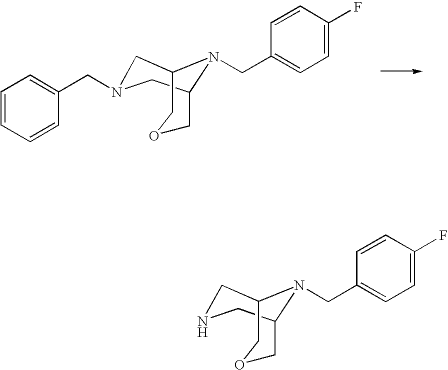

7-Benzyl-9-(4-fluorobenzyl)-3-oxa-7,9-diazabicyclo[3.3.1]nonane (20 g; 61 mmol) in EtOAc (300 ml) and HOAc (8 ml) are hydrogenated over Pd/C for 20 min., filtered, the solvent evaporated and the resulting acetate salt dissolved in EtOAc (40 ml) and crystallized by adding TBME (40 ml) under cooling (13.4 g; 74%).

1H-NMR (400 MHz; DMSO-d6), δ (ppm): 2.26(s, 2H); 2.81(d, 2H); 3.17(d, 2H); 3.79(d, 2H); 3.95(s, 2H); 4.00(d, 2H); 7.13(t, 2H); 7.40(dd, 2H). (free base)

MS (m/z) ES+: 237.1(MH+, 100).

### h) 6-(4-Fluoro-benzyl-3-oxa-6,8-diaza-bicyclo[3.2.2]nonane

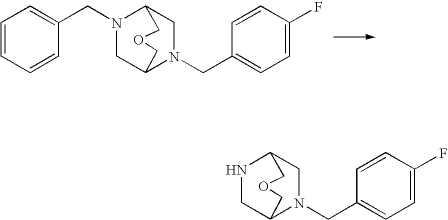

6-Benzyl-8-(4-fluoro-benzyl)-3-oxa-6,8-diaza-bicyclo[3.2.2]nonane (1 g; 3.06 mmol) in EtOAc (150 ml) and HOAc (0.5 ml) is hydrogenated over Pd/C for 2 h, filtered and evaporated to dryness, taken up in water and washed with TBME. The aq. phase is adjusted to pH ˜10 by the addition of solid K2CO3 and extracted with TBME three times. The combined organic phases are dried over Na2SO4, filtered and evaporated to dryness to deliver the target compound as slightly yellow oil (538 mg; 74%).

1H-NMR (400 MHz; DMSO-d6), δ (ppm): 2.75-2.90 (m, 3H); 3.00 (d, 2H); 3.20 (dd, 1H); 3.60 (dd, 2H); 3.70-3.80 (m, 3H); 3.93 (dd, 1H); 7.11 (t, 2H); 7.37 (dd, 2H).

MS (m/z) ES+: 237 (MH+).

### i) 9-Benzyl-3-oxa-7,9-diazabicyclo[3.3.1]nonane-7-carboxylic acid tert-butyl ester

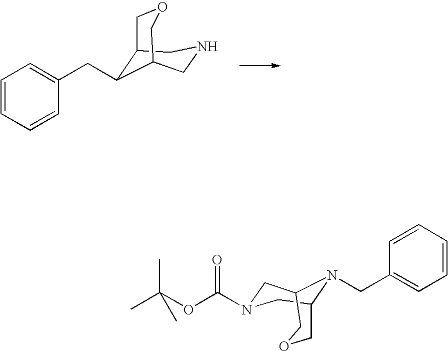

9-Benzyl-3-oxa-7,9-diazabicyclo[3.3.1]nonane (1.08 g; 4.9 mmol) in TBME (50 ml) is treated with (BOC)2O (1.1 g; 5.0 mmol) in TBME (4 ml) for 1 h at room temp. The reaction mixture is evaporated and purified via chromatography (TBME/hexanes 2/8 to 317) to yield the title compound as colorless crystals (1.45 g; 92%).

1H-NMR (400 MHz; DMSO-d6), δ (ppm): 1.39(s, 9H); 2.50(s, 2H); 3.26(d, 1H); 3.44(d, 1H); 3.69(dd, 2H); 3.75(d, 2H); 3.82(d, 2H); 3.92(s, 2H); 7.23(t, 1H); 7.32(t, 2H); 7.37(d, 2H).

MS (m/z) ES+: 319.2 (MH+, 100).

### j) 3-Oxa-7,9-diazabicyclo[3.3.1]nonane-7-carboxylic acid tert-butyl ester

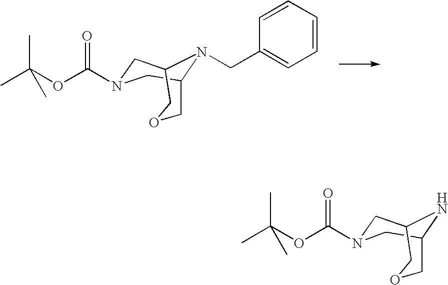

9-Benzyl-3-oxa-7,9-diazabicyclo[3.3.1]nonane-7-carboxylic acid tert-butyl ester (100 mg; 0.3 mmol) in EtOH (150 ml) is hydrogenated over Pd/C (10%; 250 mg) at 1 atm and room temp. for 1 h. After filtration and evaporation of the solvent, the residue is purified via chromatography (TBME/MeOH/NH3conc 95/5/0.5 to 90/10/2) to yield the title compound as colorless crystals (53 mg; 74%).

1H-NMR (400 MHz; DMSO-d6), δ (ppm): 1.38(s, 9H); 2.64(bd, 2H); 3.03 (bd, 1H); 3.17(bd, 1H); 3.71(m, 4H); 3.92(d, 1H); 3.99(d, 1H); 6.67(bs, 0.5H); 7.27(bs, 0.5H).

MS (m/z) ES+: 229.1(MH+, 100).

### k) 9-(4-Fluorobenzyl)-3-oxa-7,9-diazabicyclo[3.3.1]nonane-7-carboxylic acid tert-butyl ester

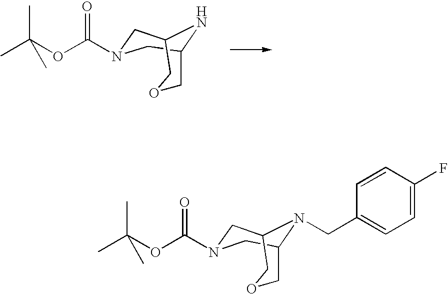

3-Oxa-7,9-diazabicyclo[3.3.1]nonane-7-carboxylic acid tert-butyl ester (97 mg; 0.4 mmol) in EtOH (4 ml) is combined with 4-fluorbenzylchlorid (0.051 ml; 0.4 mmol) and NaHCO3 (179 mg; 2.1 mmol) and refluxed for 1.5 h. The reaction mixture is evaporated, taken up in TBME, filtered and purified via chromatography (TBME/hexanes 2/8 to 3/7) to yield the title compound as colorless crystals (95 mg; 67%)

1H-NMR (400 MHz; DMSO-d6), δ (ppm): 1.40(s, 9H); 2.50(s, 2H); 3.28(d, 1H); 3.42(d, 1H); 3.68(dd, 2H); 3.73-3.88(m, 4H); 3.91(s, 2H); 7.13(t, 2H); 7.41(dd, 2H).

MS (m/z) ES+: 337.2(MH+, 100).

### l) 9-(4-Fluorobenzyl)-3-oxa-7,9-diazabicyclo[3.3.1]nonane

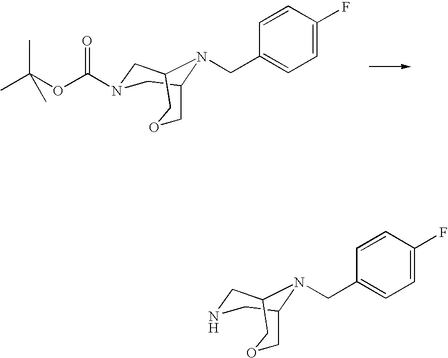

9-(4-Fluorobenzyl)-3-oxa-7,9-diazabicyclo[3.3.1]nonane-7-carboxylic acid tert-butyl ester (90 mg; 0.26 mmol) is dissolved in EtOH (4 ml) and treated with HCl conc (6 ml) for 5 min. The reaction mixture is poured on 2N NaOH/brine and extracted with TBME/THF (1:1) three times. The organic phases are combined, dried over K2CO3 and evaporated to dryness to yield the title compound as yellow resin (78 mg; 88%)

1H-NMR (400 MHz; DMSO-d6), δ (ppm): 2.26(s, 2H); 2.81(d, 2H); 3.17(d, 2H); 3.79(d, 2H); 3.95(s, 2H); 4.00(d, 2H); 7.13(t, 2H); 7.40(dd, 2H).

MS (m/z) ES+: 237.1(MH+, 100).

### m) 2-Chloro-1-[9-(4-fluorobenzyl)-3-oxa-7,9-diazabicyclo[3.3.1]non-7-yl]-ethanone

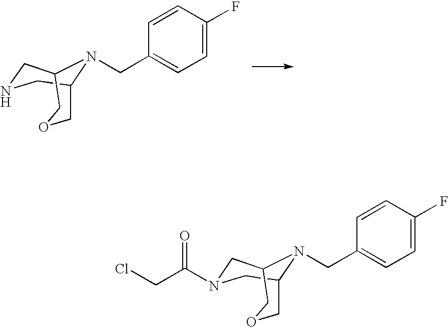

9-(4-Fluorobenzyl)-3-oxa-7,9-diazabicyclo[3.3.1]nonane (Example 103g or Example 103l) (75 mg; 0.26 mmol) in CH2Cl2 (4 ml) is treated with chloroacetylchloride (0.022 ml; 0.26 mmol) for 5 min., poured on 2N Na2CO3 and extracted with TBME three times. The combined organic phases are dried over Na2SO4, filtered and evaporated to dryness to yield the title compound as yellow foam (93 mg; 100%), which is used in the next step without further purification.

1H-NMR (400 MHz; DMSO-d6), δ (ppm): 2.60(s, 2H); 3.22(d, 1H); 3.62(d, 1H); 3.69(s, 2H); 3.80(d, 2H); 3.87(d, 1H); 3.93(s, 2H); 4.16(d, 1H); 4.30(d, 1H); 4.43(d, 1H); 7.15(t, 2H); 7.43(dd, 2H).

MS (m/z) ES+: 313.1(MH+, 30).

### n) N-(5-Chloro-2-{2-[9-(4-fluorobenzyl)-3-oxa-7,9-diazabicyclo[3.3.1]non-7-yl]-2-oxoethoxy}-phenyl]acetamide

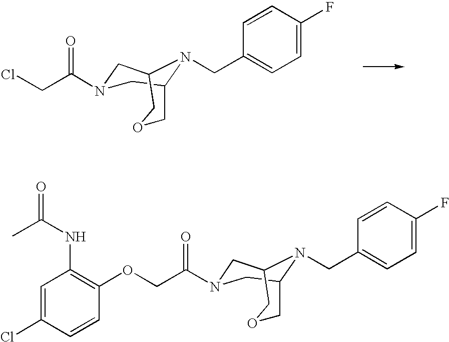

2-Chloro-1-[9-(4-fluorobenzyl)-3-oxa-7,9-diazabicyclo[3.3.1]non-7-yl]-ethanone (90 mg; 0.29 mmol) is reacted with N-(5-chloro-2-hydroxyphenyl)-acetamide as described in Example 102f to yield the title compound as colorless foam (83 mg; 62%).

1H-NMR (400 MHz; DMSO-d6), δ (ppm): 2.12(s, 3H); 3.22(d, 1H); 3.66(m, 4H); 3.78-3.90(m, 4H); 3.95(s, 2H); 4.20(d, 1H); 4.87(d, 1H); 5.00(d, 1H); 6.97-7.07(m, 2H); 7.16(t, 2H); 7.42(dd, 2H); 8.15(bs, 1H); 9.68(s, 1H).

MS (m/z) ES+: 462.2(MH+, 30).

### Example 104

### (E)-N-(5-Chloro-2-{3-[9-(4-fluorobenzyl)-3-oxa-7,9-diazabicyclo[3.3.1]non-7-yl]-3-oxopropenyl}-phenyl)-acetamide

### a) (E)-(5-Chloro-2-{3-[9-(4-fluorobenzyl)-3-oxa-7,9-diazabicyclo[3.3.1]non-7-yl]-3-oxopropenyl}-phenyl)-carbamic acid tert-butyl ester

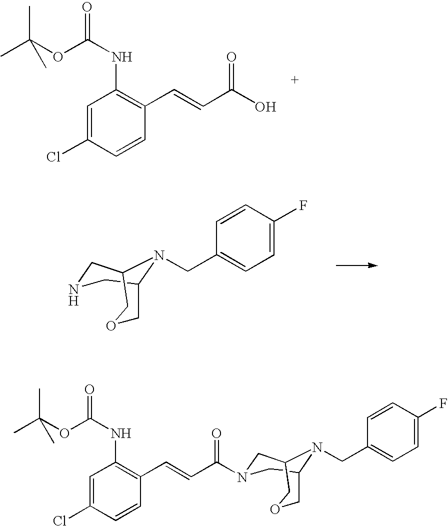

9-(4-Fluorobenzyl)-3-oxa-7,9-diazabicyclo[3.3.1]nonane (Example 103g or Example 103l) (100 mg; 0.44 mmol) and (E)-3-(2-tert-butoxycarbonylamino-4-chlorophenyl)-acrylic acid (Example 1b) (133 mg; 0.44 mmol) are combined in CH2Cl2 (4 ml) and treated with EDCl.HCl (85 mg; 0.4 mmol) over night at room temp. The reaction mixture is poured on a column of SiO2 and chromatographed (acetone/hexanes 3/7) to yield the title compound as a colorless foam (167 mg; 73%).

1H-NMR (400 MHz; DMSO-d6), δ (ppm): 1.48(s, 9H); 2.64(bd, 2H); 3.28(bd, 2H); 3.65-3.78(m, 2H); 3.83(m, 2H); 3.97(s, 2H); 4.20(d, 1H); 4.36(d, 1H); 7.13-7.20(m, 3H); 7.25(dd, 1H); 7.46(m, 3H); 7.63(d, 1H); 7.88(d, 1H); 9.25(s, 1H).

MS (m/z) ES+: 516.1 (MH+, 30).

### b) (E)-3-(2-Amino-4-chlorophenyl)-1-[9-(4-fluorobenzyl)-3-oxa-7,9-diazabicyclo[3.3.1]non-7-yl]-propenone

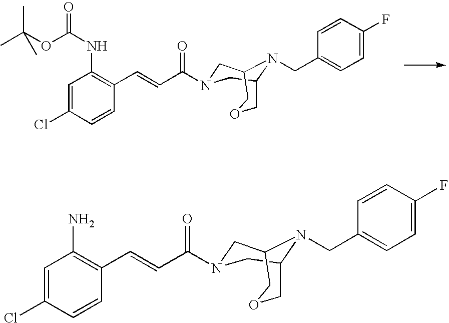

(E)-(5-Chloro-2-{3-[9-(4-fluorobenzyl)-3-oxa-7,9-diazabicyclo[3.3.1]non-7-yl]-3-oxopropenyl}-phenyl)-carbamic acid tert-butyl ester (40 mg; 0.08 mmol) is dissolved in EtOH (1 ml) and treated with HClconc (1 ml) for 2 min. at room temp. The reaction mixture is poured on a saturated solution of Na2CO3 and extracted with TBME three times. The combined organic phases are dried over Na2SO4, filtered and evaporated to dryness yield the title compound as a yellow foam (24 mg; 75%).

1H-NMR (400 MHz; DMSO-d6), δ (ppm): 2.65(bd, 2H); 3.30(m, 2H); 3.70(bt, 2H); 3.82(m, 2H); 3.98(s, 2H); 4.13(d, 1H); 4.34(d, 1H); 5.72(s, 2H, NH2); 6.55(dd, 1H); 6.73(d, 1H); 7.00(d, 1H); 7.17(t, 2H); 7.45(dd, 2H); 7.53(d, 1H); 7.62(d, 1H).

MS (m/z) ES+: 416.1 (MH+, 50).

### c) (E)-N-(5-Chloro-2-{3-[9-(4-fluorobenzyl)-3-oxa-7,9-diazabicyclo[3.3.1]non-7-yl]-3-oxopropenyl}-phenyl)-acetamide

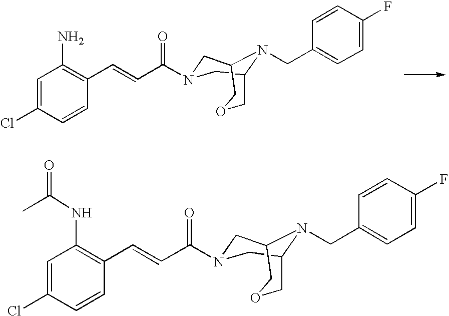

(E)-3-(2-Amino-4-chlorophenyl)-1-[9-(4-fluorobenzyl)-3-oxa-7,9-diazabicyclo[3.3.1]non-7-yl]-propenone (30 mg; 0.07 mmol) is reacted with acetylchloride and worked up as described in Example 1f to yield the title compound as colorless crystals (13 mg; 41%).

1H-NMR (400 MHz; DMSO-d6), δ (ppm): 2.08(s, 3H); 2.63(bd, 2H); 3.68(bt, 2H); 3.81(m, 4H); 3.95(s, 2H); 4.13(d, 1H); 4.32(d, 1H); 7.13-7.32(m, 4H); 7.43(m, 2H); 7.55(s, 1H); 7.58(d, 1H); 7.88(d, 1H); 9.90(s, 1H).

MS (m/z) ES+: 458.2(MH+, 50).

### Example 105

### (E)-N-(5-Chloro-2-{3-[7-(4-fluorobenzyl)-3-oxa-7,9-diazabicyclo[3.3.1]non-9-yl]-3-oxopropenyl}-phenyl)-acetamide

### a) (E)-(5-Chloro-2-{3-[7-(4-fluorobenzyl)-3-oxa-7,9-diazabicyclo[3.3.1]non-9-yl]-3-oxopropenyl}-phenyl)-carbamic acid tert-butyl ester

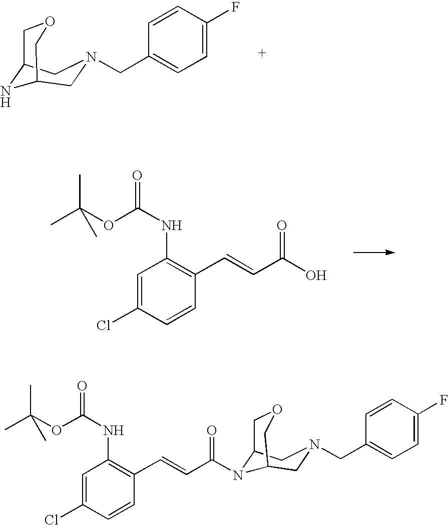

3-Oxa-7,9-diazabicyclo[3.3.1]nonane-7-carboxylic acid tert-butyl ester (Example 102d) (154 mg; 0.65 mmol) and (E)-3-(2-tert-butoxycarbonylamino-4-chlorophenyl)-acrylic (Example 1b) (193 mg; 0.65 mmol) in CH2Cl2 (4 ml) are combined with EDCl.HCl and kept overnight at room temp., poured on a silica gel column and chromatographed (acetone/hexanes 2/8) to yield the title compound as colorless crystals (286 mg; 85%).

1H-NMR (400 MHz; DMSO-d6), δ (ppm): 1.48(s, 9H); 2.28(d, 1H); 2.37(d, 1H); 2.98(bt, 2H); 3.45(dd, 2H); 3.62(bd, 1H); 3.68(bd, 1H); 3.88(d, 2H); 4.45(bd, 2H); 7.13-7.22(m, 3H); 7.26(dd, 1H); 7.39(dd, 2H); 7.47(s, 1H); 7.22(d, 1H); 7.90(d, 1H); 9.25(s, 1H).

MS (m/z) ES+: 516.1(MH+, 100).

### b) (E)-3-(2-Amino-4-chlorophenyl)-1-[7-(4-fluorobenzyl)-3-oxa-7,9-diazabicyclo[3.3.1]non-9-yl]-propenone

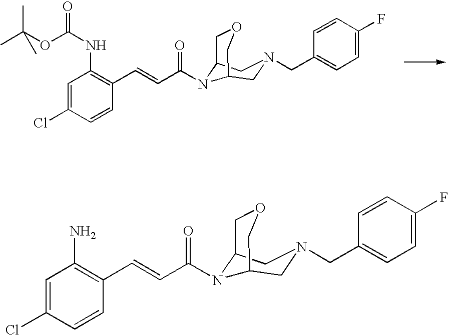

(E)-(5-Chloro-2-{3-[7-(4-fluorobenzyl)-3-oxa-7,9-diazabicyclo[3.3.1]non-9-yl]-3-oxopropenyl}-phenyl)-carbamic acid tert-butyl ester (280 mg; 0.54 mmol) is dissolved in EtOH (2 ml) and treated with HClconc (2 ml) and kept at room temp. for 2 min. The reaction mixture is poured on a saturated solution of Na2CO3 and extracted with TBME three times. The combined organic phases are dried over Na2SO4, filtered and evaporated to dryness yield the title compound as a yellow foam (229 mg; 100%).

1H-NMR (400 MHz; DMSO-d6), δ (ppm): 2.27(d, 1H); 2.35(d, 1H); 2.97(dd, 2H); 3.43(dd, 2H); 3.63(d, 1H); 3.69(d, 1H); 3.88(dd, 2H); 4.38(s, 1H); 4.45(s, 1H); 5.78(s, 2H, NH2); 6.54(dd, 1H); 6.73(d, 1H); 6.98(d, 1H); 7.17(t, 2H); 7.40(dd, 2H); 7.56(d, 1H); 7.71 (d, 1H).

MS (m/z) ES+: 416.1(MH+, 100).

### c) (E)-N-(5-Chloro-2-{3-[7-(4-fluorobenzyl)-3-oxa-7,9-diazabicyclo[3.3.1]non-9-yl]-3-oxopropenyl}-phenyl)-acetamide

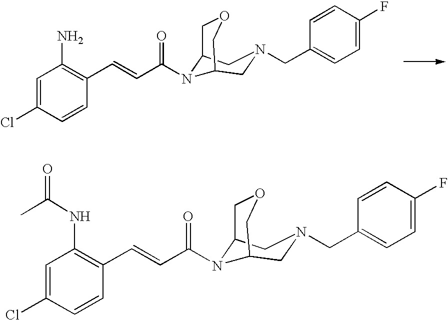

(E)-3-(2-Amino-4-chlorophenyl)-1-[7-(4-fluorobenzyl)-3-oxa-7,9-diazabicyclo[3.3.1]non-9-yl]-propenone (280 mg; 0.5 mmol) is reacted with acetylchloride and worked up as described in Example 1f to yield the title compound as colorless crystals (20 mg; 36%).

1H-NMR (400 MHz; DMSO-d6), δ (ppm): 2.21 (s, 3H); 2.28(d, 1H); 2.37(d, 1H); 2.98(t, 2H); 3.43(dd, 2H); 3.63(d, 1H); 3.68(d, 1H); 3.88(d, 2H); 4.45(bd, 2H); 7.15-7.22(m, 3H); 7.30(dd, 1H); 7.40(dd, 2H); 7.58(d, 1H); 7.71(d, 1H); 7.94(d, 1H); 9.93(s, 1H).

MS (m/z) ES+: 458.2 (MH+, 100).

### Example 106

### (E)-N-(5-Chloro-2-{3-[7-(4-fluorobenzyl)-3-oxa-7,9-diazabicyclo[3.3.1]non-9-yl]-3-oxopropenyl}-phenyl)-urea

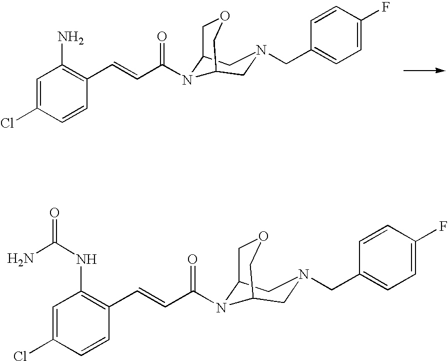

(E)-3-(2-Amino-4-chlorophenyl)-1-[7-(4-fluorobenzyl)-3-oxa-7,9-diazabicyclo[3.3.1]non-9-yl]-propenone (Example 105b) (50 mg; 0.12 mmol) is reacted with NaOCN and worked up as described in Example 4 to yield the title compound as colorless crystals (23 mg; 43%).

1H-NMR (400 MHz; DMSO-d6), δ (ppm): 2.28(d, 1H); 2.38(d, 1H); 2.97(dd, 2H); 3.43(dd, 2H); 3.63(d, 1H); 3.66(d, 1H); 3.88(d, 2H); 4.45 (m, 2H); 6.28(s, 2H, NH2); 7.07(dd, 1H); 7.13-7.21(m, 3H); 7.49(dd, 2H); 7.73(d, 1H); 7.78(d, 1H); 7.97(d, 1H); 8.43(s, 1H).

MS (m/z) ES+: 459.2 (MH+, 100).

### Example 107

### (E)-N-(5-Chloro-2-{3-[7-(4-fluorobenzyl)-3-oxa-7,9-diazabicyclo[3.3.1]non-9-yl]-3-oxopropenyl}-phenyl)-N′cyanoguanidine

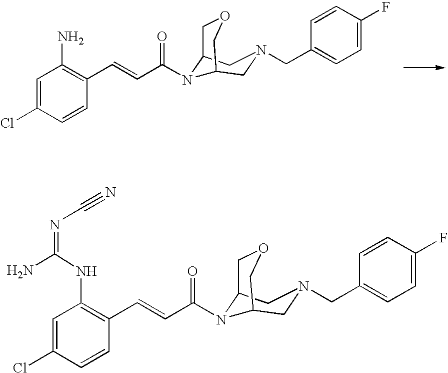

(E)-3-(2-Amino-4-chlorophenyl)-1-[7-(4-fluorobenzyl)-3-oxa-7,9-diazabicyclo[3.3.1]non-9-yl]-propenone (Example 105b) (50 mg; 0.12 mmol) is reacted with NaN(CN)2 as described in Example 2 and yielded the title compound as colorless crystals (15 mg; 26%).

1H-NMR (400 MHz; DMSO-d6), δ (ppm): 2.25(d, 1H); 2.33(d, 1H); 2.96(bt, 2H); 3.41(d, 2H); 3.60(d, 1H); 3.67(d, 1H); 3.85(d, 2H); 4.42(m, 2H); 7.17(t, 2H); 7.23(d, 1H); 7.32-7.42(m, 3H); 7.47(s, 1H); 7.60(d, 1H); 7.94(d, 1H); 9.00(s, 1H).

MS (m/z) ES+: 483.1(MH+, 100).

### Example 108

### (E)-(5-Chloro-2-{3-[9-(4-fluorobenzyl)-3-oxa-7,9-diazabicyclo[3.3.1]non-7-yl]-3-oxopropenyl}-phenyl)-urea

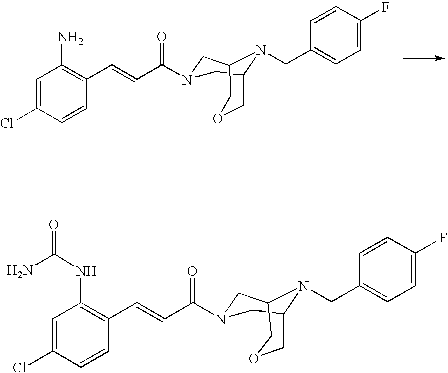

(E)-3-(2-Amino-4-chlorophenyl)-1-[9-(4-fluorobenzyl)-3-oxa-7,9-diazabicyclo[3.3.1]non-7-yl]-propenone (Example 104b) (30 mg; 0.07 mmol) is reacted with NaOCN and worked up as described in Example 4 to yield the title compound as colorless crystals (35 mg; 37%).

1H-NMR (400 MHz; DMSO-d6), δ (ppm): 2.63(d, 2H); 3.30(d, 2H); 3.67-3.76(m, 2H); 3.82(m, 2H); 3.98(s, 2H); 4.17(d, 1H); 4.37(d, 1H); 6.25(s, 2H, 7.07(dd, 1H); 7.13-7.21(m, 3H); 7.45(dd, 2H); 7.67(d, 1H); 7.77(d, 1H); 7.99(d, 1H); 8.41(s, 1H).

MS (m/z) ES+: 459.2(MH+, 100).

### Example 109

### N-(5-Chloro-2-{(E)-3-[9-(4-fluorobenzyl)-3-oxa-7,9-diazabicyclo[3.3.1]non-7-yl]-3-oxopropenyl}-4-methoxyphenyl)-acetamide

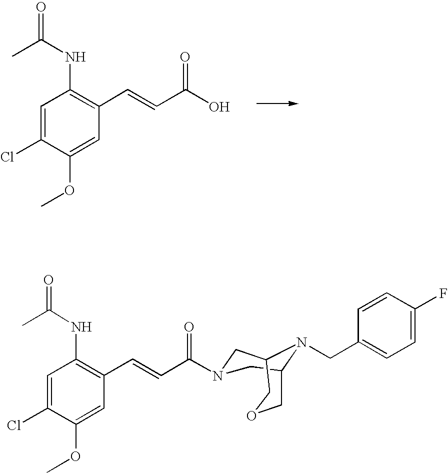

(E)-3-(2-Acetylamino-4-chloro-5-methoxy-phenyl)-acrylic acid (Example 23e) and 9-(4-fluoro-benzyl)-3-oxa-7,9-diaza-bicyclo[3.3.1]nonane (Example 103g or 103l) are coupled according to Example 55f to yield the title compound purified via chromatography (SiO2, acetone/hexanes 3/7 to 8/2) and crystallized from acetone/TBME (98 mg; 54%).

1H-NMR (400 MHz; DMSO-d6), δ (ppm): 2.05 (s, 3H); 2.65 (bs, 2H); 3.28 (bd, 1H); 3.56-3.87 (m, 5H); 3.93 (s, 3H); 3.95 (s, 2H); 4.15 (d, 1H); 4.34 (d, 1H); 7.13 (t, 2H); 7.23 (d, 1H); 7.39-7.47 (m, 4H); 7.55 (d, 1H); 9.72 (s, 1H).

MS (m/z) ES+: 488 (MH+).

### Example 111

### N-(5-Chloro-2-{(E)-3-[7-(4-fluorobenzyl)-3-oxa-7,9-diazabicyclo[3.3.1]non-9-yl]-3-oxopropenyl}-4-methoxyphenyl)-acetamide

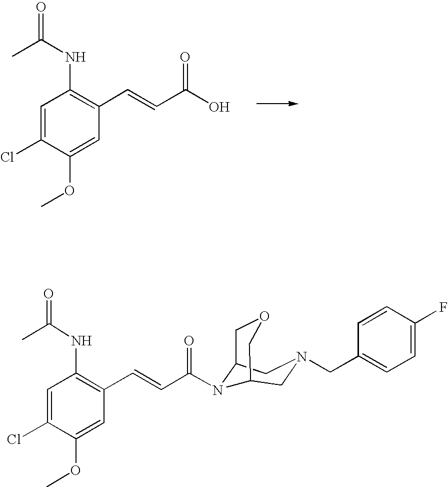

(E)-3-(2-Acetylamino-4-chloro-5-methoxy-phenyl)-acrylic acid (Example 23e) and 7-(4-Fluoro-benzyl)-3-oxa-7,9-diaza-bicyclo[3.3.1]nonane (Example 102d) are coupled according to the conditions described in Example 55f to yield the desired product purified via chromatography (SiO2, acetone/hexanes 4/6 to 6/4) to generate the title compound as yellow foam crystallized from TBME (93 mg; 61%).

1H-NMR (400 MHz; DMSO-d6), δ (ppm): 2.04 (s, 3H); 2.28 (d, 1H); 2.38 (d, 1H); 2.95 (d, 1H); 3.02 (d, 1H); 3.43 (q, 2H); 3.63 (d, 1H); 3.70 (d, 1H); 3.88 (d, 2H); 3.90 (s, 3H); 4.43 (bd, 2H); 7.10-7.22 (m, 3H); 7.35 (dd, 2H); 7.43 (s, 1H); 7.47 (s, 1H); 7.63 (d, 1H); 9.72 (s, 1H).

MS (m/z) ES+: 488.1 (MH+).

### Example 112

### N-(5-Chloro-2-{(E)-3-[7-(4-fluorobenzyl)-3-oxa-7,9-diaza-bicyclo[3.3.1]non-9-yl]-3-oxopropenyl}-4-methoxyphenyl)-methanesulfonamide

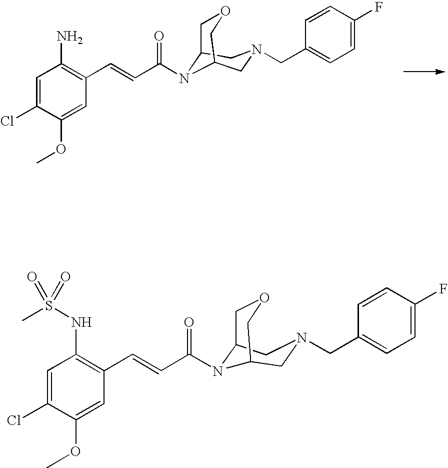

(E)-3-(2-Amino-4-chloro-5-methoxy-phenyl)-1-[7-(4-fluoro-benzyl)-3-oxa-7,9-diazabicyclo[3.3.1]non-9-yl]-propenone (obtained from Example 23c and Example 102d coupled according to conditions described in Example 23d) is treated with methanesulfonyl chloride as described in Example 70a to yield the title product purified via chromatography (SiO2, acetone/hexanes 4/6 to 1/1) crystallized from EtOH/TBME/hexanes (89 mg; 54%).

1H-NMR (400 MHz; DMSO-d6), δ (ppm): 2.28 (d, 1H); 2.38 (d, 1H); 2.95 (d, 1H); 2.97 (s, 3H); 3.02 (d, 1H); 3.43 (q, 2H); 3.63 (d, 1H); 3.70 (d, 1H); 3.88 (d, 2H); 3.90 (s, 3H); 4.43 (bd, 2H); 7.10-7.22 (m, 3H); 7.35 (dd, 2H); 7.43 (s, 1H); 7.47 (s, 1H); 7.63 (d, 1H); 9.72 (s, 1H).

MS (m/z) ES−: 522.1 (MH−).

### Example 113

### 5-Chloro-2-{(E)-3-[7-(4-fluoro-benzyl)-3-oxa-7,9-diaza-bicyclo[3.3.1]non-9-yl]-3-oxo-propenyl}-4-methoxy-phenyl)-urea

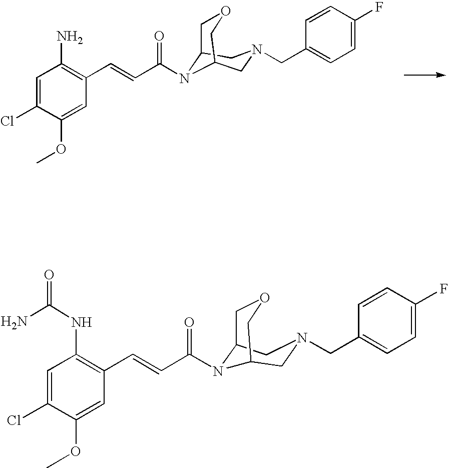

(E)-3-(2-Amino-4-chloro-5-methoxy-phenyl)-1-[7-(4-fluoro-benzyl)-3-oxa-7,9-diazabicyclo[3.3.1]non-9-yl]-propenone (obtained from Example 23c and Example 102d coupled according to conditions described in Example 23d) is treated according to Example 4 and purified via chromatography (SiO2, acetone/hexanes 6/4 to 1/0) to yield the title compound as colorless crystals (64 mg; 59%).

1H-NMR (400 MHz; DMSO-d6), δ (ppm): 2.28 (d, 1H); 2.39 (d, 1H); 2.96 (d, 1H); 3.00 (d, 1H); 3.43 (q, 2H); 3.63 (d, 1H); 3.70 (d, 1H); 3.89 (s, 3H); 3.91 (d, 2H); 4.42 (bs, 1H); 4.47 (bs, 1H); 6.03 (s, 2H); 7.11-7.19 (m, 3H); 7.35-7.40 (m, 3H); 7.69 (d, 1H); 7.70 (s, 1H); 8.18 (s, 1H).

MS (m/z) ES+: 489.2 (MH+).

### Example 114

### 1-(5-Chloro-2-{(E)-3-[7-(4-fluoro-benzyl)-3-oxa-7,9-diaza-bicyclo[3.3.1]non-9-yl]-3-oxo-propenyl}-4-methoxy-phenyl)-3-methyl-urea

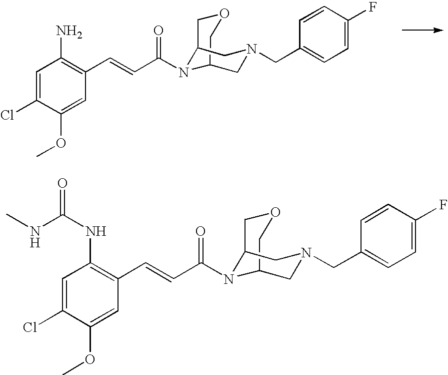

(E)-3-(2-Amino-4-chloro-5-methoxy-phenyl)-1-[7-(4-fluoro-benzyl)-3-oxa-7,9-diazabicyclo[3.3.1]non-9-yl]-propenone (obtained from Example 23c and Example 102d coupled according to conditions described in Example 23d) is treated according to Example 23f and purified via chromatography (SiO2, acetone/hexanes 6/4 to 1/0) to yield the title compound as colorless crystals (40 mg; 57%).

1H-NMR (400 MHz; DMSO-d6), δ (ppm):): 2.28 (d, 1H); 2.39 (d, 1H); 2.63 (d, 3H); 2.96 (d, 1H); 3.02 (d, 1H); 3.43 (q, 2H); 3.63 (d, 1H); 3.70 (d, 1H); 3.89 (s, 3H); 3.91 (d, 2H); 4.42 (bs, 1H); 4.47 (bs, 1H); 6.27 (q, 1H); 7.11-7.19 (m, 3H); 7.35-7.40 (m, 3H); 7.68 (s, 1H); 7.69 (d, 1H); 8.18 (s, 1H).

MS (m/z) ES+: 503 (MH+, 60); 446 (100), 428 (20).

### Example 115

### 1-(5-Chloro-2-{(E)-3-[7-(4-fluoro-benzyl)-3-oxa-7,9-diaza-bicyclo[3.3.1]non-9-yl]-3-oxo-propenyl}-4-methoxy-phenyl)-3-cyclopropyl-urea

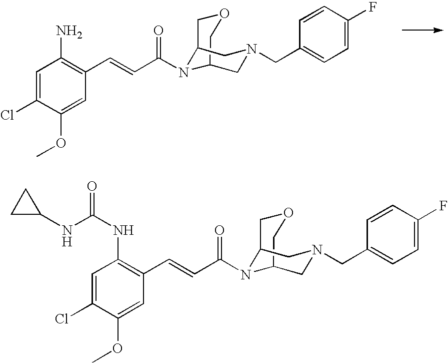

(E)-3-(2-Amino-4-chloro-5-methoxy-phenyl)-1-[7-(4-fluoro-benzyl)-3-oxa-7,9-diazabicyclo[3.3.1]non-9-yl]-propenone (obtained from Example 23c and Example 102d coupled according to conditions described in Example 23d) is treated according to Example 23f and purified via chromatography (SiO2, acetone/hexanes 6/4 to 1/0) to yield the title compound as colorless crystals (26 mg; 35%).

1H-NMR (400 MHz; DMSO-d6), δ (ppm): 0.45 (m, 2H); 0.66 (m, 2H); 2.28 (d, 1H); 2.39 (d, 1H); 2.53 (m, 1H); 2.96 (d, 1H); 3.02 (d, 1H); 3.43 (q, 2H); 3.63 (d, 1H); 3.70 (d, 1H); 3.89 (s, 3H); 3.91 (d, 2H); 4.42 (bs, 1H); 4.47 (bs, 1H); 6.65 (bd, 1H); 7.11-7.15 (m, 3H); 7.36-7.40 (m, 3H); 7.68 (d, 1H); 7.70 (s, 1H); 8.02 (s, 1H).

MS (m/z) ES+: 529 (MH+).

### Example 116

### 5-Chloro-2-{(E)-3-[7-(4-fluoro-benzyl)-3-oxa-7,9-diaza-bicyclo[3.3.1]non-9-yl]-3-oxo-propenyl}-4-methoxy-N,N-dimethyl-benzenesulfonamide

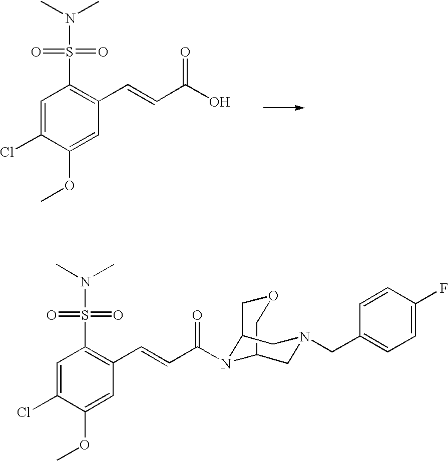

(E)-3-(4-Chloro-2-dimethylsulfamoyl-5-methoxy-phenyl)-acrylic acid (Example 30d) and 7-(4-fluoro-benzyl)-3-oxa-7,9-diaza-bicyclo[3.3.1]nonane (Example 102d) are coupled according to Example 30e to provide the title compound as colorless crystals (91 mg; 67%).

1H-NMR (400 MHz; DMSO-d6), δ (ppm): 2.28 (d, 1H); 2.37 (d, 1H); 2.68 (s, 6H); 2.96 (d, 1H); 3.00 (d, 1H); 3.43 (dd, 2H); 3.63 (d, 1H); 3.70 (d, 1H); 3.89 (dd, 2H); 4.05 (s, 3H); 4.38 (bs, 1H); 4.42 (bs, 1H); 7.13 (bt, 2H); 7.22 (d, 1H); 7.36 (m, 2H); 7.57 (s, 1H); 7.82 (s, 1H); 8.23 (d, 1H).

MS (m/z) ES+:

### Example 117

### N-(3-Chloro-6-{(E)-3-[9-(4-fluoro-benzyl)-3-oxa-7,9-diaza-bicyclo[3.3.1]non-7-yl]-3-oxo-propenyl}-2,4-dimethoxy-phenyl)-acetamide

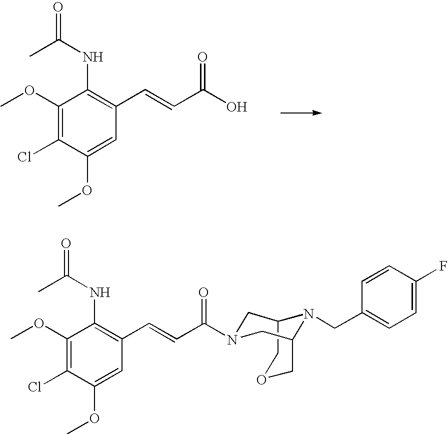

9-(4-Fluoro-benzyl)-3-oxa-7,9-diaza-bicyclo[3.3.1]nonane (Example 103g or 103l) and (E)-3-(2-acetylamino-4-chloro-3,5-dimethoxy-phenyl)-acrylic acid (Example 59d) are coupled according to Example 59e to obtain the title compound after chromatography (SiO2, CH2Cl2/MeOH 1/0 to 96/4) as colorless crystals (90 mg; 86%).

1H-NMR (400 MHz; DMSO-d6), δ (ppm): 2.05 (s, 3H); 2.62 (bs, 2H); 3.28 (d, 2H); 3.69 (s, 3H); 3.71 (d, 1H); 3.77 (d, 1H); 3.81 (m, 2H); 3.95 (s, 3H); 3.96 (s, 2H); 4.13 (d, 1H); 4.30 (d, 1H); 7.13 (t, 2H); 7.22 (d, 1H); 7.31 (s, 1H); 7.38-7.45 (m, 3H); 9.43 (s, 1H).

MS (m/z) ES+: 518.3 (MH+).

### Example 118

### N-(3-Chloro-6-{(E)-3-[9-(4-fluoro-benzyl)-3-oxa-7,9-diaza-bicyclo[3.3.1]non-7-yl]-3-oxo-propenyl}-2-methoxy-phenyl)-acetamide

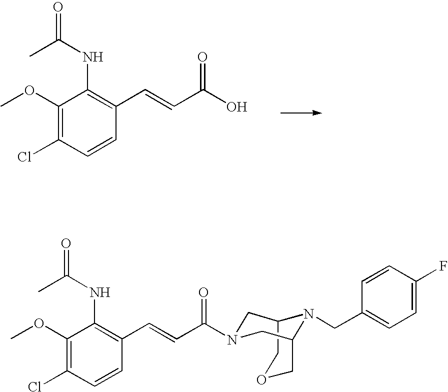

(E)-3-(2-Acetylamino-4-chloro-3-methoxy-phenyl)-acrylic acid (obtained by bromination of 3-chloro-2-methoxy-phenylamine followed by Stille coupling, hydrolysis and acylation as described for Example 59d) and 9-(4-fluoro-benzyl)-3-oxa-7,9-diaza-bicyclo[3.3.1]nonane (Example 103g or 103l) are coupled according to Example 59e to deliver the title compound after chromatography (SiO2, CH2Cl2/MeOH 1/0 to 96/4) as colorless crystals (91 mg; 93%).

1H-NMR (400 MHz; DMSO-d6), δ (ppm): 2.18 (s, 3H); 2.62 (bd, 2H); 3.29 (bd, 2H); 3.68 (d, 1H); 3.72 (d, 1H); 3.75 (s, 3H); 3.80 (bs, 2H); 3.94 (s, 2H); 4.15 (d, 1H); 4.32 (d, 1H); 7.13 (t, 2H); 7.20 (d, 1H); 7.42 (dd, 2H); 7.70 d, 1H); 7.73 (d, 1H); 8.05 (d, 1H); 9.58 (s, 1H).

MS (m/z) ES+: 488.3 (MH+).

### Example 119

### N-(5-Chloro-2-{(E)-3-[9-(4-fluoro-benzyl)-3-oxa-7,9-diaza-bicyclo[3.3.1]non-7-yl]-3-oxo-propenyl}-4-methoxy-phenyl)-methanesulfonamide

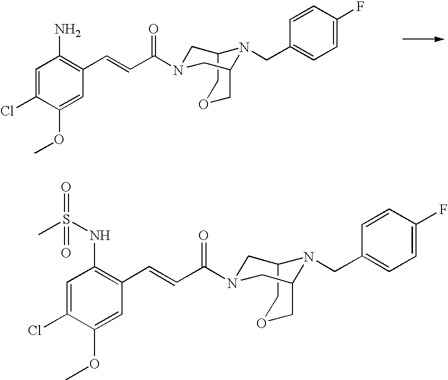

(E)-3-(2-Amino-4-chloro-5-methoxy-phenyl)-1-[9-(4-fluoro-benzyl)-3-oxa-7,9-diaza-bicyclo[3.3.1]non-7-yl]-propenone (obtained by coupling Example 23c and Example 103g or Example 103l according to Example 23d) is treated with methanesulfonyl chloride according to Example 70a to yield the title compound as colorless crystals (35 mg; 29%).

1H-NMR (400 MHz; DMSO-d6), δ (ppm): 2.63 (bs, 2H); 2.94 (s, 3H); 3.28 (bd, 2H); 3.69 (d, 1H); 3.73 (bd, 1H); 4.31 (bs, 2H); 3.95 (s, 3H); 3.96 (s, 2H); 4.16 (d, 1H); 4.32 (d, 1H); 7.13 (t, 2H); 7.23 (d, 1H); 7.36 (s, 1H); 7.42 (dd, 2H); 7.51 (s, 1H); 7.78 (d, 1H); 9.43 (s, 1H).

MS (m/z) ES+: 524 (MH+).

### Example 120

### (5-Chloro-2-{(E)-3-[9-(4-fluoro-benzyl)-3-oxa-7,9-diaza-bicyclo[3.3.1]non-7-yl]-3-oxo-propenyl}-4-methoxy-phenyl)-urea

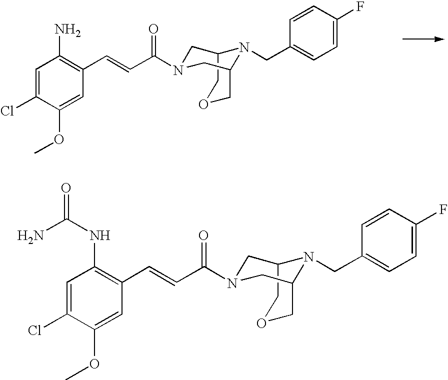

(E)-3-(2-Amino-4-chloro-5-methoxy-phenyl)-1-[9-(4-fluoro-benzyl)-3-oxa-7,9-diaza-bicyclo[3.3.1]non-7-yl]-propenone (obtained by coupling Example 23c and Example 103g or Example 103l according to Example 23d) is treated according to Example 4 to yield the title compound as colorless crystals (43 mg; 49%).

1H-NMR (400 MHz; DMSO-d6), δ (ppm): 2.65 (bd, 2H); 3.28 (bd, 2H); 3.69 (d, 1H); 3.75 (d, 1H); 3.81 (bs, 2H); 3.88 (s, 3H); 3.95 (s, 2H); 4.15 (d, 1H); 4.35 (d, 1H); 6.02 (s, 2H); 7.13 (t, 2H); 7.19 (d, 1H); 7.37 (s, 1H); 7.41 (dd, 2H); 7.60 (d, 1H); 7.70 (s, 1H); 8.18 (s, 1H).

MS (m/z) ES+: 489 (MH+, 100); 446 (30); 279 (75); 237 (40); 210 (40).

### Example 121

### Cyclopropanecarboxylic acid (5-chloro-2-{(E)-3-[9-(4-fluoro-benzyl)-3-oxa-7,9-diaza-bicyclo[3.3.1]non-7-yl]-3-oxo-propenyl}-4-methoxy-phenyl)-amide

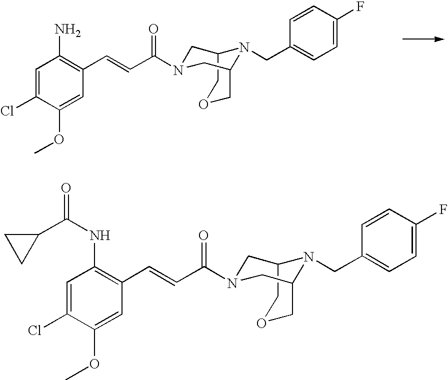

(E)-3-(2-Amino-4-chloro-5-methoxy-phenyl)-1-[9-(4-fluoro-benzyl)-3-oxa-7,9-diaza-bicyclo[3.3.1]non-7-yl]-propenone is treated with cyclopropane carboxylic acid chloride according to Example 1f yielding the title compound as colorless crystals (31 mg; 67%).

1H-NMR (400 MHz; DMSO-d6), δ (ppm): 0.80 (bd, 4H); 1.82-1.20 (m, 1H); 2.65 (bs, 2H); 3.25 (d, 2H); 3.67 (d, 1H); 3.75 (d, 1H); 3.81 (bs, 2H); 3.92 (s, 3H); 3.95 (s, 2H); 4.15 (d, 1H); 4.33 (d, 1H); 7.13 (t, 2H); 7.22 (d, 1H); 7.39-7.48 (m, 4H); 7.59 (d, 1H); 9.95 (s, 1H).

MS (m/z) ES+: 514.2 (MH+).

### Example 122

### N-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-acetamide

### a) (E)-3-(2-Amino-4-chloro-5-trifluoromethoxy-phenyl)-1-[7-(4-fluoro-benzyl)-3-oxa-7,9-diaza-bicyclo[3.3.1]non-9-yl]-propenone

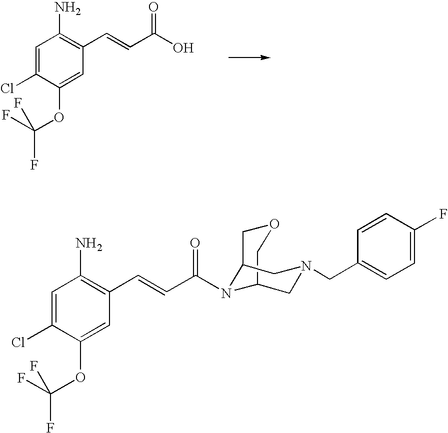

(E)-3-(2-Amino-4-chloro-5-trifluoromethoxy-phenyl)-acrylic acid (Example 35c) is converted to the acid chloride as described in Example 35d and combined with 7-(4-Fluoro-benzyl)-3-oxa-7,9-diaza-bicyclo[3.3.1]nonane (Example 102d) as described in Example 35e. The title compound is obtained via chromatography (XTerra, RP18, 7 μm, MeCN/water 40/60 to 100/0) as yellow foam (249 mg; 35%).

1H-NMR (400 MHz; DMSO-d6), δ (ppm): 2.25 (bd, 1H); 2.35 (bd, 1H); 2.96 (t, 2H); 3.41 (dd, 2H); 3.60 (d, 1H); 3.67 (d, 1H); 3.88 (dd, 2H); 4.40 (bd, 2H); 5.97 (bs, 2H); 6.84 (s, 1H); 7.05 (d, 1H); 7.13 (t, 2H); 7.35 (dd, 2H); 7.63 (d, 1H); 7.70 (s, 1H).

MS (m/z) ES+: 500 (MH+).

### b) N-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-acetamide

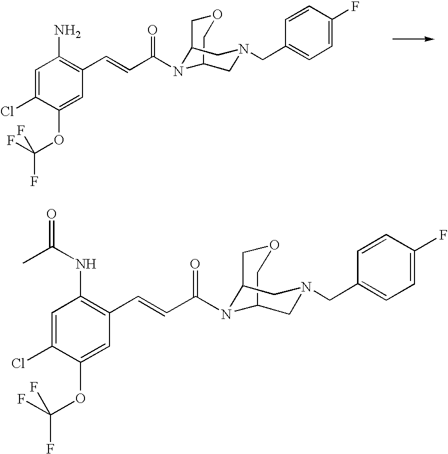

(E)-3-(2-Amino-4-chloro-5-trifluoromethoxy-phenyl)-1-[7-(4-fluoro-benzyl)-3-oxa-7,9-diaza-bicyclo[3.3.1]non-9-yl]-propenone from above is treated according to Example 1f and the title compound obtained as colorless crystals (40 mg; 37%).

1H-NMR (400 MHz; DMSO-d6), δ (ppm): 2.10 (s, 3H); 2.26 (d, 1H); 2.37 (d, 1H); 2.97 (dd, 2H); 3.41 (dd, 2H); 3.61 (d, 1H); 3.68 (d, 1H); 3.89 (bt, 2H); 4.43 (bs, 2H); 7.15 (bt, 2H); 7.29 (d, 1H); 7.37 (m, 2H); 7.65 (d, 1H); 7.30 (s, 1H); 8.08 (s, 1H); 10.02 (s, 1H).

MS (m/z) ES+: 542 (MH+).

### Example 123

### (5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-urea

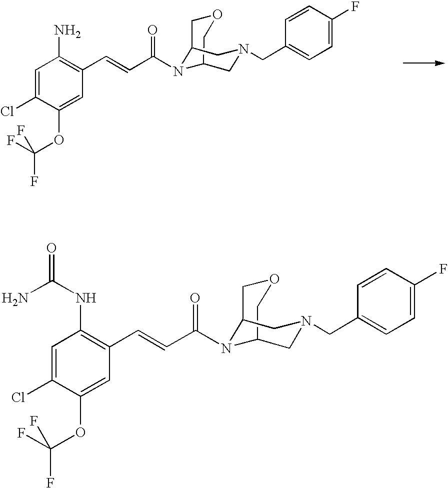

(E)-3-(2-Amino-4-chloro-5-trifluoromethoxy-phenyl)-1-[7-(4-fluoro-benzyl)-3-oxa-7,9-diaza-bicyclo[3.3.1]non-9-yl]-propenone from above is treated according to Example 4 and the title compound obtained as colorless crystals (24 mg; 22%).

1H-NMR (400 MHz; DMSO-d6), δ (ppm): 2.28 (d, 1H); 2.37 (d, 1H); 2.97 (bt, 2H); 3.42 (dd, 2H); 3.61 (d, 1H); 3.67 (d, 1H); 3.35-3.92 (m, 2H); 4.42 (bd, 2H); 6.30 (bs, 2H); 7.13 (bt, 2H); 7.22 (d, 1H); 7.36 (bt, 2H); 7.67 (d, 1H); 7.93 (s, 1H); 8.14 (s, 1H); 8.54 (s, 1H).

MS (m/z) ES+: 543 (MH+).

### Example 124

### 1-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-3-methyl-urea

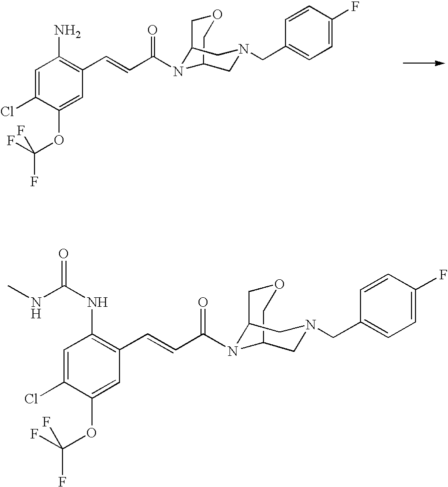

(E)-3-(2-Amino-4-chloro-5-trifluoromethoxy-phenyl)-1-[7-(4-fluoro-benzyl)-3-oxa-7,9-diaza-bicyclo[3.3.1]non-9-yl]-propenone from above is treated according to Example 23f and the title compound obtained after purification via chromatography (XTerra, RP18, 7 μm, MeCN/water 40/60 to 100/0) as colorless crystals (48 mg; 45%).

1H-NMR (400 MHz; DMSO-d6), δ (ppm): rotamers at room temperature; decomposes when heated to 120° C.

MS (m/z) ES+: 57 (MH+, 100); 500 (60).

### Example 125

### N-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-isobutyramide

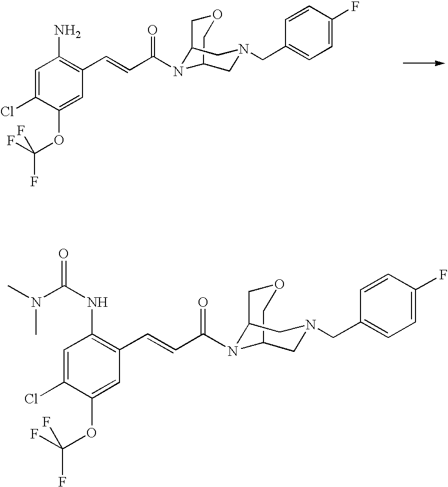

(E)-3-(2-Amino-4-chloro-5-trifluoromethoxy-phenyl)-1-[7-(4-fluoro-benzyl)-3-oxa-7,9-diaza-bicyclo[3.3.1]non-9-yl]-propenone from above is treated according to Example 29 and the title compound obtained after purification via chromatography (XTerra, RP18, 7 μm, MeCN/water 40/60 to 10010) as colorless crystals (60 mg; 53%).

1H-NMR (400 MHz; DMSO-d6), δ (ppm): 2.25 (d, 1H); 2.36 (d, 1H); 2.93 (s, 6H); 2.94 (d, 1H); 3.00 (d, 1H); 3.41 (dd, 2H); 3.61 (d, 1H); 3.67 (d, 1H); 3.88 (t, 2H); 4.43 (bs, 2H); 7.14 (t, 2H); 7.22 (d, 1H); 7.36 (dd, 2H); 7.55 (d, 1H); 7.56 (s, 1H); 8.07 (s, 1H); 8.42 (s, 1H).

MS (m/z) ES+: 571 (MH+, 70); 500 (100).

### Example 126

### 5-Chloro-2-{(E)-3-[7-(4-fluoro-benzyl)-3-oxa-7,9-diaza-bicyclo[3.3.1]non-9-yl]-3-oxo-propenyl-}N,N-dimethyl-4-trifluoromethoxy-benzenesulfonamide

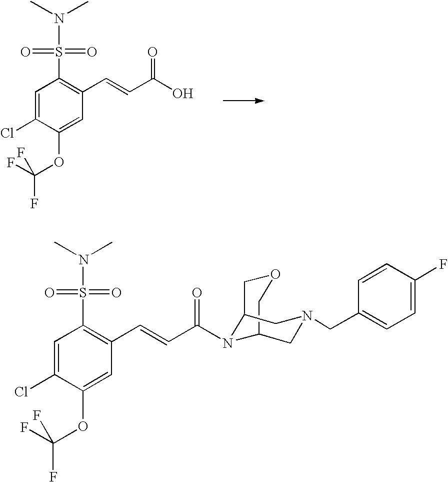

(E)-3-(4-Chloro-2-dimethylsulfamoyl-5-trifluoromethoxy-phenyl)-acrylic acid (Example 40c) and 7-(4-fluoro-benzyl)-3-oxa-7,9-diaza-bicyclo[3.3.1]nonane (Example 102d) are coupled according to Example 40d and the title compound obtained as colorless crystals (78 mg; 62%).

1H-NMR (400 MHz; DMSO-d6), δ (ppm): 2.28 (d, 1H); 2.36 (d, 1H); 2.76 (s, 6H); 2.94 (d, 1H); 2.98 (d, 1H); 3.43 (dd, 2H); 3.62 (d, 1H); 3.68 (d, 1H); 3.88 (t, 2H); 4.40 (bs, 2H); 7.13 (t, 2H); 7.30 (d, 1H); 7.35 (dd, 2H); 8.03 (s, 1H); 8.20 (d, 1H); 8.25 (s, 1H).

MS (m/z) ES+: 592 (MH+).

### Example 127

### N-(5-Chloro-2-{(E)-3-[7-(4-fluoro-benzyl)-3-oxa-7,9-diaza-bicyclo[3.3.1]non-9-yl]-3-oxo-propenyl}-4-trifluoromethoxy-phenyl)-N,N-dimethylsulfonylurea

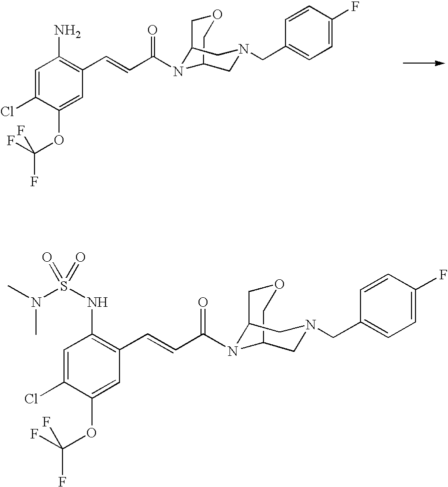

(E)-3-(2-Amino-4-chloro-5-trifluoromethoxy-phenyl)-1-[7-(4-fluoro-benzyl)-3-oxa-7,9-diaza-bicyclo[3.3.1]non-9-yl]-propenone (Example 122a) is treated according to Example 39 to yield the title compound as colorless crystals (93 mg; 75%).

1H-NMR (400 MHz; DMSO-d6), δ (ppm): 2.23-2.41 (m, 2H); 2.71 (s, 6H); 2.94-3.07 (m, 2H); 3.40-3.50 (m, 2H); 3.62 (d, 1H); 3.69 (d, 1H); 3.89 (bt, 2H); 4.43 (bs, 2H); 7.14 (bt, 2H); 7.28 (bd, 1H); 7.38 (bs, 2H); 7.57 (bs, 1H); 7.90 (bd, 1H); 8.08 (bs, 1H); 9.95 (bs, 1H).

MS (m/z) ES+: 607 (MH+).

### Example 128

### 1-(5-Chloro-4-cyclopropylmethoxy-2-{(E)-3-[7-(4-fluoro-benzyl)-3-oxa-7,9-diaza-bicyclo[3.3.1]non-9-yl]-3-oxo-propenyl}-phenyl)-3-methyl-urea

### a) 5-Bromo-2-chlorophenol

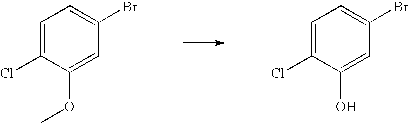

BBr3 (8.2 ml; 84.9 mmol) is added under stirring at 0-5° C. to a solution of 5-bromo-2-chloroanisol (18.26 g; 82.4 mmol) in CH2Cl2 (45 ml). The reaction mixture is stirred for 4 h at room temp., then poured on 2N NaOH/ice and washed with TBME twice. The aqueous phase is acidified with 2N HCl and extracted with TBME twice. The combined organic phases are dried over Na2SO4, evaporated to dryness and rendered the title compound as colorless crystals (16.35 g; 95%).

1H-NMR (400 MHz; DMSO-d6), δ (ppm): 6.97 (dd, 1H); 7.09 (d, 1H); 7.26 (d, 1H); 10.65 (bs, 1H, OH).

MS (m/z) ES+: 210 (15); 208 (70, M+); 206 (50); 179 (20); 177 (15); 63 (100).

### b) 5-Bromo-2-chloro-4-nitrophenol

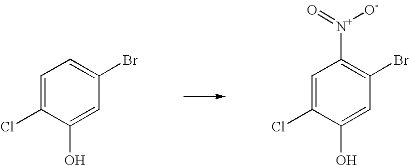

HNO3 (100%; 3.3 ml; 78.8 mmol) is added under stirring at 0-5° C. within 10 min. to a solution of 5-bromo-2-chlorophenol (16.35 g; 78.8 mmol) in CHCl3 (160 ml). The orange colored reaction mixture is kept at 0-5° C. for 1 h, then poured on hexanes (400 ml), stirred for 15 min. at 0-5° C. and filtered to yield a first batch of the title compound (7.0 g; 35%) as yellow crystals. The filtrate is evaporated and purified via chromatography (SiO2; hexanes/acetone 4:1) to yield the second batch of the title compound (9.3 g; 47%). Total yield: 16.3 g; 82%.

1H-NMR (400 MHz; DMSO-d6), δ (ppm): 7.40 (s, 1H); 8.25 (s, 1H).

MS (m/z) ES+: 253 (90, M+); 251 (80); 223 (100); 221 (80); 179 (60); 177 (50); 62 (85).

### c) 1-Bromo-4-chloro-5-cyclopropylmethoxy-2-nitrobenzene

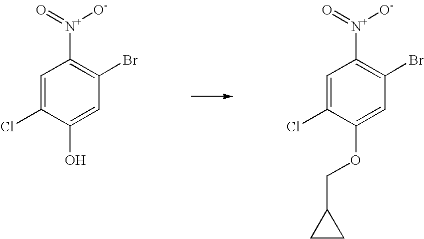

5-Bromo-2-chloro-4-nitrophenol (5.0 g; 19.8 mmol) in DMF (125 ml), Cs2CO3 (12.9 g; 39.6 mmol) and (bromomethyl)cyclopropane (2.3 ml; 23.8 mmol) is heated to 100° C. for 3.5 h. The reaction mixture is poured on 25% aq. NH4Cl and extracted with EtOAc three times. The combined organic phases are dried over Na2SO4, evaporated to dryness and purified via chromatography (SiO2; hexanes, hexanes/acetone 9:1) to yield the title compound as yellow crystals (3.8 g; 63%).

1H-NMR (400 MHz; DMSO-d6), δ (ppm): 0.38 (m, 2H); 0.62 (m, 2H); 1.26 (m, 1H); 4.08 (d, 2H); 7.57 (s, 1H); 8.25 (s, 1H).

MS (m/z) ES−: 306 (60; MH−); 304 (45); 242 (100).

### d) 2-Bromo-5-chloro-4-cyclopropylmethoxyphenylamine

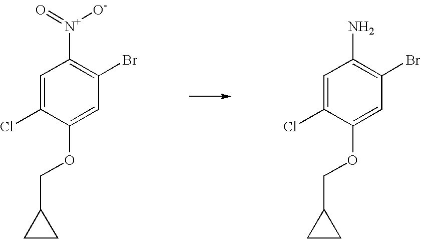

1-Bromo-4-chloro-5-cyclopropylmethoxy-2-nitrobenzene (3.8 g; 12.5 mmol) is dissolved in EtOH/HClconc (60 ml/20 ml) and heated with SnCl2 (11.85 g; 62.5 mmol) for 60 min at 45-50° C. The reaction mixture is poured on a saturated solution of Na2CO3 and extracted twice with TBME. The combined organic phases are dried over Na2SO4, evaporated to dryness and purified via chromatography (SiO2; hexanes/TBME 3:1) to yield the title compound as yellow oil (3.08 g; 89%).

1H-NMR (400 MHz; DMSO-d6), δ (ppm): 0.30 (m, 2H); 0.54 (m, 2H); 1.15 (m, 1H); 3.75 (d, 2H); 5.05 (s, 2H, NH2); 6.88 (s, 1H); 7.13 (s, 1H).

MS (m/z) ES+: 277 (45, M+); 275 (35); 223 (100); 221 (80); 78 (60); 55 (100).

### e) (E)-3-(2-Amino-4-chloro-5-cyclopropylmethoxyphenyl)-acrylic acid ethyl ester

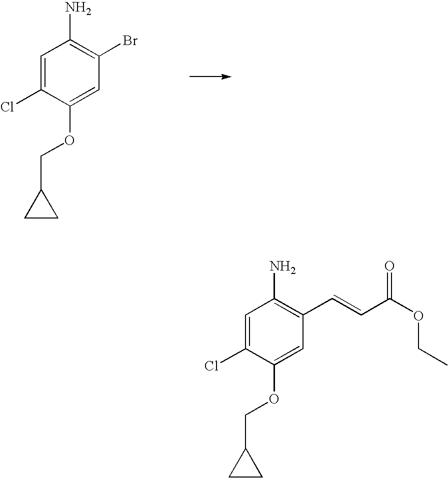

2-Bromo-5-chloro-4-cyclopropylmethoxyphenylamine (3.08 g; 11.1 mmol) and ethyl-(E)-3-tributylstannyl)-propenoate (5.18 g; 13.3 mmol) are dissolved in DMF (30 ml). PdCl2(PPh3)2 (0.16 g; 0.22 mmol) in DMF (6 ml) is added and the reaction mixture heated to 140° C. for 90 min. under argon. The reaction mixture is evaporated and purified via chromatography (SiO2; hexanes/TBME 3:2) to yield the desired compound which is recrystallised from hexanes/TBME to yield the title compound as yellow crystals (1.92 g; 58%).

1H-NMR (400 MHz; DMSO-d6), δ (ppm): 0.31 (m, 2H); 0.53 (m, 2H); 1.18 (m, 1H); 1.25 (t, 3H); 3.80 (d, 2H); 4.17 (q, 2H); 5.40 (s, 2H, NH2); 6.47 (d, 1H); 6.78 (s, 1H); 7.16 (s, 1H); 7.75 (d, 1H).

MS (m/z) ES+: 296 (100, MH+).

### f) (E)-3-(2-Amino-4-chloro-5-cyclopropylmethoxy-phenyl)-acrylic acid

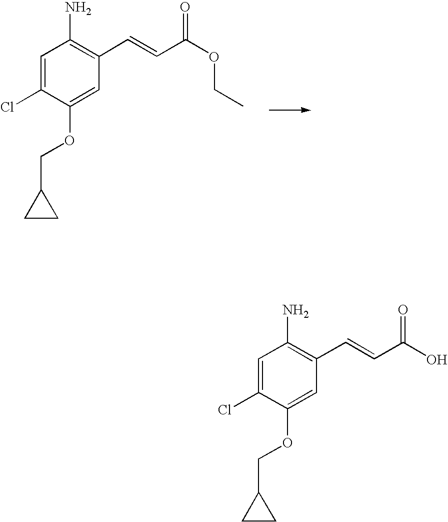

(E)-3-(2-Amino-4-chloro-5-cyclopropylmethoxyphenyl)-acrylic acid ethyl ester (4.9 g; 16.7 mmol) dissolved in EtOH (75 ml) and 2N NaOH (12.5 ml) is heated at 50° C. for 1.5 h. The reaction mixture is diluted with water, more 2N NaOH added and the mixture washed with TBME twice. The aq. phase is acidified by adding 2N HCl and extracted with TBME three times. The combined organic phases are dried over Na2SO4, filtered and evaporated to dryness to yield the title compound as a brownish solid (3.5 g; 78%).

1H-NMR (400 MHz; DMSO-d6), δ(ppm): 0.30 (m, 2H); 0.54 (m, 2H); 1.12-1.25 (m, 1H); 3.79 (d, 2H); 5.43 (bs, 2H); 6.37 (d, 1H); 6.79 (s, 1H); 7.12 (s, 1H); 7.69 (d, 1H); 12.15 (bs, 1H).

MS (m/z) ES−: 266 (MH−, 100); 167 (80).

### g) E)-3-(2-Amino-4-chloro-5-cyclopropylmethoxy-phenyl)-1-[7-(4-fluoro-benzyl)-3-oxa-7,9-diaza-bicyclo[3.3.1]non-9-yl]-propenone

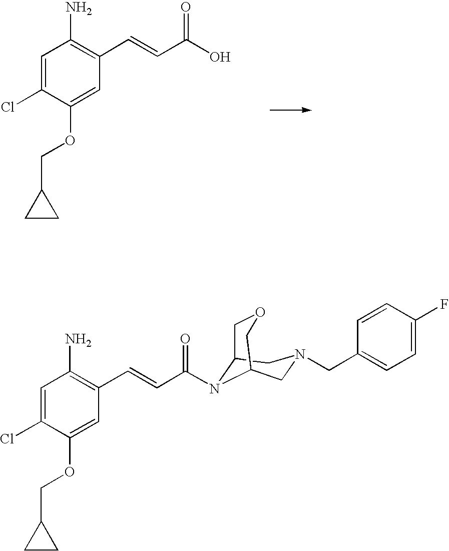

(E)-3-(2-Amino-4-chloro-5-cyclopropylmethoxy-phenyl)-acrylic acid (Example 128f) and 7-(4-fluoro-benzyl)-3-oxa-7,9-diaza-bicyclo[3.3.1]nonane (Example 102d) are coupled according to Example 23d to yield the title compound as yellow foam (319 mg; 86%).

1H-NMR (400 MHz; DMSO-d6), δ (ppm): 0.30 (m, 2H); 0.54 (m, 2H); 1.12-1.25 (m, 1H); 2.25 (d, 1H); 2.35 (d, 1H); 2.93 (d, 1H); 2.98 (d, 1H); 3.41 (dd, 2H); 3.60 (d, 1H); 3.65 (d, 1H); 3.78 (d, 2H); 3.87 (d, 2H); 4.35 (bs, 1H); 4.42 (bs, 1H); 5.27 (bs, 2H); 6.74 (d, 1H); 6.92 (d, 1H); 7.12 (s, 1H); 7.13 (d, 1H); 7.20 (s, 1H); 7.35 (d, 1H); 7.36 (s, 1H); 7.66 (d, 1H);

MS (m/z) ES+: 486 (MH+).

### h) 1-(5-Chloro-4-cyclopropylmethoxy-2-{(E)-3-[7-(4-fluoro-benzyl)-3-oxa-7,9-diaza-bicyclo[3.3.1]non-9-yl]-3-oxo-propenyl}-phenyl)-3-methyl-urea

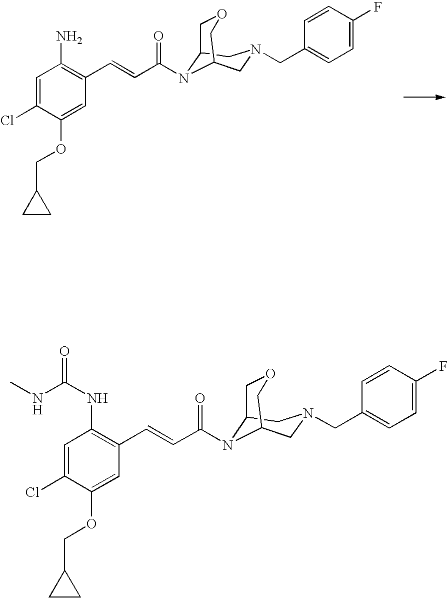

E)-3-(2-Amino-4-chloro-5-cyclopropylmethoxy-phenyl)-1-[7-(4-fluoro-benzyl)-3-oxa-7,9-diaza-bicyclo[3.3.1]non-9-yl]-propenone is treated according to Example 7 and yielded the title compound as off-white crystals (43%; 51%).

1H-NMR (400 MHz; DMSO-d6), δ (ppm): 0.35 (m, 2H); 0.60 (m, 2H); 1.20-1.30 (m, 1H); 2.26 (bd, 1H); 2.37 (bd, 1H); 2.62 (d, 3H); 2.95 (d, 1H); 3.00 (d, 1H); 3.42 (dd, 2H); 3.52 (d, 1H); 3.68 (d, 1H); 3.88 (t, 2H); 3.95 (d, 2H); 4.41 (d, 2H); 6.28 (q, 1H); 7.09-7.18 (m, 3H); 7.36 (m, 3H); 7.56 (s, 1H); 7.58 (d, 1H); 8.13 (s, 1H).

MS (m/z) ES+: 543 (MH+).

### Example 129

### N-(5-Chloro-4-cyclopropylmethoxy-2-{(E)-3-[7-(4-fluoro-benzyl)-3-oxa-7,9-diaza-bicyclo[3.3.1]non-9-yl]-3-oxo-propenyl}-phenyl)-acetamide

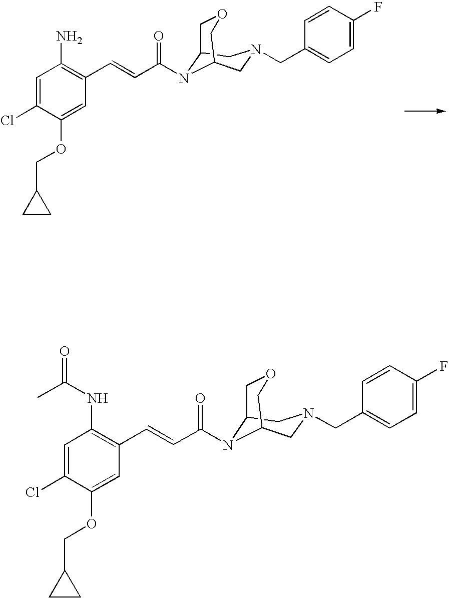

E)-3-(2-Amino-4-chloro-5-cyclopropylmethoxy-phenyl)-1-[7-(4-fluoro-benzyl)-3-oxa-7,9-diaza-bicyclo[3.3.1]non-9-yl]-propenone is treated according to Example 1f and yielded the title compound after chromatography (SiO2, acetone/hexanes 1:1) as colorless crystals (44 mg; 67%).

1H-NMR (400 MHz; DMSO-d6), δ (ppm): 0.36 (m, 2H); 0.60 (m, 2H); 1.20-1.30 (m, 1H); 2.04 (s, 3H); 2.27 (d, 1H); 2.37 (d, 1H); 2.95 (d, 1H); 3.00 (d, 1H); 2.93 (dd, 2H); 3.62 (d, 2H); 3.68 (d, 1H); 3.89 (t, 2H); 3.98 (d, 2H); 4.42 (d, 2H); 7.15 (t, 3H); 7.37 (dd, 2H); 7.42 (d, 1H); 7.51 (d, 1H); 9.70 (s, 1H).

MS (m/z) ES+: 528 (MH+).

### Example 130

### N-(5-Chloro-2-{(E)-3-[7-(4-fluoro-benzyl)-3-oxa-7,9-diaza-bicyclo[3.3.1]non-9-yl]-3-oxo-propenyl}-4-methyl-phenyl)-acetamide

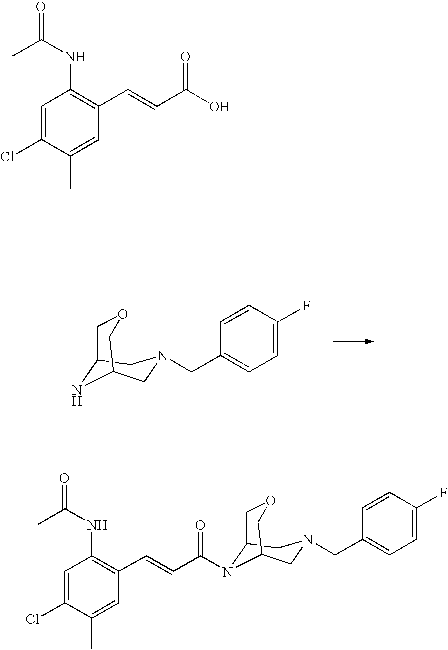

(E)-3-(2-Acetylamino-4-chloro-5-methyl-phenyl)-acrylic acid (obtained from Example 41c, which is acylated according to Example 23e and 7-(4-fluoro-benzyl)-3-oxa-7,9-diaza-bicyclo[3.3.1]nonane (Example 102d) are coupled according to Example 59e to yield the title compound after purification via chromatography (SiO2, acetone/hexanes 3/7 to 4/6) as pale yellow foam (105 mg; 80%).

1H-NMR (400 MHz; DMSO-d6), δ (ppm): 2.07 (s, 3H); 2.28 (d, 1H); 2.32 (s, 3H); 2.36 (d, 1H); 2.97 (bt, 2H); 3.42 (dd, 2H); 3.61 (d, 1H); 3.68 (d, 1H); 3.88 (m, 2H); 4.40 (bd, 2H); 7.10-7.18 (m, 3H); 7.37 (dd, 2H); 7.50 (s, 1H); 7.64 (d, 1H); 7.85 (s, 1H); 9.80 (s, 1H).

MS (m/z) ES+: 472.2 (MH+).

### Example 131

### N-(5-Chloro-2-{(E)-3-[9-(4-fluoro-benzyl)-3-oxa-7,9-diaza-bicyclo[3.3.1]non-7-yl]-3-oxo-propenyl}-4-methyl-phenyl)-acetamide

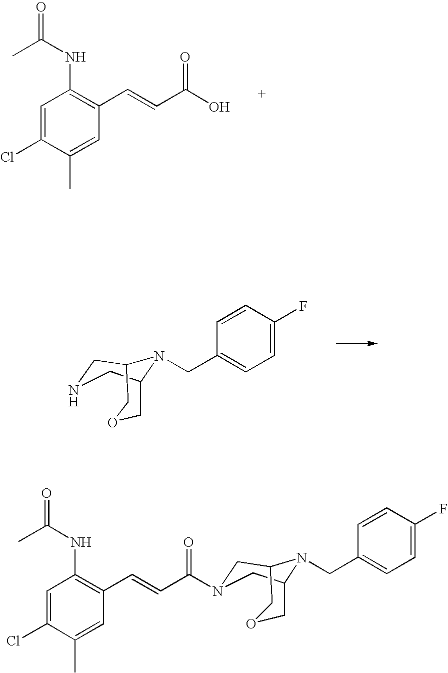

(E)-3-(2-Acetylamino-4-chloro-5-methyl-phenyl)-acrylic acid (obtained from Example 41c, which is acylated according to Example 23e) and 9-(4-fluoro-benzyl)-3-oxa-7,9-diaza-bicyclo[3.3.1]nonane (Example 103g or 103l) are coupled according to Example 59e to yield the title compound after purification via chromatography (SiO2, acetone/hexanes 4/6 to 1/1) as pale yellow foam (59 mg; 45%).

1H-NMR (400 MHz; DMSO-d6), δ (ppm): 2.06 (s, 3H); 2.33 (s, 3H); 2.62 (bs, 2H); 3.28 (m, 2H); 3.67 (d, 1H); 3.73 (bd, 1H); 3.82 (bs, 2H); 3.95 (s, 2H); 4.15 (d, 1H); 4.32 (d, 1H); 7.11-7.20 (m, 3H); 7.42 (dd, 2H); 7.49 (s, 1H); 7.57 (d, 1H); 7.85 (s, 1H); 9.78 (s, 1H).

MS (m/z) ES−: 470.2 (MH−).

### Example 132

### N-(5-Chloro-2-{(E)-3-[7-(4-fluoro-benzyl)-3-oxa-7,9-diaza-bicyclo[3.3.1]non-9-yl]-3-oxo-propenyl}-4-pyrazin-2-yl-phenyl)-acetamide

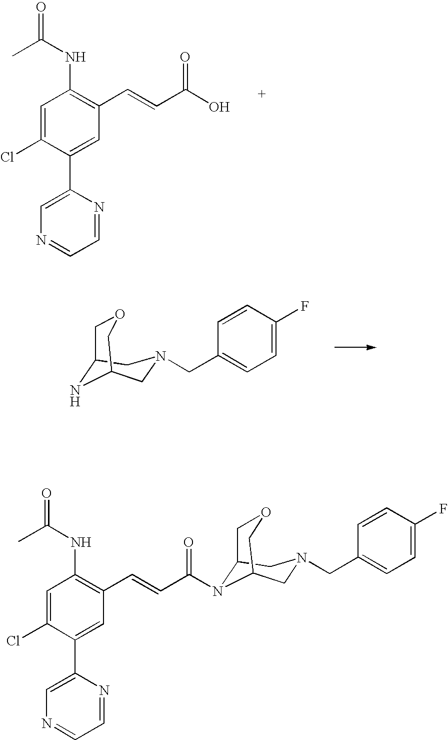

(E)-3-(2-Acetylamino-4-chloro-5-pyrazin-2-yl-phenyl)-acrylic acid (Example 55e) and 7-(4-fluoro-benzyl)-3-oxa-7,9-diaza-bicyclo[3.3.1]nonane (Example 102d) are coupled according to Example 55f and yielded the title compound after purification via chromatography (SiO2, acetone/TBME 20/80) as yellow crystals (77 mg; 70%).

1H-NMR (400 MHz; DMSO-d6), δ (ppm): 2.13 (s, 3H); 2.25 (d, 1H); 2.32 (d, 1H); 2.95 (d, 2H); 3.40 (dd, 2H); 3.62 (bt, 2H); 3.86 (bt, 2H); 4.43 (bd, 2H); 7.11 (t, 2H); 7.25 (d, 1H); 7.35 (dd, 2H); 7.75 (d, 1H); 7.29 (s, 1H); 8.10 (s, 1H); 8.68 (d, 1H); 8.78 (m, 1H); 8.92 (d, 1H); 10.03 (s, 1H).

MS (m/z) ES+: 536 (MH+).

### Example 133

### N-(5-Chloro-2-{(E)-3-[9-(4-fluoro-benzyl)-3-oxa-7,9-diaza-bicyclo[3.3.1]non-7-yl]-3-oxo-propenyl}-4-pyrazin-2-yl-phenyl)-acetamide

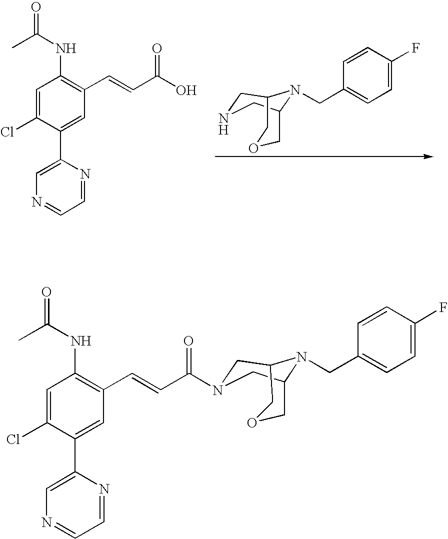

(E)-3-(2-Acetylamino-4-chloro-5-pyrazin-2-yl-phenyl)-acrylic acid (Example 55e) and 9-(4-Fluoro-benzyl)-3-oxa-7,9-diaza-bicyclo[3.3.1]nonane (Example 103g or 103l) are coupled according to Example 55f and yielded the title compound after purification via chromatography (SiO2, acetone/ethyl acetate 20/80) as yellow foam (76 mg; 69%).

1H-NMR (400 MHz; DMSO-d6), δ (ppm): 2.08 (s, 3H); 2.58 (bs, 1H); 2.53 (bs, 1H); 3.20-3.30 (m, 2H); 3.63-3.71 (m, 2H); 3.75-3.83 (m, 2H); 3.93 (s, 2H); 4.13 (d, 1H); 4.32 (d, 1H); 7.12 (t, 2H); 7.27 (d, 1H); 7.40 (m, 2H); 7.66 (d, 1H); 7.77 (s, 1H); 8.09 (s, 1H); 8.68 (d, 1H); 8.78 (m, 1H); 8.92 (s, 1H). 10.03 (bs, 1H).

MS (m/z) ES+: 536 (MH+).

### Example 134

### N-(5-Chloro-2-{(E)-3-[9-(4-fluoro-benzyl)-3-oxa-7,9-diaza-bicyclo[3.3.1]non-7-yl]-3-oxo-propenyl}-4-pyridin-2-yl-phenyl)-acetamide

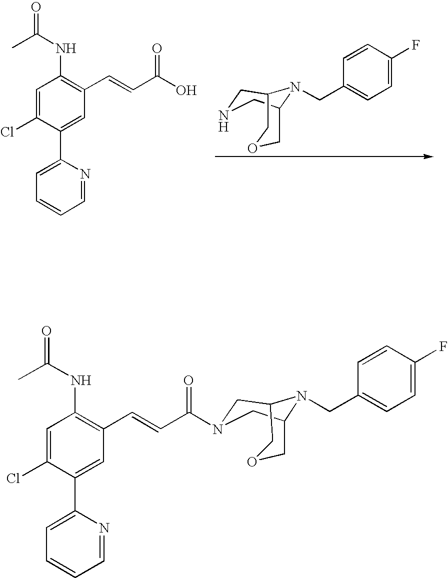

(E)-3-(2-Acetylamino-4-chloro-5-pyridin-2-yl-phenyl)-acrylic acid (obtained in analogy to Example 55e from 3-chloro-4-iodoaniline and 2-(tri-n-butylstannyl)pyridine) and 9-(4-fluoro-benzyl)-3-oxa-7,9-diaza-bicyclo[3.3.1]nonane (Example 103g or 103l) are coupled according to Example 55f and yielded the title compound after purification via chromatography (SiO2, acetone/hexanes 1/1) as colorless foam (86 mg; 73%).

1H-NMR (400 MHz; DMSO-d6), δ (ppm): 2.11 (s, 3H); 2.62 (bd, 2H); 3.23 (m, 2H); 3.69 (m, 2H); 3.78 (bd, 2H); 3.92 (s, 2H); 4.15 (d, 1H); 4.30 (d, 1H); 7.12 (t, 2H); 7.25 (d, 1H); 7.40 (m, 3H); 7.63 (m, 1H); 7.69 (s, 1H); 7.92 (m, 2H); 7.99 (s, 1H); 8.67 (d, 1H); 9.98 (s, 1H).

MS (m/z) ES+: 535 (MH+).

### Example 135

### N-(5-Chloro-2-{(E)-3-[7-(4-fluoro-benzyl)-3-oxa-7,9-diaza-bicyclo[3.3.1]non-9-yl]-3-oxo-propenyl}-4-pyridin-2-yl-phenyl)-acetamide

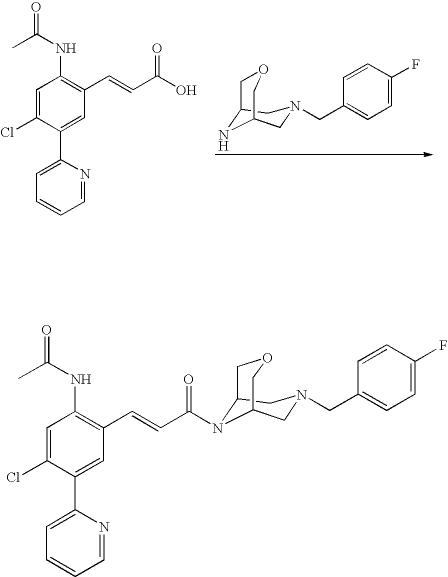

(E)-3-(2-Acetylamino-4-chloro-5-pyridin-2-yl-phenyl)-acrylic acid (obtained in analogy to Example 55e from 3-chloro-4-iodoaniline and 2-(tri-n-butylstannyl)pyridine) and 7-(4-Fluoro-benzyl)-3-oxa-7,9-diaza-bicyclo[3.3.1]nonane (Example 102d) are coupled according to Example 55f and yielded the title compound after purification via chromatography (SiO2, acetone/hexanes 1/1) as colorless crystals (112 mg; 68%).

1H-NMR (400 MHz; DMSO-d6), δ(ppm): 2.12 (s, 3H); 2.26 (bd, 1H); 2.32 (bd, 1H); 2.94 (bd, 2H); 3.40 (dd, 2H); 3.61 (bt, 2H); 3.83 (t, 2H); 4.41 (bd, 2H); 7.12 (t, 2H); 7.22 (d, 1H); 7.34 (dd, 2H); 7.41 (dd, 1H); 7.62 (dd, 1H); 7.72 (m, 1H); 7.76 (d, 1H); 7.90 (dt, 1H); 8.02 (s, 1H); 8.67 (bd, 1H); 9.97 (bs, 1H).

MS (m/z) ES+: 535 (MH+).

### Example 136

### N-(5-Chloro-2-{(E)-3-[(1S,3R,5R)-3-(4-fluoro-phenylamino)-8-aza-bicyclo[3.2.1]oct-8yl]-3-oxo-propenyl}-4-pyrazin-2-yl-phenyl)-acetamide

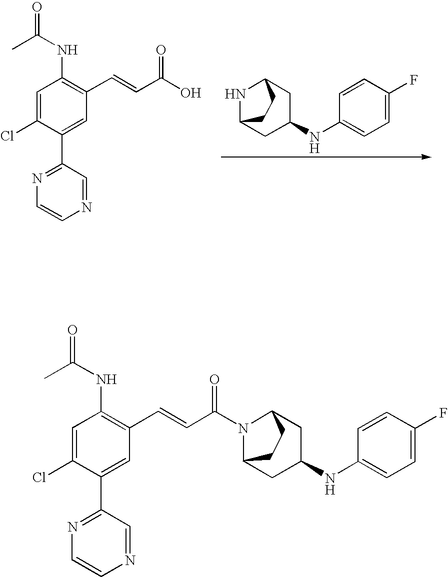

(E)-3-(2-Acetylamino-4-chloro-5-pyrazin-2-yl-phenyl)-acrylic acid (Example 55e) and (1S,3R,5R)-8-Aza-bicyclo[3.2.1]oct-3-yl-(4-fluoro-phenyl)-amine (described in WO 2004009588) are coupled according to Example 55f and yielded the title compound after chromatography (SiO2, ethyl acetate as eluent) as colorless foam (141 mg; 75%).

1H-NMR (400 MHz; DMSO-d6), δ (ppm): 1.74-1.86 (bt, 3H); 1.90-2.03 (m, 2H); 2.09-2.23 (m, 3H); 2.15 (s, 3H); 3.46 (bs, 1H); 4.53 (bs, 1H); 4.70 (bs, 1H); 5.58 (d, 1H); 6.50 (dd, 2H); 6.90 (t, 2H); 7.18 (d, 1H); 7.72 (d, 1H); 7.79 (s, 1H); 8.13 (s, 1H); 8.70 (d, 1H); 8.78 (dd, 1H); 8.94 (d, 1H); 10.03 (bs, 1H).

MS (m/z) ES+: 520 (MH+).

### Example 137

### N-(5-Chloro-2-{(E)-3-[(1S,3R,5R)-3-(4-fluoro-Phenylamino)-8-aza-bicyclo[3.2.1]oct-8-yl]-3-oxo-propenyl}-4-pyridin-2-yl-phenyl-acetamide

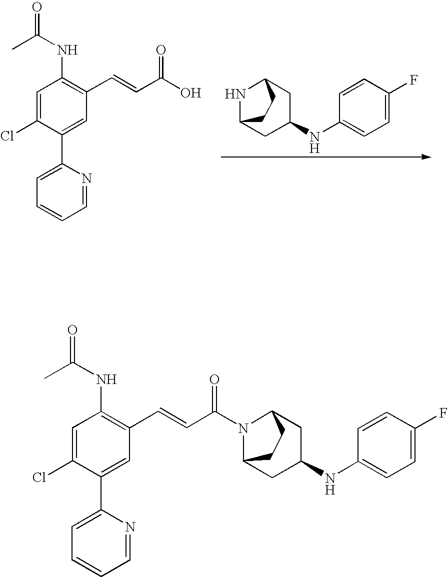

(E)-3-(2-Acetylamino-4-chloro-5-pyridin-2-yl-phenyl)-acrylic acid (obtained in analogy to Example 55e from 3-chloro-4-iodoaniline and 2-(tri-n-butylstannyl)pyridine) and (1S,3R,5R)-8-aza-bicyclo[3.2.1]oct-3-yl-(4-fluoro-phenyl)-amine (described in WO 2004009588) are coupled according to Example 55f and yielded the title compound after chromatography (SiO2, acetone/hexanes 2/3) as brownish foam (59 mg; 36%).

1H-NMR (400 MHz; DMSO-d6), δ (ppm): 1.74-1.86 (m, 3H); 1.90-2.03 (m, 2H); 2.09 (s, 3H); 2.08-2.23 (m, 3H); 3.47 (bs, 1H); 4.53 (bs, 1H); 4.70 (bs, 1H); 5.59 (bs, 1H); 6.50 (dd, 2H); 6.98 (t, 2H); 7.13 (d, 1H); 7.42 (dd, 1H); 7.65 (dd, 1H); 7.70 (d, 1H); 7.71 (s, 1H); 7.91 (dt, 1H); 8.04 (s, 1H); 8.70 (bd, 1H); 9.98 (bs, 1H).

MS (m/z) ES+: 519 (MH+).

### Example 138

### (5-Chloro-2-{(E)-3-[(1R,3R,5S)-3-(4-fluoro-phenylamino)-8-aza-bicyclo[3.2.1]oct-8-yl]-3-oxo-propenyl}-4-methoxy-phenyl)-urea

### a) (E)-3-(2-Amino-4-chloro-5-methoxy-phenyl)-1-[(1R,3R,5S)-3-(4-fluoro-phenylamino)-8-aza-bicyclo[3.2.1]oct-8-yl]-propenone

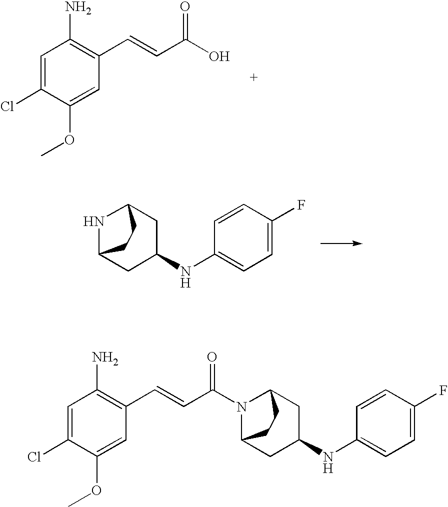

3-(2-Amino-4-chloro-5-methoxy-phenyl)-acrylic acid (Example 23c) and (1S,3R,5R)-8-aza-bicyclo[3.2.1]oct-3-yl-(4-fluoro-phenyl)-amine (described in WO 2004009588) are coupled according to Example 23d to yield the title compound as a yellow solid (158 mg; 81%).

1H-NMR (400 MHz; DMSO-d6), δ (ppm):

MS (m/z) ES+: 424 (MH+).

### b) (5-Chloro-2-{(E)-3-[(1R,3R,5S)-3-(4-fluoro-phenylamino)-8-aza-bicyclo[3.2.1]oct-8-yl]-3-oxo-propenyl}-4-methoxy-phenyl)-urea

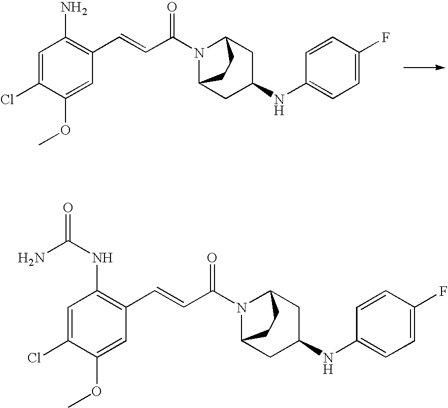

(E)-3-(2-Amino-4-chloro-5-methoxy-phenyl)-1-[(1R,3R,5S)-3-(4-fluoro-phenylamino)-8-aza-bicyclo[3.2.1]oct-8-yl]-propenone is treated according to Example 4 to yield the title compound after chromatography (SiO2, TBME/MeOH/NH3conc 95/5/0.6) as colorless crystals (22 mg; 52%).

1H-NMR (400 MHz; DMSO-d6), δ (ppm): 1.75-1.91 (m, 3H); 1.95-2.08 (m, 2H); 2.07-2.30 (m, 3H); 3.48 (bs, 1H); 3.91 (s, 3H); 4.53 (bs, 1H); 4.72 (bs, 1H); 5.61 (d, 1H); 6.03 (s, 2H); 6.52 (dd, 2H); 6.91 (t, 2H); 7.10 (d, 1H); 7.40 (s, 1H); 7.68 (d, 1H); 7.71 (s, 1H); 8.21 (s, 1H).

MS (m/z) ES+: 473 (MH+, 100); 430 (45); 412 (40); 210 (70); 152 (50).

### Example 139

### N-(5-Chloro-2-{(E)-3-[(1R,3R,5S)-3-(4-fluoro-phenylamino)-8-aza-bicyclo[3.2.1]oct-8-yl]-3-oxo-propenyl}-4-methoxy-phenyl)-acetamide

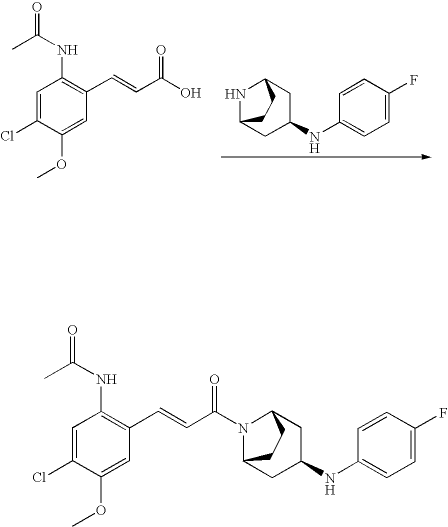

(E)-3-(2-Acetylamino-4-chloro-5-methoxy-phenyl)-acrylic acid (Example 23e) and (1S,3R,5R)-8-aza-bicyclo[3.2.1]oct-3-yl-(4-fluoro-phenyl)-amine (described in WO 2004009588) are coupled according to Example 59e and purified via chromatography (SiO2, acetone/TBME 1/20) to yield the desired product as colorless crystals (93 mg; 53%).

1H-NMR (400 MHz; DMSO-d6), δ (ppm): 1.77-1.91 (m, 3H); 1.97-2.03 (m, 2H); 2.05 (s, 3H); 2.10-2.28 (m, 3H); 3.48 (bs, 1H); 3.94 (s, 3H); 4.53 (bs, 1H); 4.72 (bs, 1H); 5.60 (bs, 1H); 6.51 (dd, 2H); 6.90 (t, 2H); 7.12 (d, 1H); 7.43 (s, 1H); 7.48 (s, 1H); 7.61 (d, 1H). 9.72 (bs, 1H).

MS (m/z) ES+: 472 (MH+).

### Example 140

### (5-Chloro-2-{(E)-3-[(1R,3R,5S)-3-(4-fluoro-phenylamino)-8-aza-bicyclo[3.2.1]oct-8-yl]-3-oxo-propenyl}-4-trifluoromethoxy-phenyl)-urea

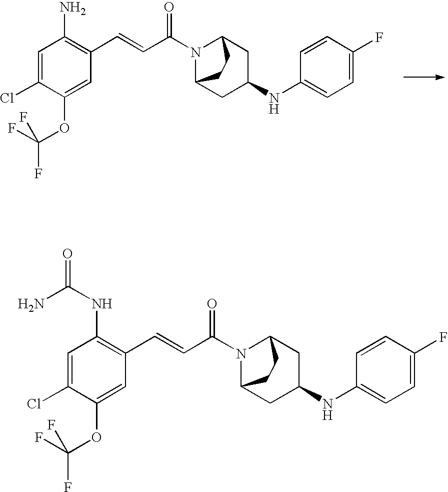

(E)-3-(2-Amino-4-chloro-5-trifluoromethoxy-phenyl)-1-[(1R,3R,5S)-3-(4-fluoro-phenylamino)-8-aza-bicyclo[3.2.1]oct-8-yl]-propenone [obtained from (E)-3-(2-amino-4-chloro-5-trifluoromethoxy-phenyl)-acrylic acid (Example 35c) and (1S,3R,5R)-8-aza-bicyclo[3.2.1]oct-3-yl-(4-fluoro-phenyl)-amine, which are coupled according to Example 23d] is treated according to Example 4 to yield the title compound after chromatography (SiO2, TBME/MeOH/NH3conc 95/5/50.6) and crystallization from TBME/hexanes (22 mg; 28%).

1H-NMR (400 MHz; DMSO-d6), δ (ppm): 1.77-1.92 (m, 3H); 1.92-2.05 (m, 2H); 2.05-2.28 (m, 3H); 3.49 (bs, 1H); 4.53 (bs, 1H); 4.72 (bs, 1H); 5.60 (bs, 1H); 6.30 (bs, 2H); 6.51 (dd, 2H); 6.89 (t, 2H); 7.16 (d, 1H); 7.65 (d, 1H); 7.97 (s, 1H); 8.13 (s, 1H); 8.53 (s, 1H).

MS (m/z) ES+: 527 (MH+).

### Example 141

### N-(5-Chloro-2-{(E)-3-[(1R,3R,5S)-3-(4-fluoro-phenylamino)-8-aza-bicyclo[3.2.1]oct-8-yl]-3-oxo-propenyl}-4-trifluoromethoxy-phenyl)-acetamide

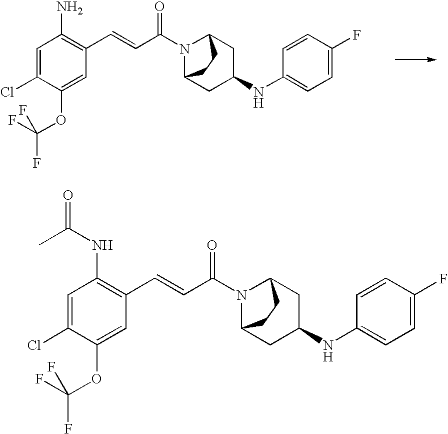

The reaction is performed in analogy to Example 1f and the title product purified via chromatography (SiO2, TBME/MeOH/NH3conc 98/2/0.2) to yield the desired product as colorless crystals (40 mg; 62%).

1H-NMR (400 MHz; DMSO-d6), δ (ppm): 1.76-1.90 (m, 3H); 1.93-2.07 (m, 2H); 2.08-2.28 (m, 3H); 2.11 (s, 3H); 3.47 (bs, 1H); 4.53 (bs, 1H); 4.73 (bs, 1H); 5.60 (bs, 1H); 6.52 (dd, 2H); 6.90 (t, 2H); 7.22 (d, 1H); 7.63 (d, 1H); 7.80 (s, 1H); 8.11 (bs, 1H); 10.02 (bs, 1H).

MS (m/z) ES+: 526 (MH+).

### Example 142

### 5-Chloro-2-{(E)-3-[(1R,3R,5S)-3-(4-fluoro-phenylamino)-8-aza-bicyclo[3.2.1]oct-8-yl]-3-oxo-propenyl}-4-methoxy-N,N-dimethyl-benzenesulfonamide

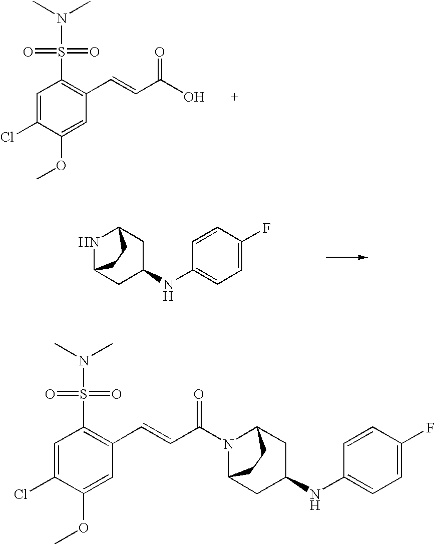

(E)-3-(4-Chloro-2-dimethylsulfamoyl-5-methoxy-phenyl)-acrylic acid (Example 30d) and (1S,3R,5R)-8-aza-bicyclo[3.2.1]oct-3-yl-(4-fluoro-phenyl)-amine (described in WO 2004009588) are coupled according to Example 30e to yield the title compound after chromatography (SiO2, TBME/MeOH/NH3conc 96/4/0.2) as colorless crystals (50 mg; 61%).

1H-NMR (400 MHz; DMSO-d6), δ (ppm): 1.75-1.90 (m, 3H); 1.96-2.05 (m, 2H); 2.08-2.28 (m, 3H); 2.68 (s, 6H); 3.51 (bs, 1H); 4.06 (s, 3H); 4.52 bs, 1H); 4.70 (bs, 1H); 5.60 (d, 1H); 6.52 (d, 2H); 6.90 (t, 2H); 7.17 (d, 1H); 7.58 (s, 1H); 7.82 (s, 1H); 8.23 (d, 1H);

MS (m/z) ES+: 522 (MH+).

### Example 143

### N-(5-Chloro-2-{(E)-3-[(1S,5R,8S)-8-(4-fluoro-phenylamino)-3-aza-bicyclo[3.2.1]oct-3-yl]-3-oxo-propenyl}-4-methoxy-phenyl)-acetamide

### a) ((1S,5R,8S)-3-Benzyl-3-aza-bicyclo[3.2.1]oct-8-yl)-(4-fluoro-phenyl)-amine

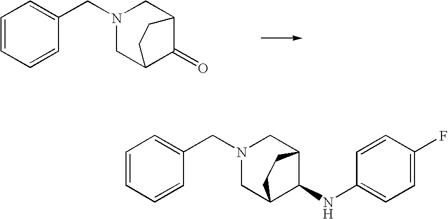

3-Benzyl-3-aza-bicyclo[3.2.1]octan-8-one (J. Med. Chem. (1994), 37, 2831) (1.00 g; 4.64 mmol), 4-fluoroaniline (464 mg; 4.18 mmol) and NaBH(OAc)3 (1.38 g; 6.50 mmol) in CH2Cl2 (10 ml) and HOAc (0.345 ml; 6.03 mmol) are kept at room temp. for 4 days. 2N HCl is added to the reaction mixture, which is washed with CH2Cl2 twice. The aq. phase is poured on saturated Na2CO3 solution and extracted with CH2Cl2 three times. The combined organic phases are dried over Na2SO4, filtered and evaporated to dryness and the residue purified via chromatography (SiO2, EtOAc/hexanes 20/80) to yield the title compound as colorless glass (431 mg; 30%).

1H-NMR (400 MHz; DMSO-d6), δ (ppm): 1.69-1.78 (m, 4H); 2.13 (bs, 2H); 2.35 (dd, 2H); 2.54 (d, 2H); 3.23 (dd, 1H); 3.48 (s, 2H); 5.53 (d, 1H); 6.63 (dd, 2H); 6.87 (t, 2H); 7.17 (m, 1H); 7.28-7.35 (m, 4H).

MS (m/z) ES+: 311 (MH+).

### b) (1S,5R,8S)-3-Aza-bicyclo[3.2.1]oct-8-yl-(4-fluoro-phenyl)-amine

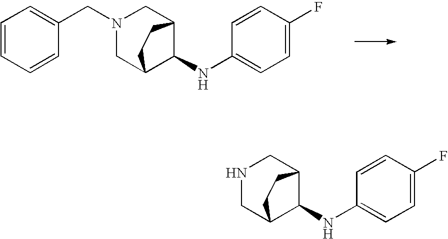

((1S,5R,8S)-3-Benzyl-3-aza-bicyclo[3.2.1]oct-8-yl)-(4-fluoro-phenyl)-amine (425 mg; 1.37 mmol) and ammoniumformate (428 mg; 6.78 mmol) and Pd/C (10%; 43 mg) are refluxed in MeOH (20 ml) for 2 h. The mixture is filtered, evaporated, saturated solution of NaHCO3 added and extracted with CH2Cl2 three times. The combined organic phases are dried over Na2SO4, filtered and evaporated to dryness to deliver the target compound as brownish oil (240 mg; 80%).

1H-NMR (400 MHz; DMSO-d6), δ (ppm): 1.65-1.82 (m, 4H); 2.08 (bs, 2H); 2.42 (dd, 2H); 3.10 (d, 2H); 3.31 (dd, 1H); 5.48 (d, 1H); 6.62 (dd, 2H); 6.88 (t, 2H).

MS (m/z) ES+: 221 (MH+).

### c) N-(5-Chloro-2-{(E)-3-[(1S,5R,8S)-8-(4-fluoro-phenylamino)-3-aza-bicyclo[3.2.1]oct-3-yl]-3-oxo-propenyl}-4-methoxy-phenyl)-acetamide

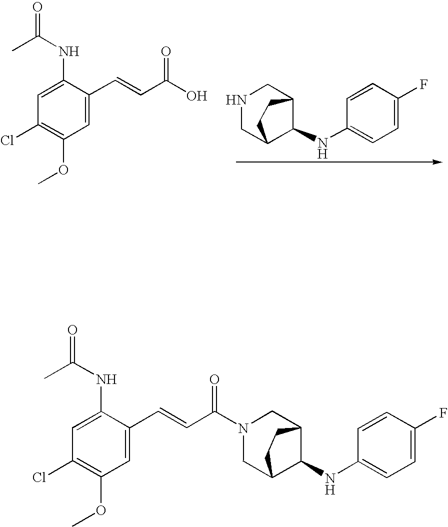

(1S,5R,8S)-3-Aza-bicyclo[3.2.1]oct-8-yl-(4-fluoro-phenyl)-amine and (E)-3-(2-acetylamino-4-chloro-5-methoxy-phenyl)-acrylic acid (Example 23e) are coupled according to Example 59e and yielded the title compound after chromatography (SiO2, acetone/hexanes 30/60) as brownish crystals (71 mg; 67%).

1H-NMR (400 MHz; DMSO-d6), δ(ppm): 1.40-1.47 (m, 1H); 1.52-1.58 (m, 1H); 1.81 (bs, 2H); 2.03 (s, 3H); 2.30 (bs, 2H); 3.15 (d, 1H); 3.40 (dd, 1H); 3.55 (d, 1H); 3.83 (bd, 1H); 3.93 (s, 3H); 4.05 (bd, 1H); 5.82 (d, 1H); 6.70 (dd, 2H); 6.91 (t, 2H); 7.23 (d, 1H); 7.41 (d, 1H); 7.46 (s, 1H); 7.54 (d, 1H); 9.70 (s, 1H).

MS (m/z) ES+: 472 (MH+).

### Example 144

### (5-Chloro-2-{(E)-3-[(1S,5R,9S)-9-(4-fluoro-Phenylamino)-3-oxa-7-aza-bicyclo[3.3.1]non-7-yl]-3-oxo-propenyl}-4-trifluoromethoxy-phenyl)-urea

### a) ((1S,5R,9S)-7-Benzyl-3-oxa-7-aza-bicyclo[3.3.1]non-9-yl)-(4-fluoro-phenyl)-amine (Z) and ((1S,5R,9R)-7-Benzyl-3-oxa-7-aza-bicyclo[3.3.1]non-9-yl)-(4-fluoro-phenyl)-amine (E)

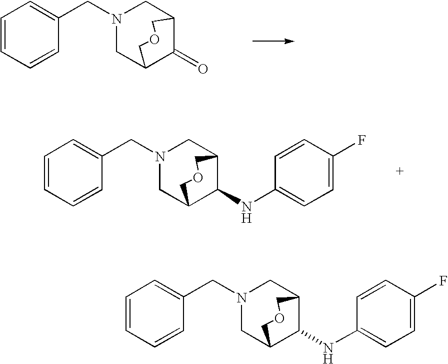

7-Benzyl-3-oxa-7-aza-bicyclo[3.3.1]nonan-9-one (J. Org. Chem. 1981, 46, 3196) is reductively aminated with 4-fluoroaniline as described in Example 143a. Both isomers are separated by chromatography (SiO2, acetone/hexanes 30/60). ((1S,5R,9S)-7-Benzyl-3-oxa-7-aza-bicyclo[3.3.1]non-9-yl)-(4-fluoro-phenyl)-amine (Z) is eluted first and is obtained as colorless crystals (390 mg; 25%) followed by the second isomer ((1S,5R,9R)-7-benzyl-3-oxa-7-aza-bicyclo[3.3.1]non-9-yl)-(4-fluoro-phenyl)-amine (E), also obtained as colorless crystals (106 mg; 7%).

### ((1S,5R,9S)-7-Benzyl-3-oxa-7-aza-bicyclo[3.3.1]non-9-yl)-(4-fluoro-phenyl)-amine (Z)

1H-NMR (400 MHz; DMSO-d6), δ (ppm): 1.73 (s, 2H); 2.47 (m, 3H); 3.03 (d, 2H); 3.49 (s, 2H); 3.60 (d, 2H); 3.95 (d, 2H); 5.67 (bd, 1H); 6.59 (m, 2H); 6.89 (t, 2H); 7.21 (m, 1H); 7.32 (m, 4H).

MS (m/z) ES+: 327 (MH+).

### ((1S,5R,9R)-7-Benzyl-3-oxa-7-aza-bicyclo[3.3.1]non-9-yl)-(4-fluoro-phenyl)-amine (E)

1H-NMR (400 MHz; DMSO-d6), δ (ppm): 1.81 (s, 2H); 2.50 (s, 2H); 2.65 (s, 2H); 3.43 (s, 2H); 3.47 (m, 1H); 3.77 (d, 2H); 3.87 (d, 2H); 5.47(d, 1H); 6.64 (m, 2H); 6.89 (t, 2H); 7.20 (m, 1H); 7.32 (m, 4H).

MS (m/z) ES+: 327 (MH+).

### b) (4-Fluoro-phenyl)-(1S,5R,9S)-3-oxa-7-aza-bicyclo[3.3.1]non-9-yl-amine

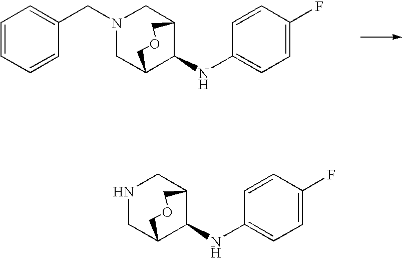

((1S,5R,9S)-7-Benzyl-3-oxa-7-aza-bicyclo[3.3.1]non-9-yl)-(4-fluoro-phenyl)-amine (Z) (300 mg; 0.92 mmol), ammoniumformate (290 mg; 4.6 mmol), Pd/C (10%; 30 mg) in MeOH (10 ml) are refluxed for 1.5 h, filtered, evaporated, a saturated solution of NaHCO3 added and the aq. phase extracted with TBME three times. The combined organic phases are dried over Na2SO4, filtered and evaporated to dryness to deliver the target compound as colorless crystals (216 mg; 99%).

1H-NMR (400 MHz; DMSO-d6), δ (ppm): 1.50 (bs, 2H); 2.05 (bs, 1H); 2.93 (d, 2H); 3.18 (d, 2H); 3.43 (m, 1H); 3.71 (d, 2H); 4.05 (d, 2H); 5.71 (d, 1H); 6.61 (dd, 2H); 6.88 (t, 2H).

MS (m/z) ES+: 237 (MH+).

### c) (E)-3-(2-Amino-4-chloro-5-trifluoromethoxy-phenyl)-[1-(1S,5R,9S)-9-(4-fluoro-phenylamino)-3-oxa-7-aza-bicyclo[3.3.1]non-7-yl]-propenone

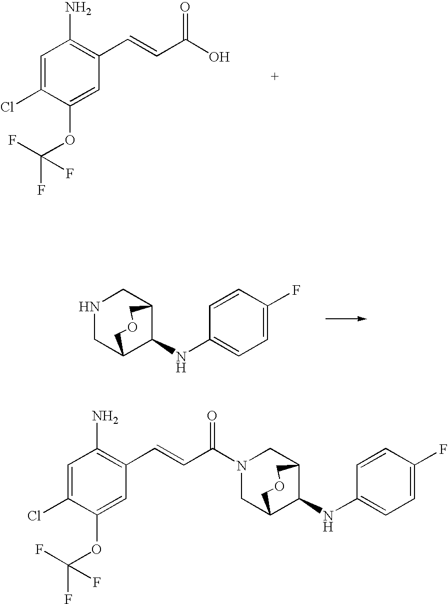

(E)-3-(2-Amino-4-chloro-5-trifluoromethoxy-phenyl)-acrylic acid (Example 35c) and (4-Fluoro-phenyl)-(1S,5R,9S)-3-oxa-7-aza-bicyclo[3.3.1]non-9-yl-amine are coupled according to Example 23d. The product is purified via chromatography (SiO2, TBME/MeOH/NH3conc 99/1/0.1 to 96/4/0.6) to yield the desired compound as yellow foam (321 mg; 95%).

1H-NMR (400 MHz; DMSO-d6), δ (ppm): 1.80 (bd, 2H); 3.11 (bd, 1H); 3.51-3.62 (m, 3H); 3.74 (d, 1H); 3.96 (bt, 2H); 4.51 (d, 1H); 4.75 (d, 1H); 5.81 (d, 1H); 5.92 (s, 2H); 6.68 (m, 2H); 6.85 (s, 1H); 6.92 (t, 2H); 7.17 (d, 1H); 7.53 (d, 1H); 7.70 (s, 1H).

MS (m/z) ES+: 500 (MH+).

### d) (5-Chloro-2-{(E)-3-[(1S,5R,9S)-9-(4-fluoro-phenylamino)-3-oxa-7-aza-bicyclo[3.3.1]non-7-yl]-3-oxo-propenyl}-4-trifluoromethoxy-phenyl)-urea

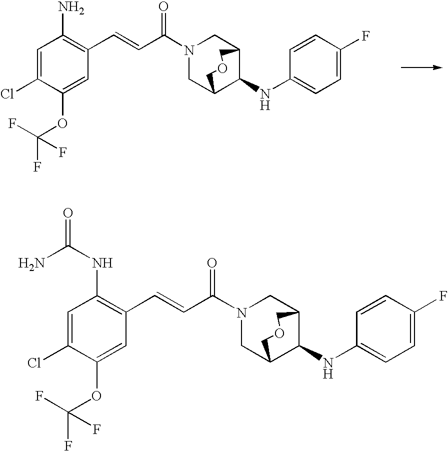

The reaction is performed in analogy to Example 4 and the product purified via chromatography (SiO2, TBME/MeOH/NH3conc 95/5/0.6) to yield the desired product as yellow foam, which is crystallized from TBME/hexanes (39 mg; 36%).

1H-NMR (400 MHz; DMSO-d6), δ (ppm): 1.81 (bd, 2H); 3.13 (bd, 1H); 3.53-3.66 (m, 3H); 3.75 (d, 1H); 3.97 (t, 2H); 4.53 (d, 1H); 4.78 (d, 1H); 5.82 (d, 1H); 6.31 (bs, 2H); 6.68 (m, 2H); 6.92 (t, 2H); 7.34 (d, 1H); 7.59 (d, 1H); 7.95 (s, 1H); 8.17 (s, 1H); 8.50 (s, 1H).

MS (m/z) ES+: 543 (MH+).

### Example 145

### N-(5-Chloro-2-{(E)-3-[(1S,5R,9S)-9-(4-fluoro-phenylamino)-3-oxa-7-aza-bicyclo[3.3.1]non-7-yl]-3-oxo-propenyl}-4-trifluoromethoxy-phenyl)-acetamide

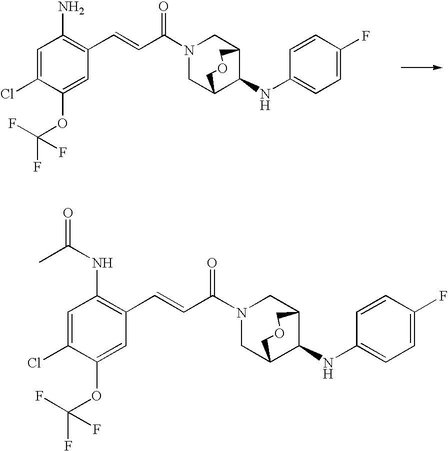

The reaction is performed in analogy to Example 1f and the product purified via chromatography (SiO2, TBME/MeOH/NH3conc 96/4/0.5) to yield the desired product as yellow foam, which is crystallized from TBME (80 mg; 75%).

1H-NMR (400 MHz; DMSO-d6), δ (ppm): 1.80 (bd, 2H); 2.10 (s, 3H); 3.12 (bd, 1H); 3.53-3.64 (m, 3H); 3.75 (d, 1H); 3.95 (d, 1H); 4.00 (d, 1H); 4.53 (d, 1H); 4.78 (d, 1H); 5.81 (d, 1H); 6.68 (m, 2H); 6.90 (t, 2H); 7.38 (d, 1H); 7.55 (d, 1H); 7.78 (s, 1H); 8.10 (s, 1H); 10.10 (s, 1H).

MS (m/z) ES+: 542 (MH+).

### Example 146

### N-(5-Chloro-2-{(E)-3-[(1S,5R,9R)-9-(4-fluoro-phenylamino)-3-oxa-7-aza-bicyclo[3.3.1]non-7-yl]-3-oxo-propenyl}-4-trifluoromethoxy-phenyl)-acetamide

### a) (4-Fluoro-phenyl)-(1S,5R,9R)-3-oxa-7-aza-bicyclo[3.3.1]non-9-yl-amine

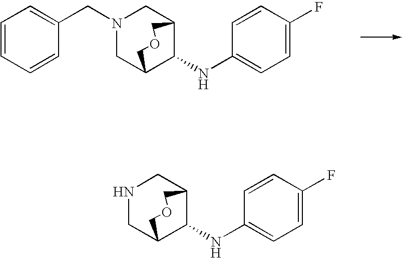

((1S,5R,9R)-7-Benzyl-3-oxa-7-aza-bicyclo[3.3.1]non-9-yl)-(4-fluoro-phenyl)-amine is debenzylated in analogy to Example 144b to deliver the target compound as colorless crystals (65 mg; 94%).

1H-NMR (400 MHz; DMSO-d6), δ (ppm): 1.50 (bs, 2H); 2.05 (bs, 1H); 2.81 (bd, 2H); 3.13 (bd, 2H); 3.62 (m, 1H); 3.86 (bd, 2H); 4.05 (bd, 2H); 5.66 (d, 1H); 6.65 (m, 2H); 6.88 (m, 2H).

MS (m/z) ES+: 237 (MH+).

### b) (E)-3-(2-Amino-4-chloro-5-trifluoromethoxy-phenyl)-[1-(1S,5R,9R)-9-(4-fluorophenylamino)-3-oxa-7-aza-bicyclo[3.3.1]non-7-yl]-propenone

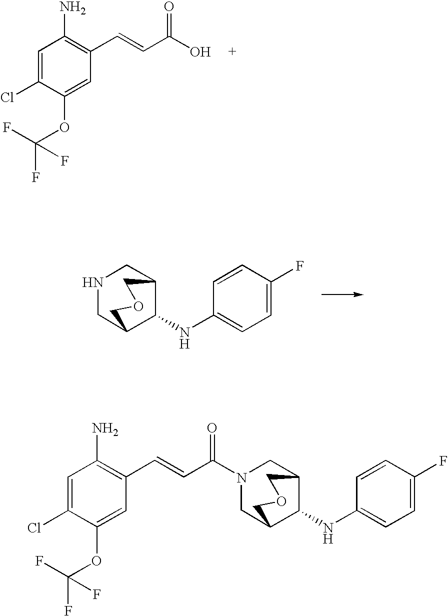

The coupling reaction is performed in analogy to Example 144c to deliver the target compound as yellow crystals (102 mg; 81%).

1H-NMR (400 MHz; DMSO-d6), δ (ppm): 1.87 (bs, 2H); 3.23 (bd, 1H); 3.62 (bd, 2H); 3.72 (bt, 2H); 3.87 (d, 1H); 4.01 (d, 1H); 4.18 (d, 1H); 4.41 (d, 1H); 5.70 (d, 1H); 5.90 (bs, 2H); 6.69 (dd, 2H); 6.84 (s, 1H); 6.91 (t, 2H); 7.13 (d, 1H); 7.50 (d, 1H); 7.67 (s, 1H).

MS (m/z) ES+: 500 (MH+).

### c) N-(5-Chloro-2-{(E)-3-[(1S,5R,9R)-9-(4-fluoro-phenylamino)-3-oxa-7-aza-bicyclo[3.3.1]non-7-yl]-3-oxo-propenyl}-4-trifluoromethoxy-phenyl)-acetamide

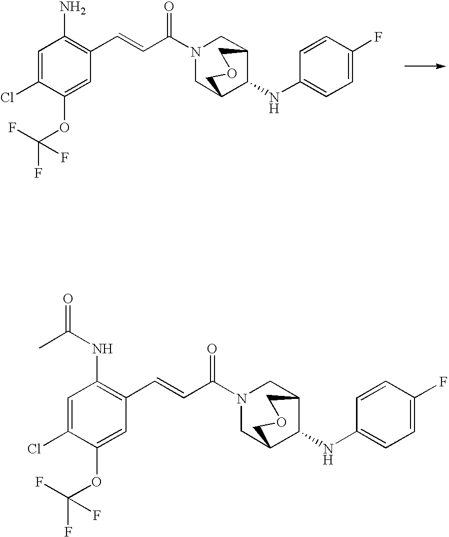

The coupling reaction is performed in analogy to Example if to deliver the target compound as colorless crystals (34 mg; 80%).

1H-NMR (400 MHz; DMSO-d6), δ (ppm): 1.90 (bs, 2H); 2.10 (s, 3H); 3.25 (d, 1H); 3.60-3.78 (m, 4H); 3.90 (bd, 1H); 4.04 (bd, 1H); 4.23 (bd, 1H); 4.44 (bd, 1H); 5.71 (bd, 1H); 6.71 (dd, 2H); 6.93 (t, 2H); 7.33 (d, 1H); 7.54 (d, 1H); 7.78 (s, 1H); 8.07 (s, 1H); 10.00 (s, 1H).

MS (m/z) ES+:

### Example 147

### N-(5-Chloro-2-{(E)-3-[(1S,5R,9R)-9-(4-fluoro-phenylamino)-3-oxa-7-aza-bicyclo[3.3.1]non-7-yl]-3-oxo-propenyl}-4-methoxy-phenyl)-acetamide

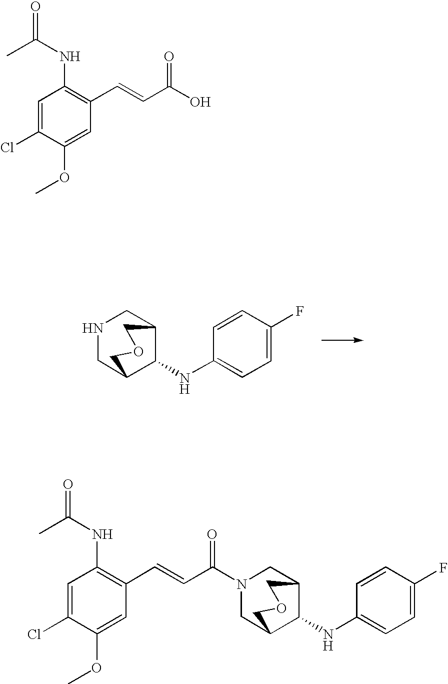

(E)-3-(2-Acetylamino-4-chloro-5-methoxy-phenyl)-acrylic acid (Example 23e) and (4-Fluoro-phenyl)-(1S,5R,9R)-3-oxa-7-aza-bicyclo[3.3.1]non-9-yl-amine are coupled according to conditions described in Example 59e. The target compound is obtained as colorless crystals (37 mg; 45%).

1H-NMR (400 MHz; DMSO-d6), δ (ppm): 1.90 (bs, 2H); 2.05 (s, 3H); 3.25 (bd, 1H); 3.60-3.78 (m, 4H); 3.97 (d, 1H); 3.90 (s, 3H); 4.03 (d, 1H); 4.20 (d, 1H); 4.43 (d, 1H); 5.71 (d, 1H); 6.70 (dd, 2H); 6.92 (t, 2H); 7.24 (d, 1H); 7.40 (s, 1H); 7.46 (s, 1H); 7.51 (d, 1H); 9.70 (s, 1H).

MS (m/z) ES+: 488 (MH+).

### Example 148

### N-(5-Chloro-2-{(E)-3-[(1S,5R,9S)-9-(4-fluoro-phenylamino)-3-oxa-7-aza-bicyclo[3.3.1]non-7-yl]-3-oxo-propenyl}-4-methoxy-phenyl)-acetamide

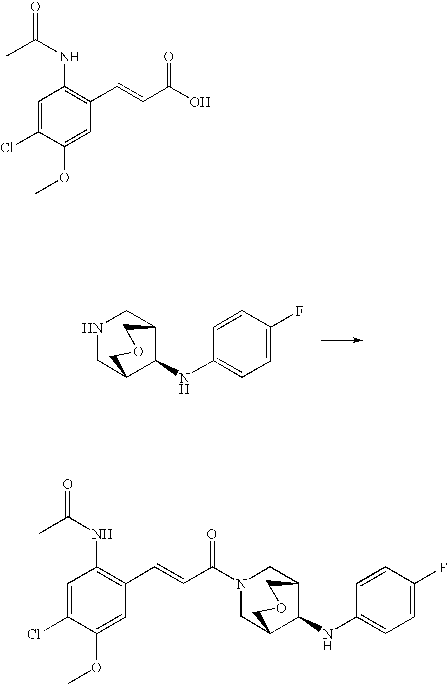

(E)-3-(2-Acetylamino-4-chloro-5-methoxy-phenyl)-acrylic acid (Example 23e) and (4-Fluoro-phenyl)-(1S,5R,9S)-3-oxa-7-aza-bicyclo[3.3.3]non-9-yl-amine are coupled according to conditions described in Example 59e. The target compound is obtained as colorless crystals (53 mg; 64%).

1H-NMR (400 MHz; DMSO-d6), δ (ppm): 1.81 (bs, 2H); 2.03 (s, 3H); 3.13 (bd, 1); 3.56-3.65 (m, 3H); 3.73 (d, 1H); 3.93 (s, 3H); 3.97 (m, 2H); 4.52 (bd, 1H); 4.78 (d, 1H); 5.82 (d, 1H); 6.68 (dd, 2H); 6.92 (t, 2H); 7.30 (d, 1H); 7.40 (s, 1H); 7.47 (s, 1H); 7.52 (d, 1H); 9.70 (s, 1H).

MS (m/z) ES+: 488 (MH+).

### Example 149

### N-(5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-methoxy-phenyl)-acetamide

### a) ((1S,5R,7S)-9-Benzyl-3-oxa-9-aza-bicyclo[3.3.1]non-7-yl)-(4-fluoro-phenyl)-amine

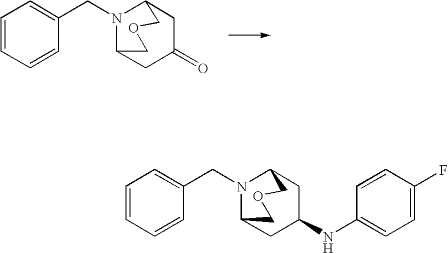

9-Benzyl-3-oxa-9-aza-bicyclo[3.3.1]nonan-7-one (J. Med. Chem., 1994, 37, 2831) (1.38 g; 5.97 mmol) is reductively aminated with 4-fluorobenzylamine as described in Example 144a. Only one isomer is isolated as slightly colored crystals (722 mg; 37%).

1H-NMR (400 MHz; DMSO-d6), δ (ppm): 1.42 (d, 2H); 2.28-2.39 (m, 2H); 2.60 (d, 2H); 3.55 (d, 2H); 3.76-3.85 (m, 5H); 5.95 (d, 1H); 6.49-6.55 (m, 2H); 6.90 (t, 2H); 7.22 (t, 1H); 7.28-7.37 (m, 4H).

MS (m/z) ES+: 327 (MH+).

### b) (4-Fluoro-phenyl)-(1S,5R,7S)-3-oxa-9-aza-bicyclo[3.3.1]non-7-yl-amine

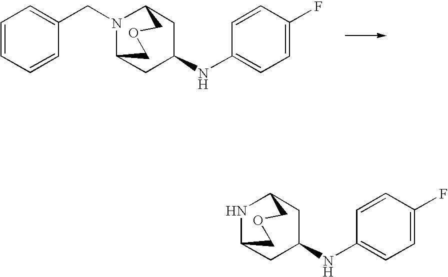

Debenzylation is performed as described in Example 144b and delivered the target compound as slightly colored crystals (500 mg; 97%).

1H-NMR (400 MHz; DMSO-d6), δ (ppm): 1.48-1.52 (m, 2H); 2.10-2.18 (m, 2H); 2.28 (bs, 1H); 2.83 (bd, 2H); 3.53 (bd, 2H); 3.63 (md, 2H); 5.75 (dd, 2H); 6.51 (dd, 2H); 6.88 (dd, 2H).

MS (m/z) ES+: 237 (MH+).

### c) N-(5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-methoxy-phenyl)-acetamide

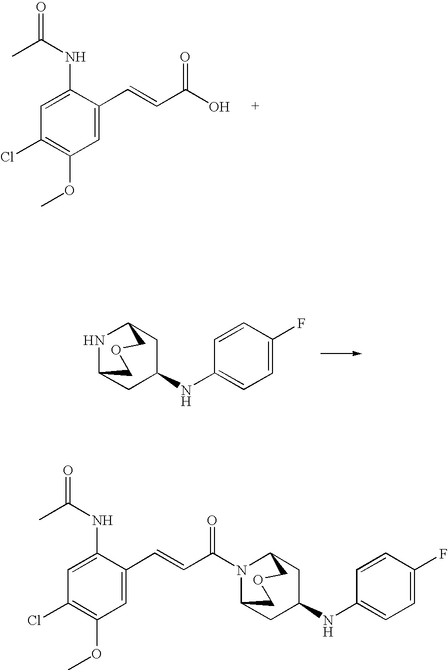

Acid (Example 23) and amine (Example 149b) are coupled according to Example 59e to deliver the target compound as colorless crystals (85 mg; 86%).

1H-NMR (400 MHz; DMSO-d6), δ (ppm): 1.70 (bt, 2H); 2.07 (s, 3H); 2.14-2.35 (m, 2H); 3.43 (m, 1H); 3.52 (bd, 1H); 3.62 (bd, 1H); 3.76 (t, 2H); 3.92 (s, 3H); 4.50 (bd, 1H); 4.53 (bd, 1H); 5.73 (d, 1H); 6.53 (dd, 2H); 6.89 (t, 2H); 7.21 (d, 1H); 7.41 (s, 1H); 7.48 (s, 1H); 7.62 (d, 1H); 9.72 (s, 1H).

MS (m/z) ES+: 488 (MH+).

### Example 150

### N-(5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-trifluoromethoxy-phenyl)-acetamide

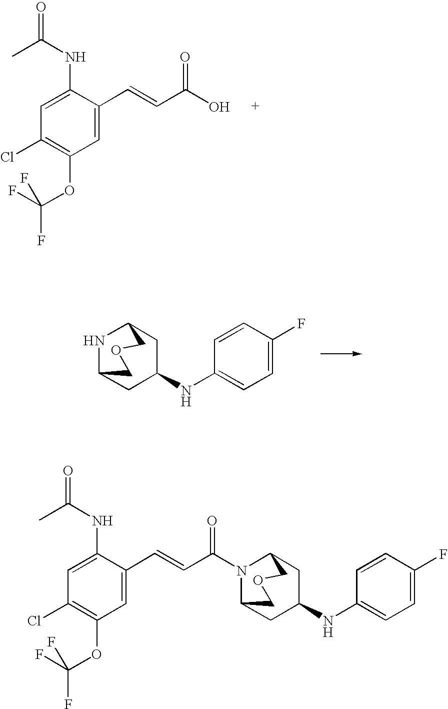

(E)-3-(2-Acetylamino-4-chloro-5-trifluoromethoxy-phenyl)-acrylic acid (obtained from Example 35c according to the method described in Example 23e) and (4-fluoro-phenyl)-(1S,5R,9S)-3-oxa-7-aza-bicyclo[3.3.1]non-9-yl-amine are coupled according to Example 59e. The target compound is obtained as slightly colored foam (126 mg; 91%).

1H-NMR (400 MHz; DMSO-d6), δ (ppm): 1.70 (bt, 2H); 2.12 (s, 3H); 2.14-2.35 (m, 2H); 3.43 (m, 1H); 3.52 (bd, 1H); 3.62 (bd, 1H); 3.76 (dd, 2H); 4.51 (bd, 1H); 4.53 (bd, 1H); 5.73 (d, 1H); 6.53 (dd, 2H); 6.89 (t, 2H); 7.31 (d, 1H); 7.65 (d, 1H); 7.80 (s, 1H); 8.11 (s, 1H); 10.03 (s, 1H).

MS (m/z) ES+: 542 (MH+).

### Example 151

### 3-(5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-methoxy-phenyl)-1,1-dimethyl-urea

### a) (E)-3-(2-Amino-4-chloro-5-methoxy-phenyl)-1-[(1S,5R,7S)-7-(4-fluoro-phenylamino)-3-oxa-9-aza-bicyclo[3.3.1]non-9-yl]-propenone

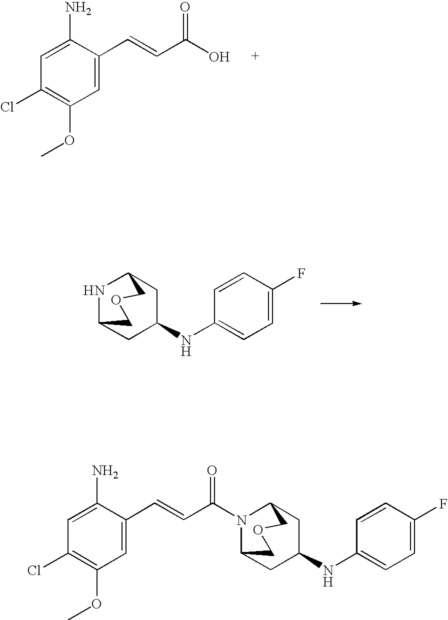

(E)-3-(2-Amino-4-chloro-5-methoxy-phenyl)-acrylic acid (Example 23c) and (4-fluoro-phenyl)-(1S,5R,9S)-3-oxa-7-aza-bicyclo[3.3.1]non-9-yl-amine are coupled according to Example 23d and delivered the target compound after chromatography (SiO2, EtOAc/hexanes 20/80 to 50/50) as yellow foam (266 mg; 94%).

1H-NMR (400 MHz; DMSO-d6), δ (ppm): 1.70 (bt, 2H); 2.14-2.35 (m, 2H); 3.43 (m, 1H); 3.52 (bd, 1H); 3.62 (bd, 1H); 3.76 (bd, 2H); 3.78 (s, 3H); 4.45 (bd, 1H); 4.56 (bd, 1H); 5.30 (s, 2H); 5.73 (d, 1H); 6.53 (dd, 2H); 6.79 (s, 1H); 6.90 (t, 2H); 7.00 (d, 1H); 7.21 (s, 1H); 7.70 (d, 1H).

MS (m/z) ES+: 4436 (MH+).

### b) 3-(5-Chloro-2-{(E)-3-[(1S,5R,7S)-7-(4-fluoro-phenylamino)-3-oxa-9-aza-bicyclo[3.3.1]non-9-yl]-3-oxo-propenyl}-4-methoxy-phenyl)-1,1-dimethyl-urea

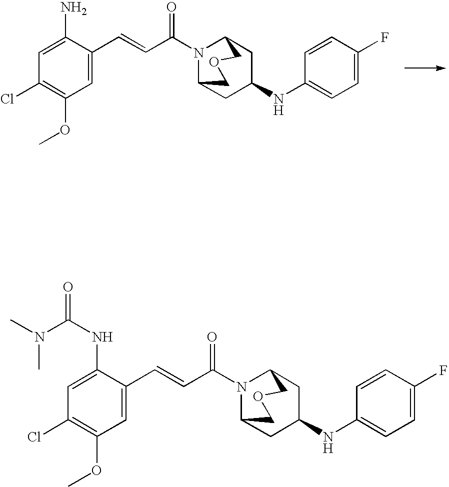

(E)-3-(2-Amino-4-chloro-5-methoxy-phenyl)-1-[(1S,5R,7S)-7-(4-fluoro-phenylamino)-3-oxa-9-aza-bicyclo[3.3.1]non-9-yl]-propenone is treated according to Example 29 and the target compound obtained after chromatography (SiO2, acetone/TBME 1/3) as colorless crystals (98 mg; 65%).

1H-NMR (400 MHz; DMSO-d6), δ (ppm): 1.70 (bt, 2H); 2.14-2.35 (m, 2H); 2.91 (s, 6H); 3.43 (m, 1H); 3.52-3.62 (m, 2H); 3.78 (t, 2H); 3.92 (s, 3H); 4.53 (bd, 2H); 5.73 (d, 1H); 6.53 (dd, 2H); 6.90 (t, 2H); 7.15 (d, 1H); 7.27 (s, 1H); 7.48 (s, 1H); 7.60 (d, 1H); 8.15 (s, 1H).

MS (m/z) ES+: 517 (MH+).

### Example 152

### 5-Chloro-2-{(E)-3-[(1S,5R,7S)-7-(4-fluoro-Phenylamino)-3-oxa-9-aza-bicyclo[3.3.1]non-9-yl]-3-oxo-propenyl}-4-methoxy-N,N-dimethyl-benzenesulfonamide

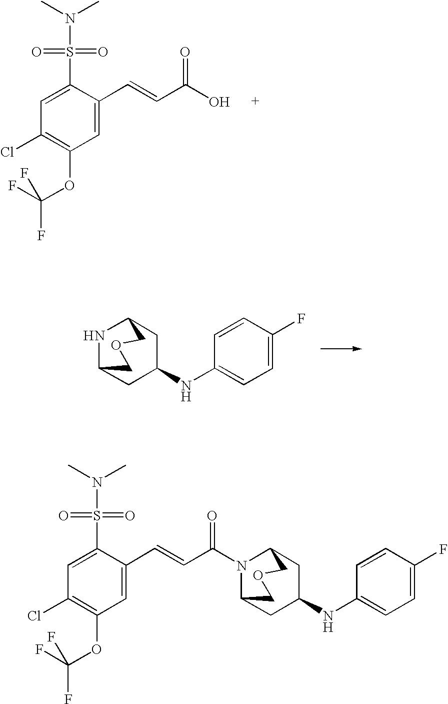

(4-Fluoro-phenyl)-(1S,5R,7S)-3-oxa-9-aza-bicyclo[3.3.1]non-7-yl-amine and (E)-3-(4-Chloro-2-dimethylsulfamoyl-5-trifluoromethoxy-phenyl)-acrylic acid (Example 40c) are coupled according to Example 40d and purified via chromatography (SiO2, acetone/hexanes 50/100) to yield the title compound as brownish crystals (74 mg; 97%).

1H-NMR (400 MHz; DMSO-d6), δ (ppm): 1.70 (bt, 2H); 2.16-2.35 (m, 2H); 2.78 (s, 6H); 3.43 (m, 1H); 3.52-3.62 (m, 2H); 3.76 (d, 1H); 3.79 (d, 1H); 4.53 (bd, 2H); 5.73 (d, 1H); 6.53 (dd, 2H); 6.90 (t, 2H); 7.33 (d, 1H); 7.93 (s, 1H); 8.21 (d, 1H); 8.38 (s, 1H).

MS (m/z) ES+: 592 (MH+).

### Example 153

### N-(5-Chloro-4-fluoro-2-{(E)-3-[3-(4-fluoro-benzyl)-3,8-diaza-bicyclo[3.2.1]oct-8-yl]-3-oxo-propenyl}-phenyl)-acetamide

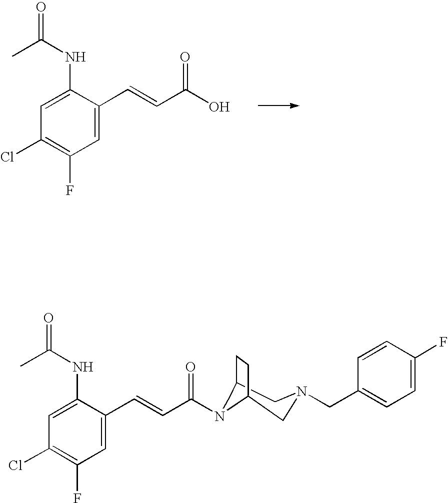

A mixture of (E)-3-(2-Acetylamino-4-chloro-5-fluoro-phenyl)-acrylic acid (50 mg), 3-(4-fluorobenzyl)-3,8-diazabicyclo[3.2.1]octane (47 mg), EDCl (45 mg) and hydroxy-benztriazole (31 mg) in 5 ml DMF is stirred at room temperature for 16 hours. 20 ml of water are added, the mixture is extracted with ethyl acetate, and the organic extract is washed with water and brine. The crude product, obtained after removal of the solvent is purified by RP-HPLC to give 17 mg (19%) of the title compound.

MS (m/z) ESI+: 460 (100, MH+)

### Example 154

### N-(5-Chloro-4-fluoro-2-{(E)-3-[3-(4-fluoro-benzyl)-7-methyl-3,7,9-triaza-bicyclo[3.3.1]non-9-yl]-3-oxo-propenyl}-phenyl)-acetamide

### a) E)-3-(2-Acetylamino-4-chloro-5-fluoro-phenyl)-acrylic acid

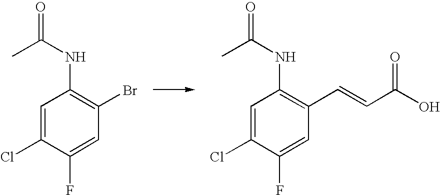

To a solution of t-butyl arylate (1.15 g, 9.0 mmol), 2-acetamino-4-chloro-5-fluoroaniline (2.0 g, 7.5 mmol)(prepared as described in WO 2004/037796) and triethylamine (3.1 ml, 22.5 mmol) in 60 ml DMF was added P(oTol)3 (228 mg) and palladium acetate (358 mg). The mixture was stirred and heated to 100 oC for 16 hours. Water was added and the product was extracted into ethylacetate. The crude product was titurated with ether/hexanes (1:1) to give 1.6 g of the t-butyl ester which was directly converted into the corresponding acid by treatment with 40 ml of a 1:1 mixture of TFA and dichloromethane for 30 minutes. Removal of the solvent and washings of the crude product with methanol provided 1.33 g (68%) of the desired acid.

1H-NMR (400 MHZ, DMSO-d6), δ (ppm): 2.08 (s, 3H), 6.58 (d, 1H), 7.61 (d, 1H), 7.65 (d, 1H), 7.91 (d, 1H), 12.55 (s, 1H).

MS (m/z) ES+: 258 (MH+)

### b) N-(5-Chloro-4-fluoro-2-{(E)-3-[3-(4-fluoro-benzyl)-3,8-diaza-bicyclo[3.2.1]oct-8-yl]-3-oxo-propenyl}-phenyl)-acetamide

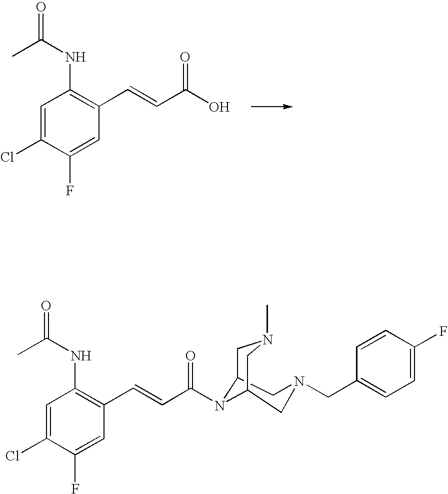

The title compound is prepared in 10% yield in analogy to Example 153 by amide coupling using EDCl in DMF starting from (E)-3-(2-Acetylamino-4-chloro-5-fluoro-phenyl)-acrylic acid and 3-(4-Fluoro-benzyl)-7-methyl-3,7,9-triaza-bicyclo[3.3.1]nonane.

MS (m/z) ESI+: 489 (100, MH+)

### Example 155

### 6-(5-Chloro-4-fluoro-2-{(E)-3-[3-(4-fluoro-benzyl)-3,8-diaza-bicyclo[3.2.1]oct-8-yl]-3-oxo-propenyl}-phenyl)-4,6-diaza-spiro[2,4]heptane-5,7-dione

### a) 1-[3-(4-Fluoro-benzyl)-3,8-diaza-bicyclo[3.2.1]oct-8-yl]-propenone

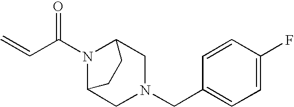

To a solution of 3-(4-fluorobenzyl)-3,8-diazabicyclo[3.2.1]octane (2.0 g, 9.1 mmol) and triethylamine (1.30 ml, 9.1 mmol) in 60 ml dichloromethane is added acryloylchlodde (0.74 ml, 9.1 mmol) at 0° C. After stirring at 0° C. for 90 minutes, the reaction is quenched by addition of NaHCO3 solution, the aqueous phase is extracted with dichloromethane, the organic phase dried over sodium sulfate and the solvent is evaporated. 2.3 g (8.4 mmol, 92%) of crude amide are obtained which are used in the subsequent steps without further purification.

1H-NMR (400 MHz; DMSO-d6), δ (ppm): 1.62-1.95 (m, 4H), 2.10 (dd, 2H), 2.63 (dd, 2H), 3.45 (s, 2H), 4.40-4.50 (m, 2H), 5.65 (dd, 1H), 6.14 (dd, 1H), 6.67 (dd, 1H), 7.12 (t, 2H), 7.31 (dd, 2H).

MS (m/z) ESI+: 275 (100, MH+).

### b) 6-(2-Bromo-5-chloro-4-fluoro-phenyl)-4,6-diaza-spiro[2,4]heptane-5,7-dione

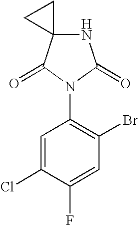

To a solution of 2-bromo-5-chloro-4-fluoroaniline (0.6 g, 2.7 mmol) and triethylamine (0.93 ml, 6.7 mmol) in 20 ml chloroform is added triphosgene (0.32 g, 1.07 mmol) in one portion. After stirring at room temperature for 5 hours first triethylamine (0.45 ml, 3.2 mmol) then 1-aminocyclopropane-1-carboxylic acid ethyl ester×HCl (0.44 g, 2.7 mmol) and the mixture is stirred at 65° C. for 16 hours. The crude product obtained after aquous workup is dissolved in 20 ml dioxane, potassium carbonate (0.37 g, 2.7 mmol) is added and the mixture heated to 120° C. for 16 hours. Addition of water, extraction with ethylacetate, drying and concentration gave the crude hydantoin which is further purified by RP-HPLC to yield 0.69 g (2.1 mmol, 77%) of the title compound.

1H-NMR (400 MHz; DMSO-d6), δ (ppm): 1.37 (s, 4H), 7.97 (d, 1H), 8.04 (d, 1H), 8.71 (s, 1H).

MS (m/z) ESI+: 333 (100, MH+).

### c) 6-(5-Chloro-4-fluoro-2-{(E)-3-[3-(4-fluoro-benzyl)-3,8-diaza-bicyclo[3.2.1]oct-8-yl]-3-oxo-propenyl}-phenyl)-4,6-diaza-spiro[2.4]heptane-5,7-dione

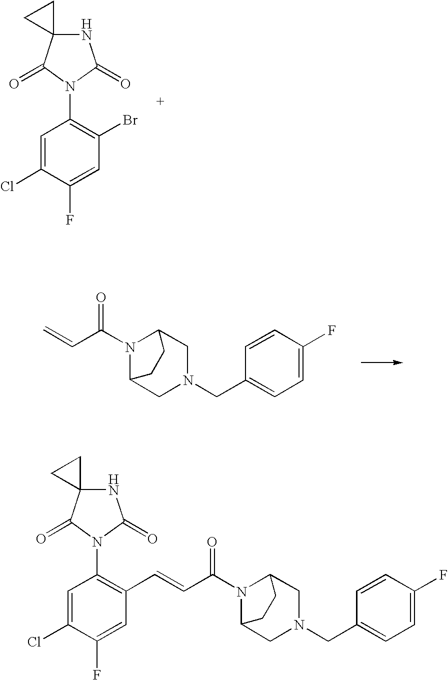

1-[3-(4-Fluoro-benzyl)-3,8-diaza-bicyclo[3.2.1]oct-8-yl]-propenone (136 mg, 0.49 mmol) and 6-(2-Bromo-5-chloro-4-fluoro-phenyl)-4,6-diaza-spiro[2,4]heptane-5,7-dione (150 mg, 0.45 mmol) are dissolved in DMF (4 ml). Triethylamine (0.188 ml, 1.35 mmol), tri-o-tolyl phosphine (14 mg, 0.05 mmol) and palladium acetate (11 mg, 0.05 mmol) are added. The mixture is heated to 120° C. for 3 hours, then poured onto saturated sodium carbonate solution, extracted into ethylacetate and dried over sodium sulfate. Purification by RP-HPLC (Acetonitrile/Water gradient) gave 56 mg (0.11 mmol, 24%) of the title compound.

1H-NMR (400 MHz; DMSO-d6), δ (ppm): 1.38 (s, 4H), 1.67-1.96 (m, 4H), 2.15 (t, 2H), 2.67 (dd, 2H), 3.47 (s, 2H), 4.49 (d, 1H), 4.69 (br s, 1H), 7.13 (t, 2H), 7.16 (d, 1H), 7.26 (d, 1H), 7.31 (dd, 2H), 7.79 (d, 1H), 8.22 (d, 1H), 8.78 (s, 1H).

MS (m/z) ESI+: 527 (100, MH+).

### Example 156

### 6-(5-Chloro-2-{(E)-3-[3-(4-fluoro-benzyl)-3,8-diaza-bicyclo[3.2.1]oct-8-yl]-3-oxo-ropenyl}-4-methoxy-phenyl)-4,6-diaza-spiro[2.4]heptane-5,7-dione

### a) 6-(2-Bromo-5-chloro-4-methoxy-phenyl)-4,6-diaza-spiro[2.4]heptane-5,7-dione

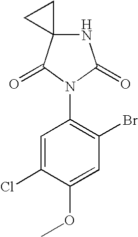

Using the method described in Example 155b the title compound is obtained starting from 2-bromo-5-chloro-4-methoxy-aniline in 90% yield.

1H-NMR (400 MHz; DMSO-d6), δ (ppm): 1.34 (s, 4H), 3.93 (s, 3H), 7.52 (s, 1H), 7.69 (s, 1H), 8.61 (s, 1H).

MS (m/z) ESI+: 345 (100, MH+).

### b) 6-(5-Chloro-2-{(E)-3-[3-(4-fluoro-benzyl)-3,8-diaza-bicyclo[3.2.1]oct-8-yl]-3-oxo-ropenyl}-4-methoxy-phenyl)-4,6-diaza-spiro[2,4]heptane-5,7-dione

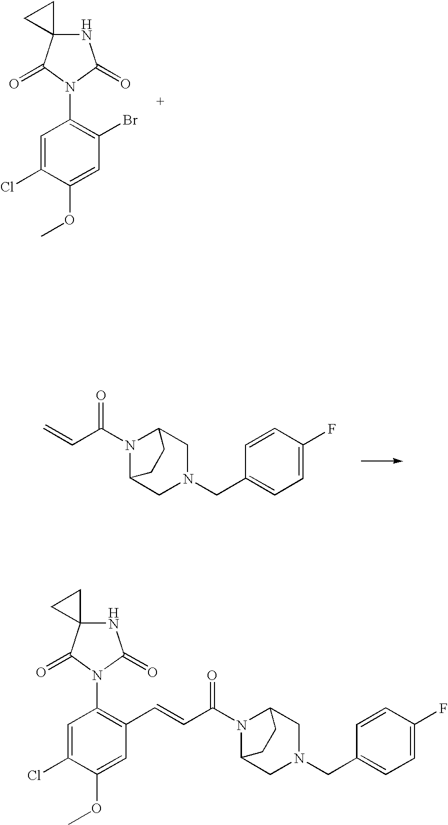

The title compound is prepared as described in Example 155c. After RP-HPLC purification 42 mg (18%) of the product are obtained.

1H-NMR (400 MHz; DMSO-d6), δ (ppm): 1.36 (s, 4H), 1.82-1.95 (m, 4H), 2.20-2.40 (m, 2H), 2.83 (dd, 2H), 3.46 (s, 2H), 3.99 (s, 3H), 4.45-4.52 (m, 1H), 4.67 (br s, 1H), 7.08-7.20 (m, 4H), 7.34 (dd, 2H), 7.54 (s, 1H), 7.61 (s, 1H), 8.67 (s, 1H).

MS (m/z) ESI+: 539 (100, MH+).

### Example 157

### 6-(5-Chloro-2-{(E)-3-[3-(4-fluoro-benzyl)-3,8-diaza-bicyclo[3.2.1]oct-8-yl]-3-oxo-ropenyl}-4-trifluoromethoxy-phenyl)-4,6-diaza-spiro[2,4]heptane-5,7-dione

### a) 6-(2-Bromo-5-chloro-4-trifluoromethoxy-phenyl)-4,6-diaza-spiro[2,4]heptane-5,7-dione

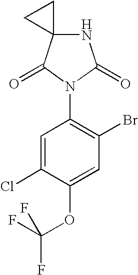

Using the method described in Example 155b the title compound is obtained starting from 2-bromo-5-chloro-4-trifluoromethoxy-aniline in 90% yield.

1H-NMR (400 MHz; DMSO-d6), δ (ppm): 1.48 (s, 4H), 8.09 (s, 1H), 8.14 (s, 1H), 8.80 (s (broad), 1H).

MS (m/z) ESI+: 397 (80, M−H), 399 (100, M−H).

### b) 6-(5-Chloro-2-{(E)-3-[3-(4-fluoro-benzyl)-3,8-diaza-bicyclo[3.2.1]oct-8-yl]-3-oxo-ropenyl}-4-trifluoromethoxy-phenyl)-4,6-diaza-spiro[2,4]heptane-5,7-dione

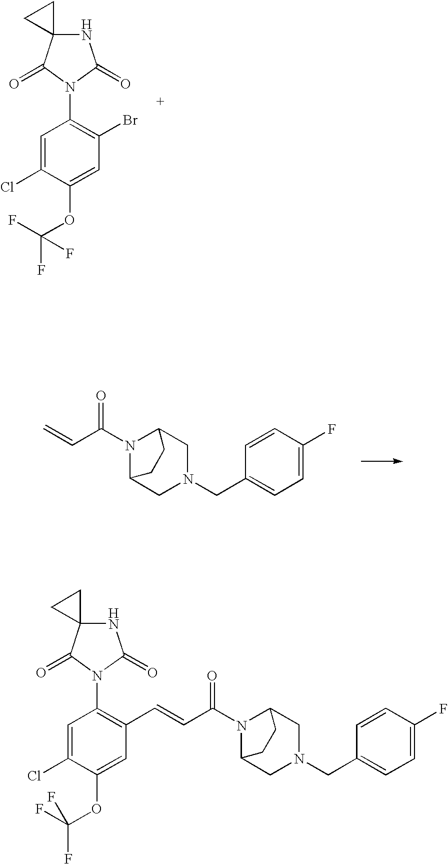

The title compound is prepared as described in Example 155c. After RP-HPLC purification 39 mg (27%) of the product are obtained.

1H-NMR (400 MHz; DMSO-d6), δ (ppm): 1.40 (s, 4H), 1.65-1.78 (m, 1H), 1.81-1.98 (m, 3H), 2.17 (t, 2H), 2.68 (dd, 2H), 3.45 (s, 2H), 4.48 (d, 1H), 4.67 (d, 1H), 7.12 (t, 2H), 7.18 (d, 1H), 7.29 (d, 1H), 7.32 (dd, 2H), 7.92 (s, 1H), 8.29 (s, 1H), 8.80 (s, 1H).

MS (m/z) ESI+: 593 (100, MH+).

### Example 158

### (R,S)-3-(5-Chloro-2-{(E)-3-[3-(4-fluoro-benzyl)-3,8-diaza-bicyclo[3.2.1]oct-8-yl]-3-oxo-ropenyl}-4-trifluoromethyl-phenyl)-5-methyl-imidazolidine-2,4-dione

### a) (R)-2-[3-(2-Bromo-5-chloro-4-trifluoromethyl-phenyl)-ureido]-propionic acid methyl ester

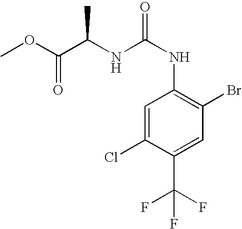

To a solution of 2-bromo-5-chloro-4-trifluoromethoxyaniline (0.5 g, 1.8 mmol) and triethylamine (0.63 ml, 4.6 mmol) in 12 ml chloroform is added triphosgene (0.22 g, 0.73 mmol) in one portion. After stirring at room temperature for 5 hours first triethylamine (0.30 ml, 2.2 mmol) then D-alanine methyl ester×HCl (0.25 g, 1.8 mmol) is added and the mixture is stirred at 65° C. for 16 hours. The reaction mixture is poured onto sodium bicarbonate solution followed by extraction with ethylacetate, drying and concentration. Chromatography gave 0.34 g (0.9 mmol, 50%) of the title compound.

1H-NMR (400 MHz; DMSO-d6), δ (ppm): 1.34 (d, 3H), 3.66 (s, 3H), 4.25 (dq, 1H), 7.94 (d, 1H), 7.99 (s, 1H), 8.42 (s, 1H), 8.48 (s, 1H).

MS (m/z) ESI−: 401 (100, M−H).

### b) (R,S)-3-(5-Chloro-2-{(E)-3-[3-(4-fluoro-benzyl)-3,8-diaza-bicyclo[3.2.1]oct-8-yl]-3-oxo-ropenyl}-4-trifluoromethyl-phenyl)-5-methyl-imidazolidine-2,4-dione

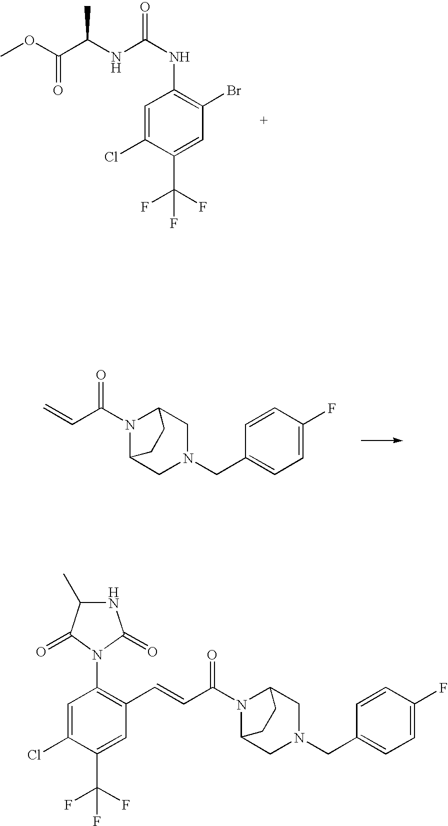

1-[3-(4-Fluoro-benzyl)-3,8-diaza-bicyclo[3.2.1]oct-8-yl]-propenone (77 mg, 0.29 mmol) and (R)-2-[3-(2-bromo-5-chloro-4-trifluoromethyl-phenyl)-ureido]-propionic acid methyl ester (100 mg, 0.26 mmol) are dissolved in DMF (4 ml). Triethylamine (0.107 ml, 0.78 mmol), tri-o-tolyl phosphine (8 mg, 0.026 mmol) and palladium acetate (6 mg, 0.026 mmol) are added. The mixture is heated to 120° C. for 16 hours, then poured onto saturated sodium carbonate solution, extracted into ethylacetate and dried over sodium sulfate. Purification by RP-HPLC (Acetonitrile/Water gradient) gave 34 mg (0.06 mmol, 23%) of the title compound as racemate.

1H-NMR (400 MHz; DMSO-d6), δ (ppm): 1.40 (t, 3H), 1.66-1.95 (m, 4H), 2.15 (t, 2H), 2.67 (dd, 2H), 3.45 (s, 2H), 4.30 (q, 1H), 4.49 (d, 1H), 4.73 (br s, 1H), 7.12 (t, 2H), 7.23 (d, 1H), 7.28-7.34 (m, 3H), 7.88 (d, 1H), 8.42-8.47 (m, 1H), 8.69 (s, 1H).

MS (m/z) ESI+: 565 (100, MH+).

### Example 159

### (R,S)-3-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-5-methyl-imidazolidine-2,4-dione

### a) (R)-2-[3-(2-Bromo-5-chloro-4-trifluoromethoxy-phenyl)-ureido]-propionic acid methyl ester

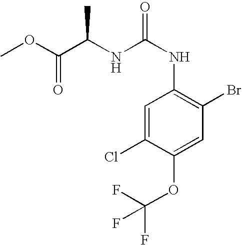

Using the method described in Example 158a the title compound is obtained starting from 2-bromo-5-chloro-4-trifluoromethoxy-aniline in 90% yield.

1H-NMR (400 MHz; DMSO-d6), δ (ppm): 1.34 (d, 3H), 3.67 (s, 3H), 4.26 (dq, 1H), 7.81 (d, 1H), 7.91 (s, 1H), 8.28 (s, 1H), 8.40 (s, 1H).

MS (m/z) ESI−: 419 (100, M−H).

### b) (R,S)-3-(5-Chloro-2-{(E)-3-[3-(4-fluoro-benzyl)-3,8-diaza-bicyclo[3.2.1]oct-8-yl]-3-oxo-propenyl}-4-trifluoromethoxy-phenyl)-5-methyl-imidazolidine-2,4-dione

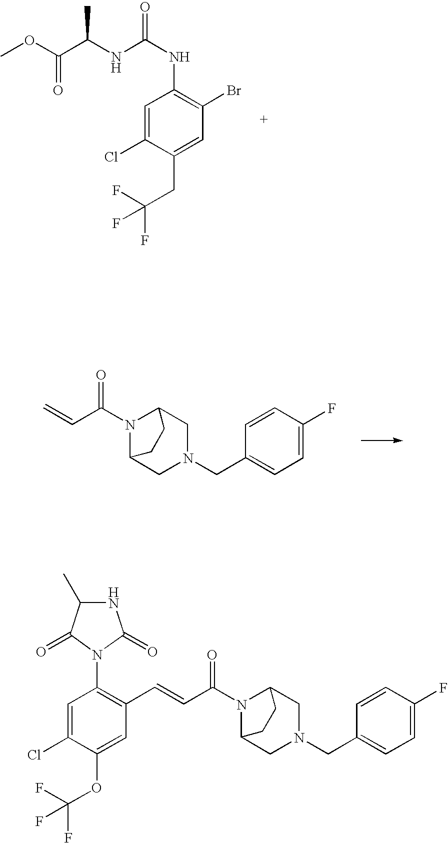

The title compound is prepared as described in Example 158b. After RP-HPLC purification 73 mg (35%) of the product are obtained.

1H-NMR (400 MHz; DMSO-d6), δ (ppm): 1.40 (t, 3H), 1.65-1.95 (m, 4H), 2.10-2.19 (m, 2H), 2.65 (dd, 2H), 3.45 (s, 2H), 4.29 (q, 1H), 4.48 (d, 1H), 4.69 (br s, 1H), 7.12 (s, 1H), 7.13 (dd, 2H), 7.27 (d, 1H), 7.30 (dd, 2H), 7.85 (d, 1H), 8.29 (s, 1H), 8.66 (s, 1H).

MS (m/z) ESI+: 581 (100, MH+).

### Example 160

### (R,S)-3-(5-Chloro-2-{(E)-3-[3-(4-fluoro-benzyl)-3,8-diaza-bicyclo[3.2.1]oct-8-yl]-3-oxo-propenyl}-4-methoxy-phenyl)-5-methyl-imidazolidine-2,4-dione

### a) (R)-2-[3-(2-Bromo-5-chloro-4-methoxy-phenyl)-ureido]-propionic acid methyl ester

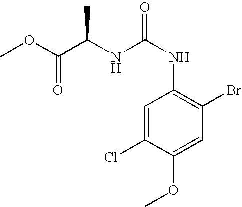

Using the method described in Example 158a the title compound is obtained starting from 2-bromo-5-chloro-4-methoxy-aniline in 80% yield.

1H-NMR (400 MHz; DMSO-d6), δ (ppm): 1.32 (d, 3H), 3.66 (s, 3H), 3.82 (s, 3H), 4.23 (dq, 1H), 7.35 (s, 1H), 7.45 (d, 1H), 7.96 (s, 1H), 8.04 (s, 1H).

MS (m/z) EI: 364 (100, M+).

### b) (R,S)-3-(5-Chloro-2-{(E)-3-[3-(4-fluoro-benzyl)-3,8-diaza-bicyclo[3.2.1]oct-8-yl]-3-oxo-propenyl}-4-methoxy-phenyl)-5-methyl-imidazolidine-2,4-dione

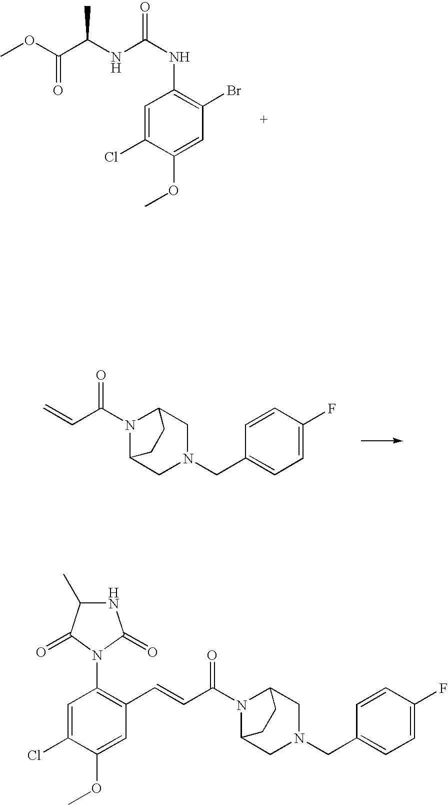

The title compound is prepared as described in Example 158b. After RP-HPLC purification 51 mg (24%) of the desired product are obtained.

1H-NMR (400 MHz; DMSO-d6), δ (ppm): 1.39 (t, 3H), 1.68-1.95 (m, 4H), 2.11-2.22 (m, 2H), 2.68 (dd, 2H), 3.46 (s, 2H), 3.99 (s, 3H), 4.26 (q, 1H), 4.50 (br s, 1H), 4.69 (br s, 1H), 7.08-7.19 (m, 4H), 7.32 (dd, 2H), 7.51 (d, 1H), 7.62 (s, 1H), 8.53 (s, 1H).

MS (m/z) ESI+: 527 (100, MH+).

Assays:

Preparation of Membranes from CHO Cells Expressing hCCR1:

Membranes were prepared from CHO-K1 cells stably transfected with a plasmid coding for the full-length human CCR1.

Cells were grown in large cell culture dishes (30×30 cm) to a confluency of between 80 and 90% (˜30×107 cells); in one experiment cells were grown to confluency without loss in receptor density of the membrane preparation.

All subsequent steps to prepare the membranes were performed at 4° C. or on ice. After discarding the medium, 30 ml ice-cold PBS containing 1 mM EDTA were added and the cells removed from the dishes using a scraper. After centrifugation at 10'000 rpm at 40° C. for 10 minutes in a SS34 rotor the supernatant was discarded and the cells resuspended in 10 mL buffer A (20 mM HEPES, 10 mM EDTA, pH 7.4) containing protease inhibitor cocktail (Roche, Complete). The cell suspension was homogenized using a Polytron homogenizer at 28'000 rpm at two intervals of 30 seconds each. In order to collect the membranes the homogenate was centrifuged at 18'000 rpm for 20 minutes at 4° C. using a SS34 rotor. The supernatant was discarded and the pellet resuspended by vortexing in 10 mL buffer B (20 mM HEPES, 0.1 mM EDTA, pH 7.4) containing protease inhibitors followed by a second round of homogenization (2×30 sec at 28'000 rpm, Polytron). After another centrifugation step (20 min at 4° C., 18'000 rpm) the pellet was resuspended in 5 mL buffer B by vortexing and subsequent homogenization (Polytron, 10 sec).

The protein concentration of the membrane preparation was determined using the BioRAD Protein Assay and human IgG as standard. The protein concentration of the membrane preparation was adjusted to 1-3 mg/mL and either aliquoted into Eppendorf tubes and quickfrozen in liquid nitrogen or, alternatively, the membrane preparation was added dropwise (by a peristaltic pump) into liquid nitrogen where it collects as frozen pellets (50-100 μL) at the bottom of the Dewar vessel. The membranes were stored at −80° C.

SPA-Binding Assay:

125 μg hCCR1 membranes were thawed and diluted into 340 μl ice-cold Buffer 2 (75 mM HEPES; pH 7.4, 300 mM NaCl, 6 mM CaCl₂, 15 mM MgCl₂, 1.5% BSA, Protease inhibitor cocktail (Complete Mini, Roche #61540601), 1 tablet in 10 mL). The final volume was adjusted to 1 mL with ice-cold Buffer 3 (20 mM HEPES, 0.1 mM EDTA, pH 7.4). The suspension was homogenized with three strokes and kept on ice.

The assay was performed in a final volume of 200 μl per well in OptiPlate-96 well plates. The components were added per well in the following order:
50 μL—CCR1—membranes (2.5 μg/well) diluted as described above
50 μL—WGA-SPA beads (1 mg/well) in Buffer 1 (HBSS (1×) (Gibco#1 4025-050), 10 mM HEPES; pH 7.4, 0.1% BSA (Fluka #05480))
inhibitor diluted in Buffer 1
50 μL-80 pM [125I]MIP-1α, diluted in Buffer 1 (to give a final concentration of 20 pM in the well)

After the addition of all components the plates were sealed with Top-Seal and incubated at RT for 120 minutes with constant shaking. Following incubation, the plates were centrifuged for 10 minutes at 3000 rpm and counted within 10 hours for 3 minutes per well with a TOP COUNT instrument (Packard).

Compounds of the invention demonstrated inhibition of binding of MIP1α to the human CCR1 receptor with IC50s ranging from 0.1 nM to 1000 nM.

Calcium Flux:

THP-1 cells are cultured in RPMI 1640 medium supplemented with 10% FCS. The cells are harvested, spun down and resuspended at about 2.106 cells per ml in HBSS 20 mM Hepes in absence of BSA. They are loaded in presence of 2 μM Fluo4 for 30 min at 37° C. in a waterbath. After two washes with HBSS 20 mM Hepes, they are resuspended at 0.67×106 cells/ml in the same buffer supplemented with 0.1% BSA and 150 μl containing 105 cells are distributed per well in a black/clear bottom 96-well plate.

The test compounds are prepared from stock solutions at 20 mM in pure DMSO to reach final concentrations ranking 10-5M to 10-11M in HBSS 20 mM Hepes supplemented with 0.1% BSA The agonist rh-MIP-1α is prepared as an eight-fold concentrated solution in the same buffer. Usually a final concentration of 3 nM is used for the screening.

Twenty-five microliters of the compounds are mixed to the 150 μl cells and the plates are let standing for an additional half an hour at RT in the dark to allow cell sedimentation and interaction with the compounds. Then the plate are transferred to the Flexstation (Molecular Devices fluorometer) where the fluo-4 fluorescence of the cells is measured continuously for 2 min in total but after 16 sec. of the base line measurement, 25 μl of the MIP1α solution are injected to the cells at a rate of one (about 26 μl/sec) and a height of 160 μl with two mixing cycles using a volume of 25 μl at a height of 150 μl and a rate of one.

The calcium response expressed as the maximal fluorescence in relative fluorescence unit is plotted versus the compound concentration to determine IC₅₀ concentrations.

Compounds of the invention demonstrated inhibition of Ca²⁺ mobilisation in response to MIP1α with IC₅₀s ranging from 0.1 nM to 1000 nM

As indicated in the above assays Agents of the Invention potently block the effects of MIP1α, and CCR1. Accordingly, the Agents of the Invention have pharmaceutical utility as follows:

Agents of the Invention are useful for the prophylaxis and treatment of CCR1 or MIP1α mediated diseases or medical conditions. CCR1 and MIP1α play an important role in leukocyte trafficking, in particular in monocyte migration to inflammatory sites and thus the Agents of the Invention may be used to inhibit monocyte migration e.g. in the treatment of inflammatory conditions, allergies and allergic conditions, autoimmune diseases, graft rejection, cancers which involve leukocyte infiltration, stenosis or restenosis, atherosclerosis, myocarditis, renal diseases, rheumatoid arthritis and osteoarthritis.

Diseases or conditions which may be treated with the Agents of the Invention include: Inflammatory or allergic conditions, including respiratory allergic diseases such as asthma, allergic rhinitis, COPD, hypersensitivity lung diseases, hypersensitivity pneumonitis, interstitial lung disease (ILD), (e.g. idiopathic pulmonary fibrosis, or ILD associated with autoimmune diseases such as RA, SLE, etc.); anaphylaxis or hypersensitivity responses, drug allergies (e.g. to penicillins or cephalosporins), and insect sting allergies; inflammatory bowel diseases, such as Crohn's disease and ulcerative colitis; spondyloarthropathies, sclerodoma; psoriasis and inflammatory dermatoses such as dermatitis, eczema, atopic dermatitis, allergic contact dermatitis, uticaria; vasculitis;

Autoimmune diseases, in particular autoimmune diseases with an aetiology including an inflammatory component such as arthritis (for example rheumatoid arthritis, arthritis chronica progrediente, psoriatic arthritis and arthritis deformans) and rheumatic diseases, including inflammatory conditions and rheumatic diseases involving bone loss, inflammatory pain, hypersensitivity (including both airways hypersensitivity and dermal hypersensitivity) and allergies. Specific autoimmune diseases for which Agents of the Invention may be employed include autoimmune haematological disorders (including e.g. hemolytic anaemia, aplastic anaemia, pure red cell anaemia and idiopathic thrombocytopenia), systemic lupus erythematosus, polychondritis, sclerodoma, Wegener granulomatosis, dermatomyositis, chronic active hepatitis, myasthenia gravis, psoriasis, Steven-Johnson syndrome, idiopathic sprue, autoimmune inflammatory bowel disease (including e.g. ulcerative colitis, Crohn's disease and Irritable Bowel Syndrome), autoimmune thyroiditis, Behcet's disease, endocrine opthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, and glomerulonephritis (with and without nephrotic syndrome, e.g. including idiopathic nephrotic syndrome or minimal change nephropathy);

graft rejection (e.g. in transplantation including heart, lung, combined heart-lung, liver, kidney, pancreatic, skin, or corneal transplants) including allograft rejection or xenograft rejection or graft-versus-host disease, and organ transplant associated arteriosclerosis; atherosclerosis;

cancer with leukocyte infiltration of the skin or organs;

stenosis or restenosis of the vasculature, particularly of the arteries, e.g. the coronary artery, including stenosis or restenosis which results from vascular intervention, as well as neointimal hyperplasia;

and other diseases or conditions involving inflammatory responses including reperfusion injury, hematologic malignancies, cytokine induced toxicity (e.g. septic shock or endotoxic shock), polymyositis, dermatomyositis, and granulomatous diseases including sarcoidosis.

Furthermore, the compounds pass the blood-brain barrier. Accordingly, the Agents of the Invention containing a radioisotope have pharmaceutical utility as markers in neuroimaging, for example in the treatment diagnosis of diseases such as Alzheimer's disease.

The term “treatment” as used herein is to be understood as including both therapeutic and prophylactic modes of therapy e.g. in relation to the treatment of neoplasia, therapy to prevent the onset of clinically or preclinically evident neoplasia, or for the prevention of initiation of malignant cells or to arrest or reverse the progression of premalignant to malignant cells, as well as the prevention or inhibition of neoplasia growth or metastasis. In this context, the present invention is, in particular, to be understood as embracing the use of compounds of the present invention to inhibit or prevent development of skin cancer, e.g. squamus or basal cell carcinoma consequential to UV light exposure, e.g. resultant from chronic exposure to the sun.

Agents of the Invention are particularly useful for treating diseases of bone and cartilage metabolism including osteoarthritis, osteoporosis and other inflammatory arthritides, e.g. rheumatoid arthritis, and bone loss in general, including age-related bone loss, and in particular periodontal disease.

The Agents of the Invention may also be used in ocular applications which include the treatment of ocular disorders, in particular of ocular inflammatory disorders, of ocular pain including pain associated with ocular surgery such as PRK or cataract surgery, of ocular allergy, of photophobia of various etiology, of elevated intraocular pressure (in glaucoma) by inhibiting the production of trabecular meshwork inducible glucocorticoid response (TIGR) protein, and of dry eye disease.

For the above indications, the appropriate dosage will, of course, vary depending upon, for example, the particular Agent of the Invention to be employed, the subject to be treated, the mode of administration and the nature and severity of the condition being treated. However, in prophylactic use, satisfactory results are generally indicated to be obtained at dosages from about 0.01 mg to about 10 mg, more preferably from about 0.05 mg to about 10 mg per kilogram body weight. Agent of the Invention is conveniently administered orally, parenterally, intravenously, e.g. into the antecubital or other peripheral vein, intramuscularly, or subcutaneously. For example, treatment typically comprises administering the Agent of the Invention once daily up to 3 times a day.

The compounds of the invention may also be administered simultaneously, separately or sequentially in combination with one or more other suitable active agents selected from the following classes of agents: anti-TNF agents, e.g. Enbrel (etanercept), Remicade (infliximab), Humira (adalimumab); anti IL-1 agents, e.g: Anakinra; anti cytokine receptor agents, e.g. anti IL-6 R Ab; B-cell and T-cell modulating drugs, e.g. anti CD20 Ab; disease-modifying anti-rheumatic agents (DMARDs), e.g. methotrexate, sulfasalazine; and non-steroidal anti inflammatories (NSAIDs), e.g. COX-2 inhibitors.

Pharmaceutical compositions of the invention may be manufactured in conventional manner The Agents of the Invention may be administered by any conventional route, e.g. orally, for example in the form of solutions for drinking, tablets or capsules or parenterally, for example in the form of injectable solutions or suspensions. Normally for systemic administration oral dosage forms are preferred, although for some indications the Agents of the Invention may also be administered topically or dermally, e.g. in the form of a dermal cream or gel or like preparation or, for the purposes of application to the eye, in the form of an ocular cream, gel or eye-drop preparation; or may be administered by inhalation, e.g., for treating asthma. Suitable unit dosage forms for oral administration comprise e.g. from 25 to 1000 mg of Agent of the Invention per unit dosage.

In accordance with the foregoing the present invention also provides in a further series of embodiments:

A. A method of inhibiting Chemokine Receptor 1 (CCR-1) or of reducing inflammation in a subject (i.e., a mammal, especially a human) in need of such treatment which method comprises administering to said subject an effective amount of an Agent of the Invention, or a method of treating any of the above mentioned conditions, particularly a method of treating an inflammatory or autoimmune disease or condition, e.g. rheumatoid arthritis, or alleviating one or more symptoms of any of the above mentioned conditions.

B. An Agent of the Invention for use as a pharmaceutical, e.g. for use as an immunosuppressant or antiinflammatory agent or for use in the prevention, amelioration or treatment of any disease or condition as described above, e.g., an autoimmune or inflammatory disease or condition.

C. A pharmaceutical composition comprising an Agent of the Invention in association with a pharmaceutically acceptable diluent or carrier, e.g., for use as an immunosuppressant or anti-inflammatory agent or for use in the prevention, amelioration or treatment of any disease or condition as described above, e.g., an autoimmune or inflammatory disease or condition.

D. Use of an Agent of the Invention in the manufacture of a medicament for use as an immunosuppressant or anti-inflammatory agent or for use in the prevention, amelioration or treatment of any disease or condition as described above, e.g., an autoimmune of inflammatory disease or condition.

E. An Agent of the Invention containing a radiolabel for use as a marker in neuroimaging, for example in the diagnosis of Alzheimer's disease.

F. Use of an Agent of the Invention containing a radiolabel as a marker in neuroimaging, for example in the diagnosis of Alzheimer's disease.

G. Use of an Agent of the Invention containing a radiolabel in the manufacture of a medicament for the diagnosis of Alzheimer's disease.

1. A compound of formula I, or a pharmaceutically acceptable salt or ester thereof, 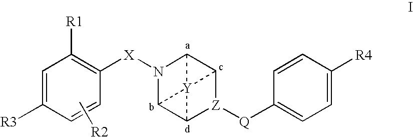
wherein
R1, R2 and R3 are independently selected from the group consisting of hydrogen, cyano, halo, nitro or optionally substituted oxy, C₁₋₇ alkyl, C₂₋₇ alkyenyl, C₂₋₇ alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
R4 is selected from the group consisting of hydrogen, cyano, halo, nitro or optionally substituted oxy, C₁₋₇ alkyl, C₂₋₇ alkyenyl, C₂₋₇ alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
X is —CH═CHCO—;
Y is —(CH₂)ₙ— where n is 1-6, —CH₂OCH₂— or —CH₂NRCH₂— and is bonded to two of the ring carbon atoms, bonding being to either the ring carbon atoms a and b or the ring carbon atoms c and d; wherein R is selected from the group consisting of H, optionally substituted: C₁₋₇ alkyl, carbonyl, acyl, acetyl or sulfonyl;
Z is N or —CH—;
Q is —CH₂—, —NH— or —O—;
wherein when Z is N, Q is CH, and when Z is —CH—, Q is —NH— or —O—;
the optional substitutents on R1-R4 are one or more, e.g. 1-3 substitutents, independently selected from the group consisting of hydrogen, oxo, cyano, halo, nitro or optionally substituted oxy, C₁₋₇ alkyl, C₂₋₇ alkenyl, C₂₋₇ alkynyl, aryl, heteroaryl, amino, sulfur, sulfinyl, sulfonyl;
wherein the optionally substituted substitutents are optionally substituted once or more by, e.g. 1-6 substitutents, a substitutent independently selected from the group consisting of hydrogen, oxo, cyano, halo, nitro, oxy, C₁₋₇ alkyl, C₂₋₇ alkyenyl, C₂₋₇ alkynyl, amino, sulfur, cycloalkyl, heterocyloalkyl, aryl, heteroaryl.

2. A compound of formula I as defined in claim 1 wherein R1 is an optionally substituted amino, amide, guanidino, sulfonyl, sulfonamide or heterocycloalkyl group, the optional substitutents being selected from the group consisting of hydrogen, oxo, cyano, halo, nitro or optionally substituted oxy, C₁₋₇ alkyl, C₂₋₇ alkenyl, C₂₋₇ alkynyl, heterocycloalkyl, amino, sulfur, sulfinyl, sulfonyl;
wherein the optionally substituted substitutents are optionally substituted once or more by a substitutent independently selected from the group consisting of
hydrogen, oxo, cyano, halo, nitro, oxy, C₁₋₇ alkyl, C₂₋₇ alkyenyl, C₂₋₇ alkynyl, amino, sulfur, cycloalkyl, heterocyloalkyl, aryl.

3. A compound of formula I according to claim 1 wherein R2 is selected from the group consisting of methoxy, trifluoromethoxy, aryl, heteroaryl, C₁₋₇ alkyl.

4. A compound according to claim 1, having the formula II, or a pharmaceutically acceptable salt or ester thereof: 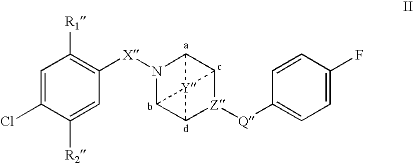
wherein
R₁″ and R₂″ are independently selected from the group consisting of hydrogen, cyano, halo, nitro or optionally substituted oxy, C₁₋₇ alkyl, C₂₋₇ alkyenyl, C₂₋₇ alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
X″ is —CH═CHCO—;
Y″ is —(CH₂)ₙ— where n is 1-6, —CH₂OCH₂— or —CH₂NRCH₂— and is bonded to two of the ring carbon atoms, bonding being to either the ring carbon atoms a and b or the ring carbon atoms c and d; wherein R is selected from the group consisting of H, optionally substituted; C₁₋₇ alkyl, carbonyl, acyl, acetyl or sulfonyl;
Z″ is N or —CH—;
Q″ is —CH₂—, —NH— or —O—;
wherein when Z″ is N, Q″ is CH, and when Z″ is —CH—, Q″ is —NH— or —O—;
the optional substitutents on R₁″ and R₂″ are one or more, e.g. 1-3 substitutents, independently selected from the group consisting of hydrogen, oxo, cyano, halo, nitro or optionally substituted oxy, C₁₋₇ alkyl, C₂₋₇ alkenyl, C₂₋₇ alkynyl, aryl, heteroaryl, amino, sulfur, sulfinyl, sulfonyl;
wherein the optionally substituted substitutents are optionally substituted once or more by, e.g. 1-6 substitutents, a substitutent independently selected from the group consisting of
hydrogen, oxo, cyano, halo, nitro, oxy, C₁₋₇ alkyl, C₂₋₇ alkyenyl, C₂₋₇ alkynyl, amino, sulfur, cycloalkyl, heterocyloalkyl, aryl, heteroaryl.

5. A compound of formula Ia, or a pharmaceutically acceptable salt or ester thereof, 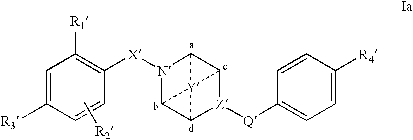
wherein
R₁′, R₂′ and R₃′ are independently selected from the group consisting of hydrogen, cyano, halo, nitro or optionally substituted oxy, C₁₋₇ alkyl, C₂₋₇ alkyenyl, C₂₋₇ alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
R₄′ is selected from the group consisting of hydrogen, cyano, halo, nitro or optionally substituted oxy, C₁₋₇ alkyl, C₂₋₇ alkyenyl, C₂₋₇ alkynyl, carbonyl, amino, sulfur, cycloalkyl, heterocycloalkyl, aryl, heteroaryl or a substitutent forming a bicyclic ring system of which the phenyl ring to which it is attached forms part of the bicycle for example butadiene forming napthyl, or heterobutadiene forming quinolinyl, quinoxalinyl or isoquinolinyl;
X′ is —OCH₂CO— or —NHCH₂CO—;
Y′ is —(CH₂)ₙ— where n is 1-6, —CH₂OCH₂— or —CH₂NRCH₂— and is bonded to two of the ring carbon atoms, bonding being to either the ring carbon atoms a and b or the ring carbon atoms c and d; wherein R is selected from the group consisting of H, optionally substituted: C₁₋₇ alkyl, carbonyl, acyl, acetyl or sulfonyl;
Z′ is N;
Q′ is —CH₂—;
the optional substitutents on R₁′—R₄′ being one or more substitutents, independently selected from the group consisting of hydrogen, oxo, cyano, halo, nitro or optionally substituted oxy, C₁₋₇ alkyl, C₂₋₇ alkenyl, C₂₋₇ alkynyl, aryl, heteroaryl, amino, sulfur, sulfinyl, sulfonyl;
wherein the optionally substituted substitutents are optionally substituted once or more by a substitutent independently selected from the group consisting of hydrogen, oxo, cyano, halo, nitro, oxy, C₁₋₇ alkyl, C₂₋₇ alkyenyl, C₂₋₇ alkynyl, amino, sulfur, cycloalkyl, heterocyloalkyl, aryl, heteroaryl.

6. A compound of formula Ia according to claim 5 wherein Y′ is —CH₂OCH₂— or —CH₂NRCH₂—.

7. A compound of formula I, Ia, II, Ib or IIb wherein the compound includes a radioisotope selected from the group of ¹¹C, ¹⁸F, ⁷⁵Br, ⁷⁶Br, ⁸⁰Br, ¹²³I, ¹²⁵I, ¹²⁸I, ¹³¹I, ¹³N, ¹⁵O.

8. A compound according to claim 1 for use as a pharmaceutical.

9. A compound according to claim 1 for use in the treatment of inflammation.

10. A compound according to claim 7 for use as a marker in neuroimaging.

11. A method of inhibiting chemokine receptors or of reducing inflammation in a mammal in need of such treatment which method comprises administering to said subject an effective amount of a compound according to claim 1.

12. Use of a compound according to claim 7 as a marker in neuroimaging.

13. A pharmaceutical composition comprising a compound according to claim 1 in association with a pharmaceutically acceptable diluent or carrier, for use as an immunosuppressant or anti-inflammatory agent.

14. Use of a compound according to claim 1 in the manufacture of a medicament for use as an immunosuppressant or anti-inflammatory agent or for use in the prevention, amelioration or treatment of an autoimmune of inflammatory disease or condition.

15. Use of a compound according to claim 7 in the manufacture of a medicament for the diagnosis of Alzheimer's disease.

16. A pharmaceutical composition comprising a compound according to claim 7 in association with a pharmaceutically acceptable diluent or carrier, for use as a marker in neuroimaging.

17. A process for the preparation of a compound of formula I, II, Ia, Ib or IIb including the step of:
(a) where the compound is of formula I or II, or of formula Ib or IIb wherein X is —CH═CHCO—, condensing a compound of formula IV with a compound of formula V in the presence of a suitable amide coupling agent, and, where Y is N, deprotection to give the desired compound of formula I (or corresponding compound of formula II, Ib or IIb): 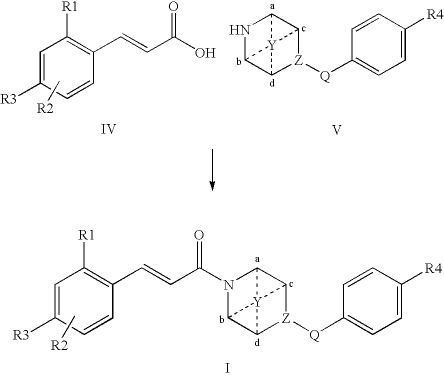
(b) where the compound is of formula Ia or II, or a compound of formula Ib or IIb wherein X is —OCH₂CO—, or —NCH₂CO—, reacting a compound of formula X with a compound of formula IX in the presence of a strong base in an inert organic solvent: 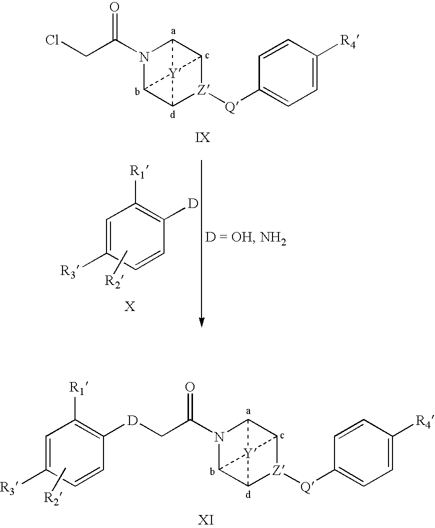
or
(c) where the compound is of formula I or II, or of formula Ib or IIb wherein X is —CH═CHCO—, reacting a compound of formula X with a compound of formula XII in the presence of a suitable reagent such as a palladium catalyst and a base to produce the desired compound of formula I: 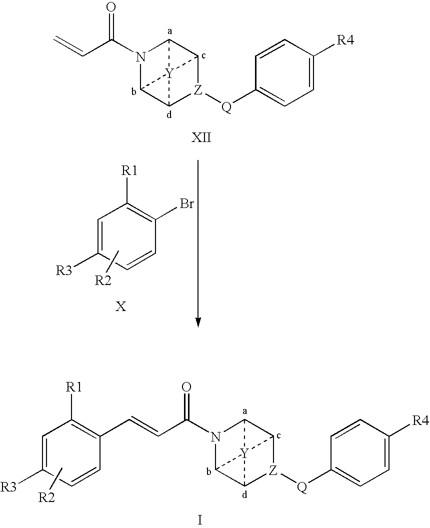
or
(d) where the compound is a compound wherein R1, R₁′ or R₁″ is denoted by a group of the following formula: 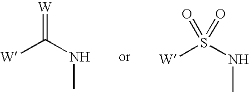
wherein W is O or a nitrogen carrying optional substitutents and W′ represents optional substitutents,
reacting a corresponding compound of formula XII or XIII: 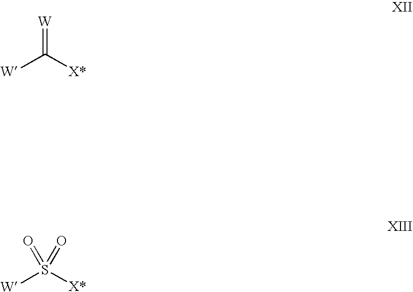
wherein X* represents a leaving group, for example chloro,
with a compound of formula XV: 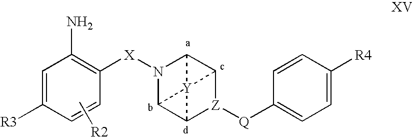
to produce the desired compound.

18. A process according to claim 17, further including the step of temporarily protecting any interfering reactive groups and/or then isolating the resulting compound of the invention.
